(19) United States
(12) Patent Application Publication
(10) Pub. No.: US 2022/0009918 A1
(43) Pub. Date: Jan. 13, 2022

(54) Chemical Compounds

(71) Applicant: GlaxoSmithKline Intellectual Property Development Limited, Brentford, Middlesex (GB)

(72) Inventors: Rodolfo Cadilla, Collegeville, PA (US); David Norman Deaton, Collegeville, PA (US); Andrew L. Larkin, Collegeville, PA (US); Christie Schulte, Collegeville, PA (US); Terrence L. Smalley, Research Triangle Park, NC (US)

(21) Appl. No.: 17/291,087

(22) PCT Filed: Nov. 6, 2019
(86) PCT No.: PCT/IB2019/059517

§ 371 Date: May 4, 2021

Related U.S. Application Data
(60) Provisional application No. 62/757,205, filed on Nov. 8, 2018.

(51) Int. Cl. C07D 417/14; C07D 417/12; C07D 491/048; C07D 491/107; C07D 421/12

(52) U.S. Cl. CPC C07D 417/14; C07D 417/12; C07D 421/12; C07D 491/107; C07D 491/048

(57) ABSTRACT

A compound of formula (I), wherein Ar¹, R²¹, R²³, R²⁴, R²⁵, R²⁶, R²⁷, A, X, Y and W are as defined herein. The compounds of the present invention are inhibitors of hematopoietic prostaglandin D synthase (H-PGDS) and can be useful in the treatment of Duchenne muscular dystrophy. Accordingly, the invention is further directed to pharmaceutical compositions comprising a compound of the invention. The invention is still further directed to methods of inhibiting H-PGDS activity and treatment of disorders associated therewith using a compound of the invention or a pharmaceutical composition comprising a compound of the invention.

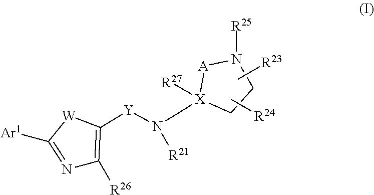

### FIELD OF THE INVENTION

The present invention relates to novel compounds, to the use of the compounds as Hematopoietic Prostaglandin D Synthase (H-PGDS) inhibitors, to pharmaceutical compositions comprising the compounds and to the use of the compounds in therapy, especially in the treatment of conditions for which a H-PGDS inhibitor is indicated, such as neurodegenerative diseases and musculoskeletal diseases, including Duchenne muscular dystrophy, where PGD₂ is considered to play a pathological role, for the use of a compound in the manufacture of a medicament for the treatment of conditions in which an inhibitor of H-PGDS is indicated, and a method for the treatment or prophylaxis of disorders in which inhibition of H-PGDS is indicated, in a human.

### BACKGROUND OF THE INVENTION

Prostaglandin D₂ (PGD₂) is a product of arachidonic acid metabolism, and is the major prostanoid mediator synthesised by mast cells in response to stimulation via multiple mechanisms and cellular activation pathways, including allergen-mediated cross-linking of high affinity IgE receptors (Lewis et al. (1982) Prostaglandin D₂ generation after activation of rat and human mast cells with anti-IgE. J. Immunol, 129, 1627-1631). Other cells such as dendritic cells, Tₕ2 cells, and epithelial cells also produce PGD₂, but at lower levels than mast cells. PGD₂ mediates its effects via activation of the specific G-protein coupled receptors DP₁ (Boie et al. (1995) Molecular cloning and characterization of the human prostanoid DP receptor. J. Biol. Chem., 270, 18910-18916) and DP₂ (CRTH2) (Abe et al. (1999), Molecular cloning, chromosome mapping and characterization of the mouse CRTH2 gene, a putative member of the leukocyte chemo-attractant receptor family. Gene, 227, 71-77) and also acts via the receptor for thromboxane A₂ (TXA₂), the TP receptor, on target cells.

Prostaglandin D synthase (PGDS) is the enzyme responsible for the catalytic isomerase conversion of prostaglandin endoperoxide PGH₂ to PGD₂. PGD₂ is generated by the action of either H-PGDS (hematopoietic-type or H-type) or L-PGDS (lipocalin-type or L-type) enzymes (Urade et al., (2000) Prostaglandin D synthase structure and function. Vitamins and hormones, 58, 89-120). H-PGDS activity is dependent on glutathione and plays an important role in the generation of PGD₂ by immune and inflammatory cells, including mast cells, antigen-presenting cells (e.g. dendritic cells), macrophages, and Tₕ2 cells, which are all key cells in the pathology of allergic disease. In contrast, L-type is glutathione-independent and is primarily located in the central nervous system, genital organs, and heart. These two isoforms of PGDS appear to have distinct catalytic properties, tertiary structure, and cellular and tissue distribution.

Using the small molecule inhibitor HQL-79, H-PGDS has been demonstrated to play a modulatory role in diseases such as Duchenne muscular dystrophy (Nakagawa et al. (2013) A prostaglandin D₂ metabolite is elevated in the urine of Duchenne muscular dystrophy patients and increases further from 8 years old, Clinica Chimica Acta 423, 10-14) and (Mohri et al. (2009), Inhibition of prostaglandin D synthase suppresses muscular necrosis, Am. J. Pathol. 174, 1735-1744) and (Okinaga et al. (2002), Induction of hematopoietic prostaglandin D synthase in hyalinated necrotic muscle fibers: its implication in grouped necrosis, Acta Neuropathologica 104, 377-84), spinal cord contusion injury (Redensek et al. (2011) Expression and detrimental role of hematopoietic prostaglandin D synthase in spinal cord contusion injury, Glia 59, 603-614), neuroinflammation (Mohri et al. (2006) Prostaglandin D₂-mediated microglia/astrocyte interaction enhances astrogliosis and demyelination in twitcher. J. Neurosci. 26, 4383-4393), and neurodegenerative disease (Ikuko et al. (2007) Hematopoietic prostaglandin D synthase and DP₁ receptor are selectively upregulated in microglia and astrocytes within senile plaques from human patients and in a mouse model of Alzheimer disease. J. Neuropath. Exp. Neur. 66, 469-480). H-PGDS has also been implicated to play a role in metabolic diseases such as diabetes and obesity, since PGD₂ is converted to 15-deoxy-Δ^{12,14}PGJ₂, a potent ligand for PPARγ which is able to drive adipogenesis (Tanaka et al (2011) Mast cells function as an alternative modulator of adipogenesis through 15-deoxy-delta-12, 14-prostaglandin J₂. Am. J. Physiol. Cell Physiol. 301, C1360-C1367). PGD₂ has been implicated to play a role in niacin-induced skin flushing (Papaliodis et al (2008) Niacin-induced “flush” involves release of prostaglandin D₂ from mast cells and serotonin from platelets: Evidence from human cells in vitro and an animal model. JPET 327:665-672).

Weber et al. (2010), Identification and characterisation of new inhibitors for the human hematopoietic prostaglandin D₂ synthase. Eur. J. Med. Chem. 45, 447-454, Carron et al. (2010), Discovery of an Oral Potent Selective Inhibitor of Hematopoietic Prostaglandin D Synthase (H-PGDS). ACS Med. Chem. Lett. 1, 59-63; Christ et al. (2010), Development and Characterization of New Inhibitors human and Mouse Hematopoietic Prostaglandin D₂ Synthases, J. Med. Chem., 53, 5536-5548; and Hohwy et al. (2008), Novel Prostaglandin D Synthase Inhibitors Generated by Fragment-Based Drug Design. J. Med. Chem., 51, 2178-2186 are also of interest.

Based on this evidence, chemical inhibitors of H-PGDS, which inhibit PGD₂ formation, simultaneously inhibit the biological actions of PGD₂ and its metabolites at multiple receptors, offer the potential for therapeutic benefit in the treatment of a range of diseases where PGD₂ is considered to play a pathological role.

International Patent Applications WO2005/094805, WO2007/007778, WO2007/041634, WO2008/121670, WO2008/122787, WO2009/153720, WO2009/153721, WO2010/033977, WO2010/104024, WO2011/043359, WO2011044307, WO2011/090062, Japanese Patent Application 2007-51121 and US Patent Application 2008/0146569 disclose certain H-PGDS inhibitors and their use in the treatment of diseases associated with the activity of H-PGDS.

Edfeldt, F. et al (Bioorganic & Medicinal Chemistry Letters 2015, 25, 2496-2500) discloses indole inhibitors of human hematopoietic prostaglandin D2 synthase (hH-PGDS). WO 2013079425 A1 discloses 2H-indazoles as EP2 receptor antagonists. Carron, C. P. et al (ACS Medicinal Chemistry Letters 2010, 1, 59-63) discloses a selective inhibitor of Hematopoietic Prostaglandin D Synthase (HPGDS). WO 2009133831 A1 discloses 3-[3-(indazol-5-yl)phenyl]propionic acids as type-4 PLA2 inhibitors for inhibiting production of prostaglandin and/or leukotriene.

It is an object of the invention to provide further H-PGDS inhibitors, suitably for the treatment of muscular dystrophy.

### SUMMARY OF THE INVENTION

The invention is directed to compounds according to Formula I:

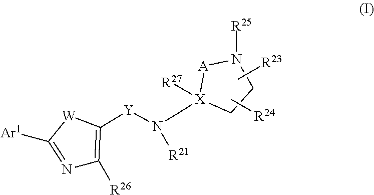

wherein Ar¹, R²¹, R²³, R²⁴, R²⁵, R²⁶, R²⁷, A, X, Y and W are as defined below.

Compounds of Formula (I) and their pharmaceutically acceptable salts have H-PGDS activity and are believed to be of use for the treatment or prophylaxis of certain disorders.

Accordingly, in another aspect of the invention there is provided a pharmaceutical composition comprising a compound of Formula (I) according to the first aspect, or a pharmaceutically acceptable salt thereof and one or more pharmaceutically acceptable carriers or excipients.

In some embodiments, the pharmaceutical composition is for the treatment or prophylaxis of a disorder in which inhibition of H-PGDS is beneficial.

In a further aspect, the invention provides a compound of Formula (I) or a pharmaceutically acceptable salt thereof according to the first aspect of the invention for use in therapy.

The invention also provides a compound of Formula (I) or a pharmaceutically acceptable salt thereof, for use in the treatment of a condition for which an H-PGDS inhibitor is indicated.

This invention also relates to a method for the treatment of disorders in which inhibition of H-PGDS is beneficial in a human comprising administering to the human in need thereof a therapeutically effective amount of a compound of Formula (I) or a pharmaceutically acceptable salt thereof.

This invention also relates to a method of treating Duchenne muscular dystrophy, which comprises administering to a subject in need thereof an effective amount of a H-PGDS inhibiting compound of Formula (I).

This invention also relates to a method of treating congenital myotonia, which comprises administering to a subject in need thereof an effective amount of a H-PGDS inhibiting compound of Formula (I).

This invention also relates to a method of treating muscle injury, which comprises administering to a subject in need thereof an effective amount of a H-PGDS inhibiting compound of Formula (I).

This invention also relates to a method of treating tendon injury, which comprises administering to a subject in need thereof an effective amount of a H-PGDS inhibiting compound of Formula (I).

This invention also relates to a method of treating muscle lacerations, which comprises administering to a subject in need thereof an effective amount of a H-PGDS inhibiting compound of Formula (I).

This invention also relates to a method of treating chronic muscle strains, which comprises administering to a subject in need thereof an effective amount of a H-PGDS inhibiting compound of Formula (I).

This invention also relates to a method of treating myotonic dystrophy type I, which comprises administering to a subject in need thereof an effective amount of a H-PGDS inhibiting compound of Formula (I).

This invention also relates to a method of treating myotonic dystrophy type II, which comprises administering to a subject in need thereof an effective amount of a H-PGDS inhibiting compound of Formula (I).

This invention also relates to a method of treating asthma, which comprises administering to a subject in need thereof an effective amount of a H-PGDS inhibiting compound of Formula (I).

This invention also relates to a method of treating chronic obstructive pulmonary disease, which comprises administering to a subject in need thereof an effective amount of a H-PGDS inhibiting compound of Formula (I).

This invention also relates to a method of treating rheumatoid arthritis, which comprises administering to a subject in need thereof an effective amount of a H-PGDS inhibiting compound of Formula (I).

This invention also relates to a method of treating systemic lupus erythematosus (SLE), which comprises administering to a subject in need thereof an effective amount of a H-PGDS inhibiting compound of Formula (I).

This invention also relates to a method of treating inflammatory bowel disease, which comprises administering to a subject in need thereof an effective amount of a H-PGDS inhibiting compound of Formula (I).

This invention also relates to a method of treating osteoarthritis, which comprises administering to a subject in need thereof an effective amount of a H-PGDS inhibiting compound of Formula (I).

This invention also relates to a method of treating psoriasis, which comprises administering to a subject in need thereof an effective amount of a H-PGDS inhibiting compound of Formula (I).

This invention also relates to a method of treating atopic dermatitis, which comprises administering to a subject in need thereof an effective amount of a H-PGDS inhibiting compound of Formula (I).

This invention also relates to a method of treating a muscle degenerative disorder, which comprises administering to a subject in need thereof an effective amount of a H-PGDS inhibiting compound of Formula (I).

This invention also relates to a method of treating muscular dystrophy, which comprises administering to a subject in need thereof an effective amount of a H-PGDS inhibiting compound of Formula (I).

Also included in the present invention are methods of co-administering the presently invented H-PGDS inhibiting compounds with further active ingredients.

The invention also relates to a compound of Formula (I) or a pharmaceutically acceptable salt thereof for use in the treatment of Duchenne muscular dystrophy.

The invention also relates to a compound of Formula (I) or a pharmaceutically acceptable salt thereof for use in the treatment of congenital myotonia.

The invention also relates to a compound of Formula (I) or a pharmaceutically acceptable salt thereof for use in the treatment of muscle injury.

The invention also relates to a compound of Formula (I) or a pharmaceutically acceptable salt thereof for use in the treatment of tendon injury.

The invention also relates to a compound of Formula (I) or a pharmaceutically acceptable salt thereof for use in the treatment of muscle lacerations.

The invention also relates to a compound of Formula (I) or a pharmaceutically acceptable salt thereof for use in the treatment of chronic muscle strains.

The invention also relates to a compound of Formula (I) or a pharmaceutically acceptable salt thereof for use in the treatment of myotonic dystrophy type 1.

The invention also relates to a compound of Formula (I) or a pharmaceutically acceptable salt thereof for use in the treatment of myotonic dystrophy type II.

The invention also relates to a compound of Formula (I) or a pharmaceutically acceptable salt thereof for use in the treatment of asthma.

The invention also relates to a compound of Formula (I) or a pharmaceutically acceptable salt thereof for use in the treatment of chronic obstructive pulmonary disease.

The invention also relates to a compound of Formula (I) or a pharmaceutically acceptable salt thereof for use in the treatment of rheumatoid arthritis.

The invention also relates to a compound of Formula (I) or a pharmaceutically acceptable salt thereof for use in the treatment of systemic lupus erythematosus (SLE).

The invention also relates to a compound of Formula (I) or a pharmaceutically acceptable salt thereof for use in the treatment of inflammatory bowel disease.

The invention also relates to a compound of Formula (I) or a pharmaceutically acceptable salt thereof for use in the treatment of osteoarthritis.

The invention also relates to a compound of Formula (I) or a pharmaceutically acceptable salt thereof for use in the treatment of psoriasis.

The invention also relates to a compound of Formula (I) or a pharmaceutically acceptable salt thereof for use in the treatment of atopic dermatitis.

The invention also relates to a compound of Formula (I) or a pharmaceutically acceptable salt thereof for use in the treatment of a muscle degenerative disorder.

The invention also relates to a compound of Formula (I) or a pharmaceutically acceptable salt thereof for use in the treatment of muscular dystrophy.

The invention provides for the use of a compound of Formula (I) or a pharmaceutically acceptable salt thereof in the treatment of conditions in which an inhibitor of H-PGDS is indicated.

The invention provides for the use of a compound of Formula (I) or a pharmaceutically acceptable salt thereof in the treatment of a muscle degenerative disorder.

The invention provides for the use of a compound of Formula (I) or a pharmaceutically acceptable salt thereof in the manufacture of a medicament for the treatment of conditions in which an inhibitor of H-PGDS is indicated.

The invention further provides a method for the treatment or prophylaxis of disorders in which inhibition of H-PGDS is indicated, in a human, which comprises administering a human in need thereof a therapeutically effective amount of a compound of Formula (I) or a pharmaceutically acceptable salt thereof.

### BRIEF DESCRIPTION OF THE FIGURE

FIG. 1. Effect of different doses of an H-PGDS inhibitor, Example 16, on limb force following eccentric (lengthening) contraction-induced muscle injury in normal C57Bl6/N mice. Doses were administered 10 minutes prior to damage and QD thereafter. H-PGDS Inhibition Accelerates Functional Repair Following Limb Injury in Normal WT Mice FIG. 1 depicts the protection and acceleration of functional repair dose response curves of H-PGDS inhibition, using the compound of Example 16, following limb muscle injury in male C57Bl/6N mice.

FIG. 2. H-PGDS Inhibition Causes Dose-Dependent Reduction in Peritoneal Lavage Fluid PGD₂ Following Compound 48/80 Challenge FIG. 2 depicts the effects of different doses of the H-PGDS inhibitor of Example 16 on prostaglandin D₂ generation, following 48/80-induced mast cell degranulation in normal C57Bl6/N mice.

### DETAILED DESCRIPTION OF THE INVENTION

This invention relates to compounds of Formula (I) and to the use of compounds of Formula (I) in the methods of the invention:

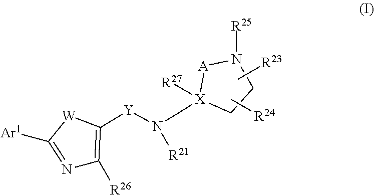

wherein:
Ar¹ is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
W is selected from: S and Se,
X is selected from: C and N;
Y is selected from: —C(O)—, —C(S)—, —C(Se)—, —S(O)—, and —S(O₂)—;
A is selected from: —C(O)—, —C(S)—, —C(Se)—, and —S(O₂)—;
R²¹ is selected from: hydrogen and —CH₃;
R²³ and R²⁴ are attached to the same or different carbon atoms and are independently selected from:
   hydrogen,
   C₁₋₅alkyl,
   C₁₋₅alkyl substituted with from one to four substituents independently selected from: —OH, oxo, —NH₂ and fluoro, or
   R²³ and R²⁴ are attached to the same carbon and are taken together to form: cyclopropyl, cyclobutyl, cyclopentyl, oxetanyl, tetrahydrofuran, or tetrahydropyran, or
R²³ and R²⁴ are attached to different carbon atoms and are taken together to form: cyclopropyl, cyclobutyl, cyclopentyl, oxetanyl, tetrahydrofuran, or tetrahydropyran;
R²⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylaryl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro;
R²⁶ is selected from: hydrogen and —CH₃; and
R²⁷ is absent when X is N or selected from: hydrogen and —CH₃;
or a pharmaceutically acceptable salt thereof.

This invention relates to compounds of Formula (I) and to the use of compounds of Formula (I) in the methods of the invention:

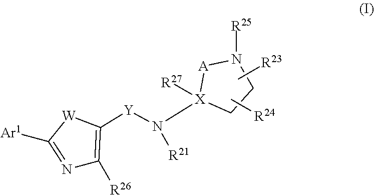

wherein:
Ar¹ is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents
   independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
W is selected from: S and Se,
X is selected from: C and N;
Y is selected from: —C(O)—, —C(S)—, —C(Se)—, —S(O)—, and —S(O₂)—;
A is selected from: —C(O)—, —C(S)—, —C(Se)—, and —S(O₂)—;
R²¹ is selected from: hydrogen and —CH₃;
R²³ and R²⁴ are attached to the same or different carbon atoms and are independently selected from:
   hydrogen,
   C₁₋₅alkyl,
   C₁₋₅alkyl substituted with from one to four substituents independently selected from: —OH, oxo, —NH₂ and fluoro, or
   R²³ and R²⁴ are attached to the same carbon and are taken together to form: cyclopropyl, cyclobutyl, cyclopentyl, oxetanyl, tetrahydrofuran, or tetrahydropyran, or
   R²³ and R²⁴ are attached to different carbon atoms and are taken together to form: cyclopropyl, cyclobutyl, cyclopentyl, oxetanyl, tetrahydrofuran, or tetrahydropyran;
R²⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylaryl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro;
R²⁶ is selected from: hydrogen and —CH₃; and
R²⁷ is absent when X is N or selected from: hydrogen and —CH₃;

Suitably in the compounds of Formula (I), Ar¹ is phenyl. Suitably in the compounds of Formula (I), Ar¹ is phenyl optionally substituted with from 1 to 3 substituents independently selected from: fluoro, chloro, bromo, iodo, C₁₋₃alkyl (optionally substituted with from 1 to 4F), —CN, —OH, cyclopropyl and —OCH₃. Suitably in the compounds of Formula (I), Ar¹ is phenyl substituted with from 1 to 3 substituents independently selected from: fluoro, chloro, bromo, iodo, —CH₃, —CH₂F, —CHF₂, —CF₃, —CH₂CH₃, —CH₂CF₃, —CH₂CFH₂, —CH₂CF₂H, —CN, —OH, cyclopropyl and —OCH₃. Suitably in the compounds of Formula (I), W is S. Suitably in the compounds of Formula (I), Y is C(O). Suitably in the compounds of Formula (I), X is C. Suitably in the compounds of Formula (I), R²¹ is selected from: hydrogen and —CH₃. Suitably in the compounds of Formula (I), R²¹ is hydrogen. Suitably in the compounds of Formula (I), R²¹ is —CH₃. Suitably in the compounds of Formula (I), A is selected from: C(O), C(S) and C(Se). Suitably in the compounds of Formula (I), R²⁵ is selected from: hydrogen, —CH₃, —CH₂C(O)NH₂, and —CH₂-phenyl-O—CH₃. Suitably in the compounds of Formula (I), R²⁶ is hydrogen. Suitably in the compounds of Formula (I), R²⁷ is hydrogen.

Suitably in the compounds of Formula (I), R²³ and R²⁴ are independently selected from:
hydrogen, —CH₃, and —CH₂CH₃, or
R²³ and R²⁴ are attached to the same carbon and are taken together to form:
cyclopropyl, cyclobutyl, or oxetanyl, or
R²³ and R²⁴ are attached to different carbon atoms and are taken together to
form: cyclopentyl, or tetrahydrofuranyl.

This invention relates to compounds of Formula (IA) in the methods of the invention:

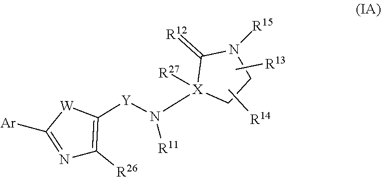

wherein:
Ar is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
W is selected from S and Se;
X is selected from C and N;
Y is selected from C═O, C═S and S(O)₂;
R is selected from: hydrogen and —CH
R¹² is selected from: O, S and Se;
R¹³ and R¹⁴ are attached to the same or different carbon atoms and are independently selected from:
   hydrogen,
   C₁₋₅alkyl,
   C₁₋₅alkyl substituted with from one to four substituents independently selected from: —OH, oxo, —NH₂ and fluoro, or
   R¹³ and R¹⁴ are attached to the same carbon and are taken together to form: cyclopropyl, cyclobutyl, cyclopentyl, oxetanyl, tetrahydrofuran, or tetrahydropyran, or
   R¹³ and R¹⁴ are attached to different carbon atoms and are taken together to
      form: cyclopropyl, cyclobutyl, cyclopentyl, oxetanyl, tetrahydrofuran, or
      tetrahydropyran;
R¹⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylphenyl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro;
R²⁶ is selected from H and —CH₃; and
R₂₇ is selected from H and —CH₃; or is absent when X is N;
or a pharmaceutically acceptable salt thereof.

This invention relates to compounds of Formula (IA) in the methods of the invention:

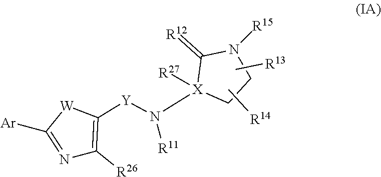

wherein:
Ar is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
W is selected from S and Se;
X is selected from C and N;
Y is selected from C═O, C═S and S(O)₂;
R¹¹ is selected from: hydrogen and —CH₃;
R¹² is selected from: O, S and Se;
R¹³ and R¹⁴ are attached to the same or different carbon atoms and are independently
   selected from:
   hydrogen,
   C₁₋₅alkyl,
   C₁₋₅alkyl substituted with from one to four substituents independently selected from: —OH, oxo, —NH₂ and fluoro, or
   R¹³ and R¹⁴ are attached to the same carbon and are taken together to form: cyclopropyl, cyclobutyl, cyclopentyl, oxetanyl, tetrahydrofuran, or tetrahydropyran, or
   R¹³ and R¹⁴ are attached to different carbon atoms and are taken together to
      form: cyclopropyl, cyclobutyl, cyclopentyl, oxetanyl, tetrahydrofuran, or
      tetrahydropyran;
R¹⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylphenyl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro;
R²⁶ is selected from H and —CH₃; and
R₂₇ is selected from H and —CH₃; or is absent when X is N.

This invention relates to compounds of Formula (II) and to the use of compounds of Formula (II) in the methods of the invention:

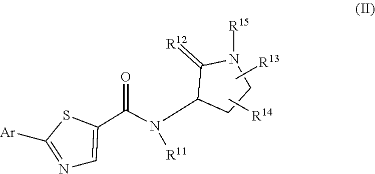

wherein:
Ar is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
R¹¹ is selected from: hydrogen and —CH₃;
R¹² is selected from: O, S and Se;
R¹³ and R¹⁴ are attached to the same or different carbon atoms and are independently
   selected from:
   hydrogen,
   C₁₋₅alkyl,
   C₁₋₅alkyl substituted with from one to four substituents independently selected from: —OH, oxo, —NH₂ and fluoro, or
   R¹³ and R¹⁴ are attached to the same carbon and are taken together to form: cyclopropyl, cyclobutyl, cyclopentyl, oxetanyl, tetrahydrofuran, or tetrahydropyran, or
   R¹³ and R¹⁴ are attached to different carbon atoms and are taken together to
      form: cyclopropyl, cyclobutyl, cyclopentyl, oxetanyl, tetrahydrofuran, or
      tetrahydropyran; and
R¹⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylphenyl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro;
or a pharmaceutically acceptable salt thereof.

This invention relates to compounds of Formula (II) and to the use of compounds of Formula (II) in the methods of the invention:

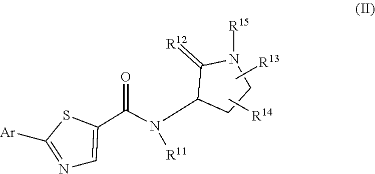

wherein:
Ar is selected from: phenyl, benzofuranyl, pyrazolyl, imidazolyl, pyridinyl, and pyrimidinyl, each of which is optionally substituted with from 1 to 4 substituents independently selected from:
   fluoro,
   chloro,
   bromo,
   iodo,
   C₁₋₃alkyl,
   C₁₋₃alkyl substituted with from one to four substituents independently selected from: —OH, oxo, and fluoro,
   —CN,
   —OH,
   cyclopropyl,
   C₁₋₃alkoxy, and
   C₁₋₃alkoxy substituted with from one to four substituents independently selected from: —OH, oxo, and fluoro;
R¹¹ is selected from: hydrogen and —CH₃;
R¹² is selected from: O, S and Se;
R¹³ and R¹⁴ are attached to the same or different carbon atoms and are independently
   selected from:
   hydrogen,
   C₁₋₅alkyl,
   C₁₋₅alkyl substituted with from one to four substituents independently selected from: —OH, oxo, —NH₂ and fluoro, or
   R¹³ and R¹⁴ are attached to the same carbon and are taken together to form: cyclopropyl, cyclobutyl, cyclopentyl, oxetanyl, tetrahydrofuran, or tetrahydropyran, or
   R¹³ and R¹⁴ are attached to different carbon atoms and are taken together to
      form: cyclopropyl, cyclobutyl, cyclopentyl, oxetanyl, tetrahydrofuran, or
      tetrahydropyran; and
R¹⁵ is selected from:
   hydrogen,
   C₁₋₅alkyl,
   C₁₋₅alkyl substituted with from one to four substituents independently selected from: —OH, oxo, —NH₂ and fluoro, and
   C₁₋₅alkylaryl, and
   C₁₋₅alkylphenyl substituted with from 1 to 3 substituents independently selected from
      fluoro,
      chloro,
      bromo,
      iodo,
      C₁₋₃alkyl,
      C₁₋₃alkyl substituted with from one to four substituents independently selected from: —OH, oxo, and fluoro,
      —CN,
      —OH,
      cyclopropyl,
      C₁₋₃alkoxy, and
      C₁₋₃alkoxy substituted with from one to four substituents independently selected from: —OH, oxo, and fluoro.

Suitably in the compounds of Formula (II), Ar is phenyl. Suitably in the compounds of Formula (II) Ar¹ is phenyl optionally substituted with from 1 to 3 substituents independently selected from: fluoro, chloro, bromo, iodo, C₁₋₃alkyl (optionally substituted with from 1 to 4F), —CN, —OH, cyclopropyl and —OCH₃. Suitably in the compounds of Formula (II), Ar is phenyl substituted with from 1 to 3 substituents independently selected from: fluoro, chloro, bromo, iodo, —CH₃, —CH₂F, —CHF₂, —CF₃, —CH₂CH₃, —CH₂CF₃, —CH₂CFH₂, —CH₂CF₂H, —CN, —OH, cyclopropyl and —OCH₃. Suitably in the compounds of Formula (II), R¹¹ is selected from: hydrogen and —CH₃. Suitably in the compounds of Formula (II), R¹¹ is hydrogen. Suitably in the compounds of Formula (II), R¹¹ is —CH₃. Suitably in the compounds of Formula (II), R¹² is selected from: O, S and Se. Suitably in the compounds of Formula (II), R¹² is O. Suitably in the compounds of Formula (II), R¹² is S. Suitably in the compounds of Formula (II), R¹² is Se. Suitably in the compounds of Formula (II), R¹⁵ is selected from: hydrogen, —CH₃, —CH₂C(O)NH₂, and —CH₂— phenyl-O—CH₃. Suitably in the compounds of Formula (II), R¹⁵ is hydrogen.

Suitably in the compounds of Formula (II), R¹³ and R¹⁴ are independently selected from:
hydrogen, —CH₃, and —CH₂CH₃, or
R¹³ and R¹⁴ are attached to the same carbon and are taken together to form: cyclopropyl, cyclobutyl, or oxetanyl, or
R¹³ and R¹⁴ are attached to different carbon atoms and are taken together to
form: cyclopentyl, or tetrahydrofuranyl.
   Suitably in the compounds of Formula (II), R¹³ and R¹⁴ are —CH3.

This invention relates to compounds of Formula (IIb) and to the use of compounds of Formula (IIb) in the methods of the invention:

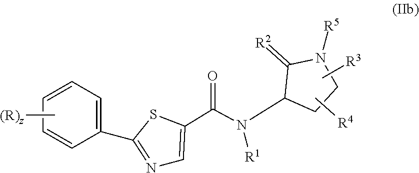

wherein:
R is selected from: fluoro, chloro, bromo, iodo, C₁₋₃alkyl (optionally substituted by 1 to 4F), —CN, —OH, cyclopropyl and
   —OCH₃;
R¹ is selected from: hydrogen and —CH₃;
R² is selected from: O, S and Se;
R³ and R⁴ are attached to the same or different carbon atoms and are independently selected from: hydrogen, —CH₃, and —CH₂CH₃, or
   R³ and R⁴ are attached to the same carbon and are taken together to form: cyclopropyl, cyclobutyl, or oxetanyl, or
   R³ and R⁴ are attached to different carbon atoms and are taken together to form: cyclopentyl, or tetrahydrofuranyl;
R⁵ is selected from: hydrogen, —CH₃, —CH₂C(O)NH₂, and —CH₂-phenyl-O—CH₃; and
Z is an integer from 0 to 3;

or a pharmaceutically acceptable salt thereof.

This invention relates to compounds of Formula (IIb) and to the use of compounds of Formula (IIb) in the methods of the invention:

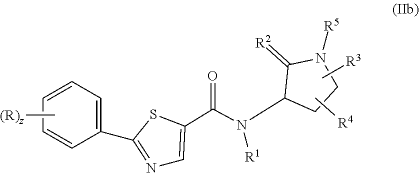

wherein:
R is selected from: fluoro, chloro, bromo, iodo, C₁₋₃alkyl (optionally substituted by 1 to 4F), —CN, —OH, cyclopropyl and
   —OCH₃;
R¹ is selected from: hydrogen and —CH₃;
R² is selected from: O, S and Se;
R³ and R⁴ are attached to the same or different carbon atoms and are independently selected from: hydrogen, —CH₃, and —CH₂CH₃, or
   R³ and R⁴ are attached to the same carbon and are taken together to form: cyclopropyl, cyclobutyl, or oxetanyl, or
   R³ and R⁴ are attached to different carbon atoms and are taken together to form: cyclopentyl, or tetrahydrofuranyl;
R⁵ is selected from: hydrogen, —CH₃, —CH₂C(O)NH₂, and —CH₂-phenyl-O—CH₃; and
Z is an integer from 0 to 3.

This invention relates to compounds of Formula (III) and to the use of compounds of Formula (III) in the methods of the invention:

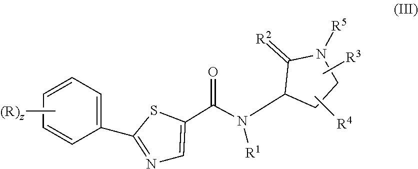

wherein:
R is selected from: fluoro, chloro, bromo, iodo, —CH₃, —CH₂F, —CHF₂, —CF₃, —CH₂CH₃, —CH₂CF₃, —CH₂CFH₂, —CH₂CF₂H, —CN, —OH, cyclopropyl and —OCH₃;
R¹ is selected from: hydrogen and —CH₃;
R² is selected from: O, S and Se;
R³ and R⁴ are attached to the same or different carbon atoms and are independently selected from: hydrogen, —CH₃, and —CH₂CH₃, or
   R³ and R⁴ are attached to the same carbon and are taken together to form: cyclopropyl, cyclobutyl, or oxetanyl, or
   R³ and R⁴ are attached to different carbon atoms and are taken together to form: cyclopentyl, or tetrahydrofuranyl;
R⁵ is selected from: hydrogen, —CH₃, —CH₂C(O)NH₂, and —CH₂-phenyl-O—CH₃; and
Z is an integer from 0 to 3;

or a pharmaceutically acceptable salt thereof.

This invention relates to compounds of Formula (III) and to the use of compounds of Formula (III) in the methods of the invention:

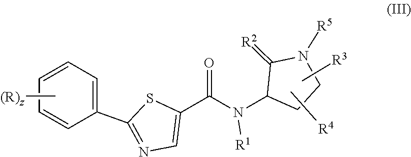

wherein:
R is selected from: fluoro, chloro, bromo, iodo, —CH₃, —CH₂F, —CHF₂, —CF₃, —CH₂CH₃, —CH₂CF₃, —CH₂CFH₂, —CH₂CF₂H, —CN, —OH, cyclopropyl and —OCH₃;
R¹ is selected from: hydrogen and —CH₃;
R² is selected from: O, S and Se;
R³ and R⁴ are attached to the same or different carbon atoms and are independently selected from: hydrogen, —CH₃, and —CH₂CH₃, or
   R³ and R⁴ are attached to the same carbon and are taken together to form: cyclopropyl, cyclobutyl, or oxetanyl, or
   R³ and R⁴ are attached to different carbon atoms and are taken together to form: cyclopentyl, or tetrahydrofuranyl;
R⁵ is selected from: hydrogen, —CH₃, —CH₂C(O)NH₂, and —CH₂-phenyl-O—CH₃; and
Z is an integer from 0 to 3.

Suitably in the compounds of Formula (III), R is selected from: fluoro, chloro, and bromo. Suitably in the compounds of Formula (III), R¹ is selected from: hydrogen and —CH₃. Suitably in the compounds of Formula (III), R¹ is H. Suitably in the compounds of Formula (III), R¹ is —CH₃. Suitably in the compounds of Formula (III), R² is selected from: O, S and Se. Suitably in the compounds of Formula (III), R² is O. Suitably in the compounds of Formula (III), R² is S. Suitably in the compounds of Formula (III), R² is Se. Suitably in the compounds of Formula (III), R⁵ is selected from: hydrogen and —CH₃. Suitably R⁵ is H. Suitably in the compounds of Formula (III), R³ and R⁴ are independently selected from: hydrogen, —CH₃, and —CH₂CH₃. Suitably R³ and R⁴ are —CH₃.

This invention relates to compounds of Formula (IV) and to the use of compounds of Formula (IV) in the methods of the invention:

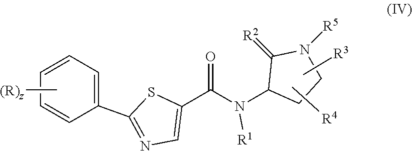

wherein:
R is independently selected from: fluoro, chloro, bromo, and iodo;
R¹ is selected from: hydrogen and —CH₃;
R² is O;
R³ and R⁴ are attached to the same or different carbon atoms and are independently selected from: hydrogen, —CH₃, and —CH₂CH₃;
R⁵ is selected from: hydrogen, and —CH₃; and
Z is an integer from 0 to 3;

or a pharmaceutically acceptable salt thereof.

This invention relates to compounds of Formula (IV) and to the use of compounds of Formula (IV) in the methods of the invention:

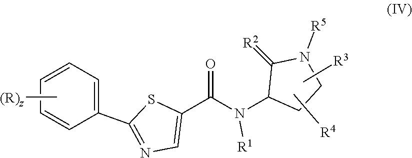

wherein:
R is independently selected from: fluoro, chloro, bromo, and iodo;
R¹ is selected from: hydrogen and —CH₃;
R² is O;
R³ and R⁴ are attached to the same or different carbon atoms and are independently selected from: hydrogen, —CH₃, and —CH₂CH₃;
R⁵ is selected from: hydrogen, and —CH₃; and
Z is an integer from 0 to 3.

Suitably in the compounds of Formula (IV), R is independently selected from chloro and fluoro. Suitably in the compounds of Formula (IV), R¹ is H. Suitably in the compounds of Formula (IV), R³ and R⁴ are attached to the same carbon atom. Suitably in the compounds of Formula (IV), R³ and R⁴ are —CH₃. Suitably in the compounds of Formula (IV), R⁵ is H. Suitably in the compounds of Formula (IV), z is a integer chosen from 1 or 2. Suitably in the compounds of Formula (IV), z is 2.

Included in the compounds of Formula (I) and in the methods of the invention are:
(S)-2-(Benzofuran-7-yl)-N-(2-oxopyrrolidin-3-yl)thiazole-5-carboxamide;
Racemic 2-(3-Chlorophenyl)-N-(2-oxopyrrolidin-3-yl)thiazole-5-carboxamide;
(S)-2-(3-Chlorophenyl)-N-(2-oxopyrrolidin-3-yl)thiazole-5-carboxamide;
(S)-N-(2-Oxopyrrolidin-3-yl)-2-(3-(trifluoromethyl)phenyl)thiazole-5-carboxamide;
(S)-N-(2-Oxopyrrolidin-3-yl)-2-(m-tolyl)thiazole-5-carboxamide;
(S)-2-(3-Chloro-5-fluorophenyl)-N-(2-oxopyrrolidin-3-yl)thiazole-5-carboxamide;
(S)-2-(5-Chloro-2-fluorophenyl)-N-(2-oxopyrrolidin-3-yl)thiazole-5-carboxamide;
(S)-2-(3-Chloro-2-fluorophenyl)-N-(2-oxopyrrolidin-3-yl)thiazole-5-carboxamide;
(S)-2-(3,5-Dichlorophenyl)-N-(2-oxopyrrolidin-3-yl)thiazole-5-carboxamide;
(S)-2-(3-(Difluoromethyl)phenyl)-N-(2-oxopyrrolidin-3-yl)thiazole-5-carboxamide;
2-(3-Chloro-5-fluorophenyl)-N-((3S,4R)-4-methyl-2-oxopyrrolidin-3-yl)thiazole-5-carboxamide;
(S)-2-(3-(Difluoromethyl)-5-fluorophenyl)-N-(2-oxopyrrolidin-3-yl)thiazole-5-carboxamide;
2-(3-(Difluoromethyl)-5-fluorophenyl)-N-((3S,4R)-4-methyl-2-oxopyrrolidin-3-yl)thiazole-5-carboxamide;
Racemic 2-(3-Chloro-5-fluorophenyl)-N-(5,5-dimethyl-2-oxopyrrolidin-3-yl)thiazole-5-carboxamide;
(R)-2-(3-Chloro-5-fluorophenyl)-N-(5,5-dimethyl-2-oxopyrrolidin-3-yl)thiazole-5-carboxamide;
(S)-2-(3-Chloro-5-fluorophenyl)-N-(5,5-dimethyl-2-oxopyrrolidin-3-yl)thiazole-5-carboxamide;
(S)-N-(1-(2-Amino-2-oxoethyl)-2-oxopyrrolidin-3-yl)-2-(3,5-difluorophenyl)thiazole-5-carboxamide;
Racemic 2-(3-(Difluoromethyl)-5-fluorophenyl)-N-(5,5-dimethyl-2-oxopyrrolidin-3-yl)thiazole-5-carboxamide;
(S)-2-(3-Chloro-5-fluorophenyl)-N-(4,4-dimethyl-2-oxopyrrolidin-3-yl)thiazole-5-carboxamide;
Racemic 2-(3-Chloro-5-fluorophenyl)-N-(5-oxo-4-azaspiro[2.4]heptan-6-yl)thiazole-5-carboxamide;
(S)-2-(3-Chloro-5-fluorophenyl)-N-(5-oxo-4-azaspiro[2.4]heptan-6-yl)thiazole-5-carboxamide;
(R)-2-(3-Chloro-5-fluorophenyl)-N-(5-oxo-4-azaspiro[2.4]heptan-6-yl)thiazole-5-carboxamide;
Racemic 2-(3-Chloro-5-fluorophenyl)-N-(4,4-diethyl-2-oxopyrrolidin-3-yl)thiazole-5-carboxamide;
(R)-2-(3-Chloro-5-fluorophenyl)-N-(4,4-diethyl-2-oxopyrrolidin-3-yl)thiazole-5-carboxamide;
(S)-2-(3-Chloro-5-fluorophenyl)-N-(4,4-diethyl-2-oxopyrrolidin-3-yl)thiazole-5-carboxamide;
(R)-2-(3-Chloro-5-fluorophenyl)-N-(2-oxopyrrolidin-3-yl)thiazole-5-carboxamide;
Racemic 2-(3-Chloro-5-fluorophenyl)-N-(6-oxo-5-azaspiro[3.4]octan-7-yl)thiazole-5-carboxamide;
(R)-2-(3-Chloro-5-fluorophenyl)-N-(6-oxo-5-azaspiro[3.4]octan-7-yl)thiazole-5-carboxamide;
(S)-2-(3-Chloro-5-fluorophenyl)-N-(6-oxo-5-azaspiro[3.4]octan-7-yl)thiazole-5-carboxamide;
(S)-2-(3-Chlorophenyl)-4-methyl-N-(2-oxopyrrolidin-3-yl)thiazole-5-carboxamide;
(S)-2-(4-Methyl-1H-pyrazol-1-yl)-N-(2-oxopyrrolidin-3-yl)thiazole-5-carboxamide;
(S)-2-(4-Methyl-1H-imidazol-1-yl)-N-(2-oxopyrrolidin-3-yl)thiazole-5-carboxamide;
(S)-N-(2-Oxopyrrolidin-3-yl)-2-phenylthiazole-5-carboxamide;
Racemic 2-(3-Chloro-5-fluorophenyl)-N-((3R,4S,5S)-4,5-dimethyl-2-oxopyrrolidin-3-yl)thiazole-5-carboxamide and 2-(3-Chloro-5-fluorophenyl)-N-((3S,4R,5R)-4,5-dimethyl-2-oxopyrrolidin-3-yl)thiazole-5-carboxamide;
2-(3-Chloro-5-fluorophenyl)-N-((3R,4S,5S)-4,5-dimethyl-2-oxopyrrolidin-3-yl)thiazole-5-carboxamide;
2-(3-Chloro-5-fluorophenyl)-N-((3S,4R,5R)-4,5-dimethyl-2-oxopyrrolidin-3-yl)thiazole-5-carboxamide;
(S)-2-(3-Bromophenyl)-N-(2-oxopyrrolidin-3-yl)thiazole-5-carboxamide;
(S)-N-(2-Oxopyrrolidin-3-yl)-2-(pyridin-4-yl)thiazole-5-carboxamide;
(S)-N-(2-Oxopyrrolidin-3-yl)-2-(pyridin-2-yl)thiazole-5-carboxamide;
(S)-N-(2-Oxopyrrolidin-3-yl)-2-(pyridin-3-yl)thiazole-5-carboxamide;
2-(3-Chloro-5-fluorophenyl)-N-((3R,5S)-5-methyl-2-oxopyrrolidin-3-yl)thiazole-5-carboxamide;
(S)-2-(4-Methylpyrimidin-2-yl)-N-(2-oxopyrrolidin-3-yl)thiazole-5-carboxamide;
(S)-2-(3-Cyanophenyl)-N-(2-oxopyrrolidin-3-yl)thiazole-5-carboxamide;
(S)-N-(2-Oxopyrrolidin-3-yl)-2-(p-tolyl)thiazole-5-carboxamide;
(S)-2-(3-Fluorophenyl)-N-(2-oxopyrrolidin-3-yl)thiazole-5-carboxamide;
(S)-2-(6-Methylpyridin-2-yl)-N-(2-oxopyrrolidin-3-yl)thiazole-5-carboxamide;
(S)-2-(4-Methylpyridin-2-yl)-N-(2-oxopyrrolidin-3-yl)thiazole-5-carboxamide;
(S)-2-(3-(Difluoromethyl)-5-methylphenyl)-N-(2-oxopyrrolidin-3-yl)thiazole-5-carboxamide;
Racemic 2-(3-Chloro-5-fluorophenyl)-N-((3R,3aS,6aR)-2-oxooctahydrocyclopenta[b]pyrrol-3-yl)thiazole-5-carboxamide and 2-(3-Chloro-5-fluorophenyl)-N-((3S,3aR,6aS)-2-oxooctahydrocyclopenta[b]pyrrol-3-yl)thiazole-5-carboxamide;
2-(3-Chloro-5-fluorophenyl)-N-((3R,3aS,6aR)-2-oxooctahydrocyclopenta[b]pyrrol-3-yl)thiazole-5-carboxamide;
2-(3-Chloro-5-fluorophenyl)-N-((3S,3aR,6aS)-2-oxooctahydrocyclopenta[b]pyrrol-3-yl)thiazole-5-carboxamide;
Racemic 2-(3-Chloro-5-fluorophenyl)-N-((3S,3aR,6aR)-2-oxohexahydro-1H-furo[3,4-b]pyrrol-3-yl)thiazole-5-carboxamide and 2-(3-Chloro-5-fluorophenyl)-N-((3R,3aS,6aS)-2-oxohexahydro-1H-furo[3,4-b]pyrrol-3-yl)thiazole-5-carboxamide;
2-(3-Chloro-5-fluorophenyl)-N-((3S,3aR,6aR)-2-oxohexahydro-1H-furo[3,4-b]pyrrol-3-yl)thiazole-5-carboxamide;
2-(3-Chloro-5-fluorophenyl)-N-((3R,3aS,6aS)-2-oxohexahydro-1H-furo[3,4-b]pyrrol-3-yl)thiazole-5-carboxamide;
Racemic 2-(3-Chloro-5-fluorophenyl)-N-(6-oxo-2-oxa-5-azaspiro[3.4]octan-7-yl)thiazole-5-carboxamide;
(R)-2-(3-Chloro-5-fluorophenyl)-N-(6-oxo-2-oxa-5-azaspiro[3.4]octan-7-yl)thiazole-5-carboxamide;
(S)-2-(3-Chloro-5-fluorophenyl)-N-(6-oxo-2-oxa-5-azaspiro[3.4]octan-7-yl)thiazole-5-carboxamide;
Racemic 2-(3-Chloro-5-fluorophenyl)-N-((3R,3aR,6aS)-2-oxooctahydrocyclopenta[b]pyrrol-3-yl)thiazole-5-carboxamide and 2-(3-Chloro-5-fluorophenyl)-N-((3S,3aS,6aR)-2-oxooctahydrocyclopenta[b]pyrrol-3-yl)thiazole-5-carboxamide;
2-(3-Chloro-5-fluorophenyl)-N-((3R,3aR,6aS)-2-oxooctahydrocyclopenta[b]pyrrol-3-yl)thiazole-5-carboxamide;
2-(3-Chloro-5-fluorophenyl)-N-((3S,3aS,6aR)-2-oxooctahydrocyclopenta[b]pyrrol-3-yl)thiazole-5-carboxamide;
2-(3-Chloro-5-fluorophenyl)-N-((3R,5R)-5-methyl-2-oxopyrrolidin-3-yl)thiazole-5-carboxamide;
2-(3-Chloro-5-fluorophenyl)-N-((3S,5R)-5-methyl-2-oxopyrrolidin-3-yl)thiazole-5-carboxamide;
Racemic 2-(3-Chloro-5-fluorophenyl)-N-(1-methyl-2-oxopyrrolidin-3-yl)thiazole-5-carboxamide;
(S)-2-(3-Chloro-5-fluorophenyl)-N-(1-methyl-2-oxopyrrolidin-3-yl)thiazole-5-carboxamide;
Racemic 2-(3-Chloro-5-fluorophenyl)-N-methyl-N-(2-oxopyrrolidin-3-yl)thiazole-5-carboxamide;
(S)-2-(3-Chloro-5-fluorophenyl)-N-methyl-N-(2-oxopyrrolidin-3-yl)thiazole-5-carboxamide;
(R)-2-(3-Chloro-5-fluorophenyl)-N-methyl-N-(2-oxopyrrolidin-3-yl)thiazole-5-carboxamide;
(S)-2-(3-Methoxyphenyl)-N-(2-oxopyrrolidin-3-yl)thiazole-5-carboxamide;
(S)-2-(3-Hydroxyphenyl)-N-(2-oxopyrrolidin-3-yl)thiazole-5-carboxamide;
Racemic 2-(3-Chloro-5-fluorophenyl)-N-(1-(4-methoxybenzyl)-3-methyl-2-oxopyrrolidin-3-yl)thiazole-5-carboxamide;
Racemic 2-(3-Chloro-5-fluorophenyl)-N-(3-methyl-2-oxopyrrolidin-3-yl)thiazole-5-carboxamide;
(S)-2-(3-Chloro-5-fluorophenyl)-N-(3-methyl-2-oxopyrrolidin-3-yl)thiazole-5-carboxamide;
(R)-2-(3-Chloro-5-fluorophenyl)-N-(3-methyl-2-oxopyrrolidin-3-yl)thiazole-5-carboxamide;
Racemic 2-(3-Chloro-5-fluorophenyl)-N-((3S,4S)-4-methyl-2-oxopyrrolidin-3-yl)thiazole-5-carboxamide and Racemic 2-(3-Chloro-5-fluorophenyl)-N-((3S,4R)-4-methyl-2-oxopyrrolidin-3-yl)thiazole-5-carboxamide;
2-(3-Chloro-5-fluorophenyl)-N-((3S,4S)-4-methyl-2-oxopyrrolidin-3-yl)thiazole-5-carboxamide;
2-(3-Chloro-5-fluorophenyl)-N-((3R,4R)-4-methyl-2-oxopyrrolidin-3-yl)thiazole-5-carboxamide;
(S)-2-(3-Chloro-5-fluorophenyl)-N-(2-thioxopyrrolidin-3-yl)thiazole-5-carboxamide;
(S)-2-(3-chloro-5-fluorophenyl)-N-(2-selenoxopyrrolidin-3-yl)thiazole-5-carboxamide;
2-(3-Chloro-5-fluorophenyl)-N-(2-oxoimidazolidin-1-yl)thiazole-5-carboxamide;
(S)-2-(3-chloro-5-fluorophenyl)-N-(2-oxopyrrolidin-3-yl)thiazole-5-carbothioamide;
2-(3-Chloro-5-fluorophenyl)-N-((3S,5S)-5-methyl-2-oxopyrrolidin-3-yl)thiazole-5-carboxamide;
Racemic 2-(3-Chloro-5-fluorophenyl)-N-(5,5-dimethyl-2-oxopyrrolidin-3-yl)-1,3-selenazole-5-carboxamide;
Racemic 2-(3-Chloro-5-fluorophenyl)-N-(5,5-dimethyl-2-thioxopyrrolidin-3-yl)thiazole-5-carboxamide;
(R)-2-(3-chloro-5-fluorophenyl)-N-(5,5-dimethyl-2-thioxopyrrolidin-3-yl)thiazole-5-carboxamide;
(S)-2-(3-chloro-5-fluorophenyl)-N-(5,5-dimethyl-2-thioxopyrrolidin-3-yl)thiazole-5-carboxamide; and
(S)-N-(2-oxopyrrolidin-3-yl)-2-phenylthiazole-5-sulfonamide,
   or a pharmaceutically acceptable salt thereof.

Suitably the compound of Formula (I) is (S)-2-(3-Chloro-5-fluorophenyl)-N-(5,5-dimethyl-2-oxopyrrolidin-3-yl)thiazole-5-carboxamide

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I). The invention is further directed to free or unsalted compounds of Formula (I).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p-chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

The compounds according to Formula (I) and pharmaceutically acceptable salts thereof may contain isotopically-labelled compounds, which are identical to those recited in Formula (I) and following, but for the fact that one or more atoms are replaced by an atom having an atomic mass or mass number different from the atomic mass or mass number usually found in nature. Examples of such isotopes include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I, and ¹²⁵I.

Isotopically-labelled compounds, for example those into which radioactive isotopes such as ³H or ¹⁴C are incorporated, are useful in drug and/or substrate tissue distribution assays. Tritium, i.e., ³H, and carbon-14, i.e. ¹⁴C isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), both are useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds can generally be prepared by substituting a readily available isotopically labelled reagent for a non-isotopically labelled reagent.

The compounds according to Formula (I) may also contain double bonds or other centers of geometric asymmetry. Where the stereochemistry of a center of geometric asymmetry present in Formula (I), or in any chemical structure illustrated herein, is not specified, the structure is intended to encompass the trans (E) geometric isomer, the cis (Z) geometric isomer, and all mixtures thereof. Likewise, all tautomeric forms are also included in Formula (I) whether such tautomers exist in equilibrium or predominately in one form.

The compounds of the invention may exist in solid or liquid form. In solid form, compound of the invention may exist in a continuum of solid states ranging from fully amorphous to fully crystalline. The term ‘amorphous’ refers to a state in which the material lacks long range order at the molecular level and, depending upon the temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may have the ability to crystallize in more than one form, a characteristic, which is known as polymorphism (“polymorphs”). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

The compounds of Formula (I) may exist in solvated and unsolvated forms. As used herein, the term “solvate” refers to a complex of variable stoichiometry formed by a solute (in this invention, a compound of Formula (I) or a salt) and a solvent. Such solvents, for the purpose of the invention, may not interfere with the biological activity of the solute. The skilled artisan will appreciate that pharmaceutically acceptable solvates may be formed for crystalline compounds wherein solvent molecules are incorporated into the crystalline lattice during crystallization. The incorporated solvent molecules may be water molecules or non-aqueous such as ethanol, isopropanol, DMSO, acetic acid, ethanolamine, and ethyl acetate molecules. Crystalline lattice incorporated with water molecules are typically referred to as “hydrates”. Hydrates include stoichiometric hydrates as well as compositions containing variable amounts of water.

It is also noted that the compounds of Formula (I) may form tautomers. ‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. It is understood that all tautomers and mixtures of tautomers of the compounds of the present invention are included within the scope of the compounds of the present invention.

While aspects for each variable have generally been listed above separately for each variable this invention includes those compounds in which several or each aspect in Formula (I) is selected from each of the aspects listed above. Therefore, this invention is intended to include all combinations of aspects for each variable.

### Definitions

It will be appreciated that the following definitions apply to each of the aforementioned formulae and to all instances of these terms, unless the context dictates otherwise.

“Alkyl” refers to a hydrocarbon chain having the specified number of “carbon atoms”. For example, C₁-C₆ alkyl refers to an alkyl groin having from 1 to 6 carbon atoms. Alkyl groups may be saturated, unsaturated, straight or branched. Representative branched alkyl groups have one, two, or three branches. Alkyl includes but is not limited to: methyl, ethyl, ethylene, ethynyl, propyl (n-propyl and isopropyl), butene, butyl (n-butyl, isobutyl, and t-butyl), pentyl and hexyl.

“Alkoxy” refers to an —O-alkyl group wherein “alkyl” is as defined herein. For example, C₁-C₄alkoxy refers to an alkoxy group having from 1 to 4 carbon atoms. Representative branched alkoxy groups have one, two, or three branches. Examples of such groups include methoxy, ethoxy, propoxy, t-butoxy and butoxy.

“Halogen” refers to the halogen radicals fluoro, chloro, bromo, and iodo.

“Heteroatom” refers to a nitrogen, sulfur or oxygen atom.

### Abbreviations

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
Ac (acetyl);
Ac₂O (acetic anhydride);
ACN (acetonitrile);
AIBN (azobis(isobutyronitrile));
BINAP (2,2′-bis(diphenylphosphino)-1,1′-binaphthyl);
BMS (borane—dimethyl sulphide complex);
Bn (benzyl);
Boc (tert-Butoxycarbonyl);
Boc₂O (di-tert-butyl dicarbonate);
BOP (Benzotriazole-1-yl-oxy-tris-(dimethylamino)-phosphonium hexafluorophosphate);
CAN (cerric ammonium nitrate);
Cbz (benzyloxycarbonyl);
CSI (chlorosulfonyl isocyanate);
CsF (cesium fluoride);
DABCO (1,4-Diazabicyclo[2.2.2]octane);
DAST (Diethylamino)sulfur trifluoride);
DBU (1,8-Diazabicyclo[5.4.0]undec-7-ene);
DCC (Dicyclohexyl Carbodiimide);
DCE (1,2-dichloroethane);
DDQ (2,3-Dichloro-5,6-dicyano-1,4-benzoquinone);
ATP (adenosine triphosphate);
Bis-pinacolatodiboron (4,4,4′,4′,5,5,5′,5′-Octamethyl-2,2′-bi-1,3,2-dioxaborolane);
BSA (bovine serum albumin);
C18 (refers to 18-carbon alkyl groups on silicon in HPLC stationary phase);
CH₃CN (acetonitrile);
Cy (cyclohexyl);
DCM (dichloromethane);
DIEA (Hunig's base, N,N-Diisopropylethylamine, N-ethyl-N-(1-methylethyl)-2-propanamine);
Dioxane (1,4-dioxane);
DMAP (4-dimethylaminopyridine);
DME (1,2-dimethoxyethane);
DMEDA (N,N′-dimethylethylenediamine);
DMF (N,N-dimethylformamide);
DMSO (dimethylsulfoxide);
DPPA (diphenyl phosphoryl azide);
EDC (N-(3-dimethylaminopropyl)-N′ethylcarbodiimide);
EDTA (ethylenediaminetetraacetic acid);
EtOAc (ethyl acetate);
EtOH (ethanol);
Et₂O (diethyl ether);
HEPES (4-(2-hydroxyethyl)-1-piperazine ethane sulfonic acid);
HATU (O-(7-Azabenzotriazol-1-yl)-N,N,N′,N′tetramethyluronium hexafluorophosphate, 1-((dimethylamino)(dimethyliminio)methyl)-1H-[1,2,3]triazolo[4,5-b]pyridine 3-oxide hexafluorophosphate(V));
HOAt (1-hydroxy-7-azabenzotriazole);
HOBt (1-hydroxybenzotriazole);
HOAc (acetic acid);
HPLC (high pressure liquid chromatography);
HMDS (hexamethyldisilazide);
IPA (isopropyl alcohol);
Indoline (2,3-dihydro-1H-indole);
KHMDS (potassium hexamethyldisilazide);
LAH (lithium aluminum hydride);
LDA (lithium diisopropylamide);
LHMDS (lithium hexamethyldisilazide)
MeOH (methanol);
MTBE (methyl tert-butyl ether);
mCPBA (m-chloroperoxybenzoic acid);
NaHMDS (sodium hexamethyldisilazide);
NBS (N-bromosuccinimide);
PE (petroleum ether);
Pd₂(dba)₃ (Tris(dibenzylideneacetone)dipalladium(0);
Pd(dppf)Cl₂-DCM Complex([1,1′-Bis(diphenylphosphino)ferrocene]dichloropalladium(II).dichloromethane complex);
PyBOP (benzotriazol-1-yl-oxytripyrrolidinophosphonium hexafluorophosphate);
PyBrOP (bromotripyrrolidinophosphonium hexafluorophosphate);
RP-HPLC (reverse phase high pressure liquid chromatography);
RT (room temperature);
Sat. (saturated)
SFC (supercritical fluid chromatography);
SGC (silica gel chromatography);
SM (starting material);
TLC (thin layer chromatography);
TEA (triethylamine);
TEMPO (2,2,6,6-Tetramethylpiperidine 1-oxyl, free radical);
TFA (trifluoroacetic acid); and
THE (tetrahydrofuran).

All references to ether are to diethyl ether and brine refers to a saturated aqueous solution of NaCl.

### Compound Preparation

The compounds according to Formula (I) are prepared using conventional organic synthetic methods. A suitable synthetic route is depicted below in the following general reaction schemes. All of the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

As used in the Schemes, “R” groups represent corresponding positional groups on any of Formulas I to III.

In one method of preparation, the thiazole amides can be prepared as shown in Scheme 1. First, 2-bromo-5-carboxy-thiazoles can be utilized in Suzuki or Negishi cross coupling reactions with arylboranes or arylzinc reagents, respectively, to afford 2-arylthiazole esters. These esters can then be hydrolyzed and the resulting carboxylic acids coupled with amines to provide the thiazole amides.

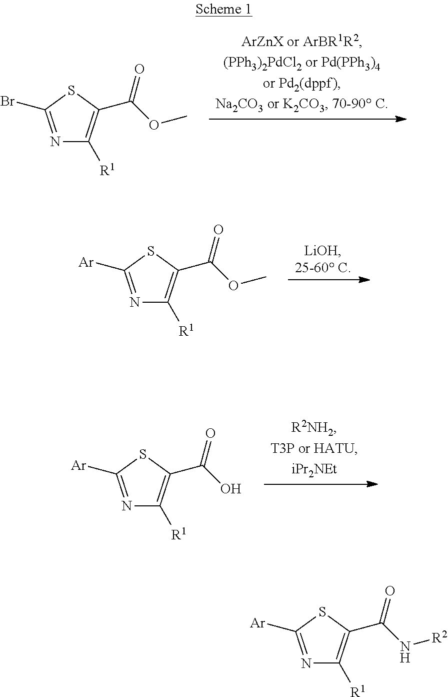

In an alternative method of preparation, the thiazole amides can be prepared as shown in Scheme 2. First, (5-(tert-butoxycarbonyl)thiazol-2-yl)zinc(II) bromide can be cross coupled via the Negishi protocol with aryl halides to afford 2-arylthiazole esters. These tert-butyl esters can then be deprotected with trifluoroacetic acid and the resulting carboxylic acids coupled with amines to provide the thiazole amides.

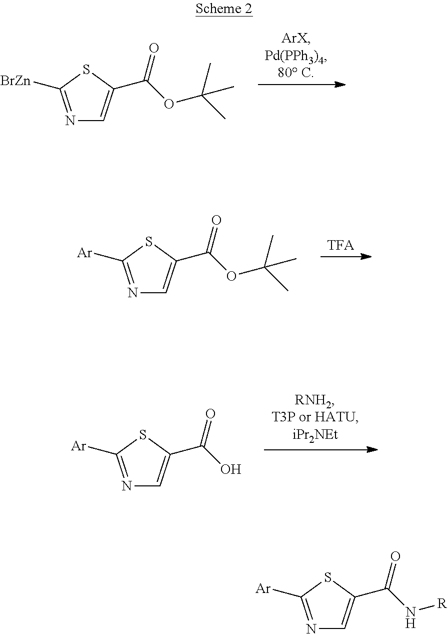

In another method of preparation, the thiazole amides can be prepared as shown in Scheme 3. First, 2-bromothiazole-5-carboxylic acid can be coupled to amines. Then the resulting amides can be cross coupled with aryl boronates to provide the desired thiazole amides.

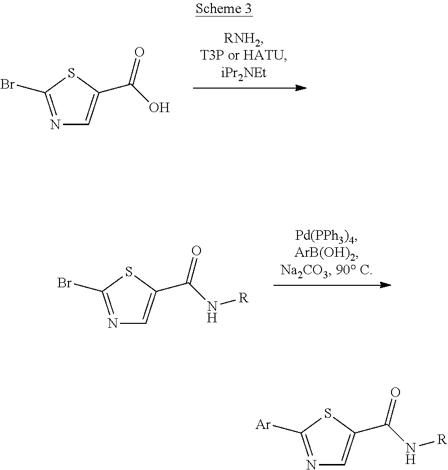

In another method of preparation, the thiazole amides can be prepared as shown in Scheme 4. First, aryl nitriles can be converted into aryl thioamides with Lawesson's reagent. These thioamides can then be condensed with ethyl 2-chloro-3-oxopropanoate to provide the thiazole esters. Then, hydrolysis of these esters and amide coupling of the resulting carboxylic acids with suitable amines gives the desired thiazole amides.

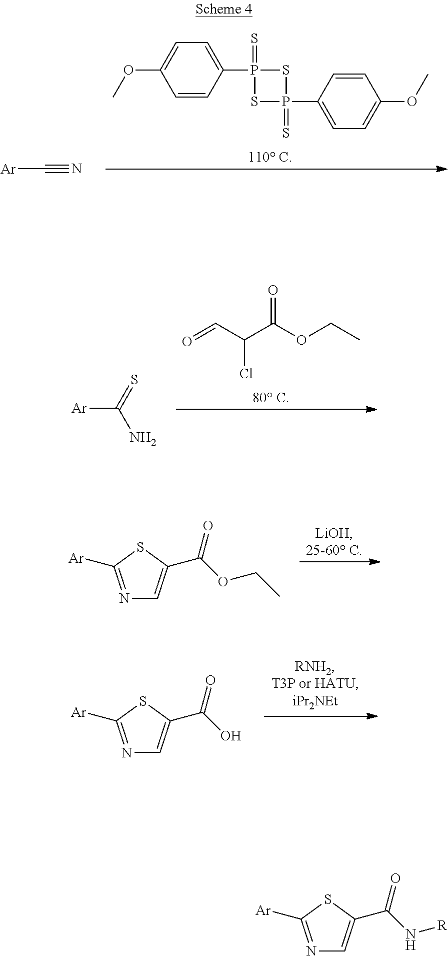

In one method of preparation, the intermediate 2-aminolactams can be synthesized as depicted in Scheme 5. First, oxidation of suitably protected aminoalcohols to their corresponding aldehydes or ketones, followed by Horner-Wadsworth-Emmons coupling with the suitably protected 2-amino-2-(dimethoxyphosphoryl)acetate provides the alkenes. Subsequent palladium catalyzed olefin hydrogenation, benzyloxycarbonyl protecting group cleavage, and cyclization affords the protected lactams. Finally, acid catalyzed unmasking of the amine gives the 2-aminolactam intermediates.

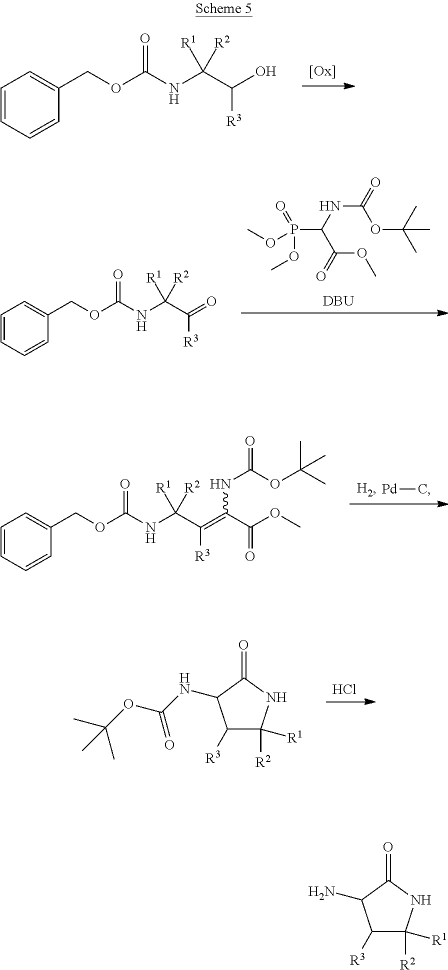

Other compounds of the invention can be prepared by analogous methods of preparation known to a person of ordinary skill in the art.

### Methods of Use

The inventors have shown that inhibitors of Hematopoietic Prostaglandin D Synthase (H-PGDS) reduce muscle damage and preserve muscle function when administered prior to muscle injury in an in vivo assay for muscle function. Furthermore, the inventors have shown that when an H-PGDS inhibitor is administered after muscle damage in the same assay, recovery of muscle function is enhanced. These results support a role for the use of H-PGDS inhibitors in the treatment of muscle degenerative disorders and muscle injury.

In one aspect, the invention provides a method of treating a muscle degenerative disorder comprising administering to a human an H-PGDS inhibitor of Formula (I) or a pharmaceutically acceptable salt thereof.

The invention also provides a method of treating a muscle degenerative disorder comprising administering to a human a compound of Formula (I) or a pharmaceutically acceptable salt thereof.

The invention also provides a method of treating a muscle degenerative disorder comprising administering to a human a compound of Formula (I).

The invention also provides a compound of Formula (I) or a pharmaceutically acceptable salt thereof for use in treating a muscle degenerative disorder.

In particular embodiments, the muscle degenerative disorder is muscular dystrophy, myotonic dystrophy, polymyositis, dermatomyositis, or inclusion body myositis.

For example, the compounds of Formula (I) or a pharmaceutically acceptable salt thereof may be used to treat a muscular dystrophy disorder selected from Duchenne MD, Becker MD, congenital MD (Fukuyama), Emery Dreifuss MD, limb girdle MD, and fascioscapulohumeral MD.

The compounds of Formula (I) or a pharmaceutically acceptable salt thereof may also be used to treat myotonic dystrophy type I (DM1 or Steinert's), myotonic dystrophy type II (DM2 or proximal myotonic myopathy), or congenital myotonia.

In some embodiments, the muscle injury is a surgery-related muscle injury, a traumatic muscle injury, a work-related skeletal muscle injury, or an overtraining-related muscle injury.

Non-limiting examples of surgery-related muscle injuries include muscle damage due to knee replacement, anterior cruciate ligament (ACL) repair, plastic surgery, hip replacement surgery, joint replacement surgery, tendon repair surgery, surgical repair of rotator cuff disease and injury, and amputation.

In one embodiment, the muscle injury is a surgery-related muscle injury and the treatment method provides for administration of at least one dose of an H-PGDS inhibitor of Formula (I) or a pharmaceutically acceptable salt thereof prior to the surgery (for example, within one day before the surgery) followed by periodic administration of a dose of the H-PGDS inhibitor during the recovery period.

In another embodiment, the muscle injury is a surgery-related muscle injury and the treatment method provides for administration of at least one dose of a compound of Formula (I) or a pharmaceutically acceptable salt thereof prior to the surgery (for example, within one day before the surgery) followed by periodic administration of a dose of the compound of Formula (I) or a pharmaceutically acceptable salt thereof during the recovery period.

In another embodiment, the muscle injury is a surgery-related muscle injury and the treatment method provides for administration of at least one high dose of an H-PGDS inhibitor of Formula (I) or a pharmaceutically acceptable salt thereof within one day to one week following the surgery.

In another embodiment, the muscle injury is a surgery-related muscle injury and the treatment method provides for administration of at least one high dose of a compound of Formula (I) or a pharmaceutically acceptable salt thereof within one day to one week following the surgery.

In yet another embodiment, the muscle injury is a surgery-related muscle injury and the treatment method provides for administration of at least one high dose of an H-PGDS inhibitor of Formula (I) or a pharmaceutically acceptable salt thereof within one day to one week following the surgery, followed by periodic administration of a dose of the H-PGDS inhibitor during the recovery period.

In yet another embodiment, the muscle injury is a surgery-related muscle injury and the treatment method provides for administration of at least one high dose of a compound of Formula (I) or a pharmaceutically acceptable salt thereof within one day to one week following the surgery, followed by periodic administration of a dose of the compound of Formula (I) or a pharmaceutically acceptable salt thereof during the recovery period.

Non-limiting examples of traumatic muscle injuries include battlefield muscle injuries, auto accident-related muscle injuries, and sports-related muscle injuries. Traumatic injury to the muscle can include lacerations, blunt force contusions, shrapnel wounds, muscle pulls or tears, burns, acute strains, chronic strains, weight or force stress injuries, repetitive stress injuries, avulsion muscle injury, and compartment syndrome.

In one embodiment, the muscle injury is a traumatic muscle injury and the treatment method provides for administration of at least one dose of an H-PGDS inhibitor of Formula (I) or a pharmaceutically acceptable salt thereof, immediately after the traumatic injury (for example, within one day of the injury) followed by periodic administration of a dose of the H-PGDS inhibitor during the recovery period.

In one embodiment, the muscle injury is a traumatic muscle injury and the treatment method provides for administration of at least one dose of a compound of Formula (I) or a pharmaceutically acceptable salt thereof, immediately after the traumatic injury (for example, within one day of the injury) followed by periodic administration of a dose of the compound of Formula (I) or a pharmaceutically acceptable salt thereof during the recovery period.

Non-limiting examples of work-related muscle injuries include injuries caused by highly repetitive motions, forceful motions, awkward postures, prolonged and forceful mechanical coupling between the body and an object, and vibration.

Overtraining-related muscle injuries include unrepaired or under-repaired muscle damage coincident with a lack of recovery or lack of an increase of physical work capacity.

In an additional embodiment, the muscle injury is exercise or sports-induced muscle damage including exercise-induced delayed onset muscle soreness (DOMS).

In some embodiments, the invention encompasses a therapeutic combination in which the H-PGDS inhibitor of Formula (I) or a pharmaceutically acceptable salt thereof is administered in a subject in combination with the implantation of a biologic scaffold (e.g. a scaffold comprising extracellular matrix) that promotes muscle regeneration. Such scaffolds are known in the art. See, for example, Turner and Badylack (2012) Cell Tissue Res. 347(3):759-74 and U.S. Pat. No. 6,576,265. Scaffolds comprising non-crosslinked extracellular matrix material are preferred.

In some embodiments, the invention encompasses a therapeutic combination in which the compound of Formula (I) or a pharmaceutically acceptable salt thereof is administered in a subject in combination with the implantation of a biologic scaffold (e.g. a scaffold comprising extracellular matrix) that promotes muscle regeneration. Scaffolds comprising non-crosslinked extracellular matrix material are preferred.

In another aspect, the invention provides a method of treating tendon damage where the method comprises administering a compound of Formula (I) or a pharmaceutically acceptable salt thereof to a subject in need thereof.

In a particular embodiment, the invention includes a method of enhancing the formation of a stable tendon-bone interface. In a related embodiment, the invention provides a method of increasing the stress to failure of tendons, for example surgically-repaired tendons. In an additional embodiment, the invention provides a method of reducing fibrosis at the repair site for surgically-repaired tendons. In a particular embodiment, the invention provides a method of treating tendon damage associated with rotator cuff injury, or tendon damage associated with surgical repair of rotator cuff injury.

In another aspect, the invention provides a method of treating a disease state selected from: allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, systemic lupus erythematosus (SLE), pulmonary fibrosis, respiratory distress syndrome, bronchiolitis, sinusitis, cystic fibrosis, actinic keratosis, skin dysplasia, chronic urticaria, eczema and all types of dermatitis including atopic dermatitis or contact dermatitis in a subject in need thereof comprising administering to the subject a therapeutically effective amount of a compound of Formula (I) or a pharmaceutically acceptable salt thereof.

The methods of treatment of the invention comprise administering a safe and effective amount of a compound of Formula (I), or a pharmaceutically acceptable salt thereof to a mammal, suitably a human, in need thereof.

In one aspect, the invention provides a compound of Formula (I) or a pharmaceutically acceptable salt thereof for use in therapy.

In another embodiment, the muscle injury is a surgery-related muscle injury and the invention provides for a compound of Formula (I) or a pharmaceutically acceptable salt for use in treating surgery-related muscle injury.

Non-limiting examples of traumatic muscle injuries include battlefield muscle injuries, auto accident-related muscle injuries, and sports-related muscle injuries. Traumatic injury to the muscle can include lacerations, blunt force contusions, shrapnel wounds, muscle pulls or tears, burns, acute strains, chronic strains, weight or force stress injuries, repetitive stress injuries, avulsion muscle injury, and compartment syndrome.

In one embodiment, the muscle injury is a traumatic muscle injury and the invention provides for a compound of Formula (I) or a pharmaceutically acceptable salt thereof for use in treating the treating traumatic muscle injury.

Non-limiting examples of work-related muscle injuries include injuries caused by highly repetitive motions, forceful motions, awkward postures, prolonged and forceful mechanical coupling between the body and an object, and vibration.

Overtraining-related muscle injuries include unrepaired or under-repaired muscle damage coincident with a lack of recovery or lack of an increase of physical work capacity.

In an additional embodiment, the muscle injury is exercise or sports-induced muscle damage including exercise-induced delayed onset muscle soreness (DOMS).

In another aspect, the invention provides a compound of Formula (I) or a pharmaceutically acceptable salt thereof for use in treating tendon damage.

In a particular embodiment, the invention provides a compound of Formula (I) or a pharmaceutically acceptable salt thereof for use in enhancing the formation of a stable tendon-bone interface.

In a related embodiment, the invention provides a compound of Formula (I) or a pharmaceutically acceptable salt thereof for use in increasing the stress to failure of tendons, for example surgically-repaired tendons.

In an additional embodiment, the invention provides a compound of Formula (I) or a pharmaceutically acceptable salt thereof for use in reducing fibrosis at the repair site for surgically-repaired tendons.

In a particular embodiment, the invention provides a compound of Formula (I) or a pharmaceutically acceptable salt thereof for use in treating tendon damage associated with rotator cuff injury, or tendon damage associated with surgical repair of rotator cuff injury.

In another aspect, the invention provides a compound of formula (I) or a pharmaceutically acceptable salt thereof for use in treating a disease state selected from: allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, systemic lupus erythematosus (SLE), pulmonary fibrosis, respiratory distress syndrome, bronchiolitis, sinusitis, cystic fibrosis, actinic keratosis, skin dysplasia, chronic urticaria, eczema and all types of dermatitis including atopic dermatitis or contact dermatitis.

In another embodiment, the muscle injury is a surgery-related muscle injury and the invention provides for the use of a compound of Formula (I) or a pharmaceutically acceptable salt in the manufacture of a medicament for use in treating surgery-related muscle injury.

Non-limiting examples of traumatic muscle injuries include battlefield muscle injuries, auto accident-related muscle injuries, and sports-related muscle injuries. Traumatic injury to the muscle can include lacerations, blunt force contusions, shrapnel wounds, muscle pulls or tears, burns, acute strains, chronic strains, weight or force stress injuries, repetitive stress injuries, avulsion muscle injury, and compartment syndrome.

In one embodiment, the muscle injury is a traumatic muscle injury and the invention provides for the use of a compound of Formula (I) or a pharmaceutically acceptable salt thereof in the manufacture of a medicament for use in treating the treating traumatic muscle injury.

Non-limiting examples of work-related muscle injuries include injuries caused by highly repetitive motions, forceful motions, awkward postures, prolonged and forceful mechanical coupling between the body and an object, and vibration.

Overtraining-related muscle injuries include unrepaired or under-repaired muscle damage coincident with a lack of recovery or lack of an increase of physical work capacity.

In an additional embodiment, the muscle injury is exercise or sports-induced muscle damage including exercise-induced delayed onset muscle soreness (DOMS).

In another aspect, the invention provides the use of a compound of Formula (I) or a pharmaceutically acceptable salt thereof in the manufacture of a medicament for use in treating tendon damage.

In a particular embodiment, the invention provides the use of a compound of Formula (I) or a pharmaceutically acceptable salt thereof in the manufacture of a medicament for use in enhancing the formation of a stable tendon-bone interface.

In a related embodiment, the invention provides the use of a compound of Formula (I) or a pharmaceutically acceptable salt thereof in the manufacture of a medicament for use in increasing the stress to failure of tendons, for example surgically-repaired tendons.

In an additional embodiment, the invention provides the use of a compound of Formula (I) or a pharmaceutically acceptable salt thereof in the manufacture of a medicament for use in reducing fibrosis at the repair site for surgically-repaired tendons.

In a particular embodiment, the invention provides the use of a compound of Formula (I) or a pharmaceutically acceptable salt thereof for in the manufacture of a medicament for use in treating tendon damage associated with rotator cuff injury, or tendon damage associated with surgical repair of rotator cuff injury.

In another aspect, the invention provides the use of a compound of formula (I) or a pharmaceutically acceptable salt thereof in the manufacture of a medicament for use in treating a disease state selected from: allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, systemic lupus erythematosus (SLE), pulmonary fibrosis, respiratory distress syndrome, bronchiolitis, sinusitis, cystic fibrosis, actinic keratosis, skin dysplasia, chronic urticaria, eczema and all types of dermatitis including atopic dermatitis or contact dermatitis.

The invention also provides a compound of Formula (I) or a pharmaceutically acceptable salt thereof for use in treating tendon damage.

The invention also provides a the use of a compound of Formula (I) or a pharmaceutically acceptable salt thereof in the manufacture of a medicament for use in treating tendon damage.

As used herein, “treat”, and derivatives thereof, in reference to a condition means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The term “treating” and derivatives thereof refers to therapeutic therapy. Therapeutic therapy is appropriate to alleviate symptoms or to treat at early signs of disease or its progression.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of Formula (I), or a pharmaceutically acceptable salt thereof, means an amount of the compound sufficient to treat the patient's condition but low enough to avoid serious side effects (at a reasonable benefit/risk ratio) within the scope of sound medical judgment. A safe and effective amount of the compound will vary with the particular route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient”, and derivatives thereof refers to a human or other mammal, suitably a human.

The subject to be treated in the methods of the invention is typically a mammal in need of such treatment, preferably a human in need of such treatment.

### Compositions

The pharmaceutically active compounds within the scope of this invention are useful as inhibitors of H-PGDS in mammals, particularly humans, in need thereof.

The present invention therefore provides a method of treating neurodegenerative diseases, musculoskeletal diseases and other conditions requiring H-PGDS inhibition, which comprises administering an effective amount of a compound of Formula (I) or a pharmaceutically acceptable salt thereof. The compounds of Formula (I) also provide for a method of treating the above indicated disease states because of their demonstrated ability to act as H-PGDS inhibitors. The drug may be administered to a patient in need thereof by any conventional route of administration, including, but not limited to, intravenous, intramuscular, oral, topical, subcutaneous, intradermal, intraocular and parenteral. Suitably, a H-PGDS inhibitor may be delivered directly to the brain by intrathecal or intraventricular route, or implanted at an appropriate anatomical location within a device or pump that continuously releases the H-PGDS inhibitor drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-500 mg/kg of active compound, preferably 0.001-100 mg/kg. When treating a human patient in need of a H-PGDS inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular H-PGDS inhibitor in use, the strength of the preparation, the mode of administration, and the advancement of the disease condition. Additional factors depending on the particular patient being treated will result in a need to adjust dosages, including patient age, gender, ethnicity, weight, diet, and time of administration.

When administered to prevent organ damage in the transportation of organs for transplantation, a compound of Formula (I) is added to the solution housing the organ during transportation, suitably in a buffered solution.

The method of this invention of inducing H-PGDS inhibitory activity in mammals, including humans, comprises administering to a subject in need of such activity an effective H-PGDS inhibiting amount of a pharmaceutically active compound of the present invention.

The invention also provides for the use of a compound of Formula (I) or a pharmaceutically acceptable salt thereof in the manufacture of a medicament for use as a H-PGDS inhibitor.

The invention also provides for the use of a compound of Formula (I) or a pharmaceutically acceptable salt thereof for use in the treatment of a condition for which a H-PGDS inhibitor is indicated.

The invention also provides for the use of a compound of Formula (I) or a pharmaceutically acceptable salt thereof in the manufacture of a medicament for use in therapy.

The invention also provides for the use of a compound of Formula (I) or a pharmaceutically acceptable salt thereof in the manufacture of a medicament for treating musculoskeletal diseases such as Duchenne muscular dystrophy, spinal cord contusion injury, neuroinflammatory diseases such as multiple sclerosis or neurodegenerative diseases such as Alzheimer's disease or amyotrophic lateral sclerosis (ALS).

The invention also provides for a pharmaceutical composition for use as a H-PGDS inhibitor which comprises a compound of Formula (I) or a pharmaceutically acceptable salt thereof and a pharmaceutically acceptable carrier.

The invention also provides for a pharmaceutical composition for use in the treatment of cancer which comprises a compound of Formula (I) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a H-PGDS inhibitor.

By the term “co-administration” as used herein is meant either simultaneous administration or any manner of separate sequential administration of a H-PGDS inhibiting compound, as described herein, and a further active agent or agents, known to be useful in the treatment of conditions in which a H-PGDS inhibitor is indicated. The term further active agent or agents, as used herein, includes any compound or therapeutic agent known to or that demonstrates advantageous properties when administered to a patient in need of H-PGDS inhibition. Preferably, if the administration is not simultaneous, the compounds are administered in a close time proximity to each other. Furthermore, it does not matter if the compounds are administered in the same dosage form, e.g. one compound may be administered by injection and another compound may be administered orally.

The invention also relates to the use of a compound of Formula (I) or a pharmaceutically acceptable salt thereof in the preparation of a medicament for the treatment of neurodegenerative diseases, musculoskeletal diseases and diseases associated with H-PGDS inhibition.

The invention also provides a pharmaceutical composition comprising from 0.5 to 1,000 mg of a compound of Formula (I) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

### Examples

The following Examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### INTERMEDIATES

Intermediate 1

### 2-(Benzofuran-7-yl)thiazole-5-carboxylic Acid

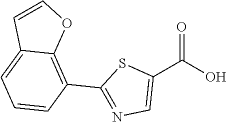 [SMILES: O=C(O)c1cnc(-c2cccc3ccoc23)s1]

### A. Ethyl 2-(benzofuran-7-yl)thiazole-5-carboxylate

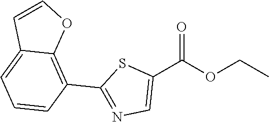 [SMILES: CCOC(=O)c1cnc(-c2cccc3ccoc23)s1]

Ethyl 2-bromothiazole-5-carboxylate (1.010 g, 4.28 mmol) and 2-(benzofuran-7-yl)-4,4,5,5-tetramethyl-1,3,2-dioxaborolane (1.105 g, 4.53 mmol) were dissolved in 1,4-dioxane (8 mL) and water (4 mL). Potassium carbonate (2M aqueous solution, 4.25 mL, 8.50 mmol) was added, followed by bis(triphenylphosphine)palladium(II) chloride (0.300 g, 0.427 mmol). The mixture was heated to 90° C. for 4 hours and was cooled to room temperature. Water (75 mL) and brine (25 mL) were added and the mixture was extracted with diethyl ether (4×20 mL). The combined organics were dried over magnesium sulfate and concentrated. The residue was purified by silica gel chromatography, eluting with ethyl acetate:heptane (1:19 to 1:6) to provide ethyl benzofuran-7-yl)thiazole-5-carboxylate (0.4297 g, 1.572 mmol, 37% yield) as a pale yellow solid. ¹H NMR (CDCl₃): δ 1.43 (t, J=7 Hz, 3H), 4.42 (q, J=7 Hz, 2H), 6.90 (d, J=2 Hz, 1H), 7.38 (t, J=8 Hz, 1H), 7.74 (d, J=7 Hz, 1H), 7.82 (d, J=2 Hz, 1H), 8.23 (d, J=8 Hz, 1H), 8.53 (s, 1H).

### B. 2-(Benzofuran-7-yl)thiazole-5-carboxylic Acid

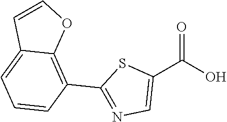 [SMILES: O=C(O)c1cnc(-c2cccc3ccoc23)s1]

Ethyl 2-(benzofuran-7-yl)thiazole-5-carboxylate (0.430 g, 1.57 mmol) was dissolved in tetrahydrofuran:methanol:water (4:1:1, 6 mL) and lithium hydroxide monohydrate (0.355 g, 8.46 mmol) was added. The mixture was stirred at room temperature for 60 minutes and was concentrated. The residue was dissolved in water (75 mL). The solution was acidified with 1M aqueous hydrochloric acid to ˜pH=2. The precipitated solid was collected by filtration, washed with water and dried to provide 2-(benzofuran-7-yl)thiazole-5-carboxylic acid (0.3391 g, 1.383 mmol, 88% yield) as a pale yellow solid. ¹H NMR (400 MHz, CD₃SOCD₃) δ 7.15 (d, J=2 Hz, 1H), 7.44 (t, J=8 Hz, 1H), 7.87 (d, J=7 Hz, 1H), 8.17 (d, J=7 Hz, 1H), 8.25 (d, J=2 Hz, 1H), 8.51 (s, 1H).

Intermediate 2

### 2-(3-Chlorophenyl)thiazole-5-carboxylic Acid

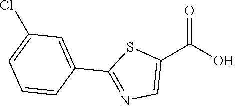 [SMILES: O=C(O)c1cnc(-c2cccc(Cl)c2)s1]

### A. Ethyl 2-(3-chlorophenyl)thiazole-5-carboxylate

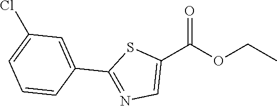 [SMILES: CCOC(=O)c1cnc(-c2cccc(Cl)c2)s1]

Tetrakis(triphenylphosphine)palladium(0) (0.251 g, 0.218 mmol) was added to ethyl 2-bromothiazole-5-carboxylate (0.65 mL, 4.35 mmol) in tetrahydrofuran (3 mL). Then, a 0.5 M solution of (3-chlorophenyl)zinc(II) iodide (11.32 mL, 5.66 mmol) was added via syringe (exothermic). The mixture was stirred in a heating block at 70° C. An additional portion of arylzinc reagent (1.2 mL) was added after 150 minutes and heating resumed. Another portion of arylzinc reagent (1.5 mL) was added after 1 hour and heating continued for 45 minutes. Upon cooling, the mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (0:1 to 1:0) to afford ethyl 2-(3-chlorophenyl)thiazole-5-carboxylate (0.970 g, 3.62 mmol, 83% yield) as a colorless solid. ¹H NMR (400 MHz, CD₃SOCD₃) δ 1.32 (t, J=7 Hz, 3H), 4.34 (q, J=7 Hz, 2H), 7.57 (dt, J=8, 1 Hz, 1H), 7.64 (ddd, J=8, 2, 1 Hz, 1H), 7.98 (dt, J=2, 1 Hz, 1H), 8.04 (dt, J=8, 1 Hz, 1H), 8.52 (s, 1H); LC-MS (LC-ES) M+H=268.

### B. 2-(3-Chlorophenyl)thiazole-5-carboxylic Acid

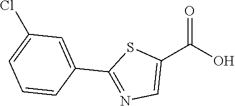 [SMILES: O=C(O)c1cnc(-c2cccc(Cl)c2)s1]

Lithium hydroxide monohydrate (0.730 g, 17.41 mmol) was added to a solution of ethyl 2-(3-chlorophenyl)thiazole-5-carboxylate (0.932 g, 3.48 mmol) in tetrahydrofuran (5 mL) and water (0.5 mL) and the reaction mixture was stirred 1 hour at room temperature, then in a heating block at 50° C. After 3 hours, the mixture was cooled, poured into water and extracted with diethyl ether (2×). The aqueous layer was acidified by addition of 1 M hydrochloric acid (17.4 mL). The precipitated solids were collected by filtration and dried to afford 2-(3-chlorophenyl)thiazole-5-carboxylic acid (0.750 g, 3.13 mmol, 90% yield) as a colorless solid. ¹H NMR (400 MHz, CD₃SOCD₃) δ 7.56 (t, J=8 Hz, 1H), 7.63 (ddd, J=8, 2, 1 Hz, 1H), 7.97 (dt, J=8, 1 Hz, 1H), 8.03 (t, J=2 Hz, 1H), 8.44 (s, 1H), 13.72 (br s, 1H); LC-MS (LC-ES) M+H=240.

Intermediate 3

### 2-(3-(Trifluoromethyl)phenyl)thiazole-5-carboxylic Acid

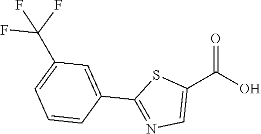 [SMILES: O=C(O)c1cnc(-c2cccc(C(F)(F)F)c2)s1]

### A. Ethyl 2-(3-(trifluoromethyl)phenyl)thiazole-5-carboxylate

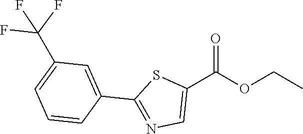 [SMILES: CCOC(=O)c1cnc(-c2cccc(C(F)(F)F)c2)s1]

A solution of isopropyl magnesium chloride:lithium chloride complex in tetrahydrofuran (4.25 mL, 5.52 mmol) was added to a solution of 1-iodo-3-(trifluoromethyl)benzene (0.724 mL, 5.02 mmol) in tetrahydrofuran (4 mL) at −78° C. dropwise over 5 minutes. The reaction mixture was stirred 30 minutes and a solution of zinc(II) bromide in tetrahydrofuran (3.37 mL, 5.86 mmol) was added dropwise. The cooling bath was removed and the mixture allowed to warm to room temperature. Then, tetrakis(triphenylphosphine)palladium(0) (0.193 g, 0.167 mmol) and ethyl 2-bromothiazole-5-carboxylate (0.791 g, 3.35 mmol) were added and the reaction mixture was stirred in a heating block at 70° C. for thirty minutes. After cooling, the reaction mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (0:1 to 1:0) to afford ethyl 2-(3-(trifluoromethyl)phenyl)thiazole-5-carboxylate (0.838 g, 2.78 mmol, 83% yield) as a pale yellow solid. ¹H NMR (400 MHz, CD₃SOCD₃) δ 1.32 (t, J=7 Hz, 3H), 4.34 (q, J=7 Hz, 2H), 7.78 (tt, J=8, 1 Hz, 1H), 7.94 (tt, J=8, 1 Hz, 1H), 8.30 (dd, J=2, 1 Hz, 1H), 8.33 (s, 1H), 8.55 (s, 1H); LC-MS (LC-ES) M+H=302.

### B. 2-(3-(Trifluoromethyl)phenyl)thiazole-5-carboxylic Acid

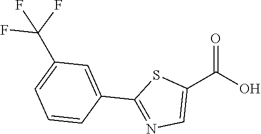 [SMILES: O=C(O)c1cnc(-c2cccc(C(F)(F)F)c2)s1]

Lithium hydroxide monohydrate (0.575 g, 13.69 mmol) was added to a solution of ethyl 2-(3-(trifluoromethyl)phenyl)thiazole-5-carboxylate (0.825 g, 2.74 mmol) in tetrahydrofuran (5 mL) and water (0.5 mL) and the reaction mixture was stirred in a heating block at 50° C. After 22 hours, the reaction mixture was cooled, poured into water and extracted with diethyl ether (2×). The aqueous layer was acidified by addition of 1M hydrochloric acid (13.7 mL) and the precipitated solids were collected by filtration, washed with water, and dried to afford 2-(3-(trifluoromethyl)phenyl)thiazole-5-carboxylic acid (0.6954 g, 2.55 mmol, 93% yield) as a colorless solid. ¹H NMR (400 MHz, CD₃SOCD₃) δ 7.78 (t, J=8 Hz, 1H), 7.93 (dd, J=8, 1 Hz, 1H), 8.29 (d, J=2 Hz, 1H), 8.31 (s, 1H), 8.47 (s, 1H), 13.76 (br s, 1H); LC-MS (LC-ES) M+H=274.

Intermediate 4

### 2-(m-Tolyl)thiazole-5-carboxylic Acid

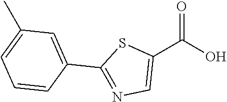

### A. Ethyl 2-(m-tolyl)thiazole-5-carboxylate

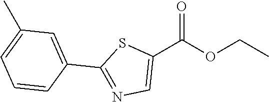 [SMILES: CCOC(=O)c1cnc(-c2cccc(C)c2)s1]

A solution of n-butyllithium in hexanes (2.176 mL, 5.44 mmol) was added, dropwise, over 3 minutes to a solution of 1-iodo-3-methylbenzene (0.671 mL, 5.22 mmol) in tetrahydrofuran (10 mL) at −78° C. The mixture was stirred 15 minutes (colorless precipitate formed) and a solution of zinc(II) bromide in rofuran (3.27 mL, 5.66 mmol) was added dropwise (homogeneous pale yellow solution). The mixture was removed from the cooling bath and allowed to warm to room temperature. Ethyl 2-bromothiazole-5-carboxylate (0.65 mL, 4.35 mmol) was added via syringe, followed by tetrakis(triphenylphosphine)palladium(0) (0.251 g, 0.218 mmol) and the vial's septum was replaced with a crimp top. The mixture was stirred in a heating block at 70° C. for 1 hour (LCMS indicated reaction had stalled (˜15% SM after 30 min)). Upon cooling, the mixture was poured into saturated ammonium chloride and extracted with ethyl acetate (3×). The combined organics were washed with water and saturated sodium chloride, dried over sodium sulfate, filtered, and concentrated in vacuo. The residue was purified by low pressure liquid chromatography, eluting with ethyl acetate:hexanes (0:1 to 1:6) to afford ethyl 2-(m-tolyl)thiazole-5-carboxylate (0.629 g, 2.54 mmol, 58.4% yield) as a colorless syrup. ¹H NMR (400 MHz, CD₃SOCD₃) δ 1.31 (t, J=7 Hz, 3H), 2.39 (s, 3H), 4.33 (q, J=7 Hz, 2H), 7.37 (ddt, J=8, 2, 1 Hz, 1H), 7.42 (t, J=8 Hz, 1H), 7.81 (ddt, J=8, 2, 1 Hz, 1H), 7.85 (dt, J=2, 1 Hz, 1H), 8.48 (s, 1H); LC-MS (LC-ES) M+H=248.

### B. 2-(m-Tolyl)thiazole-5-carboxylic Acid

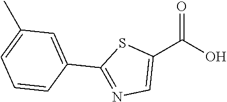

Lithium hydroxide monohydrate (0.518 g, 12.33 mmol) was added to a solution of ethyl 2-(m-tolyl)thiazole-5-carboxylate (0.610 g, 2.467 mmol) in tetrahydrofuran (5 mL) and water (0.5 mL) and the reaction mixture was stirred in a heating block at 50° C. for 3 hours. Upon cooling, the mixture was diluted with water and extracted with diethyl ether. The aqueous layer was acidified by addition of 6 M hydrochloric acid. The precipitated solids were collected by filtration and dried on the Buchner funnel to afford 2-(m-tolyl)thiazole-5-carboxylic acid (0.463 g, 2.112 mmol, 86% yield) as a colorless solid. ¹H NMR (400 MHz, CD₃SOCD₃) δ 2.39 (s, 3H), 7.37 (d, J=8 Hz, 1H), 7.42 (t, J=8 Hz, 1H), 7.81 (d, J=8 Hz, 1H), 7.84 (s, 1H), 8.41 (s, 1H), 13.62 (br s, 1H); LC-MS (LC-ES) M+H=220.

Intermediate 5

### 2-(3-Chloro-5-fluorophenyl)thiazole-5-carboxylic Acid

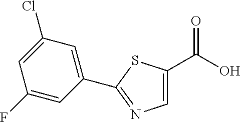 [SMILES: O=C(O)c1cnc(-c2cc(F)cc(Cl)c2)s1]

### A. Ethyl 2-(3-chloro-5-fluorophenyl)thiazole-5-carboxylate

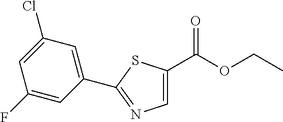 [SMILES: CCOC(=O)c1cnc(-c2cc(F)cc(Cl)c2)s1]

A 1 L 3-necked Morton flask was charged with ethyl 2-bromothiazole-5-carboxylate (37.7 g, 160 mmol), (3-chloro-5-fluorophenyl)boronic acid (41.8 g, 240 mmol), and tetrakis(triphenylphosphine)palladium(0) (7.38 g, 6.39 mmol). A reflux condensor was attached and the system was sealed with rubber septa. Toluene (300 mL), ethanol (120 mL) and sodium carbonate (240 mL, 479 mmol) were added and the mixture was degassed by sparging with nitrogen for −30 min. An internal temperature probe was attached and the mixture was heated under reflux (ca. 77° C.) for 11 hours (LC-MS indicated consumption of bromothiazole). Upon cooling the mixture was partitioned between ethyl acetate and water. The aqueous layer was extracted with ethyl acetate (2×). The combined organics were washed with water and brine, dried over sodium sulfate, filtered, and concentrated in vacuo. The residue was split into 4 equal portions and purified by flash chromatography, eluting with ethyl acetate:hexanes (0:1 to 1:9) to afford ethyl 2-(3-chloro-5-fluorophenyl)thiazole-5-carboxylate (16.6 g, 58.1 mmol, 36.4% yield) as a colorless solid. ¹H NMR (400 MHz, CDCl₃) δ 1.38 (t, J=7 Hz, 3H), 4.38 (q, J=7 Hz, 2H), 7.18 (ddd, J=8, 2, 2 Hz, 1H), 7.58 (ddd, J=9, 2, 1 Hz, 1H), 7.75 (dt, J=2, 1 Hz, 1H), 8.40 (s, 1H); LC-MS (LC-ES) M+H=286.

### B. 2-(3-Chloro-5-fluorophenyl)thiazole-5-carboxylic Acid

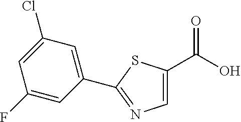 [SMILES: O=C(O)c1cnc(-c2cc(F)cc(Cl)c2)s1]

A Morton flask equipped with overhead stirring was charged with ethyl 2-(3-chloro-5-fluorophenyl)thiazole-5-carboxylate (33.3 g, 117 mmol), tetrahydrofuran (300 mL), methanol (175 mL), and a solution of lithium hydroxide monohydrate (24.45 g, 583 mmol) in water (200 mL) (thick ppt formed and slowly dissolved) (LCMS after 20 min indicated complete reaction). The volatiles were removed in vacuo. The residue was slurried in water (ca. 1.5 L total volume) and warmed to 50° C., with stirring, until complete dissolution. The mixture was acidified by addition of 6 M hydrochloric acid (100 mL, 600 mmol) and stirred overnight. The precipitated solids were collected by filtration, washed with water and diethyl ether, and dried on the Buchner funnel. Further drying in a vacuum oven (55° C./25″Hg vac) afforded 2-(3-chloro-5-fluorophenyl)thiazole-5-carboxylic acid (27.3 g, 106 mmol, 91% yield) as a colorless solid. ¹H NMR (400 MHz, CD₃SOCD₃) δ 7.65 (ddd, J=9, 2, 2 Hz, 1H), 7.84 (ddd, J=9, 2, 1 Hz, 1H), 7.91 (dt, J=2, 1 Hz, 1H), 8.45 (s, 1H), 13.77 (br s, 1H); LC-MS (LC-ES) M+H=258.

Intermediate 6

### 2-(5-Chloro-2-fluorophenyl)thiazole-5-carboxylic Acid

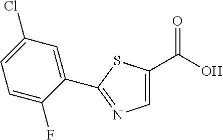 [SMILES: O=C(O)c1cnc(-c2cc(Cl)ccc2F)s1]

### A. Ethyl 2-(5-chloro-2-fluorophenyl)thiazole-5-carboxylate

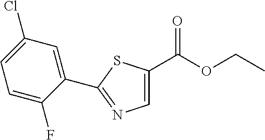 [SMILES: CCOC(=O)c1cnc(-c2cc(Cl)ccc2F)s1]

A mixture of ethyl 2-bromothiazole-5-carboxylate (0.5 g, 2.118 mmol), (5-chloro-2-fluorophenyl)boronic acid (0.480 g, 2.75 mmol), 2.0 M aqueous sodium carbonate (3.18 mL, 6.35 mmol) and tetrakis(triphenylphosphine)palladium(0) (0.15 g, 0.130 mmol) in toluene (14 mL) and ethanol (7.00 mL) was purged with nitrogen for a few minutes. Then the reaction mixture was heated at 90° C. in a sealed tube for ˜2 hours. The reaction mixture was then cooled to room temperature and filtered through a pad of Celite® that was washed with ethyl acetate. Aqueous sodium bicarbonate was added and the organic layer was separated and washed with aqueous sodium chloride, dried over sodium sulfate, filtered, and concentrated under reduced pressure. The residue was purified by silica gel chromatography, eluting with ethyl acetate:heptane (1:19 to 1:6) to give ethyl 2-(5-chloro-2-fluorophenyl)thiazole-5-carboxylate (452 mg, 1.582 mmol, 74.7% yield) as a white solid. ¹H NMR (400 MHz, CD₃SOCD₃) δ 1.34 (t, J=7 Hz, 3H), 4.37 (q, J=7 Hz, 2H), 7.60 (dd, J=11, 9 Hz, 1H), 7.72 (ddd, J=9, 4, 3 Hz, 1H), 8.25 (dd, J=6, 3 Hz, 1H), 8.64 (d, J=2 Hz, 1H); LC-MS (LC-ES) M+H=258.

### B. 2-(5-Chloro-2-fluorophenyl)thiazole-5-carboxylic Acid

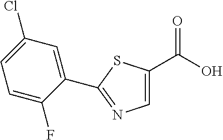 [SMILES: O=C(O)c1cnc(-c2cc(Cl)ccc2F)s1]

Lithium hydroxide monohydrate (0.075 g, 3.15 mmol) was added to ethyl 2-(5-chloro-2-fluorophenyl)thiazole-5-carboxylate (0.45 g, 1.575 mmol) in tetrahydrofuran (8 mL), ethanol (4.00 mL), and water (4.00 mL) and the reaction mixture was allowed to stir at room temperature for 2 hours before concentrating under reduced pressure. The residue was taken up in water and acidified with 6.0 M aqueous hydrochloric acid, upon addition a precipitant formed and was collected by filtration. The solid was washed with water and heptanes and dried to give 2-(5-chloro-2-fluorophenyl)thiazole-5-carboxylic acid (421 mg, 1.634 mmol, 104% yield) as a white solid. ¹H NMR (400 MHz, CD₃SOCD₃) δ 7.59 (dd, J=11, 9 Hz, 1H), 7.71 (ddd, J=9, 5, 3 Hz, 1H), 8.25 (dd, J=6, 3 Hz, 1H), 8.55 (d, J=3 Hz, 1H), 13.82 (br s, 1H); LC-MS (LC-ES) M+H=258.

### Intermediate 7

### 2-(3-Chloro-2-fluorophenyl)thiazole-5-carboxylic Acid

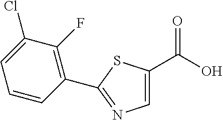 [SMILES: O=C(O)c1cnc(-c2cccc(Cl)c2F)s1]

### A. Ethyl 2-(3-chloro-2-fluorophenyl)thiazole-5-carboxylate

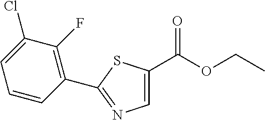 [SMILES: CCOC(=O)c1cnc(-c2cccc(Cl)c2F)s1]

A mixture of ethyl 2-bromothiazole-5-carboxylate (0.5 g, 2.118 mmol), (3-chloro-2-fluorophenyl)boronic acid (0.480 g, 2.75 mmol), 2.0 M aqueous sodium carbonate (3.2 mL, 6.40 mmol) and tetrakis(triphenylphosphine)palladium(0) (0.245 g, 0.212 mmol) in ethanol (7 mL) and toluene (14 mL) was purged with nitrogen for a few minutes. The mixture was then heated at 90° C. in a sealed tube for ˜2 hours. The reaction was then cooled to room temperature and filtered through a pad of Celite®, then washed with ethyl acetate. Aqueous sodium bicarbonate was added and the organic layer was separated and washed with aqueous sodium chloride, dried over sodium sulfate, filtered, and concentrated under reduced pressure. The residue was purified by silica gel chromatography, eluting with ethyl acetate:heptanes (1:19 to 1:6) to give ethyl 2-(3-chloro-2-fluorophenyl)thiazole-5-carboxylate (451 mg, 1.578 mmol, 74.5% yield) as a white solid. ¹H NMR (400 MHz, CD₃SOCD₃) δ 1.34 (t, J=7 Hz, 3H), 4.37 (q, J=7 Hz, 2H), 7.45 (td, J=8, 1 Hz, 1H), 7.84 (td, J=8, 2 Hz, 1H), 8.25 (ddd, J=8, 7, 2 Hz, 1H), 8.64 (d, J=2 Hz, 1H); LC-MS (LC-ES) M+H=286.

### B. 2-(3-Chloro-2-fluorophenyl)thiazole-5-carboxylic Acid

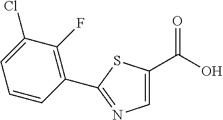 [SMILES: O=C(O)c1cnc(-c2cccc(Cl)c2F)s1]

Lithium hydroxide monohydrate (0.075 g, 3.15 mmol) was added to ethyl 2-(3-chloro-2-fluorophenyl)thiazole-5-carboxylate (0.45 g, 1.575 mmol) in tetrahydrofuran (8.0 mL), ethanol (4.00 mL), and water (4.00 mL). The reaction mixture was allowed to stir at room temperature for 2 hours before concentrating under reduced pressure. The residue was taken up in water and acidified with 6.0 M aqueous hydrochloric acid, upon addition a precipitant formed and was collected by filtration. The solid was washed with water, followed by heptane, to give 2-(3-chloro-2-fluorophenyl)thiazole-5-carboxylic acid (405 mg, 1.572 mmol, 100% yield) as a white solid. ¹H NMR (400 MHz, CD₃SOCD₃) δ 7.45 (td, J=8, 1 Hz, 1H), 7.82 (ddd, J=8, 7, 1 Hz, 1H), 8.25 (ddd, J=8, 7, 1 Hz, 1H), 8.56 (d, J=2 Hz, 1H), 13.79 (br s, 1H); LC-MS (LC-ES) M+H=258.

Intermediate 8

### (S)-2-Bromo-N-(2-oxopyrrolidin-3-yl)thiazole-5-carboxamide

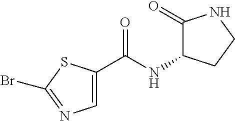 [SMILES: O=C(N[C@H]1CCNC1=O)c1cnc(Br)s1]

N,N-Diisopropylethylamine (0.302 mL, 1.730 mmol) was added to 2-bromothiazole-5-carboxylic acid (0.12 g, 0.577 mmol) and (S)-3-aminopyrrolidin-2-one (0.069 g, 0.692 mmol) in N,N-dimethylformamide (3.0 mL), then n-propylphosphonic acid anhydride (50% in ethyl acetate) (0.687 mL, 1.154 mmol) was added and the reaction mixture was allowed to stir at room temperature for 3 hours. The reaction mixture was concentrated under reduced pressure. The residue was purified by reverse phase HPLC, eluting with acetonitrile:water with 0.1% ammonium hydroxide (1:19 to 19:1) to afford (S)-2-bromo-N-(2-oxopyrrolidin-3-yl)thiazole-5-carboxamide (0.07 g, 0.241 mmol, 41.8% yield). ¹H NMR (400 MHz, CD₃OD) δ 2.10-2.24 (m, 1H), 2.50-2.62 (m, 1H), 3.36-3.48 (m, 2H), 4.68 (t, J=10 Hz, 1H), 8.13 (s, 1H); LC-MS (LC-ES) M+H=290.

Intermediate 9

### 2-(3-(Difluoromethyl)phenyl)thiazole-5-carboxylic Acid

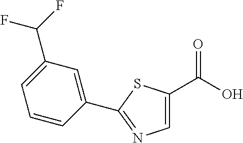 [SMILES: O=C(O)c1cnc(-c2cccc(C(F)F)c2)s1]

### A. Ethyl 2-(3-(difluoromethyl)phenyl)thiazole-5-carboxylate

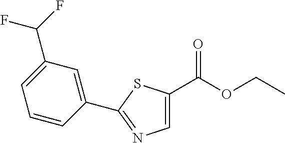 [SMILES: CCOC(=O)c1cnc(-c2cccc(C(F)F)c2)s1]

A 1.3 M solution of isopropylmagnesium chloride lithium chloride complex in tetrahydrofuran (3.20 mL, 4.16 mmol) was added, dropwise, to a solution of 1-(difluoromethyl)-3-iodobenzene (0.919 g, 3.62 mmol) in tetrahydrofuran (4 mL) at −78° C. over −5 minutes. The mixture was stirred 30 minutes and a solution of zinc(II) bromide in tetrahydrofuran (3.71 mL, 4.52 mmol) was added, dropwise. The cooling bath was removed and the mixture was warmed to room temperature.

Tetrakis(triphenylphosphine)palladium(0) (0.209 g, 0.181 mmol) and ethyl 2-bromothiazole-5-carboxylate (0.594 mL, 3.98 mmol) were added and the vial's septum was replaced with a crimp top. The mixture was stirred in a heating block at 70° C. for 17 hours.

Upon cooling, the mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:6) to give ethyl 2-(3-(difluoromethyl)phenyl)thiazole-5-carboxylate (0.872 g, 3.08 mmol, 85% yield) as a pale yellow syrup. ¹H NMR (400 MHz, CD₃SOCD₃) δ 1.32 (t, J=7 Hz, 3H), 4.35 (q, J=7 Hz, 2H), 7.16 (t, J=56 Hz, 1H), 7.70 (t, J=8 Hz, 1H), 7.77 (d, J=8 Hz, 1H), 8.19 (ddd, J=8, 2, 1 Hz, 1H), 8.23 (s, 1H), 8.54 (s, 1H); LC-MS (LC-ES) M+H=284.

### B. 2-(3-(Difluoromethyl)phenyl)thiazole-5-carboxylic Acid

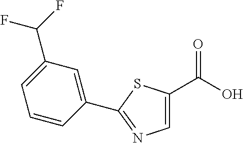 [SMILES: O=C(O)c1cnc(-c2cccc(C(F)F)c2)s1]

Lithium hydroxide monohydrate (0.646 g, 15.39 mmol) was added to a solution of ethyl 2-(3-(difluoromethyl)phenyl)thiazole-5-carboxylate (0.872 g, 3.08 mmol) in tetrahydrofuran (5 mL) and water (0.5 mL) and the reaction mixture was stirred in a heating block at 60° C. in a sealed vial. After 17.5 hours, the mixture was cooled, poured into water (50 mL) and extracted with diethyl ether (1×). The aqueous layer was acidified by addition of 1 M hydrochloric acid (15.4 mL) and the precipitated solids were collected by filtration, washed with water, and dried in a vacuum oven (50° C., 28″ Hg) overnight to afford 2-(3-(difluoromethyl)phenyl)thiazole-5-carboxylic acid (0.738 g, 2.89 mmol, 94% yield) as a colorless solid. ¹H NMR (400 MHz, CD₃SOCD₃) δ 7.16 (t, J=56 Hz, 1H), 7.70 (t, J=8 Hz, 1H), 7.76 (d, J=8 Hz, 1H), 8.14-8.20 (m, 1H), 8.21 (s, 1H), 8.45 (s, 1H), 13.78 (br s, 1H); LC-MS (LC-ES) M+H=256.

Intermediate 10

### (3S,4R)-3-Amino-4-methylpyrrolidin-2-one

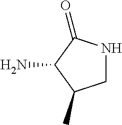

### A. (S)-2-(1,3-Dioxoisoindolin-2-yl)-3-methylbutanoyl Chloride

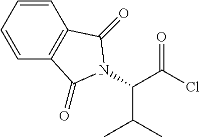 [SMILES: CC(C)[C@@H](C(=O)Cl)N1C(=O)c2ccccc2C1=O]

Thionyl chloride (3.12 mL, 42.8 mmol) was added to (S)-2-(1,3-dioxoisoindolin-2-yl)-3-methylbutanoic acid (10.57 g, 42.8 mmol) in tetrahydrofuran (214 mL) at room temperature and the reaction mixture was stirred for sixteen hours, then concentrated to give crude (S)-2-(1,3-dioxoisoindolin-2-yl)-3-methylbutanoyl chloride (11.36 g, 40.6 mmol, 95% yield), which was carried forward into the next reaction.

### B. (S)-2-(1,3-Dioxoisoindolin-2-yl)-N-(5-methoxyquinolin-8-yl)-3-methylbutanamide

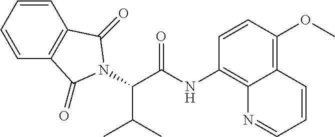 [SMILES: COc1ccc(NC(=O)[C@H](C(C)C)N2C(=O)c3ccccc3C2=O)c2ncccc12]

5-Methoxyquinolin-8-amine hydrochloride (5.57 g, 32.0 mmol) was added to (S)-2-(1,3-dioxoisoindolin-2-yl)-3-methylbutanoyl chloride (8.50 g, 32.0 mmol) in dichloromethane (160 mL) at room temperature. Then, 2,6-lutidine (7.45 mL, 64.0 mmol) was added and the reaction mixture was stirred for sixteen hours, then water was added and the reaction mixture was extracted with dichloromethane, washed with saturated sodium chloride, dried over magnesium sulfate, filtered, and concentrated. The reaction mixture was purified by silica gel chromatography, eluting with ethyl acetate:hexanes (3:7), then further purified by reverse phase HPLC, eluting with acetonitrile:water with 0.1% ammonium hydroxide (20:80 to 100:0) to give (S)-2-(1,3-dioxoisoindolin-2-yl)-N-(5-methoxyquinolin-8-yl)-3-methylbutanamide (9.47 g, 22.30 mmol, 69.7% yield). ¹H NMR (400 MHz, CD₃SOCD₃) δ 0.90 (d, J=7 Hz, 3H), 1.11 (d, J=7 Hz, 3H), 2.92-3.06 (m, 1H), 3.95 (s, 3H), 4.72 (d, J=10 Hz, 1H), 7.03 (d, J=9 Hz, 1H), 7.59 (dd, J=8, 4 Hz, 1H), 7.84-7.96 (m, 4H), 8.40 (d, J=9 Hz, 1H), 8.54 (d, J=8 Hz, 1H), 8.86 (d, J=4 Hz, 1H), 10.20 (s, 1H); LC-MS (LC-ES) M+H=404.

### C. 2-((3S,4R)-4-Methyl-2-oxopyrrolidin-3-yl)isoindoline-1,3-dione

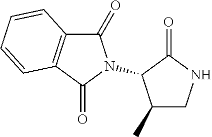 [SMILES: C[C@@H]1CNC(=O)[C@H]1N1C(=O)c2ccccc2C1=O]

Iodobenzene diacetate (18.90 g, 58.7 mmol) was added to (S)-2-(1,3-dioxoisoindolin-2-yl)-N-(5-methoxyquinolin-8-yl)-3-methylbutanamide (9.47 g, 23.47 mmol) in toluene (235 mL) at room temperature and the reaction mixture was purged with nitrogen. Then, palladium(II) acetate (0.264 g, 1.174 mmol) was added and the reaction mixture was heated to 110° C. and stirred for five hours. The reaction mixture was cooled and concentrated. The resulting residue was purified by silica gel chromatography, eluting with acetone:hexanes (2:3) to give 8-((3S,4R)-3-(1,3-dioxoisoindolin-2-yl)-4-methyl-2-oxopyrrolidin-1-yl)-5-methoxyquinolin-7-yl acetate with 2-((3S,4R)-1-(5-methoxyquinolin-8-yl)-4-methyl-2-oxopyrrolidin-3-yl)isoindoline-1,3-dione (5.96 g, 6.92 mmol, 29.5% yield), which was carried forward to the next reaction. Ceric ammonium nitrate (22.77 g, 41.5 mmol) was added to 8-((3S,4R)-3-(1,3-dioxoisoindolin-2-yl)-4-methyl-2-oxopyrrolidin-1-yl)-5-methoxyquinolin-7-yl acetate with 2-((3S,4R)-1-(5-methoxyquinolin-8-yl)-4-methyl-2-oxopyrrolidin-3-yl)isoindoline-1,3-dione (5.96 g, 6.92 mmol) in acetonitrile (58 mL) and water (12 mL) at room temperature and the reaction mixture was stirred for sixteen hours. The reaction mixture was extracted with ethyl acetate, washed with saturated sodium chloride, dried over magnesium sulfate, filtered, and concentrated. The residue was purified by reverse phase HPLC, eluting with acetonitrile:water with 0.1% ammonium hydroxide (5:95 to 100:0) to give 2-((3S,4R)-4-methyl-2-oxopyrrolidin-3-yl)isoindoline-1,3-dione (0.6383 g, 2.483 mmol, 35.9% yield). ¹H NMR (400 MHz, CD₃SOCD₃) δ 1.07 (d, J=7 Hz, 3H), 2.76-2.90 (m, 1H), 2.94 (t, J=9 Hz, 1H), 3.43 (t, J=9 Hz, 1H), 4.42 (d, J=9 Hz, 1H), 7.84-7.94 (m, 4H), 8.02 (br s, 1H); LC-MS (LC-ES) M+H=245.

### D. (3S,4R)-3-Amino-4-methylpyrrolidin-2-one

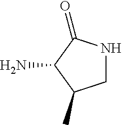

Hydrazine (0.116 mL, 3.69 mmol) was added to 2-((3S,4R)-4-methyl-2-oxopyrrolidin-3-yl)isoindoline-1,3-dione (0.3001 g, 1.229 mmol) in ethanol (12.3 mL) at room temperature and the reaction mixture was stirred for sixteen hours at reflux. The reaction mixture was concentrated. The resulting residue was purified by silica gel chromatography, eluting with methanol:dichloromethane (1:9 to 1:4) with 1% ammonium hydroxide to give (3S,4R)-3-amino-4-methylpyrrolidin 2-one (0.1240 g, 1.032 mmol, 84% yield). ¹H NMR (400 MHz, CD₃SOCD₃) δ 1.06 (d, J=7 Hz, 3H), 1.66 (br s, 2H), 1.82-1.96 (m, 1H), 2.68 (t, J=9 Hz, 1H), 2.75 (d, J=10 Hz, 1H), 3.19 (dt, J=9, 2 Hz, 1H), 7.54 (br s, 1H); LC-MS (LC-ES) M+H=115.

Intermediate 11

### 2-(3-(Difluoromethyl)-5-fluorophenyl)thiazole-5-carboxylic Acid

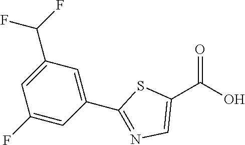 [SMILES: O=C(O)c1cnc(-c2cc(F)cc(C(F)F)c2)s1]

### A. 1-(Difluoromethyl)-3-fluoro-5-iodobenzene

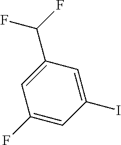 [SMILES: Fc1cc(I)cc(C(F)F)c1]

(Diethylamino)sulfur trifluoride (2.142 mL, 16.21 mmol) was added to a solution of 3-fluoro-5-iodobenzaldehyde (1.93 g, 7.72 mmol) in dichloromethane (24 mL) at 0° C., dropwise. The resulting deep yellow solution was stirred in the ice bath under nitrogen, gradually warming to room temperature. LC-MS after 24 hours indicated complete conversion to product. The mixture was cooled in an ice bath, quenched by dropwise addition of saturated sodium bicarbonate, poured into water and extracted with dichloromethane (3×). The combined organics were washed with water and saturated sodium chloride, dried over sodium sulfate, and concentrated in vacuo. The residue was purified by silica gel chromatography, eluting with ethyl acetate:hexanes (0:1 to 1:3) to give 1-(difluoromethyl)-3-fluoro-5-iodobenzene (1.59 g, 5.85 mmol, 76% yield) as a colorless liquid. ¹H NMR (400 MHz, CDCl₃) δ 6.58 (t, J=56 Hz, 1H), 7.21 (d, J=8 Hz, 1H), 7.56 (dd, J=8, 1 Hz, 1H), 7.65 (s, 1H).

### B. Ethyl 2-(3-(difluoromethyl)-5-fluorophenyl)thiazole-5-carboxylate

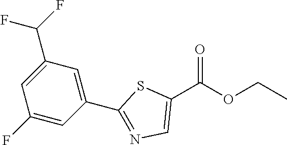 [SMILES: CCOC(=O)c1cnc(-c2cc(F)cc(C(F)F)c2)s1]

A 1.3 M solution of isopropylmagnesium chloride lithium chloride complex in tetrahydrofuran (4.59 mL, 5.97 mmol) was added to a solution of 1-(difluoromethyl)-3-fluoro-5-iodobenzene (1.476 g, 5.43 mmol) in tetrahydrofuran (20 mL) at −75° C. dropwise over 5 minutes. The resulting yellow solution was stirred for 30 minutes at −75° C., then transferred to an ice bath. After 2 hours, a solution of zinc(II) bromide in tetrahydrofuran (3.90 mL, 6.51 mmol) was added, dropwise, and the cooling bath was removed. Tetrakis(triphenylphosphine)palladium(0) (0.314 g, 0.271 mmol) and ethyl 2-bromothiazole-5-carboxylate (0.811 mL, 5.43 mmol) were added and the reaction vessel's septum was replaced with a teflon bushing. The mixture was stirred in a heating block at 80° C. After 3 hours, the mixture was cooled, poured into saturated ammonium chloride and extracted with ethyl acetate (3×). the combined organics were washed with water and saturated sodium chloride, dried over sodium sulfate, and concentrated in vacuo. The residue was purified by silica gel chromatography, eluting with ethyl acetate:hexanes (0:1 to 1:3) to give ethyl 2-(3-(difluoromethyl)-5-fluorophenyl)thiazole-5-carboxylate (1.03 g, 3.42 mmol, 63.0% yield) as a colorless solid. ¹H NMR (400 MHz, CD₃SOCD₃) δ 1.42 (t, J=7 Hz, 3H), 4.42 (q, J=7 Hz, 2H), 6.70 (t, J=56 Hz, 1H), 7.35 (dd, J=8, 1 Hz, 1H), 7.83 (dt, J=9, 2 Hz, 1H), 7.92 (s, 1H), 8.45 (s, 1H); LC-MS (LC-ES) M+H=302.

### C. 2-(3-(Difluoromethyl)-5-fluorophenyl)thiazole-5-carboxylic Acid

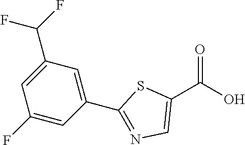 [SMILES: O=C(O)c1cnc(-c2cc(F)cc(C(F)F)c2)s1]

Lithium hydroxide monohydrate (0.717 g, 17.09 mmol) was added to a solution of ethyl 2-(3-(difluoromethyl)-5-fluorophenyl)thiazole-5-carboxylate (1.03 g, 3.42 mmol) in tetrahydrofuran (5 mL) and water (0.5 mL) are fraction vial was sealed with a screw cap and the mixture was stirred in a heating block at 50° C. After 4 hours, the mixture was diluted with water and extracted with diethyl ether. The aqueous layer was acidified by addition of 1 M hydrochloric acid (17.5 mL). The mixture was stirred −15 minutes and the precipitated solids were collected by filtration. The filter cake was dried in a vacuum oven (50° C./27″ Hg) to afford 2-(3-(difluoromethyl)-5-fluorophenyl)thiazole-5-carboxylic acid (0.875 g, 3.20 mmol, 94% yield) as a colorless solid. ¹H NMR (400 MHz, CD₃SOCD₃) δ 7.16 (t, J=55 Hz, 1H), 7.66 (dd, J=9, 1 Hz, 1H), 8.03 (dt, J=9, 1 Hz, 1H), 8.07 (d, J=1 Hz, 1H), 8.47 (s, 1H), 13.81 (br s, 1H); LC-MS (LC-ES) M+H=274.

Intermediate 12

### 3-Amino-5,5-dimethylpyrrolidin-2-one Hydrochloride

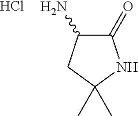

### A. 2-(((Benzyloxy)carbonyl)amino)-2-methylpropanoic Acid

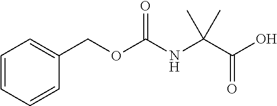 [SMILES: CC(C)(NC(=O)OCc1ccccc1)C(=O)O]

Triethylamine (81 mL, 582 mmol) and benzyl (2,5-dioxopyrrolidin-1-yl) carbonate (145 g, 582 mmol) in acetonitrile (600 mL) were added dropwise to a solution of 2-amino-2-methylpropanoic acid (60 g, 582 mmol) in water (480 mL) at 0° C. under argon. The resulting reaction mixture was stirred for 16 hours at room temperature. On completion, the solvent was evaporated under reduced pressure. The reaction mixture was diluted with aqueous sodium bicarbonate (500 mL) and washed with diethyl ether (300 mL, 3×). The aqueous layer was acidified with 1 M aqueous potassium bisulphate (pH up to ˜3, 1000 mL) and the compound was extracted with ethyl acetate (500 mL, 3×). The combined organic layers were washed with brine (300 mL) and evaporated under reduced pressure to give a crude material, which was purified by silica column chromatography, eluting with ethyl acetate:petroleum ether (1:2) to afford 2-(((benzyloxy)carbonyl)amino)-2-methylpropanoic acid (85 g, 51.1% yield) as an white solid. ¹H NMR (400 MHz, CD₃SOCD₃) δ 1.34 (s, 6H), 5.00 (s, 2H), 7.25-7.41 (m, 5H), 7.51 (br s, 1H), 12.25 (br s, 1H); LC-MS (LC-ES) M+H=224.

### B. Ethyl 2-(((benzyloxy)carbonyl)amino)-2-methylpropanoate

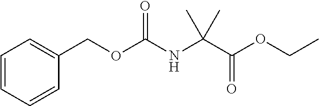 [SMILES: CCOC(=O)C(C)(C)NC(=O)OCc1ccccc1]

p-Toluenesulfonic acid monohydrate (6.65 g, 35.0 mmol) was added portion wise to a solution of 2-(((benzyloxy)carbonyl)amino)-2-methylpropanoic acid (100 g, 350 mmol) in toluene (1000 mL) and ethanol (100 mL) and the resulting reaction mixture was stirred at 80° C. for 15 hours. On completion, the solvent was evaporated under vacuum and dissolved in ethyl acetate (1000 mL), washed with saturated sodium bicarbonate (500 mL, 2×) and saturated sodium chloride (300 mL), and concentrated to afford ethyl 2-(((benzyloxy)carbonyl)amino)-2-methylpropanoate (90 g, 93% yield) as colorless liquid. ¹H NMR (400 MHz, CDCl₃) δ 1.24 (t, J=7 Hz, 3H), 1.55 (s, 6H), 4.06-4.29 (m, 2H), 5.08 (s, 2H), 5.39 (br s, 1H), 7.27-7.45 (m, 5H); LC-MS (LC-ES) M+H=266.

### C. Benzyl (1-hydroxy-2-methylpropan-2-yl)carbamate

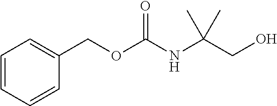 [SMILES: CC(C)(CO)NC(=O)OCc1ccccc1]

Diisobutylaluminum hydride (870 mL, 870 mmol) was added to a solution of ethyl 2-(((benzyloxy)carbonyl)amino)-2-methylpropanoate (80 g, 290 mmol) in toluene (1500 mL) at −78° C. under argon. The reaction mixture was stirred for 2 hours at −78° C. On completion, the reaction mixture was quenched with saturated aqueous Rochelle's salt solution (1000 mL) dropwise at 0° C. and stirring was continued for 1 hour. The resulting solid was filtered through Celite® and the filtrate was extracted with diethyl ether (1000 mL, 3×). The combined layers were washed with brine (300 mL), and evaporated under reduced pressure to afford benzyl (1-hydroxy-2-methylpropan-2-yl)carbamate (65 g, 80% yield) as viscous liquid. ¹H NMR (400 MHz, CD₃SOCD₃) δ 1.18 (s, 6H), 3.35 (d, J=6 Hz, 2H), 4.68 (t, J=6 Hz, 1H), 4.97 (s, 2H), 6.68 (br s, 1H), 7.26-7.42 (m, 5H); LC-MS (LC-ES) M+H=224.

Alternative Method

### Benzyl (1-hydroxy-2-methylpropan-2-yl)carbamate

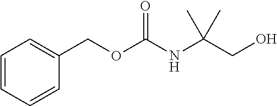 [SMILES: CC(C)(CO)NC(=O)OCc1ccccc1]

A 2 L Morton flask equipped with overhead stirring was charged with dichloromethane (200 mL), 2-amino-2-methylpropan-1-ol (10.71 mL, 112 mmol) and a saturated solution of sodium bicarbonate (200 mL) and the reaction mixture was stirred. Benzyl chloroformate (16.61 mL, 118 mmol) was added rapidly via syringe to the reaction mixture and stirring was continued (¹H NMR after 6 hours indicated complete conversion). The mixture was poured into a separatory funnel and the layers were separated. The organic layer was washed with water and saturated sodium chloride, dried over sodium sulfate, and concentrated in vacuo to afford a colorless liquid (23.5 g). This liquid was purified by silica gel chromatography, eluting with ethyl acetate:heptane (1:9 to 1:1) to afford benzyl (1-hydroxy-2-methylpropan-2-yl)carbamate (13.61 g, 61.0 mmol, 54.3% yield) as a colorless syrup. ¹H NMR (400 MHz, CD₃SOCD₃) δ 1.16 (s, 6H), 3.35 (d, J=6 Hz, 2H), 4.69 (t, J=6 Hz, 1H), 4.97 (s, 2H), 6.69 (br s, 1H), 7.26-7.42 (m, 5H); LC-MS (LC-ES) M+H=224.

### D. Benzyl (2-methyl-1-oxopropan-2-yl)carbamate

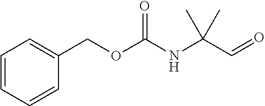 [SMILES: CC(C)(C=O)NC(=O)OCc1ccccc1]

Pyridiniumdichromate (271 g, 720 mmol) was added portionwise to a solution of benzyl (1-hydroxy-2-methylpropan-2-yl)carbamate (65 g, 233 mmol) and molecular sieves (80 g, 233 mmol) in dichloromethane (1.3 L) at 27° C. and the reaction mixture was stirred for 15 hours. On completion, the reaction mixture was filtered over Celite® and the filtrate was evaporated under reduced pressure to give an impure material, which was purified by silica column chromatography, eluting with ethyl acetate:petroleum ether (1:3) to afford benzyl (2-methyl-1-oxopropan-2-yl)carbamate (30 g, 46.6% yield) as white solid. ¹H NMR (400 MHz, CDCl₃) δ 1.38 (s, 6H), 5.09 (s, 2H), 5.21-5.43 (m, 1H), 7.26-7.42 (m, 5H), 9.43 (s, 1H); LC-MS (LC-ES) M+H=222.

Alternative Method

### Benzyl (2-methyl-1-oxopropan-2-yl)carbamate

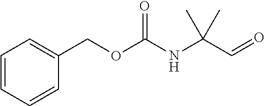 [SMILES: CC(C)(C=O)NC(=O)OCc1ccccc1]

A 1 L flask with stir bar was charged with benzyl (1-hydroxy-2-methylpropan-2-yl)carbamate (12.36 g, 55.4 mmol) and dichloromethane (250 mL) and cooled in an ice bath. 2,2,6,6-Tetramethylpiperidin-1-yl)oxyl (TEMPO) (0.432 g, 2.77 mmol) was added, followed by a solution of potassium chloride (2.77 mL, 5.54 mmol). Sodium bicarbonate (2.79 g, 33.2 mmol) was dissolved in a commercial solution of sodium hypochlorite (69.2 mL, 83 mmol). The resulting solution was added to the reaction mixture and stirring continued at 0° C. (TLC after 30 minutes indicated consumption of starting material). The reaction mixture was quenched by addition of saturated sodium thiosulfate and saturated sodium bicarbonate, extracted with dichloromethane (2×), washed with water and saturated sodium chloride, dried over sodium sulfate, and concentrated. The residue was purified by flash chromatography, eluting with ethyl acetate:heptane (1:9 to 2:3) to afford benzyl (2-methyl-1-oxopropan-2-yl)carbamate (11.06 g, 50.0 mmol, 90% yield) as a colorless solid. ¹H NMR (400 MHz, CD₃SOCD₃) δ 1.17 (s, 6H), 5.03 (s, 2H), 7.28-7.42 (m, 5H), 7.87 (br s, 1H), 9.36 (s, 1H); LC-MS (LC-ES) M+H=222.

### E. (Z)-Methyl 4-(((benzyloxy)carbonyl)amino)-2-((tert-butoxycarbonyl)amino)-4-methylpent-2-enoate

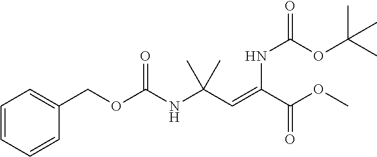 [SMILES: COC(=O)/C(=C/C(C)(C)NC(=O)OCc1ccccc1)NC(=O)OC(C)(C)C]

Methyl 2-((tert-butoxycarbonyl)amino)-2-(dimethoxyphosphoryl)acetate (56.4 g, 190 mmol) and 1,8-diazabicyclo[5.4.0]undec-7-ene (0.026 L, 170 mmol) were added to a solution of benzyl (2-methyl-1-oxopropan-2-yl)carbamate (25 g, 90 mmol) in dichloromethane (1 L) at 27° C. and the reaction mixture was stirred for 15 hours under argon. On completion, the reaction mixture was quenched with saturated aqueous ammonium chloride solution (200 mL), extracted with dichloromethane (500 mL, 2×), washed with brine (400 mL), and evaporated under reduced pressure to give an impure material, which was purified by silica column chromatography, eluting with ethyl acetate:petroleum ether (1:2) to afford (Z)-methyl 4-(((benzyloxy)carbonyl)amino)-2-((tert-butoxycarbonyl)amino)-4-methylpent-2-enoate (32 g, 90% yield) as light yellow liquid. ¹H NMR (400 MHz, CDCl₃) δ 1.43 (s, 6H), 1.47-1.60 (m, 9H), 3.77 (s, 3H), 5.05 (s, 2H), 6.29 (br s, 1H), 6.56 (br s, 1H), 7.30-7.41 (m, 5H); LC-MS (LC-ES) M+H=391.

Alternative Method

### (Z)-Methyl 4-(((benzyloxy)carbonyl)amino)-2-((tert-butoxycarbonyl)amino)-4-methylpent-2-enoate

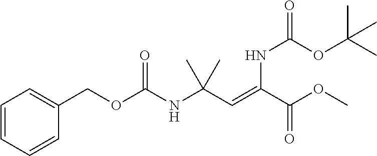 [SMILES: COC(=O)/C(=C/C(C)(C)NC(=O)OCc1ccccc1)NC(=O)OC(C)(C)C]

A 1 L flask with stir bar was charged with benzyl (2-methyl-1-oxopropan-2-yl)carbamate (11.86 g, 53.6 mmol), dichloromethane (200 mL), methyl 2-((tert-butoxycarbonyl)amino)-2-(dimethoxyphosphoryl)acetate (33.5 g, 113 mmol) and 1,8-diazabicyclo[5.4.0]undec-7-ene (16.00 mL, 107 mmol). The resulting pale yellow solution was stirred at room temperature under nitrogen (LCMS after 1 hour indicated consumption of aldehyde starting material). The reaction was quenched by addition of saturated ammonium chloride (200 mL). The layers were separated, and the aqueous layer was extracted with dichloromethane. The combined organics were washed with water and saturated sodium chloride, dried over sodium sulfate, and concentrated. The residue was purified by flash chromatography, eluting with ethyl acetate:heptane (1:9 to 1:1) to afford methyl (2)-4-(((benzyloxy)carbonyl)amino)-2-((tert-butoxycarbonyl)amino)-4-methylpent-2-enoate (14.24 g, 36.3 mmol, 67.7% yield) as a colorless gum. LC-MS (LC-ES) M+Na=415.

### F. tert-Butyl (5,5-dimethyl-2-oxopyrrolidin-3-yl)carbamate

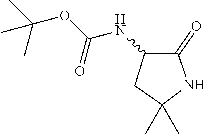 [SMILES: CC1(C)CC(NC(=O)OC(C)(C)C)C(=O)N1]

10% Palladium on carbon (15.62 g, 14.68 mmol) was added to a solution of (Z)-methyl 4-(((benzyloxy)carbonyl)amino)-2-((tert-butoxycarbonyl)amino)-4-methylpent-2-enoate (32 g, 82 mmol) in methanol (320 mL) at 27° C. under hydrogen. The reaction mixture was stirred for 4 hours. On completion, the reaction mixture was filtered over Celite® and the filtrate was evaporated under reduced pressure to obtain tert-butyl (5,5-dimethyl-2-oxopyrrolidin-3-yl)carbamate (19 g, 69.2% yield) as off white solid. ¹H NMR (400 MHz, CD₃SOCD₃) δ 1.14 (s, 3H), 1.18 (s, 3H), 1.38 (s, 9H), 1.67 (t, J=11 Hz, 1H), 2.12 (dd, J=12, 9 Hz, 1H), 4.09-4.22 (m, 1H), 6.95 (br s, 1H), 7.82 (br s, 1H); LC-MS (LC-ES) M+H=229.

Alternative Method

### tert-Butyl (5,5-dimethyl-2-oxopyrrolidin-3-yl)carbamate

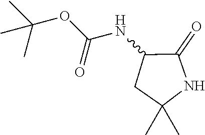 [SMILES: CC1(C)CC(NC(=O)OC(C)(C)C)C(=O)N1]

A 1000 mL flask with stir bar was charged with methyl (Z)-4-(((benzyloxy)carbonyl)amino)-2-((tert-butoxycarbonyl)amino)-4-methylpent-2-enoate (14.14 g, 36.0 mmol) and methanol (150 mL). The flask was stoppered and flushed with nitrogen. Then, 10% palladium on carbon (1.917 g, 1.801 mmol) was added and the flask was evacuated/backfilled with hydrogen (3×). The reaction mixture was stirred under a balloon of hydrogen (¹H NMR after 17 hours indicated complete conversion). The reaction mixture was filtered through a pad of Celite® and the filtrate was concentrated in vacuo. The residue was purified by silica gel chromatography, eluting with (ethanol:ethyl acetate (3:1):heptane (1:4 to 9:11) to afford tert-butyl (5,5-dimethyl-2-oxopyrrolidin-3-yl)carbamate (6.22 g, 27.2 mmol, 76% yield) as a colorless solid. ¹H NMR (400 MHz, CD₃SOCD₃) δ 1.15 (s, 3H), 1.18 (s, 3H), 1.38 (s, 9H), 1.66 (t, J=11 Hz, 1H), 2.11 (dd, J=12, 9 Hz, 1H), 4.10-4.24 (m, 1H), 6.99 (br d, J=9 Hz, 1H), 7.85 (br s, 1H).

### G. 3-Amino-5,5-dimethylpyrrolidin-2-one Hydrochloride

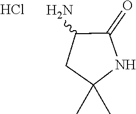

Hydrochloric acid in 1,4-dioxane (4 M, 95 mL, 380 mmol) was added to a stirred solution of tert-butyl (5,5-dimethyl-2-oxopyrrolidin-3-yl)carbamate (19 g, 82 mmol) in dichloromethane (38 mL) at 0° C. under argon and the resulting reaction mixture was stirred for 6 hours. On completion, the reaction mixture was evaporated under reduced pressure to give an impure material, which was washed with diethyl ether (50 mL) to afford 3-amino-5,5-dimethylpyrrolidin-2-one hydrochloride (12.85 g, 78 mmol, 95% yield) as off white solid. ¹H NMR (400 MHz, CD₃SOCD₃) δ 1.21 (s, 3H), 1.25 (s, 3H), 1.81 (dd, J=11, 12 Hz, 1H), 2.26-2.35 (m, 1H), 4.06 (br s, 1H), 8.43 (br s, 4H); LC-MS (LC-ES) M+H=129.

Alternative Method

### 3-Amino-5,5-dimethylpyrrolidin-2-one Hydrochloride

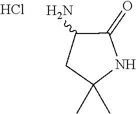

A 500 mL flask with stir bar was charged with tert-butyl (5,5-dimethyl-2-oxopyrrolidin-3-yl)carbamate (5.85 g, 25.6 mmol), dichloromethane (100 mL) and a 4 M solution of hydrochloric acid in dioxane (32.0 mL, 128 mmol). The mixture was stirred at room temperature. After 6 hours, the mixture was concentrated in vacuo. The solids were slurried in methyl tert-butyl ether, collected by filtration, and dried on a Buchner funnel overnight to afford 3-amino-5,5-dimethylpyrrolidin-2-one hydrochloride (4.60 g, 25.2 mmol, 98% yield) as a colorless solid. ¹H NMR (400 MHz, CD₃SOCD₃) δ 1.20 (s, 3H), 1.24 (s, 3H), 1.87 (dd, J=11, 12 Hz, 1H), 2.27 (dd, J=12, 9 Hz, 1H), 4.03 (t, J=9 Hz, 1H), 8.42 (br s, 3H), 8.59 (br s, 1H); LC-MS (LC-ES) M+H=129.

Intermediate 13

### (S)-3-Amino-5,5-dimethylpyrrolidin-2-one Hydrochloride

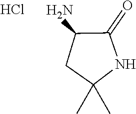

### A. Methyl (tert-butoxycarbonyl)-L-leucinate

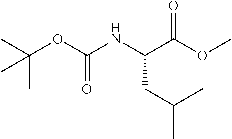 [SMILES: COC(=O)[C@H](CC(C)C)NC(=O)OC(C)(C)C]

Sodium bicarbonate (65.90 g, 784 mmol) was added portion-wise to methyl L-leucinate hydrochloride (71.11 g, 391 mmol) in water (700 mL). When the reaction mixture was no longer bubbling, a solution of di-tert-butyl dicarbonate (94.57 g, 433 mmol) in 1,4-dioxane (140 mL) was added over 97 minutes. After stirring ˜21 hours, diethyl ether (250 mL) and hexanes (250 mL) were added and the layers were separated. The aqueous layer was extracted once more with diethyl ether:hexanes (1:1, 250 mL). The combined organics were dried over magnesium sulfate, filtered, concentrated, and dried under vacuum to give the methyl (tert-butoxycarbonyl)-L-leucinate (99.50 g, 391 mmol, 100% yield assuming 96.5% pure by weight) as a very pale tan liquid. ¹H NMR (400 MHz, CD₃SOCD₃) δ 0.82 (d, J=6 Hz, 3H), 0.85 (d, J=6 Hz, 3H), 1.34-1.44 (m, 1H), 1.36 (s, 9H), 1.46-1.66 (m, 2H), 3.59 (s, 3H), 3.92-4.00 (m, 1H), 7.24 (d, J=8 Hz, 1H); LC-MS (LC-ES) M+H=268.

### B. Methyl N-(N-((benzyloxy)carbonyl)sulfamoyl)-N-(tert-butoxycarbonyl)-L-leucinate

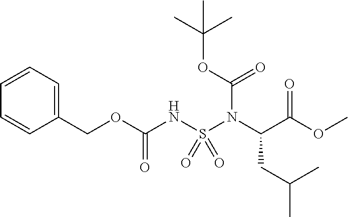 [SMILES: COC(=O)[C@H](CC(C)C)N(C(=O)OC(C)(C)C)S(=O)(=O)NC(=O)OCc1ccccc1]

Benzyl alcohol (125 mL, 1208 mmol) in dichloromethane (100 mL) was added by addition funnel over ˜72 minutes to chlorosulfonyl isocyanate (111 mL, 1275 mmol) in dichloromethane (500 mL) under nitrogen and cooled in an ice-salt bath. After −5 minutes, the ice bath was removed. After an additional 25 minutes the ice bath was put back on. After ˜10 minutes a solution of the methyl (tert-butoxycarbonyl)-L-leucinate (99.50 g, 391 mmol, 96.5% by weight) and triethylamine (180 mL, 1291 mmol) in dichloromethane (180 mL) was added by addition funnel over ˜51 minutes. After ˜5 minutes, the ice bath was removed and the reaction was allowed to stir at ambient temperature for 7 days. The reaction mixture was diluted into diethyl ether (2 L) and the mixture was stirred for several minutes. The upper layer was decanted from the grungy lower layer. The grungy lower layer was extracted again with diethyl ether (2 L). The upper layer was again decanted from the grungy residue layer and combined with the first organic extracts. Celite® was added and the mixture was filtered. The filtration concentrated to a solid/slurry. Enough diethyl ether was added to make a filterable slurry. The cream-colored solids were filtered off from the yellow liquid and rinsed with a little diethyl ether. The solids were partially air-dried then dried under vacuum overnight to give the first crop of methyl N-(N-((benzyloxy)carbonyl)sulfamoyl)-N-(tert-butoxycarbonyl)-L-leucinate (103.76 g, 220 mmol, 56% yield assuming 97% pure by weight with the remainder as triethylamine hydrochloride) as a tan/cream-colored powder. The filtrate was partially evaporated and additional solids formed. The solids were filtered off and rinsed twice with diethyl ether. The solids were partially air-dried then dried under vacuum overnight to give the second crop of methyl N-(N-((benzyloxy)carbonyl)sulfamoyl)-N-(tert-butoxycarbonyl)-L-leucinate (18.71 g, 37.1 mmol, 9% yield assuming 95% pure by weight with the remainder as triethylamine hydrochloride) as a cream-colored powder. Some of the material from the first crop of product (58.09 g) and the material from the second crop (18.71 g) were combined and triturated/stirred vigorously with 400 mL water. The solids were filtered off and rinsed with water. The solids were air-dried overnight to give the third crop of methyl N-(N-((benzyloxy)carbonyl)sulfamoyl)-N-(tert-butoxycarbonyl)-L-leucinate (68.77 g, 150 mmol, 38% yield) as a cream-colored powder. ¹H NMR (400 MHz, CD₃SOCD₃) δ 0.88 (d, J=6 Hz, 3H), 0.89 (d, J=6 Hz, 3H), 1.38 (s, 9H), 1.66-1.76 (m, 2H), 1.86-1.96 (m, 1H), 3.56 (s, 3H), 4.80 (t, J=7 Hz, 1H), 5.08-5.21 (ABq, J_{AB}=12 Hz, Δv_{AB}=35 Hz, 2H), 7.30-7.40 (m, 5H), 12.45 (br s, 1H); LC-MS (LC-ES) M+H=459.

Alternative Method

### Methyl N-(N-((benzyloxy)carbonyl)sulfamoyl)-N-(tert-butoxycarbonyl)-L-leucinate

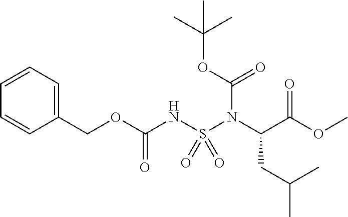 [SMILES: COC(=O)[C@H](CC(C)C)N(C(=O)OC(C)(C)C)S(=O)(=O)NC(=O)OCc1ccccc1]

A 2 L Morton flask equipped with overhead stirring and internal temperature probe was charged with dichloromethane (250 mL) and chlorosulfonyl isocyanate (51.4 mL, 591 mmol) and cooled in an ice-salt bath under nitrogen. A solution of benzyl alcohol (63.9 g, 591 mmol) in dichloromethane (50 mL) was dropping addition funnel at a rate such that the internal temperature remained <5° C. The cooling bath was removed, and the solution was stirred at room temperature for about 1 hour. The solution was cooled in an ice-salt bath. A solution of methyl (tert-butoxycarbonyl)-L-leucinate (48.29 g, 197 mmol, AstaTech) and triethylamine (88 mL, 630 mmol) in dichloromethane (50 mL) was added at a rate such that the internal temperature remained <5° C. The cooling bath was removed, and the solution was stirred at room temperature. After 168 hours, the thick solution was extracted with diethyl ether (3×). The combined organics were washed with water and saturated sodium chloride, dried over sodium sulfate, filtered, and concentrated in vacuo to afford about 95 g of crude material as a colorless, waxy solid. The solid was dissolved in dichloromethane (240 mL) and purified batch wise by silica gel chromatography, eluting with (3:1 ethyl acetate:ethanol):heptane (1:6 to 1:3) to give methyl N-(N-((benzyloxy)carbonyl)sulfamoyl)-N-(tert-butoxycarbonyl)-L-leucinate (56.5 g, 123 mmol, 62.6% yield). LC-MS (LC-ES) M+Na=481.

### C. Methyl N-(tert-butoxycarbonyl)-N-sulfamoyl-L-leucinate

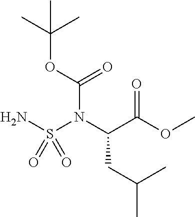 [SMILES: COC(=O)[C@H](CC(C)C)N(C(=O)OC(C)(C)C)S(N)(=O)=O]

Pearlman's catalyst (palladium hydroxide) (1.140 g) slurried in a minimal amount of water was added to methyl N-(N-((benzyloxy)carbonyl)sulfamoyl)-N-(tert-butoxycarbonyl)-L-leucinate (15.16 g, 33.06 mmol) in methanol (150 mL) and nitrogen was bubbled through the reaction mixture for ˜10 minutes. 1,4-Cyclohexadiene (30 mL, 317 mmol) was added and the nitrogen was bubbled for about another minute. The reaction mixture was heated at 60° C. After ˜20 minutes, additional Pearlman's catalyst (1.117 g) was added. The reaction began to bubble after ˜15 minutes. After ˜10 minutes, additional 1,4-cyclohexadiene (15 mL, 159 mmol) was added and the reaction began to bubble again almost immediately. After ˜10 minutes, the heating was stopped. Celite® was added to the reaction and the mixture was filtered over a pad of Celite® and rinsed with methanol, then water. Six reaction filtrates (˜247 mmol total) treated similarly were combined for the purification. The combined filtrates were concentrated. Methanol was added and the mixture was concentrated again. Dichloromethane was added to the residue and the cloudy mixture was filtered. The filtrate was absorbed onto silica gel and purified by silica gel chromatography, eluting with (3:1 ethyl acetate:ethanol):hexanes (0:1 to 1:4) to give a residue. The residue was dissolved in ethyl acetate and filtered over a bit of cotton. The mixture was concentrated to an oil that solidified to a waxy solid. The solid was scraped and dried under vacuum to give methyl N-(tert-butoxycarbonyl)-N-sulfamoyl-L-leucinate (55.71 g, 172 mmol, 70% yield) as a tan waxy solid. ¹H NMR (400 MHz, CD₃SOCD₃) δ 0.88 (d, J=7 Hz, 3H), 0.89 (d, J=7 Hz, 3H), 1.41 (s, 9H), 1.58-1.70 (m, 1H), 1.72-1.88 (m, 2H), 3.64 (s, 3H), 4.80 (dd, J=9, 6 Hz, 1H), 7.50 (s, 2H); LC-MS (LC-ES) M+Na=347.

Alternative Method

### Methyl N-(tert-butoxycarbonyl)-N-sulfamoyl-L-leucinate

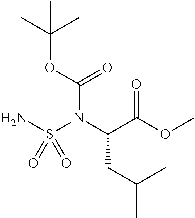 [SMILES: COC(=O)[C@H](CC(C)C)N(C(=O)OC(C)(C)C)S(N)(=O)=O]

A 1000 mL flask with stir bar was charged with methyl N-(N-((benzyloxy)carbonyl)sulfamoyl)-N-(tert-butoxycarbonyl)-L-leucinate (51.84 g, 113 mmol) and methanol (300 mL) and stirred for about 15 minutes. Ethyl acetate (50 mL) was added to effect complete dissolution. Then, Pearlman's catalyst (palladium hydroxide on carbon, 1.985 g, 2.83 mmol) was added and the flask was evacuated and backfilled with nitrogen (3×) and then hydrogen (3×). The mixture was stirred at room temperature under a balloon of hydrogen. ¹H NMR, after 4 hours, indicated a 3:1 mixture of product:starting material. The balloon was recharged with hydrogen and the reaction mixture was stirred overnight. The reaction was complete after 22 hours. The catalyst was removed by filtration through a pad of Celite® and the filtrate was concentrated in vacuo. The residue was diluted with dichloromethane (100 mL) and purified batch wise by silica gel chromatography, eluting with (3:1 ethyl acetate:ethanol):heptane (1:9 to 1:3) to give methyl N-(tert-butoxycarbonyl)-N-sulfamoyl-L-leucinate (29.26 g, 90 mmol, 80% yield) as a colorless solid. ¹H NMR (400 MHz, CD₃SOCD₃) δ 0.89 (d, J=7 Hz, 3H), 0.90 (d, J=7 Hz, 3H), 1.42 (s, 9H), 1.58-1.70 (m, 1H), 1.72-1.88 (m, 2H), 3.65 (s, 3H), 4.81 (dd, J=9, 6 Hz, 1H), 7.49 (s, 2H); LC-MS (LC-ES) M+Na=347.

### D. 2-(tert-Butyl) 3-methyl (S)-5,5-dimethyl-1,2,6-thiadiazinane-2,3-dicarboxylate 1,1-dioxide

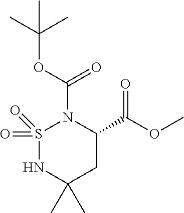 [SMILES: COC(=O)[C@@H]1CC(C)(C)NS(=O)(=O)N1C(=O)OC(C)(C)C]

Nitrogen was bubbled through the reaction mixture of methyl N-(tert-butoxycarbonyl)-N-sulfamoyl-L-leucinate (55.71 g, 172 mmol) in isopropyl acetate (1 L). Then, magnesium oxide (16.70 g, 414 mmol) and (diacetoxyiodo)benzene (60.87 g, 189 mmol) were added. Lastly, bis[rhodium(α,α,α′,α′-tetramethyl-1,3-benzenedipropionic acid)](3.279 g, 4.32 mmol) was added. After flushing with nitrogen, the reaction mixture was stirred for −15 hours, then concentrated. Ethyl acetate was added to the residue and the mixture was absorbed onto silica gel and purified by silica gel chromatography, eluting with (3:1 ethyl acetate:ethanol):hexanes (0:1 to 1:4) to give a residue. The residue was dissolved in dichloromethane and filtered over a bit of cotton, concentrated, then dried under vacuum to give 2-(tert-butyl) 3-methyl (S)-5,5-dimethyl-1,2,6-thiadiazinane-2,3-dicarboxylate 1,1-dioxide (42.48 g, 132 mmol, 77% yield) as a green goo. ¹H NMR (400 MHz, CD₃SOCD₃) δ 1.14 (s, 3H), 1.26 (s, 3H), 1.41 (s, 9H), 2.23 (ABX, 2H), 3.68 (s, 3H), 4.76 (dd, J=6, 5 Hz, 1H), 7.93 (s, 1H); LC-MS (LC-ES) M+Na=345.

Alternative Method

### 2-(tert-Butyl) 3-methyl (S)-5,5-dimethyl-1,2,6-thiadiazinane-2,3-dicarboxylate 1,1-dioxide

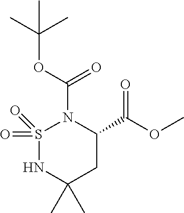 [SMILES: COC(=O)[C@@H]1CC(C)(C)NS(=O)(=O)N1C(=O)OC(C)(C)C]

A 1000 mL Morton flask with overhead stirring was charged with methyl N-(tert-butoxycarbonyl)-N-sulfamoyl-L-leucinate (29.14 g, 90 mmol) and isopropyl acetate (300 mL). The solution was degassed for about 15 minutes by sparging with nitrogen. Magnesium oxide (8.69 g, 216 mmol), (diacetoxyiodo)benzene (31.8 g, 99 mmol), and bis[rhodium(α,α,α′,α′-tetramethyl-1,3-benzenedipropionic acid)] (0.341 g, 0.449 mmol) were added and the blue-green mixture was stirred under nitrogen. ¹H NMR indicated complete conversion after 20 hours. The mixture was filtered through Celite® and the filtrate was concentrated in vacuo. The residue was diluted with dichloromethane (90 mL) and purified batch wise by silica gel chromatography, eluting with ethyl acetate:heptane (1:6 to 1:1) to give 2-(tert-butyl) 3-methyl (S)-5,5-dimethyl-1,2,6-thiadiazinane-2,3-dicarboxylate 1,1-dioxide (27.56 g, 85 mmol, 95% yield) as a pale greenish gum. ¹H NMR (400 MHz, CD₃SOCD₃) δ 1.15 (s, 3H), 1.27 (s, 3H), 1.42 (s, 9H), 2.23 (ABX, 2H), 3.69 (s, 3H), 4.77 (dd, J=6, 5 Hz, 1H), 7.96 (s, 1H); LC-MS (LC-ES) M+Na=345.

### E. tert-Butyl (S)-(5,5-dimethyl-2-oxopyrrolidin-3-yl)carbamate

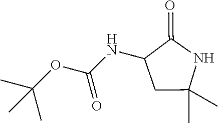 [SMILES: CC1(C)CC(NC(=O)OC(C)(C)C)C(=O)N1]

2-(tert-Butyl) 3-methyl (S)-5,5-dimethyl-1,2,6-thiadiazinane-2,3-dicarboxylate 1,1-dioxide (42.48 g, 132 mmol) in pyridine (120 ml) and water (12 mL) was stirred at 55° C. for ˜20 minutes, then 80° C. for ˜2 hours 10 minutes. The heating was stopped and the reaction was stirred at ambient temperature for 3 days. The heating at 80° C. was re-started for an additional 2 hours 25 minutes. The reaction mixture was concentrated and acetonitrile (200 mL) was added to the residue and the mixture was warmed to 55° C. The mixture was then stirred, while being cooled on an ice bath. Solids formed. The solids were filtered off and rinsed with acetonitrile. The solids were discarded. The filtrate was concentrated and dichloromethane and methanol were added to the residue and the mixture was absorbed onto silica gel and purified by silica gel chromatography, eluting with (3:1 ethyl acetate:ethanol):hexanes (0:1 to 1:13) to give a residue. The residue was dissolved in dichloromethane and filtered over a bit of cotton, concentrated, then dried under vacuum to give the tert-butyl (S)-(5,5-dimethyl-2-oxopyrrolidin-3-yl)carbamate (26.85 g, 118 mmol, 89% yield) as a tan powder. ¹H NMR (400 MHz, CD₃SOCD₃) δ 1.14 (s, 3H), 1.17 (s, 3H), 1.37 (s, 9H), 1.66 (t, J=11 Hz, 1H), 2.11 (dd, J=12, 9 Hz, 1H), 4.15 (q, J=9 Hz, 1H), 6.96 (br d, J=9 Hz, 1H), 7.82 (br s, 1H); LC-MS (LC-ES) M+H=229.

Alternative Method

### tert-Butyl (S)-(5,5-dimethyl-2-oxopyrrolidin-3-yl)carbamate

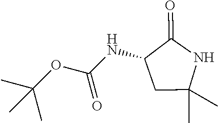 [SMILES: CC1(C)C[C@H](NC(=O)OC(C)(C)C)C(=O)N1]

A 500 mL 2 necked flask with stir bar and internal temperature probe was charged with 2-(tert-butyl) 3-methyl (S)-5,5-dimethyl-1,2,6-thiadiazinane-2,3-dicarboxylate 1,1-dioxide (27.5 g, 85 mmol), pyridine (75 mL), and water (7.5 mL). An air condenser was attached, and the orange solution was stirred at 80° C. under nitrogen. ¹H NMR, after 3 hours, indicated complete conversion. The mixture was diluted with acetonitrile (200 mL) and concentrated in vacuo (acetonitrile chase 2×). The residue was partitioned between dichloromethane:water and the aqueous layer was extracted with dichloromethane. The combined organics were washed with saturated sodium chloride, dried over sodium sulfate, filtered, and concentrated in vacuo. The residue was dissolved in a minimal amount of dichloromethane and purified by flash chromatography, eluting with (3:1 ethyl acetate:ethanol):heptane (1:4 to 1:1) to give tert-butyl (S)-(5,5-dimethyl-2-oxopyrrolidin-3-yl)carbamate (16.39 g, 71.8 mmol, 84% yield colorless foam. ¹H NMR (400 MHz, CD₃SOCD₃) δ 1.15 (s, 3H), 1.18 (s, 3H), 1.38 (s, 9H), 1.67 (t, J=11 Hz, 1H), 2.12 (dd, J=12, 9 Hz, 1H), 4.16 (q, J=9 Hz, 1H), 6.95 (br d, J=9 Hz, 1H), 7.82 (br s, 1H); LC-MS (LC-ES) M+H=229.

### F. (S)-3-Amino-5,5-dimethylpyrrolidin-2-one Hydrochloride

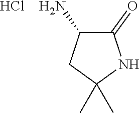

4 M Hydrochloric acid in 1,4-dioxane (200 mL, 800 mmol) was added to tert-butyl (S)-(5,5-dimethyl-2-oxopyrrolidin-3-yl)carbamate (42.342 g, 185 mmol) in dichloromethane (600 mL) and the reaction mixture was stirred for ˜55 minutes, then concentrated. Dichloromethane was added to the residue and the mixture was concentrated again, then dried under vacuum overnight. Dichloromethane (200 mL) was added to the residue, then 4 M hydrochloric acid in 1,4-dioxane (100 mL, 400 mmol) was added. After ˜30 minutes, methanol (20 mL) was added. After ˜30 minutes, the reaction mixture was concentrated. Dichloromethane was added to the residue and the mixture was concentrated again. There was an oily residue present. Hexanes (500 mL) were added to the residue and the suspension was shaken. The hexanes were carefully decanted from the solids. The solids were dried under vacuum to give the (S)-3-amino-5,5-dimethylpyrrolidin-2-one hydrochloride (30.22 g, 184 mmol, 99% yield) as a pale tan powder. ¹H NMR (400 MHz, CD₃SOCD₃) δ 1.20 (s, 3H), 1.24 (s, 3H), 1.81 (dd, J=12, 11 Hz, 1H), 2.26 (dd, J=12, 9 Hz, 1H), 4.05 (br t, J=12 Hz, 1H), 8.41 (br s, 1H), 8.47 (br s, 3H); LC-MS (LC-ES) M+H=129.

Alternative Method

### (S)-3-Amino-5,5-dimethylpyrrolidin-2-one Hydrochloride

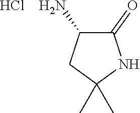

A 1000 mL flask with stir bar was charged with tert-butyl (S)-(5,5-dimethyl-2-oxopyrrolidin-3-yl)carbamate (16.36 g, 71.7 mmol) and dichloromethane (300 mL). To the resulting solution was added a 4.0 M solution of hydrochloric acid in dioxane (90 mL, 358 mmol) and the mixture was stirred at room temperature. The reaction mixture became heterogeneous (white solid) within 5 minutes. ¹H NMR indicated complete conversion after 5.5 hours. The reaction mixture was concentrated to dryness, then the solids were slurried in methyl tert-butyl ether and collected by filtration. The filter cake was dried on the Buchner funnel for about 1 hour, then in a vacuum oven (45° C./22″ Hg) overnight to obtain (S)-3-amino-5,5-dimethylpyrrolidin-2-one hydrochloride (12.19 g, 66.7 mmol, 93% yield) as a colorless solid (likely the monohydrate). ¹H NMR (400 MHz, CD₃SOCD₃) δ 1.19 (s, 3H), 1.24 (s, 3H), 1.87 (dd, J=12, 11 Hz, 1H), 2.27 (dd, J=12, 9 Hz, 1H), 4.03 (dd, J=10, 9 Hz, 1H), 8.44 (br s, 1H), 8.61 (br s, 3H); LC-MS (LC-ES) M+H=129.

Intermediate 14

### 2-(3,5-Difluorophenyl)thiazole-5-carboxylic Acid

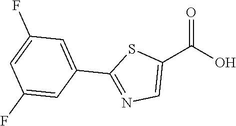 [SMILES: O=C(O)c1cnc(-c2cc(F)cc(F)c2)s1]

### A. Ethyl 2-(3,5-difluorophenyl)thiazole-5-carboxylate

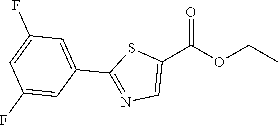 [SMILES: CCOC(=O)c1cnc(-c2cc(F)cc(F)c2)s1]

Degassed water (3 mL) and dioxane (6 mL) were added to (3,5-difluorophenyl)boronic acid (0.474 g, 3.00 mmol), 1,1′-bis(diphenylphosphino)ferrocene]dichloropalladium(II).dichloromethane complex (0.082 g, 0.100 mmol), sodium carbonate (0.636 g, 6.00 mmol), and ethyl 2-bromothiazole-5-carboxylate (0.297 mL, 2 mmol) evacuated/backfilled with nitrogen (3×). The reaction mixture was stirred in a heating block at 80° C. for 6 hours. Upon cooling, the reaction mixture was poured into water and extracted with ethyl acetate (3×). The combined organics were washed with water and saturate sodium chloride, dried over sodium sulfate, and concentrated in vacuo. The residue was purified by silica gel chromatography, eluting with ethyl acetate:hexanes (0:1 to 1:3) to give ethyl 2-(3,5-difluorophenyl)thiazole-5-carboxylate (0.218 g, 0.810 mmol, 40.5% yield) as a colorless solid. ¹H NMR (400 MHz, CD₃SOCD₃) δ 1.41 (t, J=7 Hz, 3H), 4.41 (q, J=7 Hz, 2H), 6.93 (tt, J=9, 2 Hz, 1H), 7.48-7.56 (m, 2H), 8.43 (s, 1H); LC-MS (LC-ES) M+H=270.

### B. 2-(3,5-Difluorophenyl)thiazole-5-carboxylic Acid

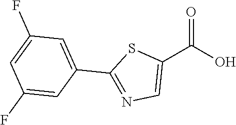 [SMILES: O=C(O)c1cnc(-c2cc(F)cc(F)c2)s1]

Lithium hydroxide monohydrate (0.165 g, 3.92 mmol) was added to a solution of ethyl 2-(3,5-difluorophenyl)thiazole-5-carboxylate (0.2113 g, 0.785 mmol) in tetrahydrofuran (5 mL) and water (0.5 mL) and the reaction mixture was stirred in a heating block at 70° C. LC-MS after 2.5 hours indicated complete conversion of starting material. Upon cooling, the reaction mixture was poured into water and extracted with diethyl ether (2×). The aqueous layer was acidified by addition of 1 M hydrochloric acid. The precipitated solids were collected by filtration and dried to give 2-(3,5-difluorophenyl)thiazole-5-carboxylic acid (0.188 g, 0.779 mmol, 99% yield) as a colorless solid. ¹H NMR (400 MHz, CD₃SOCD₃) δ 7.49 (tt, J=9, 2 Hz, 1H), 7.70-7.80 (m, 2H), 8.46 (s, 1H), 13.78 (br s, 1H); LC-MS (LC-ES) M+H=242.

Intermediate 15

### 6-Amino-4-azaspiro[2.4]heptan-5-one Hydrochloride

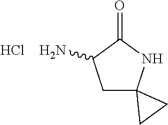

### A. Benzyl (1-formylcyclopropyl)carbamate

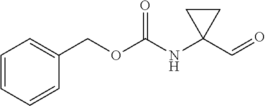 [SMILES: O=CC1(NC(=O)OCc2ccccc2)CC1]

(2,2,6,6-Tetramethylpiperidin-1-yl)oxyl (TEMPO) (0.071 g, 0.456 mmol) was added to benzyl (1-(hydroxymethyl)cyclopropyl)carbamate (2.02 g, 9.13 mmol, AstaTech) in dichloromethane (45.6 mL) at 0° C., followed by a 2M aqueous solution of potassium chloride (0.456 mL, 0.913 mmol). Then, sodium bicarbonate (0.460 g, 5.48 mmol) was dissolved in bleach (sodium hypochlorite (14.09 mL, 13.69 mmol)) and this solution was added to the reaction mixture, which was stirred for two hours at 0° C. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (0:1 to 2:3) to give benzyl (1-formylcyclopropyl)carbamate (1.33 g, 5.76 mmol, 63.1% yield). ¹H NMR (400 MHz, CD₃SOCD₃) δ 1.16-1.22 (m, 2H), 1.40-1.46 (m, 2H), 5.02 (s, 2H), 7.24-7.40 (m, 5H), 7.97 (br s, 1H), 8.93 (s, 1H); LC-MS (LC-ES) M+H=220.

### B. Methyl (Z)-3-(1-(((benzyloxy)carbonyl)amino)cyclopropyl)-2-((tert-butoxycarbonyl)amino)acrylate

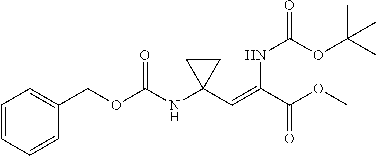 [SMILES: COC(=O)/C(=C/C1(NC(=O)OCc2ccccc2)CC1)NC(=O)OC(C)(C)C]

Methyl 2-((tert-butoxycarbonyl)amino)-2-(dimethoxyphosphoryl)acetate (3.79 g, 12.74 mmol) was added to benzyl (1-formylcyclopropyl)carbamate (1.33 g, 6.07 mmol) in dichloromethane (20.22 mL) at room temperature under a nitrogen atmosphere. Then, 1,8-diazabicyclo[5.4.0]undec-7-ene (1.724 mL, 11.53 mmol) was added and the reaction mixture was stirred for sixty-four hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 1:3) to give methyl (2)-3-(1-(((benzyloxy)carbonyl)amino)cyclopropyl)-2-((tert-butoxycarbonyl)amino)acrylate (1.98 g, 4.82 mmol, 79% yield). ¹H NMR (400 MHz, CD₃SOCD₃) δ 0.96-1.06 (m, 4H), 1.35 (s, 9H), 3.67 (s, 3H), 4.97 (s, 2H), 5.99 (br s, 1H), 7.26-7.38 (m, 5H), 7.73 (br s, 1H), 8.24 (br s, 1H); LC-MS (LC-ES) M+H=391.

### C. tert-Butyl (5-oxo-4-azaspiro[2.4]heptan-6-yl)carbamate

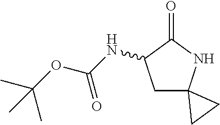 [SMILES: CC(C)(C)OC(=O)NC1CC2(CC2)NC1=O]

Palladium on carbon (0.270 g, 0.254 mmol) was added to methyl (Z)-3-(1-(((benzyloxy)carbonyl)amino)cyclopropyl)-2-((tert-butoxycarbonyl)amino)acrylate (1.98 g, 5.07 mmol) in methanol (50.7 mL) at 25° C. under nitrogen atmosphere. Then, the reaction vessel was fitted with a hydrogen balloon and the vessel was repeatedly evacuated and purged with hydrogen, then stirred for sixteen hours. Then, the vessel was repeatedly evacuated and purged with nitrogen, filtered through Celite®, and concentrated. The resulting residue was purified by silica gel chromatography, eluting with ethyl acetate:hexanes (2:3 to 1:0), then further purified by reverse phase HPLC, eluting with acetonitrile:water with 0.1% trifluoroacetic acid (0:1 to 1:0) to give tert-butyl (5-oxo-4-azaspiro[2.4]heptan-6-yl)carbamate (0.2282 g, 0.958 mmol, 18.89% yield). ¹H NMR (400 MHz, CD₃SOCD₃) δ 0.48-0.58 (m, 2H), 0.60-0.68 (m, 1H), 0.70-0.78 (m, 1H), 1.38 (s, 9H), 2.01 (dd, J=12, 11 Hz, 1H), 2.14 (t, J=11 Hz, 1H), 4.22 (q, J=10 Hz, 1H), 7.08 (br d, J=9 Hz, 1H), 7.76 (br s, 1H); LC-MS (LC-ES) M+H=227.

### D. 6-Amino-4-azaspiro[2.4]heptan-5-one Hydrochloride

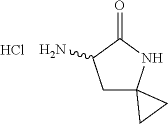

4.0 M Hydrochloric acid (1.261 mL, 5.04 mmol) in dioxane was added to tert-butyl (5-oxo-4-azaspiro[2.4]heptan-6-yl)carbamate (0.2282 g, 1.009 mmol) in methanol (1.009 mL) at room temperature and the reaction mixture was stirred for sixteen hours. The reaction mixture was concentrated to give 6-amino-4-azaspiro[2.4]heptan-5-one hydrochloride (0.1713 g, 0.579 mmol, 57.4% yield). ¹H NMR (400 MHz, CD₃SOCD₃) δ 0.58-1.00 (m, 4H), 1.82 (dd, J=15, 9 Hz, 1H), 2.22 (t, J=9 Hz, 1H), 4.08-4.18 (m, 1H), 8.42 (br s, 3H), 8.66 (br s, 1H); LC-MS (LC-ES) M+H=127.

Intermediate 16

### 7-Amino-5-azaspiro[3.4]octan-6-one Hydrochloride

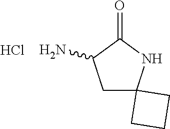

### A. Benzyl (1-(hydroxymethyl)cyclobutyl)carbamate

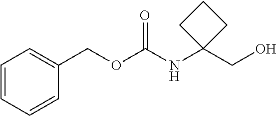 [SMILES: O=C(NC1(CO)CCC1)OCc1ccccc1]

Triethylamine (3.36 mL, 12.04 mmol) was added to 1-(((benzyloxy)carbonyl)amino)cyclobutane-1-carboxylic acid (3.00 g, 12.04 mmol) in tetrahydrofuran (53.5 mL) at 0° C., followed by isopropyl chloroformate (12.04 mL, 12.04 mmol) and the reaction mixture was stirred for 30 minutes. Then, the reaction mixture was filtered into sodium borohydride (0.592 g, 15.65 mmol) in water (6.69 mL) and the reaction mixture was stirred for five hours. The reaction mixture was filtered, saturated sodium bicarbonate added, extracted with ethyl acetate, washed with saturated sodium bicarbonate and saturated sodium chloride, dried over magnesium sulfate, filtered, and concentrated. The residue was purified by silica gel chromatography, eluting with ethyl acetate:hexanes (1:4 to 7:3) to give benzyl (1-(hydroxymethyl)cyclobutyl)carbamate (2.01 g, 8.12 mmol, 67.4% yield). ¹H NMR (400 MHz, CD₃SOCD₃) δ 1.56-1.68 (m, 1H), 1.68-1.80 (m, 1H), 1.92-2.02 (m, 2H), 2.04-2.14 (m, 2H), 3.45 (d, J=6 Hz, 2H), 4.72 (t, J=6 Hz, 1H), 4.96 (s, 2H), 7.21 (br s, 1H), 7.26-7.40 (m, 5H); LC-MS (LC-ES) M+H=236.

### B. Benzyl (1-formylcyclobutyl)carbamate

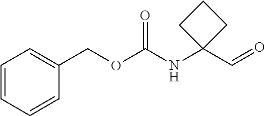 [SMILES: O=CC1(NC(=O)OCc2ccccc2)CCC1]

(2,2,6,6-Tetramethylpiperidin-1-yl)oxyl (TEMPO) (0.067 g, 0.427 mmol) was added to benzyl (1-(hydroxymethyl)cyclobutyl)carbamate (2.01 g, 8.54 mmol) in dichloromethane (42.7 mL) at 0° C., followed by a 2M aqueous solution of potassium chloride (0.427 mL, 0.854 mmol). Then, sodium bicarbonate (0.431 g, 5.13 mmol) was dissolved in bleach (sodium hypochlorite (13.18 mL, 12.81 mmol)) and this solution was added to the reaction mixture, which was stirred for twenty minutes at 0° C. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (0:1 to 2:3) to give benzyl (1-formylcyclobutyl)carbamate (1.81 g, 7.37 mmol, 86% yield). ¹H NMR (400 MHz, CD₃SOCD₃) δ 1.62-1.76 (m, 1H), 1.76-1.90 (m, 1H), 2.00-2.10 (m, 2H), 2.28-2.38 (m, 2H), 5.01 (s, 2H), 7.26-7.40 (m, 5H), 8.25 (br s, 1H), 9.45 (s, 1H); LC-MS (LC-ES) M+H=234.

### C. Methyl (Z)-3-(1-(((benzyloxy)carbonyl)amino)cyclobutyl)-2-((tert-butoxycarbonyl)amino)acrylate

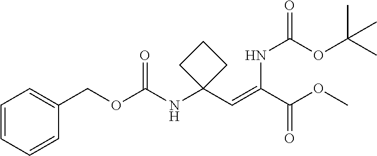 [SMILES: COC(=O)/C(=C/C1(NC(=O)OCc2ccccc2)CCC1)NC(=O)OC(C)(C)C]

Methyl 2-((tert-butoxycarbonyl)amino)-2-(dimethoxyphosphoryl)acetate (4.84 g, 16.29 mmol) was added to benzyl (1-formylcyclobutyl)carbamate (1.81 g, 7.76 mmol) in dichloromethane (38.8 mL) at room temperature under a nitrogen atmosphere. Then, 1,8-diazabicyclo[5.4.0]undec-7-ene (2.205 mL, 14.74 mmol) was added and the reaction mixture was stirred for sixteen hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 1:1) to give methyl (Z)-3-(1-(((benzyloxy)carbonyl)amino)cyclobutyl)-2-((tert-butoxycarbonyl)amino)acrylate (2.22 g, 5.21 mmol, 67.2% yield). ¹H NMR (400 MHz, CD₃SOCD₃) δ 1.37 (s, 9H), 1.66-1.76 (m, 1H), 1.88-2.00 (m, 1H), 2.14-2.30 (m, 4H), 3.65 (s, 3H), 5.00 (s, 2H), 6.06 (br s, 1H), 7.28-7.40 (m, 5H), 7.89 (s, 1H), 8.16 (br s, 1H); LC-MS (LC-ES) M+H=405.

### D. tert-Butyl (6-oxo-5-azaspiro[3.4]octan-7-yl)carbamate

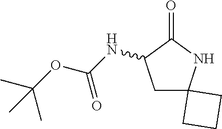 [SMILES: CC(C)(C)OC(=O)NC1CC2(CCC2)NC1=O]

Palladium on carbon (0.292 g, 0.274 mmol) was added to methyl (Z)-3-(1-(((benzyloxy)carbonyl)amino)cyclobutyl)-2-((tert-butoxycarbonyl)amino)acrylate (2.22 g, 5.49 mmol) in methanol (27.4 mL) at 25° C. under nitrogen atmosphere. Then, the reaction vessel was fitted with a hydrogen balloon and the vessel was repeatedly evacuated and purged with hydrogen, then stirred for sixty-six hours. Then, the vessel was repeatedly evacuated and purged with nitrogen, filtered through Celite®, and concentrated. The resulting residue was purified by reverse phase HPLC, eluting with acetonitrile:water with 0.1% ammonium hydroxide (0:1 to 1:0) to give tert-butyl (6-oxo-5-azaspiro[3.4]octan-7-yl)carbamate (1.17 g, 4.63 mmol, 84% yield). ¹H NMR (400 MHz, CD₃SOCD₃) δ 1.37 (s, 9H), 1.54-1.66 (m, 2H), 1.80 (t, J=12 Hz, 1H), 1.84-1.92 (m, 1H), 1.96 (q, J=10 Hz, 1H), 2.02-2.12 (m, 1H), 2.24 (q, J=10 Hz, 1H), 2.49 (t, J=10 Hz, 1H), 4.03 (dt, J=11, 9 Hz, 1H), 7.03 (br d, J=9 Hz, 1H), 8.19 (br s, 1H); LC-MS (LC-ES) M+H=241.

### E. 7-Amino-5-azaspiro[3.4]octan-6-one Hydrochloride

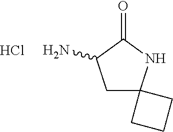

4.0 M Hydrochloric acid (6.09 mL, 24.34 mmol) in dioxane was added to tert-butyl (6-oxo-5-azaspiro[3.4]octan-7-yl)carbamate (1.17 g, 4.87 mmol) in methanol (6.09 mL) at room temperature and the reaction mixture was filtered for sixteen hours. The reaction mixture was concentrated to give 7-amino-5-azaspiro[3.4]octan-6-one hydrochloride (0.9004 g, 4.84 mmol, 99% yield). ¹H NMR (400 MHz, CD₃SOCD₃) δ 1.58-1.74 (m, 2H), 1.91 (dd, J=12, 11 Hz, 1H), 1.96-2.00 (m, 1H), 2.02 (q, J=10 Hz, 1H), 2.08-2.18 (m, 1H), 2.31 (q, J=10 Hz, 1H), 2.65 (dd, J=12, 8 Hz, 1H), 3.96 (dd, J=10, 8 Hz, 1H), 8.37 (br s, 3H), 8.79 (s, 1H); LC-MS (LC-ES) M+H=141.

Intermediate 17

### 2-(3-Chlorophenyl)-4-methylthiazole-5-carboxylic Acid

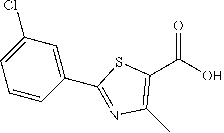 [SMILES: Cc1nc(-c2cccc(Cl)c2)sc1C(=O)O]

### A. Ethyl 2-(3-chlorophenyl)-4-methylthiazole-5-carboxylate

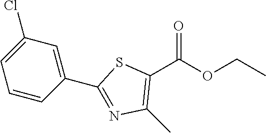 [SMILES: CCOC(=O)c1sc(-c2cccc(Cl)c2)nc1C]

Tetrakis(triphenylphosphine)palladium(0) (0.235 g, 0.204 mmol) was added to ethyl 2-bromo-4-methylthiazole-5-carboxylate (1.018 g, 4.07 mmol) in tetrahydrofuran (3 mL). Then a solution of (3-chlorophenyl)zinc(II) iodide (12.21 mL, 6.11 mmol) was added via syringe (exothermic). The reaction mixture was stirred in a heating block at 70° C. An additional portion of (3-chlorophenyl)zinc(II) iodide (1.2 mL) was added after 75 minutes and heating resumed for 45 minutes. After cooling, the reaction mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (0:1 to 1:0) to afford ethyl 2-(3-chlorophenyl)-4-methylthiazole-5-carboxylate (1.019 g, 3.62 mmol, 89% yield) as a colorless solid. ¹H NMR (400 MHz, CD₃SOCD₃) δ 1.31 (t, J=7 Hz, 3H), 2.69 (s, 3H), 4.30 (q, J=7 Hz, 2H), 7.55 (t, J=8 Hz, 1H), 7.62 (ddd, J=8, 2, 1 Hz, 1H), 7.94 (ddd, J=8, 2, 1 Hz, 1H), 8.00 (t, J=2 Hz, 1H); LC-MS (LC-ES) M+H=282.

### B. 2-(3-Chlorophenyl)-4-methylthiazole-5-carboxylic Acid

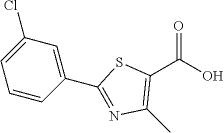 [SMILES: Cc1nc(-c2cccc(Cl)c2)sc1C(=O)O]

Lithium hydroxide monohydrate (0.748 g, 17.83 mmol) was added to a solution of ethyl 2-(3-chlorophenyl)-4-methylthiazole-5-carboxylate (1.005 g, 3.57 mmol) in tetrahydrofuran (5 mL) and water (0.5 mL) and the mixture was stirred in a heating block at 50° C. for nineteen hours. The reaction mixture was poured into water and extracted with diethyl ether (2×). The aqueous layer was acidified by addition of 1M hydrochloric acid (17.8 mL) and the precipitated solids were collected by filtration, washed with water, and dried to afford 2-(3-chlorophenyl)-4-methylthiazole-5-carboxylic acid (0.874 g, 3.44 mmol, 97% yield) as a colorless solid. ¹H NMR (400 MHz, CD₃SOCD₃) δ 2.67 (s, 3H), 7.54 (t, J=8 Hz, 1H), 7.60 (ddd, J=8, 2, 1 Hz, 1H), 7.92 (ddd, J=8, 2, 1 Hz, 1H), 7.98 (t, J=2 Hz, 1H), 13.51 (br s, 1H); LC-MS (LC-ES) M+H=254.

Intermediate 18

### 2-(4-Methyl-1H-pyrazol-1-yl)thiazole-5-carboxylic Acid

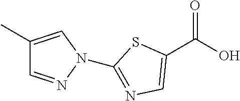

### A. Ethyl 2-(4-methyl-1H-pyrazol-1-yl)thiazole-5-carboxylate

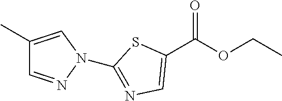 [SMILES: CCOC(=O)c1cnc(-n2cc(C)cn2)s1]

4-Methyl-1H-pyrazole (0.506 mL, 6.35 mmol) was added to ethyl 2-bromothiazole-5-carboxylate (0.633 mL, 4.24 mmol) in acetonitrile (8 mL), followed by potassium carbonate (1.464 g, 10.59 mmol) and the reaction mixture was stirred in a heating block at 120° C. for eight hours. Then, an additional portion of 4-methyl-1H-pyrazole (0.26 mL; 3.2 mmol) was added and heating resumed for eighteen hours. After the mixture was cooled, it was poured into water (50 mL) and extracted with ethyl acetate (3×). The combined organics were washed with water and saturated sodium chloride, dried over sodium sulfate, filtered, and concentrated in vacuo. The residue was purified by silica gel chromatography, eluting with ethyl acetate:hexanes (0:1 to 1:4) to afford ethyl 2-(4-methyl-1H-pyrazol-1-yl)thiazole-5-carboxylate (0.665 g, 2.80 mmol, 66.2% yield) as a colorless solid. ¹H NMR (400 MHz, CD₃SOCD₃) δ 1.30 (t, J=7 Hz, 3H), 2.10 (s, 3H), 4.30 (q, J=7 Hz, 2H), 7.79 (s, 1H), 8.25 (s, 1H), 8.34 (t, J=1 Hz, 1H); LC-MS (LC-ES) M+H=238.

### B. 2-(4-Methyl-1H-pyrazol-1-yl)thiazole-5-carboxylic Acid

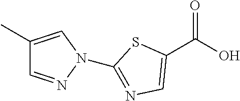

Lithium hydroxide monohydrate (0.493 g, 11.76 mmol) was added to a solution of ethyl 2-(4-methyl-1H-pyrazol-1-yl)thiazole-5-carboxylate (0.558 g, 2.352 mmol) in tetrahydrofuran (5 mL) and water (0.5 mL) and the reaction mixture was stirred in a heating block at 60° C. for eighteen hours. The reaction mixture was cooled, poured into water (50 mL), and extracted with diethyl ether. The aqueous layer was acidified by addition of 1 M hydrochloric acid (11.8 mL) and the precipitated solids were collected by filtration, washed with water, and dried in a vacuum oven (50° C., 28″Hg) overnight to afford 2-(4-methyl-1H-pyrazol-1-yl)thiazole-5-carboxylic acid (0.453 g, 2.165 mmol, 92% yield) as a colorless solid. ¹H NMR (400 MHz, CD₃SOCD₃) δ 2.10 (s, 3H), 7.78 (s, 1H), 8.16 (s, 1H), 8.33 (t, J=1 Hz, 1H), 13.62 (br s, 1H); LC-MS (LC-ES) M+H=210.

Intermediate 19

### 2-(4-Methyl-1H-imidazol-1-yl)thiazole-5-carboxylic Acid

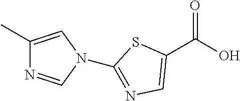

### A. Ethyl 2-(4-methyl-1H-imidazol-1-yl)thiazole-5-carboxylate and Ethyl 2-(5-methyl-1H-imidazol-1-yl)thiazole-5-carboxylate

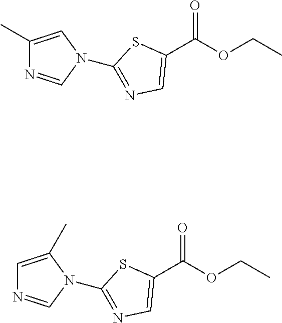 [SMILES: CCOC(=O)c1cnc(-n2cnc(C)c2)s1 ; CCOC(=O)c1cnc(-n2cncc2C)s1]

Potassium carbonate (1.620 g, 11.72 mmol) was added to ethyl 2-bromothiazole-5-carboxylate (0.70 mL, 4.69 mmol) and 4-methyl-1H-imidazole (0.577 g, 7.03 mmol) in acetonitrile (7 mL) and the reaction mixture was stirred in a heating block at 120° C. for 5 hours. After cooling, the mixture was diluted with water and extracted with ethyl acetate (3×). The combined organics were washed with water and saturated sodium chloride, dried over sodium sulfate, filtered, and concentrated in vacuo. The residue was purified by silica gel chromatography, eluting with ethyl acetate:hexanes (0:1 to 1:0) to afford ethyl 2-(4-methyl-1H-imidazol-1-yl)thiazole-5-carboxylate (0.650 g, 2.74 mmol, 58.4% yield), followed very closely by ethyl 2-(5-methyl-1H-imidazol-1-yl)thiazole-5-carboxylate (0.124 g, 0.523 mmol, 11.15% yield).

### Ethyl 2-(4-methyl-1H-imidazol-1-yl)thiazole-5-carboxylate

¹H NMR (400 MHz, CD₃SOCD₃) δ 1.30 (t, J=7 Hz, 3H), 2.16 (d, J=1 Hz, 3H), 4.32 (q, J=7 Hz, 2H), 7.60 (quin, J=1 Hz, 1H), 8.27 (s, 1H), 8.40 (d, J=1 Hz, 1H); LC-MS (LC-ES) M+H=238.

### Ethyl 2-(5-methyl-1H-imidazol-1-yl)thiazole-5-carboxylate

¹H NMR (400 MHz, CD₃SOCD₃) δ 1.31 (t, J=7 Hz, 3H), 2.45 (d, J=1 Hz, 3H), 4.34 (q, J=7 Hz, 2H), 6.92 (quin, J=1 Hz, 1H), 8.32 (d, J=1 Hz, 1H), 8.38 (s, 1H); LC-MS (LC-ES) M+H=238.

### B. 2-(4-Methyl-1H-imidazol-1-yl)thiazole-5-carboxylic Acid

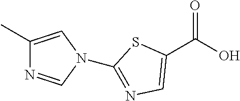

Lithium hydroxide monohydrate (0.557 g, 13.28 mmol) was added to a solution of ethyl 2-(4-methyl-1H-imidazol-1-yl)thiazole-5-carboxylate (0.630 g, 2.66 mmol) in tetrahydrofuran (5 mL) and water (0.5 mL) and the reaction mixture was stirred in a heating block at 50° C. for 22 hours. After cooling, the reaction mixture was diluted with water and extracted with diethyl ether. The aqueous layer was acidified by addition of 6 M hydrochloric acid and the precipitated solids were collected by filtration and dried on the Buchner funnel to afford 2-(4-methyl-1H-imidazol-1-yl)thiazole-5-carboxylic acid (0.396 g, 1.893 mmol, 71.3% yield) as a colorless solid. ¹H NMR (400 MHz, CD₃SOCD₃) δ 2.16 (d, J=1 Hz, 3H), 7.60 (t, J=1 Hz, 1H), 8.18 (s, 1H), 8.39 (d, J=1 Hz, 1H), 13.77 (br s, 1H); LC-MS (LC-ES) M+H=210.

Intermediate 20

### 2-Phenylthiazole-5-carboxylic Acid

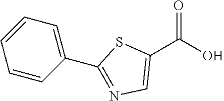 [SMILES: O=C(O)c1cnc(-c2ccccc2)s1]

### A. Ethyl 2-phenylthiazole-5-carboxylate

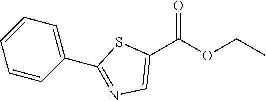 [SMILES: CCOC(=O)c1cnc(-c2ccccc2)s1]

Tetrakis(triphenylphosphine)palladium(0) (0.251 g, 0.218 mmol) was added to a solution of ethyl 2-bromothiazole-5-carboxylate (0.65 mL, 4.35 mmol) in tetrahydrofuran (3 mL) at room temperature. Then, a solution of phenylzinc(II) iodide in tetrahydrofuran (13.06 mL, 6.53 mmol) was added dropwise over 3 minutes (exothermic). The reaction mixture was stirred in a heating block at 70° C. for 40 minutes. After cooling, the reaction mixture was poured into saturated ammonium chloride and extracted with ethyl acetate (3×). The combined organics were washed with water and sodium chloride, dried over sodium sulfate, and concentrated in vacuo. The residue was purified by silica gel chromatography, eluting with ethyl acetate:hexanes (1:0 to 0:1) to afford ethyl 2-phenylthiazole-5-carboxylate (0.852 g, 3.65 mmol, 84% yield) as a colorless solid. ¹H NMR (400 MHz, CD₃SOCD₃) δ 1.32 (t, J=7 Hz, 3H), 4.34 (q, J=7 Hz, 2H), 7.50-7.62 (m, 3H), 7.98-8.08 (m, 2H), 8.51 (s, 1H); LC-MS (LC-ES) M+H=234.

### B. 2-Phenylthiazole-5-carboxylic Acid

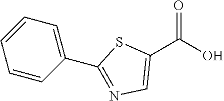 [SMILES: O=C(O)c1cnc(-c2ccccc2)s1]

Lithium hydroxide monohydrate (0.754 g, 17.96 mmol) was added to a solution of ethyl 2-phenylthiazole-5-carboxylate (0.838 g, 3.59 mmol) in tetrahydrofuran (5 mL) and water (0.5 mL) and the reaction mixture was stirred in a heating block at 50° C. for 3 hours. After cooling, the reaction mixture was diluted with water and extracted with diethyl ether. The aqueous layer was acidified by addition of 6 M hydrochloric acid and the precipitated solids were collected by filtration, and dried on the Buchner funnel to afford 2-phenylthiazole-5-carboxylic acid (0.674 g, 3.28 mmol, 91% yield) as a faintly yellow solid. ¹H NMR (400 MHz, CD₃SOCD₃) δ 7.50-7.60 (m, 3H), 7.98-8.06 (m, 2H), 8.42 (s, 1H), 13.64 (br s, 1H); LC-MS (LC-ES) M+H=206.

Intermediate 21

### Racemic (3R,4R,5S)-3-Amino-4,5-dimethylpyrrolidin-2-one Hydrochloride and (3S,4S,5R)-3-Amino-4,5-dimethylpyrrolidin-2-one Hydrochloride

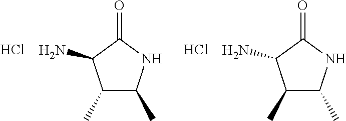

### A. Racemic tert-Butyl ((3R,4R,5R)-4,5-dimethyl-2-oxopyrrolidin-3-yl)carbamate and Tert-Butyl ((3S,4S,5S)-4,5-dimethyl-2-oxopyrrolidin-3-yl)carbamate and Racemic Tert-Butyl ((3R,4S,5S)-4,5-dimethyl-2-oxopyrrolidin-3-yl)carbamate and tert-Butyl ((3S,4R,5R)-4,5-dimethyl-2-oxopyrrolidin-3-yl)carbamate

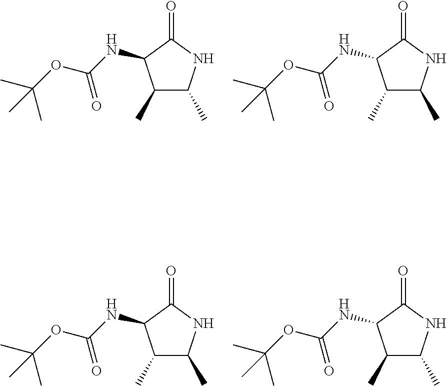

tert-Butyl 2-((diphenylmethylene)amino)acetate (15.00 g, 50.8 mmol) was added to racemic (2R,3R)-2,3-dimethyloxirane (3.63 g, 50.3 mmol) in tetrahydrofuran (250 mL) and the reaction mixture was kept under nitrogen and cooled on a dry ice/acetone bath. Then, 1 M lithium hexamethyldisilazide in tetrahydrofuran (51 mL, 51.0 mmol) was added, followed by boron trifluoride diethyl etherate (6.3 mL, 51.0 mmol). After ˜65 minutes, the ice bath was removed. After an additional ˜2 hours 35 minutes, the reaction was quenched with 10% citric acid (250 mL) and the biphasic reaction mixture was stirred for 25 days, then diluted with hexanes (250 mL). The layers were separated and the aqueous layer was washed once with hexanes (125 mL). The organic layers were discarded. The aqueous layer was cooled on an ice bath and slowly basified with 6 M sodium hydroxide (62.5 mL) to pH=˜13. The mixture was extracted with dichloromethane (125 mL, 3×). The organic layers were dried over magnesium sulfate, filtered, and the filtrate concentrated to give a diastereomerically mixed racemic tert-butyl (3R,4R)-2-amino-4-hydroxy-3-methylpentanoate (3.94 g, 19.4 mmol, 39% yield) as cloudy tan oil (LC-MS (LC-ES) M+H=204). A solution of di-tert-butyl dicarbonate (5.073 g, 23.24 mmol) in dichloromethane (10 mL) was added to the racemic tert-butyl (3R,4R)-2-amino-4-hydroxy-3-methylpentanoate (3.94 g, 19.38 mmol) in dichloromethane (190 mL). After-3 hours 40 minutes, more di-tert-butyl dicarbonate (2.622 g, 12.01 mmol) dissolved in dichloromethane (10 mL) was added to the reaction mixture. After ˜16 hours 25 minutes, the reaction mixture was concentrated. Dichloromethane was added to the residue and the mixture was absorbed onto silica gel. This residue was purified by silica gel chromatography, eluting with ethyl acetate:hexanes (1:0 to 1:1) to afford diastereomerically mixed racemic tert-butyl (3R,4R)-2-((tert-butoxycarbonyl)amino)-4-hydroxy-3-methylpentanoate (4.16 g, 13.7 mmol, 71% yield) as a thick colorless oil (LC-MS (LC-ES) M+H=304). Triphenylphosphine (3.953 g, 15.07 mmol) was added to racemic tert-butyl (3R,4R)-2-((tert-butoxycarbonyl)amino)-4-hydroxy-3-methylpentanoate (4.16 g, 13.71 mmol) in tetrahydrofuran (130 mL). The reaction mixture was kept under nitrogen and cooled on an ice bath. A solution of diisopropyl azodicarboxylate (2.93 mL, 15.07 mmol) in tetrahydrofuran (10 mL) was added over ˜10-15 minutes. After ˜20 minutes, a solution of diphenylphosphoryl azide (3.25 mL, 15.08 mmol) in tetrahydrofuran (10 mL) was added over ˜15-20 minutes. The ice bath was removed after ˜55 minutes. After stirring ˜15 hours 55 minutes, the reaction mixture was concentrated. Diethyl ether was added to the residue and the mixture was absorbed onto silica gel. This residue was purified by silica gel chromatography, eluting with ethyl acetate:hexanes (1:0 to 1:4) to afford a solid. The residue was dissolved in ethyl acetate and filtered through cotton and concentrated to give the diastereomerically mixed racemic tert-butyl (3R,4S)-4-azido-2-((tert-butoxycarbonyl)amino)-3-methylpentanoate (3.70 g, 11.3 mmol, 82% yield) as a colorless oil (LC-MS (LC-ES) M+H=329). Zinc (dust, <10 micron, 3.690 g, 56.4 mmol) was added to racemic tert-butyl (3R,4S)-4-azido-2-((tert-butoxycarbonyl)amino)-3-methylpentanoate (3.70 g, 11.27 mmol) in acetic acid (110 mL). The reaction mixture was stirred under nitrogen for ˜1 hour 10 minutes. The reaction mixture was filtered over a pad of Celite® and the filter cake was rinsed with acetic acid. The filtrate was concentrated. The residue was partitioned between dichloromethane (100 mL) and 1 M potassium carbonate (50 mL). The layers were separated and the aqueous layer was extracted once more with dichloromethane (50 mL). The organic layers were dried over magnesium sulfate, filtered, and the filtrate was concentrated. The residue was diluted with toluene (25 mL) and the suspension was heated at 80° C. for 3 days under nitrogen. The reaction mixture was concentrated and dichloromethane was added to the residue and the solids were filtered off and the filter cake was rinsed with a little dichloromethane. The solids were discarded and the filtrate was partially concentrated and more solid precipitated. These solids were also filtered off and rinsed with a little dichloromethane and discarded. The filtrate was absorbed onto silica gel. This residue was purified by silica gel chromatography, eluting with (3:1 ethyl acetate:ethanol):hexanes (1:0 to 1:1) to afford impure racemic tert-butyl ((3R,4R,5R)-4,5-dimethyl-2-oxopyrrolidin-3-yl)carbamate and tert-butyl ((3S,4S,5S)-4,5-dimethyl-2-oxopyrrolidin-3-yl)carbamate (0.148 g, 0.648 mmol) and racemic tert-butyl ((3R,4S,5S)-4,5-dimethyl-2-oxopyrrolidin-3-yl)carbamate and tert-butyl ((3S,4R,5R)-4,5-dimethyl-2-oxopyrrolidin-3-yl)carbamate (0.777 g, 3.40 min, 30% yield) as very pale tan foamy solid.

### Racemic Tert-Butyl ((3R,4R,5R)-4,5-dimethyl-2-oxopyrrolidin-3-yl)carbamate and Tert-Butyl ((3S,4S,5S)-4,5-dimethyl-2-oxopyrrolidin-3-yl)carbamate

LC-MS (LC-ES) M+H=229.

### Racemic Tert-Butyl ((3R,4S,5S)-4,5-dimethyl-2-oxopyrrolidin-3-yl)carbamate and Tert-Butyl ((3S,4R,5R)-4,5-dimethyl-2-oxopyrrolidin-3-yl)carbamate

¹H NMR (400 MHz, CD₃SOCD₃) δ 0.98 (d, J=7 Hz, 3H), 1.06 (d, J=6 Hz, 3H), 1.37 (s, 9H), 1.56-1.68 (m, 1H), 3.02-3.12 (m, 1H), 3.68 (dd, J=11, 9 Hz, 1H), 6.94 (d, J=9 Hz, 1H), 7.77 (br s, 1H); LC-MS (LC-ES) M+H=229.

### B. Racemic (3R,4R,5S)-3-Amino-4,5-dimethylpyrrolidin-2-one Hydrochloride and (3S,4S,5R)-3-Amino-4,5-dimethylpyrrolidin-2-one Hydrochloride

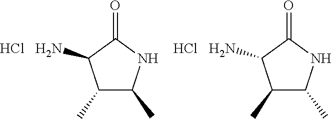

4 M Hydrochloric acid in 1,4-dioxane (6.8 mL, 27.2 mmol) was added to racemic tert-butyl ((3R,4S,5S)-4,5-dimethyl-2-oxopyrrolidin-3-yl)carbamate and tert-butyl ((3S,4R,5R)-4,5-dimethyl-2-oxopyrrolidin-3-yl)carbamate (0.771 g, 3.38 mmol) in dichloromethane (35 mL). After ˜15 hours 30 minutes, the reaction mixture was concentrated. Dichloromethane was added to the residue and the mixture was concentrated again to give racemic (3R,4R,5S)-3-amino-4,5-dimethylpyrrolidin-2-one hydrochloride and (3S,4S,5R)-3-amino-4,5-dimethylpyrrolidin-2-one hydrochloride (0.620 g, 3.38 mmol, 100% yield assuming 90% pure by weight) as a cream-colored powder. LC-MS (LC-ES) M+H=129.

Intermediate 22

### 2-(3-Bromophenyl)thiazole-5-carboxylic Acid

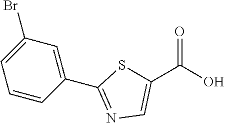 [SMILES: O=C(O)c1cnc(-c2cccc(Br)c2)s1]

### A. Ethyl 2-(3-bromophenyl)thiazole-5-carboxylate

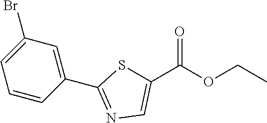 [SMILES: CCOC(=O)c1cnc(-c2cccc(Br)c2)s1]

A solution of 0.5 M (3-bromophenyl)zinc(II) iodide (11.32 mL, 5.66 mmol) was added to ethyl 2-bromothiazole-5-carboxylate (0.65 mL, 4.35 mmol), tetrakis(triphenylphosphine)palladium(0) (0.251 g, 0.218 mmol) and tetrahydrofuran (3 mL) and the reaction mixture was stirred in a heating block at 70° C. Additional portions of (3-bromophenyl)zinc(II) iodide (0.87 mL ea) were added after 90 min and 2 h. After 2.5 h, the mixture was cooled, poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:0 to 0:1) to afford ethyl 2-(3-bromophenyl)thiazole-5-carboxylate (1.074 g, 3.44 mmol, 79% yield) as a colorless solid. ¹H NMR (400 MHz, CD₃SOCD₃) δ 1.32 (t, J=7 Hz, 3H), 4.34 (q, J=7 Hz, 2H), 7.50 (t, J=8 Hz, 1H), 7.77 (ddd, J=8, 2, 1 Hz, 1H), 8.02 (ddd, J=8, 2, 1 Hz, 1H), 8.17 (t, J=2 Hz, 1H), 8.52 (s, 1H); LC-MS (LC-ES) M+H=312.

### B. 2-(3-Bromophenyl)thiazole-5-carboxylic Acid

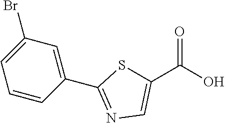 [SMILES: O=C(O)c1cnc(-c2cccc(Br)c2)s1]

Lithium hydroxide monohydrate (0.710 g, 16.93 mmol) was added to a solution of ethyl 2-(3-bromophenyl)thiazole-5-carboxylate (1.057 g, 3.39 mmol) in tetrahydrofuran (5 mL) and water (0.5 mL) and the reaction mixture was stirred in a heating block at 50° C.

After 3.5 hours, the reaction mixture was cooled, poured into water and extracted with diethyl ether (2×). The aqueous layer was acidified by addition of 1 M hydrochloric acid (16.9 mL) and stirred −10 minutes. The precipitated solids were collected by filtration, washed with water, and dried to afford 2-(3-bromophenyl)thiazole-5-carboxylic acid (0.9441 g, 3.32 mmol, 98% yield) as a colorless solid. ¹H NMR (400 MHz, CD₃SOCD₃) δ 7.50 (t, J=8 Hz, 1H), 7.76 (ddd, J=8, 2, 1 Hz, 1H), 8.01 (dt, J=8, 1 Hz, 1H), 8.17 (t, J=2 Hz, 1H), 8.44 (s, 1H), 13.75 (br s, 1H); LC-MS (LC-ES) M+H=284.

Intermediate 23

### 2-(Pyridin-4-yl)thiazole-5-carboxylic Acid

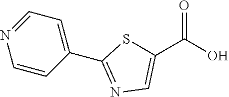 [SMILES: O=C(O)c1cnc(-c2ccncc2)s1]

### A. Ethyl 2-(pyridin-4-yl)thiazole-5-carboxylate

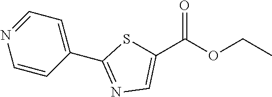 [SMILES: CCOC(=O)c1cnc(-c2ccncc2)s1]

A 1.3 M solution of isopropylmagnesium chloride lithium chloride complex in tetrahydrofuran (3.69 mL, 4.80 mmol) was added, dropwise, over −5 minutes to a solution of 4-iodopyridine (0.820 g, 4.00 mmol) in tetrahydrofuran (8 mL) at −78° C. The reaction mixture was stirred 30 min and a 1.73 M solution of zinc(II) bromide in tetrahydrofuran (3.01 mL, 5.20 mmol) was added dropwise. The cooling bath was removed and the mixture was warmed to room temperature. Tetrakis(triphenylphosphine)palladium(0) (0.231 g, 0.200 mmol) and ethyl 2-bromothiazole-5-carboxylate (0.597 mL, 4.00 mmol) were added and the reaction mixture was stirred in a heating block at 70° C. After 20 minutes, the mixture was cooled, poured into saturated ammonium chloride (50 mL) and water (10 mL) and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 0:1) to afford ethyl 2-(pyridin-4-yl)thiazole-5-carboxylate (0.595 g, 2.54 mmol, 63.5% yield) as a tan solid. ¹H NMR (400 MHz, CD₃SOCD₃) δ 1.32 (t, J=7 Hz, 3H), 4.35 (q, J=7 Hz, 2H), 7.96 (dd, J=4, 2 Hz, 2H), 8.61 (s, 1H), 8.76 (dd, J=4, 2 Hz, 2H); LC-MS (LC-ES) M+H=235.

### B. 2-(Pyridin-4-yl)thiazole-5-carboxylic Acid

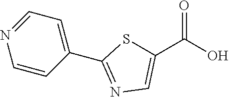 [SMILES: O=C(O)c1cnc(-c2ccncc2)s1]

Lithium hydroxide monohydrate (0.527 g, 12.55 mmol) was added to a solution of ethyl 2-(pyridin-4-yl)thiazole-5-carboxylate (0.588 g, 2.510 mmol) in tetrahydrofuran (5 mL) and water (0.5 mL) and the reaction mixture was stirred overnight in a heating block at 50° C. Upon cooling, the reaction mixture was poured into water and extracted with diethyl ether (1×). The aqueous layer was acidified by addition of 1 M hydrochloric acid (12.6 mL). The precipitated solids were collected by filtration, washed with water, and dried in a vacuum oven (50° C./28″ Hg) to obtain 2-(pyridin-4-yl)thiazole-5-carboxylic acid (0.380 g, 1.843 mmol, 73.4% yield) as a tan solid. ¹H NMR (400 MHz, CD₃SOCD₃) δ 7.96 (dd, J=4, 1 Hz, 2H), 8.52 (s, 1H), 8.76 (dd, J=5, 1 Hz, 2H), 13.83 (br s, 1H); LC-MS (LC-ES) M+H=207.

Intermediate 24

### 2-(Pyridin-2-yl)thiazole-5-carboxylic Acid

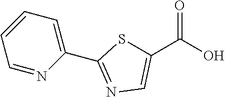 [SMILES: O=C(O)c1cnc(-c2ccccn2)s1]

### A. Ethyl 2-(pyridin-2-yl)thiazole-5-carboxylate

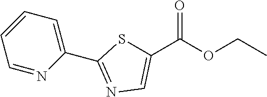 [SMILES: CCOC(=O)c1cnc(-c2ccccn2)s1]

A 1.3 M solution of isopropylmagnesium chloride lithium chloride complex in tetrahydrofuran (4.20 mL, 5.46 mmol) was added, dropwise, over −5 minutes to a solution of 2-iodopyridine (0.932 g, 4.55 mmol) in tetrahydrofuran (8 mL) at −78° C. The reaction mixture was stirred 30 minutes and a 1.73 M solution of zinc(II) bromide in tetrahydrofuran (3.42 mL, 5.91 mmol) was added dropwise. The cooling bath was removed and the mixture was warmed to room temperature. Tetrakis(triphenylphosphine)palladium(0) (0.263 g, 0.227 mmol) and ethyl 2-bromothiazole-5-carboxylate (0.679 mL, 4.55 mmol) were added and the reaction mixture was stirred in a heating block at 70° C. After 20 minutes, the reaction mixture was cooled, poured into saturated ammonium chloride (50 mL) and water (10 mL), and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:0 to 1:3) to afford slightly impure ethyl 2-(pyridin-2-yl)thiazole-5-carboxylate (0.476 g, 2.032 mmol, 44.7% yield) as a red solid (13% impurity). ¹H NMR (400 MHz, CD₃SOCD₃) δ 1.31 (t, J=7 Hz, 3H), 4.31 (q, J=7 Hz, 2H), 7.57 (ddd, J=8, 5, 1 Hz, 1H), 8.01 (dt, J=8, 2 Hz, 1H), 8.16 (dt, J=8, 1 Hz, 1H), 8.52 (s, 1H), 8.67 (ddd, J=5, 2, 1 Hz, 1H); LC-MS (LC-ES) M+H=235.

### B. 2-(Pyridin-2-yl)thiazole-5-carboxylic Acid

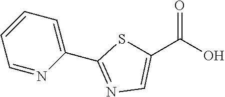 [SMILES: O=C(O)c1cnc(-c2ccccn2)s1]

Lithium hydroxide monohydrate (0.419 g, 9.99 mmol) was add to a solution of ethyl 2-(pyridin-2-yl)thiazole-5-carboxylate (0.468 g, 1.998 mmol) in tetrahydrofuran (5 mL) and water (0.5 mL) and the reaction mixture was stirred overnight in a heating block at 50° C. Upon cooling, the mixture was poured into water and extracted with diethyl ether (1×). The aqueous layer was acidified by addition of 1 M hydrochloric acid (10 mL). The precipitated solids were collected by filtration, washed with water, and dried in a vacuum oven (50° C./28″ Hg) to obtain 2-(pyridin-2-yl)thiazole-5-carboxylic acid (0.307 g, 1.489 mmol, 74.5% yield) as a light tan solid. ¹H NMR (400 MHz, CD₃SOCD₃) δ 7.57 (ddd, J=8, 5, 1 Hz, 1H), 8.02 (td, J=8, 2 Hz, 1H), 8.18 (dt, J=8, 1 Hz, 1H), 8.47 (s, 1H), 8.68 (ddd, J=8, 2, 1 Hz, 1H), 13.67 (br s, 1H); LC-MS (LC-ES) M+H=207.

Intermediate 25

### 2-(Pyridin-3-yl)thiazole-5-carboxylic Acid

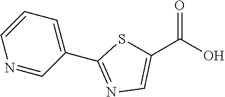 [SMILES: O=C(O)c1cnc(-c2cccnc2)s1]

### A. Ethyl 2-(pyridin-3-yl)thiazole-5-carboxylate

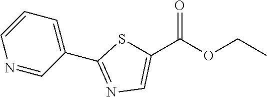 [SMILES: CCOC(=O)c1cnc(-c2cccnc2)s1]

A 1.3 M solution of isopropylmagnesium chloride lithium chloride complex in tetrahydrofuran (4.35 mL, 5.66 mmol) was added, dropwise, over −5 minutes to a solution of 3-iodopyridine (0.967 g, 4.72 mmol) in tetrahydrofuran (10 mL) at −78° C. The reaction mixture was stirred 30 minutes and a 1.81 M solution of zinc(II) bromide in tetrahydrofuran (3.39 mL, 6.13 mmol) was added dropwise. The cooling bath was removed and the mixture was warmed to room temperature. Tetrakis(triphenylphosphine)palladium(0) (0.273 g, 0.236 mmol) and ethyl 2-bromothiazole-5-carboxylate (0.704 mL, 4.72 mmol) were added and the reaction mixture was stirred in a heating block at 70° C. After 15 minutes, the reaction mixture was cooled, poured into saturated ammonium chloride (50 mL) and water (10 mL) and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:0) to afford ethyl 2-(pyridin-3-yl)thiazole-5-carboxylate (0.865 g, 3.69 mmol, 78% yield) as a tan solid. ¹H NMR (400 MHz, CD₃SOCD₃) δ 1.32 (t, J=7 Hz, 3H), 4.35 (q, J=7 Hz, 2H), 7.58 (ddd, J=8, 5, 1 Hz, 1H), 8.40 (ddd, J=8, 2, 2 Hz, 1H), 8.56 (s, 1H), 8.74 (dd, J=5, 2 Hz, 1H), 9.21 (dd, J=2, 1 Hz, 1H); LC-MS (LC-ES) M+H=235.

### B. 2-(Pyridin-3-yl)thiazole-5-carboxylic Acid

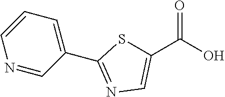 [SMILES: O=C(O)c1cnc(-c2cccnc2)s1]

Lithium hydroxide monohydrate (0.770 g, 18.35 mmol) was added to a solution of ethyl 2-(pyridin-3-yl)thiazole-5-carboxylate (0.860 g, 3.67 mmol) in tetrahydrofuran (6 mL) and water (0.6 mL) and the reaction mixture was stirred in a heating block at 50° C. After 4 hours, the mixture was cooled, poured into water and extracted with diethyl ether (1×).

The aqueous layer was acidified by addition of 1 M hydrochloric acid (18.4 mL). The precipitated solids were collected by filtration, washed with water, and dried in a vacuum oven (50° C./28″ Hg) to afford 2-(pyridin-3-yl)thiazole-5-carboxylic acid (0.603 g, 2.92 mmol, 80% yield) as a tan solid. ¹H NMR (400 MHz, CD₃SOCD₃) δ 7.58 (dd, J=8, 5 Hz, 1H), 8.38 (dt, J=8, 2 Hz, 1H), 8.48 (s, 1H), 8.73 (dd, J=5, 2 Hz, 1H), 9.20 (d, J=2 Hz, 1H), 13.76 (br s, 1H); LC-MS (LC-ES) M+H=207.

Intermediate 26

### (3R,5S)-3-Amino-5-methylpyrrolidin-2-one hydrochloride and (3S,5S)-3-Amino-5-methylpyrrolidin-2-one Hydrochloride

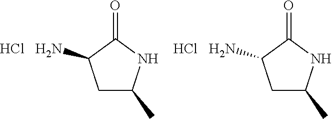

### A. Benzyl (S)-(1-oxopropan-2-yl)carbamate

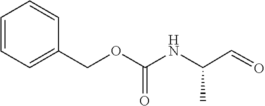

(2,2,6,6-Tetramethylpiperidin-1-yl)oxyl (TEMPO) (0.075 g, 0.480 mmol) was added to benzyl (S)-(1-hydroxypropan-2-yl)carbamate (2.01 g, 9.61 mmol) in dichloromethane (48.0 mL) at 0° C., followed by a 2 M aqueous solution of potassium chloride (0.480 mL, 0.961 mmol). Then, sodium bicarbonate (0.484 g, 5.76 mmol) was dissolved in bleach (sodium hypochlorite (14.82 mL, 14.41 mmol)) and this solution was added to the reaction mixture, which was stirred for two hours at 0° C. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (1:9 to 3:2) to give benzyl (S)-(1-oxopropan-2-yl)carbamate (0.5185 g, 2.377 mmol, 24.74% yield). ¹H NMR (400 MHz, CD₃SOCD₃) δ 1.15 (d, J=7 Hz, 3H), 3.96 (p, J=7 Hz, 1H), 5.04 (s, 2H), 7.28-7.40 (m, 5H), 7.76 (br d, J=7 Hz, 1H), 9.45 (s, 1H); LC-MS (LC-ES) M+H=208.

### B. Methyl (S,Z)-4-(((benzyloxy)carbonyl)amino)-2-((tert-butoxycarbonyl)amino)pent-2-enoate

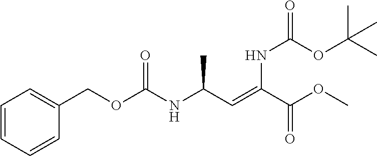 [SMILES: COC(=O)/C(=C/[C@H](C)NC(=O)OCc1ccccc1)NC(=O)OC(C)(C)C]

Methyl 2-((tert-butoxycarbonyl)amino)-2-(dimethoxyphosphoryl)acetate (1.562 g, 5.25 mmol) was added to benzyl (S)-(1-oxopropan-2-yl)carbamate (0.5185 g, 2.502 mmol) in dichloromethane (25.02 mL) at room temperature under a nitrogen atmosphere. Then, 1,8-diazabicyclo[5.4.0]undec-7-ene (0.711 mL, 4.75 mmol) was added and the reaction mixture was stirred for sixteen hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 1:1) to give methyl (S,Z)-4-(((benzyloxy)carbonyl)amino)-2-((tert-butoxycarbonyl)amino)pent-2-enoate (0.7350 g, 1.845 mmol, 73.7% yield). ¹H NMR (400 MHz, CD₃SOCD₃) δ 1.12 (d, J=7 Hz, 3H), 1.37 (s, 9H), 3.65 (s, 3H), 4.39 (q, J=7 Hz, 1H), 4.99 (s, 2H), 6.05 (br s, 1H), 7.26-7.38 (m, 5H), 7.55 (br d, J=8 Hz, 1H), 8.53 (br s, 1H); LC-MS (LC-ES) M+H−(CH₃)₃COCO+H=279.

### C. tert-Butyl ((3R,5S)-5-methyl-2-oxopyrrolidin-3-yl)carbamate and tert-Butyl ((3S,5S)-5-methyl-2-oxopyrrolidin-3-yl)carbamate

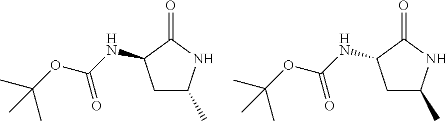

Palladium on carbon (0.103 g, 0.097 mmol) was added to methyl (S,Z)-4-(((benzyloxy)carbonyl)amino)-2-((tert-butoxycarbonyl)amino)pent-2-enoate (0.7350 g, 1.942 mmol) in methanol (19.42 mL) at 25° C. under nitrogen atmosphere. Then, the reaction vessel was fitted with a hydrogen balloon and the vessel was repeatedly evacuated and purged with hydrogen, then stirred for sixteen hours. Then, the vessel was repeatedly evacuated and purged with nitrogen, filtered through Celite®, and concentrated. The resulting residue was purified by reverse phase HPLC, eluting with acetonitrile:water with 0.1% ammonium hydroxide (0:1 to 1:0) to give tert-butyl ((3R,5S)-5-methyl-2-oxopyrrolidin-3-yl)carbamate and tert-butyl ((3S,5S)-5-methyl-2-oxopyrrolidin-3-yl)carbamate (0.3582 g, 1.588 mmol, 82% yield) as a mixture of predominantly the 3R,5S-diastereomer. A small amount of epimerization of the 5S-chiral center occurs somewhere along the synthetic sequence. ¹H NMR (400 MHz, CD₃SOCD₃) δ 1.07 (d, J=6 Hz, 3H), 1.37 (s, 9H), 1.30-1.40 (m, 1H), 2.30-2.38 (m, 1H), 3.40-3.50 (m, 1H), 4.04 (dt, J=12, 9 Hz, 1H), 6.98 (br d, J=9 Hz, 1H), 7.81 (br s, 1H); LC-MS (LC-ES) M+H=215.

### D. (3R,5S)-3-Amino-5-methylpyrrolidin-2-one hydrochloride and (3S,5S)-3-Amino-5-methylpyrrolidin-2-one Hydrochloride

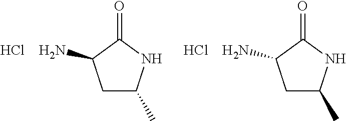

4.0 M Hydrochloric acid (2.090 mL, 8.36 mmol) in 1,4-dioxane was added to tert-butyl ((3R,5S)-5-methyl-2-oxopyrrolidin-3-yl)carbamate and tert-butyl ((3S,5S)-5-methyl-2-oxopyrrolidin-3-yl)carbamate (0.3582 g, 1.672 mmol) in methanol (2.090 mL) at room temperature and the reaction mixture was stirred for sixteen hours. The reaction mixture was concentrated to give (3R,5S)-3-amino-5-methylpyrrolidin-2-one hydrochloride and (3S,5S)-3-amino-5-methylpyrrolidin-2-one hydrochloride (0.102 g, 0.681 mmol) as a mixture of predominantly the 3R,5S-diastereomer. A small amount of epimerization of the 5S-chiral center occurs somewhere along the synthetic sequence. ¹H NMR (400 MHz, CD₃SOCD₃) δ 1.13 (d, J=6 Hz, 3H), 1.42-1.52 (m, 1H), 2.46-2.56 (m, 1H), 3.42-3.52 (m, 1H), 3.84-3.96 (m, 1H), 8.39 (br s, 3H), 8.73 (br s, 1H); LC-MS (LC-ES) M+H=115.

Intermediate 27

### 2-(4-Methylpyrimidin-2-yl)thiazole-5-carboxylic Acid

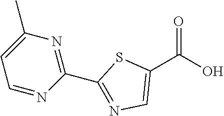

### A. tert-Butyl 2-(4-methylpyrimidin-2-yl)thiazole-5-carboxylate

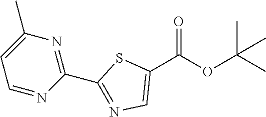 [SMILES: Cc1ccnc(-c2ncc(C(=O)OC(C)(C)C)s2)n1]

A 1.3 M solution of isopropylmagnesium chloride lithium chloride complex in tetrahydrofuran (3.05 mL, 3.97 mmol) was added to a solution of tert-butyl 2-bromothiazole-5-carboxylate (0.873 g, 3.31 mmol) in tetrahydrofuran (12 mL) at −75° C., dropwise, over 3 minutes. The resulting orange solution was stirred 30 minutes and a 1.75 M solution of zinc(II) bromide in tetrahydrofuran (2.455 mL, 4.30 mmol) was added dropwise. The cooling bath was removed and the mixture was warmed to room temperature. Tetrakis(triphenylphosphine)palladium(0) (0.191 g, 0.165 mmol) and 2-bromo-4-methylpyrimidine (0.572 g, 3.31 mmol) were added and the reaction mixture was stirred in a heating block at 80° C. LC-MS after 20 minutes indicated complete conversion. Upon cooling, the reaction mixture was poured into saturated ammonium chloride (50 mL) and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:0) to afford tert-butyl 2-(4-methylpyrimidin-2-yl)thiazole-5-carboxylate (0.864 g, 3.12 mmol, 94% yield) as an orange syrup. ¹H NMR (400 MHz, CD₃SOCD₃) δ 1.60 (s, 9H), 2.66 (s, 3H), 7.22 (d, J=5 Hz, 1H), 8.47 (s, 1H), 8.69 (d, J=5 Hz, 1H); LC-MS (LC-ES) M+H=278.

### B. 2-(4-Methylpyrimidin-2-yl)thiazole-5-carboxylic Acid

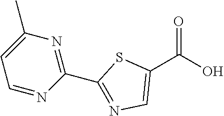

Trifluoroacetic acid (5 mL) was added to a solution of tert-butyl 2-(4-methylpyrimidin-2-yl)thiazole-5-carboxylate (0.864 g, 3.12 mmol) in dichloromethane (12 mL) and the resulting solution was stirred at room temperature. After 16 hours, the volatiles were removed in vacuo (toluene addition and reconcentrated 3×) to afford a slightly tan solid. Further drying (50° C./28″ Hg/2 h) afforded a yellow solid, which still contained trifluoroacetic acid by ¹⁹F NMR. This solid was dissolved in 1 M sodium hydroxide (9 mL), and 1 M hydrochloric acid (9 mL) was added. The precipitated solids were collected by filtration and dried in a vacuum oven (50° C./28″ Hg) to afford 2-(4-methylpyrimidin-2-yl)thiazole-5-carboxylic acid (0.507 g, 2.292 mmol, 73.6% yield) as a tan solid. ¹H NMR (400 MHz, CD₃SOCD₃) δ 2.57 (s, 3H), 7.52 (d, J=5 Hz, 1H), 8.50 (s, 1H), 8.82 (d, J=5 Hz, 1H), 13.80 (br s, 1H); LC-MS (LC-ES) M+H=222.

Intermediate 28

### 2-(3-Cyanophenyl)thiazole-5-carboxylic Acid

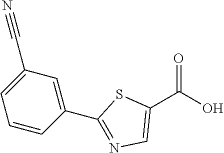 [SMILES: N#Cc1cccc(-c2ncc(C(=O)O)s2)c1]

### A. Ethyl 2-(3-cyanophenyl)thiazole-5-carboxylate

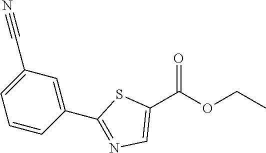 [SMILES: CCOC(=O)c1cnc(-c2cccc(C#N)c2)s1]

A solution of (3-cyanophenyl)zinc(II) iodide (13.06 mL, 6.53 mmol) was added via syringe to ethyl 2-bromothiazole-5-carboxylate (0.65 mL, 4.35 mmol), and tetrakis(triphenylphosphine)palladium(0) (0.251 g, 0.218 mmol) in tetrahydrofuran (3 mL) and the mixture was stirred in a heating block at 70° C. LC MS after 30 minutes indicated consumption of the bromothiazole. Upon cooling, the mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:0) to afford impure ethyl 2-(3-cyanophenyl)thiazole-5-carboxylate (0.8381 g, 3.24 mmol, 74.5% yield) as a colorless solid. ¹H NMR (400 MHz, CD₃SOCD₃) δ 1.32 (t, J=7 Hz, 3H), 4.34 (dq, J=7, 2 Hz, 2H), 7.70-7.80 (m, 1H), 8.00-8.06 (m, 1H), 8.30-8.38 (m, 1H), 8.42-8.48 (m, 1H), 8.52-8.58 (m, 1H); LC-MS (LC-ES) M+H=259.

### B. 2-(3-Cyanophenyl)thiazole-5-carboxylic Acid

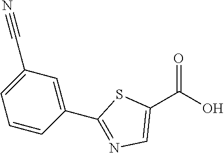 [SMILES: N#Cc1cccc(-c2ncc(C(=O)O)s2)c1]

Lithium hydroxide monohydrate (0.667 g, 15.89 mmol) was added to a solution of ethyl 2-(3-cyanophenyl)thiazole-5-carboxylate (0.821 g, 3.18 mmol) in tetrahydrofuran (5 mL) and water (0.5 mL) and the reaction mixture was stirred in a heating block at 35° C. for 3 hours 30 minutes, then at room temperature overnight. The mixture was poured into water and extracted with diethyl ether (2×). The aqueous layer was acidified by addition of 1 M hydrochloric acid (15.8 mL) and stirred −30 minutes at room temperature. The precipitated solids were collected by filtration, washed with water and dried on the Buchner funnel to afford 2-(3-cyanophenyl)thiazole-5-carboxylic acid (0.7647 g, 3.32 mmol, 104% yield) as a colorless solid. ¹H NMR (400 MHz, CD₃SOCD₃) δ 7.74 (dt, J=8, 1 Hz, 1H), 8.02 (dt, J=8, 1 Hz, 1H), 8.33 (ddd, J=8, 2, 1 Hz, 1H), 8.44 (t, J=1 Hz, 1H), 8.47 (s, 1H), 13.73 (br s, 1H); LC-MS (LC-ES) M+H=231.

Intermediate 29

### 2-(p-Tolyl)thiazole-5-carboxylic Acid

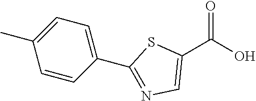

### A. Ethyl 2-(p-tolyl)thiazole-5-carboxylate

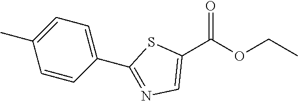 [SMILES: CCOC(=O)c1cnc(-c2ccc(C)cc2)s1]

A solution of p-tolylzinc(II) iodide (13.06 mL, 6.53 mmol) was added, dropwise, over 3 minutes to a solution of ethyl 2-bromothiazole-5-carboxylate (0.65 mL, 4.35 mmol) and tetrakis(triphenylphosphine)palladium(0) (0.251 g, 0.218 mmol) in tetrahydrofuran (3 mL) at room temperature and the reaction mixture was stirred in a heating block at 70° C. for 1 hour. Upon cooling, the reaction mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:0) to afford ethyl 2-(p-tolyl)thiazole-5-carboxylate (0.885 g, 3.58 mmol, 82% yield) as a colorless solid. ¹H NMR (400 MHz, CD₃SOCD₃) δ 1.31 (t, J=7 Hz, 3H), 2.37 (s, 3H), 4.33 (q, J=7 Hz, 2H), 7.35 (m, J=8 Hz, 2H), 7.92 (d, J=8 Hz, 2H), 8.47 (s, 1H); LC-MS (LC-ES) M+H=248.

### B. 2-(p-Tolyl)thiazole-5-carboxylic Acid

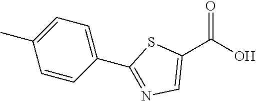

Lithium hydroxide monohydrate (0.741 g, 17.65 mmol) was added to a solution of ethyl 2-(p-tolyl)thiazole-5-carboxylate (0.873 g, 3.53 mmol) in tetrahydrofuran (5 mL) and water (0.5 mL) and the mixture was stirred in a heating block at 50° C. for 3 hours. Upon cooling, the mixture was diluted with water and extracted with diethyl ether. The aqueous layer was acidified by addition of 6 M hydrochloric acid. The precipitated solids were collected by filtration and dried on the Buchner funnel affording 2-(p-tolyl)thiazole-5-carboxylic acid (0.743 g, 3.39 mmol, 96% yield) as a colorless solid. ¹H NMR (400 MHz, CD₃SOCD₃) δ 2.37 (s, 3H), 7.35 (d, J=8 Hz, 2H), 7.91 (d, J=8 Hz, 2H), 8.38 (s, 1H), 13.60 (br s, 1H); LC-MS (LC-ES) M+H=220.

Intermediate 30

### 2-(3-Fluorophenyl)thiazole-5-carboxylic Acid

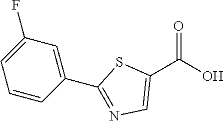 [SMILES: O=C(O)c1cnc(-c2cccc(F)c2)s1]

### A. Ethyl 2-(3-fluorophenyl)thiazole-5-carboxylate

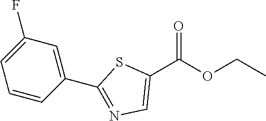 [SMILES: CCOC(=O)c1cnc(-c2cccc(F)c2)s1]

Ethyl 2-bromothiazole-5-carboxylate (0.633 mL, 4.24 mmol) in 1,4-dioxane (5.00 mL) and water (5 mL) were added to (3-fluorophenyl)boronic acid (0.889 g, 6.35 mmol), sodium bicarbonate (1.067 g, 12.71 mmol), and tetrakis(triphenylphosphine)palladium(0) (0.245 g, 0.212 mmol) and the reaction mixture was sparged with nitrogen for ˜15 minutes. Then, the reaction mixture was stirred in a heating block at 80° C. After 20 hours, the reaction mixture was cooled, poured into water and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:0) to afford ethyl 2-(3-fluorophenyl)thiazole-5-carboxylate (0.511 g, 2.034 mmol, 48.0% yield) as a colorless solid. LC-MS (LC-ES) M+H=252.

### B. 2-(3-Fluorophenyl)thiazole-5-carboxylic Acid

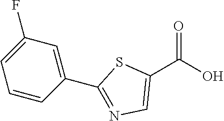 [SMILES: O=C(O)c1cnc(-c2cccc(F)c2)s1]

Lithium hydroxide monohydrate (0.549 g, 13.09 mmol) was added to a solution of ethyl 2-(3-fluorophenyl)thiazole-5-carboxylate (0.658 g, 2.62 mmol, multiple batches) in tetrahydrofuran (5 mL) and water (0.5 mL) and the reaction mixture was stirred over a weekend (went dry, LC-MS indicated complete conversion). The residue was partitioned between water and diethyl ether and the aqueous layer was extracted with diethyl ether (1×). The aqueous layer was carefully acidified by addition of 1 M hydrochloric acid. The precipitated solids were collected by filtration and dried on the Buchner funnel to afford 2-(3-fluorophenyl)thiazole-5-carboxylic acid (0.536 g, 2.401 mmol, 92% yield) as a colorless solid. ¹H NMR (400 MHz, CD₃SOCD₃) δ 7.41 (td, J=8, 2 Hz, 1H), 7.59 (dq, J=8, 6 Hz, 1H), 7.80-7.86 (m, 1H), 7.86 (dd, J=8, 1 Hz, 1H), 8.44 (s, 1H), 13.74 (br s, 1H); LC-MS (LC-ES) M+H=224.

Intermediate 31

### 2-(6-Methylpyridin-2-yl)thiazole-5-carboxylic Acid

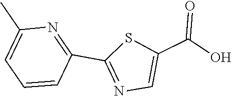

### A. Ethyl 2-(6-methylpyridin-2-yl)thiazole-5-carboxylate

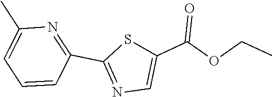 [SMILES: CCOC(=O)c1cnc(-c2cccc(C)n2)s1]

A 1.3 M solution of isopropylmagnesium chloride lithium chloride complex in tetrahydrofuran (4.08 mL, 5.30 mmol) was added, dropwise over ˜ 5 minutes to a solution of 2-iodo-6-methylpyridine (1.01 g, 4.61 mmol) in tetrahydrofuran (8 mL) at −78° C. and the reaction mixture was stirred for 30 minutes. Then, a 1.73 M solution of zinc(II) bromide in tetrahydrofuran (3.33 mL, 5.76 mmol) was added dropwise. The cooling bath was removed and the reaction mixture was warmed to room temperature. Tetrakis(triphenylphosphine)palladium(0) (0.266 g, 0.231 mmol) and ethyl 2-bromothiazole-5-carboxylate (0.689 mL, 4.61 mmol) were added and the reaction mixture was stirred in a heating block at 70° C. LC-MS after 15 minutes indicated nearly complete reaction. Upon cooling, the reaction mixture was poured into saturated ammonium chloride (50 mL) and water (10 mL) and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (1:9 to 1:4) to afford ethyl 2-(6-methylpyridin-2-yl)thiazole-5-carboxylate (0.414 g, 1.667 mmol, 36.2% yield) as an orange solid. ¹H NMR (400 MHz, CD₃SOCD₃) δ 1.32 (t, J=7 Hz, 3H), 2.54 (s, 3H), 4.33 (q, J=7 Hz, 2H), 7.44 (ddd, J=8, 2, 1 Hz, 1H), 7.89 (t, J=8 Hz, 1H), 7.98 (dd, J=8, 1 Hz, 1H), 8.51 (s, 1H); LC-MS (LC-ES) M+H=249.

### B. 2-(6-Methylpyridin-2-yl)thiazole-5-carboxylic Acid

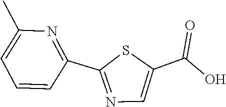

Lithium hydroxide monohydrate (0.342 g, 8.16 mmol) was added to a solution of ethyl 2-(6-methylpyridin-2-yl)thiazole-5-carboxylate (0.405 g, 1.631 mmol) in tetrahydrofuran (5 mL) and water (0.5 mL) and the reaction mixture was stirred overnight in a heating block at 50° C. Upon cooling, the reaction mixture was poured into water and extracted with diethyl ether (1×). The aqueous layer was acidified by addition of 1 M hydrochloric acid (8.2 mL). The precipitated solids were collected by filtration, washed with water, and dried in a vacuum oven (50° C./28″ Hg) to obtain 2-(6-methylpyridin-2-yl)thiazole-5-carboxylic acid (0.244 g, 1.108 mmol, 67.9% yield) as an off-white solid. ¹H NMR (400 MHz, CD₃SOCD₃) δ 2.55 (s, 3H), 7.43 (d, J=7 Hz, 1H), 7.89 (t, J=8 Hz, 1H), 7.98 (d, J=8 Hz, 1H), 8.45 (s, 1H), 13.63 (br s, 1H); LC-MS (LC-ES) M+H=221.

Intermediate 32

### 2-(4-Methylpyridin-2-yl)thiazole-5-carboxylic Acid

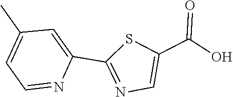

### A. Ethyl 2-(4-methylpyridin-2-yl)thiazole-5-carboxylate

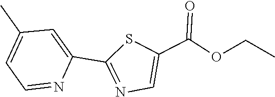 [SMILES: CCOC(=O)c1cnc(-c2cc(C)ccn2)s1]

A 1.3 M solution of isopropylmagnesium chloride lithium chloride complex in tetrahydrofuran (3.28 mL, 4.27 mmol) was added, dropwise, over ˜5 minutes to a solution of 2-iodo-4-methylpyridine (0.779 g, 3.56 mmol) in tetrahydrofuran (10 mL) at −78° C. and the reaction mixture was stirred for 30 minutes. Then, a 1.81 M solution of zinc(II) bromide in tetrahydrofuran (2.55 mL, 4.62 mmol) was added dropwise. The cooling bath was removed and the reaction mixture was warmed to room temperature. Tetrakis(triphenylphosphine)palladium(0) (0.205 g, 0.178 mmol) and ethyl 2-bromothiazole-5-carboxylate (0.531 mL, 3.56 mmol) were added and the reaction mixture was stirred in a heating block at 70° C. After 90 minutes, the reaction mixture was cooled, poured into saturated ammonium chloride (50 mL) and water (15 mL) and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (0:1 to 1:3) to afford ethyl 2-(4-methylpyridin-2-yl)thiazole-5-carboxylate (0.495 g, 1.994 mmol, 56.1% yield) as a tan solid. ¹H NMR (400 MHz, CDCl₃) δ 1.40 (t, J=7 Hz, 3H), 2.45 (s, 3H), 4.40 (q, J=7 Hz, 2H), 7.21 (ddd, J=5, 2, 1 Hz, 1H), 8.07 (t, J=1 Hz, 1H), 8.46 (s, 1H), 8.49 (d, J=5 Hz, 1H); LC-MS (LC-ES) M+H=249.

### B. 2-(4-Methylpyridin-2-yl)thiazole-5-carboxylic Acid

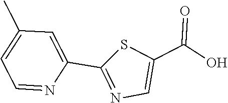

Lithium hydroxide monohydrate (0.410 g, 9.77 mmol) was added to a solution of ethyl 2-(4-methylpyridin-2-yl)thiazole-5-carboxylate (0.485 g, 1.953 mmol) in tetrahydrofuran (5 mL) and water (0.5 mL) and the reaction mixture was stirred in a heating block at 60° C. in a sealed vial. LC-MS after 2.5 hours indicated complete conversion. Upon cooling, the reaction mixture was poured into water and extracted with diethyl ether (2×). The aqueous layer was acidified by addition of 1 M hydrochloric acid (9.8 mL). The precipitated solids were collected by filtration, washed with water, and dried on the Buchner funnel to afford 2-(4-methylpyridin-2-yl)thiazole-5-carboxylic acid (0.338 g, 1.535 mmol, 79% yield) as a colorless solid. ¹H NMR (400 MHz, CD₃SOCD₃) δ 2.44 (s, 3H), 7.41 (ddd, J=5, 2, 1 Hz, 1H), 8.04 (t, J=1 Hz, 1H), 8.47 (s, 1H), 8.54 (d, J=5 Hz, 1H), 13.66 (br s, 1H); LC-MS (LC-ES) M+H=221.

Intermediate 33

### 2-(3-(Difluoromethyl)-5-methylphenyl)thiazole-5-carboxylic Acid

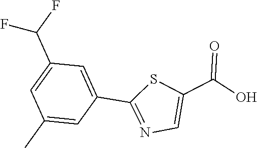 [SMILES: Cc1cc(-c2ncc(C(=O)O)s2)cc(C(F)F)c1]

### A. 1-Bromo-3-(difluoromethyl)-5-methylbenzene

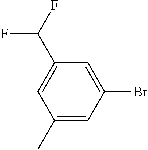

(Diethylamino)sulfur trifluoride (1.601 mL, 12.12 mmol) was added to 3-bromo-5-methylbenzaldehyde (2.01 g, 10.10 mmol) in dichloromethane (50.5 mL) at room temperature and the reaction mixture was stirred sixteen hours. Saturated sodium bicarbonate was added and the reaction mixture was extracted with dichloromethane, dried over magnesium sulfate, filtered, and concentrated. The residue was purified by silica gel chromatography, eluting with ethyl acetate:hexanes (0:1 to 1:4) to give 1-bromo-3-(difluoromethyl)-5-methylbenzene (1.61 g, 6.92 mmol, 68.5% yield). ¹H NMR (400 MHz, CD₃SOCD₃) δ 2.35 (s, 3H), 6.98 (t, J=56 Hz, 1H), 7.40 (s, 1H), 7.55 (s, 1H), 7.60 (s, 1H).

### B. Ethyl 2-(3-(difluoromethyl)-5-methylphenyl)thiazole-5-carboxylate

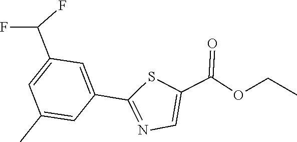 [SMILES: CCOC(=O)c1cnc(-c2cc(C)cc(C(F)F)c2)s1]

tert-Butyl lithium in pentane (1.7 M, 4.50 mL, 7.64 mmol) was added, dropwise, over 3 minutes to a solution of 1-bromo-3-(difluoromethyl)-5-methylbenzene (0.824 g, 3.73 mmol) in tetrahydrofuran (12 mL) at −75° C. and the resulting dark orange solution was stirred 30 minutes. Then, a 1.67 M solution of zinc(II) bromide in tetrahydrofuran (2.68 mL, 4.47 mmol) was added dropwise (orange color fades to yellow during addition). The cooling bath was removed and the mixture was warmed to room temperature. Tetrakis(triphenylphosphine)palladium(0) (0.215 g, 0.186 mmol) and ethyl 2-bromothiazole-5-carboxylate (0.880 g, 3.73 mmol) were added and the reaction mixture was stirred in a heating block at 80° C. LC-MS after 45 minutes indicated the reaction was complete. Upon cooling, the reaction mixture was poured into saturated ammonium chloride and extracted with ethyl acetate (3×). The combined organics were washed with water and saturated sodium chloride, dried over sodium sulfate, and concentrated in vacuo. The residue was purified by silica gel chromatography, eluting with ethyl acetate:hexanes (0:1 to 1:4) to afford ethyl 2-(3-(difluoromethyl)-5-methylphenyl)thiazole-5-carboxylate (0.861 g, 2.90 mmol, 78% yield) as a colorless syrup which slowly crystallized. ¹H NMR (400 MHz, CDCl₃) δ 1.42 (t, J=7 Hz, 3H), 2.49 (s, 3H), 4.41 (q, J=7 Hz, 2H), 6.68 (t, J=56 Hz, 1H), 7.45 (s, 1H), 7.92 (d, J=5 Hz, 2H), 8.44 (s, 1H); LC-MS (LC-ES) M+H=298.

### C. 2-(3-(Difluoromethyl)-5-methylphenyl)thiazole-5-carboxylic Acid

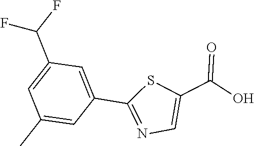 [SMILES: Cc1cc(-c2ncc(C(=O)O)s2)cc(C(F)F)c1]

Lithium hydroxide monohydrate (0.601 g, 14.33 mmol) was added to a solution of ethyl 2-(3-(difluoromethyl)-5-methylphenyl)thiazole-5-carboxylate (0.852 g, 2.87 mmol) in tetrahydrofuran (5 mL) and water (0.5 mL) and the reaction mixture was stirred in a heating block at 50° C. After 5 hours, the reaction mixture was cooled, poured into water, and extracted with diethyl ether (2×). The aqueous layer was acidified by addition of 1 M hydrochloric acid (14.4 mL). The precipitated solids were collected by filtration, washed with water, and dried in a vacuum oven (50° C./28″ Hg) to afford 2-(3-(difluoromethyl)-5-methylphenyl)thiazole-5-carboxylic acid (0.679 g, 2.52 mmol, 88% yield) as a colorless solid. ¹H NMR (400 MHz, CD₃SOCD₃) δ 2.45 (s, 3H), 7.11 (t, J=56 Hz, 1H), 7.57 (s, 1H), 8.00 (d, J=5 Hz, 2H), 8.44 (s, 1H), 13.71 (br s, 1H); LC-MS (LC-ES) M+H=270.

Intermediate 34

### Racemic (3R,3aR,6aR)-3-Aminohexahydrocyclopenta[b]pyrrol-2(1H)-one hydrochloride and (3S,3aS,6aS)-3-Aminohexahydrocyclopenta[b]pyrrol-2(1H)-one hydrochloride

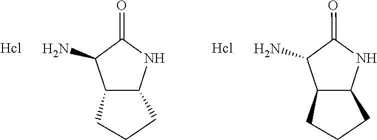 [SMILES: N[C@H]1C(=O)N[C@@H]2CCC[C@@H]21 ; N[C@@H]1C(=O)N[C@H]2CCC[C@H]21]

### A. Racemic (R)-tert-Butyl 2-amino-2-((1R,2S)-2-hydroxycyclopentyl)acetate and (S)-tert-Butyl 2-amino-2-((1S,2R)-2-hydroxycyclopentyl)acetate and Racemic (R)-tert-Butyl 2-amino-2-((1S,2R)-2-hydroxycyclopentyl)acetate and (S)-tert-Butyl 2-amino-2-((1R,2S)-2-hydroxycyclopentyl)acetate

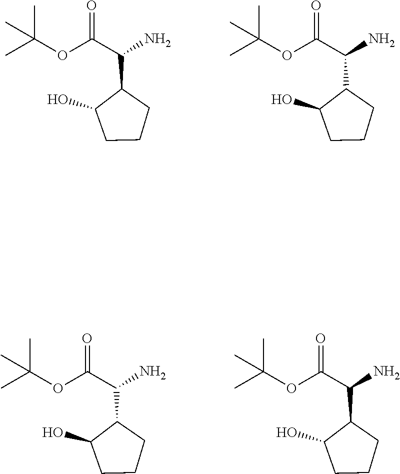

tert-Butyl 2-((diphenylmethylene)amino)acetate (6.02 g, 20.38 mmol) was added to 6-oxabicyclo[3.1.0]hexane (1.71 g, 20.33 mmol) in tetrahydrofuran (100 mL) and the reaction mixture was kept under nitrogen and cooled on a dry ice/acetone bath. Then, 1 M lithium hexamethyldisilazide in tetrahydrofuran (21 mL, 21.00 mmol) was added, followed by boron trifluoride diethyl etherate (2.5 mL, 20.26 mmol) and the reaction mixture was stirred for ˜1 hour 20 minutes, then the dry ice/acetone bath was removed. After an additional ˜2 hours 5 minutes, the reaction mixture was quenched with 10% citric acid solution (100 mL). The biphasic reaction mixture was stirred for 5 days, then diluted with hexanes (25 mL). The layers were separated. The aqueous layer was washed with hexanes (50 mL, 2×). The organic layers were discarded. The aqueous layer was cooled on an ice bath and slowly basified with 6 M sodium hydroxide (25 mL) to pH=˜12. The mixture was extracted with dichloromethane (50 mL, 3×). The organic layers were dried over magnesium sulfate, filtered, and concentrated to give a mixture of racemic (R)-tert-butyl 2-amino-2-((1R,2S)-2-hydroxycyclopentyl)acetate and (S)-tert-butyl 2-amino-2-((1 S,2R)-2-hydroxycyclopentyl)acetate as well as racemic (R)-tert-butyl 2-amino-2-((1 S,2R)-2-hydroxycyclopentyl)acetate and (S)-tert-butyl 2-amino-2-((1R,2S)-2-hydroxycyclopentyl)acetate (3.71 g, 17.2 mmol, 85% yield) as a pale tan oil which was carried forward. LC-MS (LC-ES) M+H=216.

### B. Racemic (R)-tert-Butyl 2-((tert-butoxycarbonyl)amino)-2-((1R,2S)-2-hydroxycyclopentyl)acetate and (S)-tert-Butyl 2-((tert-butoxycarbonyl)amino)-2-((1S,2R)-2-hydroxycyclopentyl)acetate and Racemic (R)-tert-Butyl 2-((tert-butoxycarbonyl)amino)-2-((1S,2R)-2-hydroxycyclopentyl)acetate and (S)-tert-Butyl 2-((tert-butoxycarbonyl)amino)-2-((1R,2S)-2-hydroxycyclopentyl)acetate

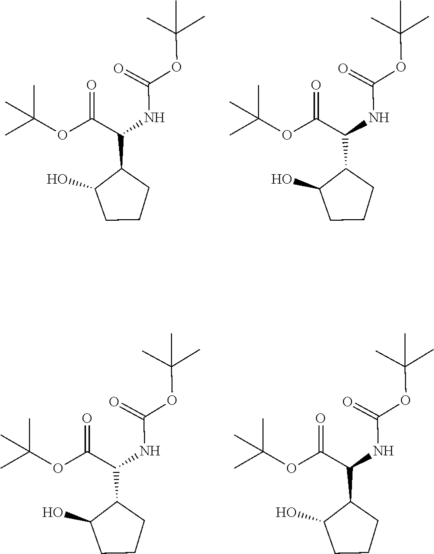 [SMILES: CC(C)(C)OC(=O)N[C@@H](C(=O)OC(C)(C)C)[C@H]1CCC[C@@H]1O ; CC(C)(C)OC(=O)N[C@H](C(=O)OC(C)(C)C)[C@@H]1CCC[C@H]1O ; CC(C)(C)OC(=O)N[C@@H](C(=O)OC(C)(C)C)[C@@H]1CCC[C@H]1O ; CC(C)(C)OC(=O)N[C@H](C(=O)OC(C)(C)C)[C@H]1CCC[C@@H]1O]

A solution of the di-tert-butyl dicarbonate (4.503 g, 20.63 mmol) in dichloromethane (10 mL) was added to a mixture of racemic (R)-tert-butyl 2-amino-2-((1R,2S)-2-hydroxycyclopentyl)acetate and (S)-tert-butyl 2-amino-2-((1 S,2R)-2-hydroxycyclopentyl)acetate as well as racemic (R)-tert-butyl 2-amino-2-((1 S,2R)-2-hydroxycyclopentyl)acetate and (S)-tert-butyl 2-amino-2-((1R,2S)-2-hydroxycyclopentyl)acetate (3.71 g, 17.23 mmol) in dichloromethane (140 mL). After stirring for ˜1 hour 30 minutes, the reaction mixture was concentrated. Dichloromethane was added to the residue and the mixture was absorbed onto silica gel. The material was purified by silica gel chromatography, eluting with ethyl acetate:hexanes (0:1 to 2:3) to give a mixture of racemic (R)-tert-butyl 2-((tert-butoxycarbonyl)amino)-2-((1R,2S)-2-hydroxycyclopentyl)acetate and (S)-tert-butyl 2-((tert-butoxycarbonyl)amino)-2-((1 S,2R)-2-hydroxycyclopentyl)acetate as well as racemic (R)-tert-butyl 2-((tert-butoxycarbonyl)amino)-2-((1 S,2R)-2-hydroxycyclopentyl)acetate and (S)-tert-butyl 2-((tert-butoxycarbonyl)amino)-2-((1R,2S)-2-hydroxycyclopentyl)acetate (4.82 g, 15.3 mmol, 89% yield) as a nearly colorless oil. LC-MS (LC-ES) M+H=316.

### C. Racemic (R)-tert-Butyl 2-((1R,2R)-2-azidocyclopentyl)-2-((tert-butoxycarbonyl)amino)acetate and (S)-tert-Butyl 2-((1S,2S)-2-azidocyclopentyl)-2-((tert-butoxycarbonyl)amino)acetate and Racemic (R)-tert-Butyl 2-((1S,2S)-2-azidocyclopentyl)-2-((tert-butoxycarbonyl)amino)acetate and (S)-tert-Butyl 2-((1R,2R)-2-azidocyclopentyl)-2-((tert-butoxycarbonyl)amino)acetate

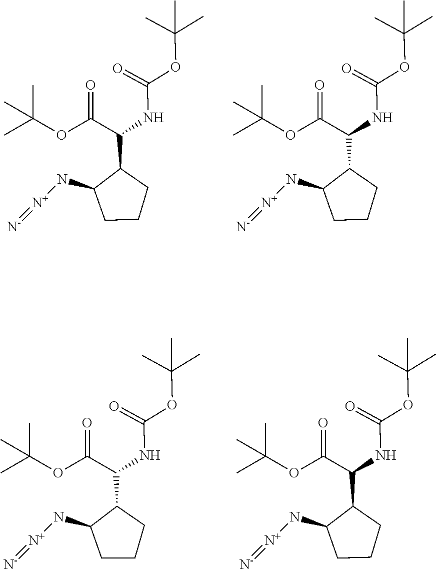 [SMILES: CC(C)(C)OC(=O)N[C@@H](C(=O)OC(C)(C)C)[C@H]1CCC[C@H]1N[NH+]=[N-] ; CC(C)(C)OC(=O)N[C@H](C(=O)OC(C)(C)C)[C@@H]1CCC[C@H]1N[NH+]=[N-] ; CC(C)(C)OC(=O)N[C@@H](C(=O)OC(C)(C)C)[C@@H]1CCC[C@H]1N[NH+]=[N-] ; CC(C)(C)OC(=O)N[C@H](C(=O)OC(C)(C)C)[C@H]1CCC[C@H]1N[NH+]=[N-]]

Triphenylphosphine (4.435 g, 16.91 mmol) was added to a mixture of racemic (R)-tert-butyl 2-((tert-butoxycarbonyl)amino)-2-((1R,2S)-2-hydroxycyclopentyl)acetate and (S)-tert-butyl 2-((tert-butoxycarbonyl)amino)-2-((1 S,2R)-2-hydroxycyclopentyl)acetate as well as racemic (R)-tert-butyl 2-((tert-butoxycarbonyl)amino)-2-((1 S,2R)-2-hydroxycyclopentyl)acetate and (S)-tert-butyl 2-((tert-butoxycarbonyl)amino)-2-((1R,2S)-2-hydroxycyclopentyl)acetate (4.82 g, 15.28 mmol) in tetrahydrofuran (150 mL). The reaction was cooled on an ice bath under nitrogen. A solution of diisopropyl azodicarboxylate (3.27 mL, 16.82 mmol) in tetrahydrofuran (10 mL) was added over-11 minutes. Then, after ˜20 minutes, a solution of the diphenylphosphoryl azide (3.62 mL, 16.80 mmol) in tetrahydrofuran (10 mL) was added, over ˜2½ minutes. The ice bath was removed after ˜1 hour 10 minutes. After stirring ˜14 hours 45 minutes, the reaction mixture was concentrated. The resulting oil was absorbed onto silica gel. The material was purified by silica gel chromatography, eluting with ethyl acetate:hexanes (0:1 to 1:4) to give a solid. The solid was dissolved in ethyl acetate and filtered through a bit of cotton, then concentrated to give a mixture of racemic (R)-tert-butyl 2-((1R,2R)-2-azidocyclopentyl)-2-((tert-butoxycarbonyl)amino)acetate and (S)-tert-butyl 2-((1 S,2S)-2-azidocyclopentyl)-2-((tert-butoxycarbonyl)amino)acetate as well as racemic (R)-tert-butyl 2-((1 S,2S)-2-azidocyclopentyl)-2-((tert-butoxycarbonyl)amino)acetate and (S)-tert-butyl 2-((1R,2R)-2-azidocyclopentyl)-2-((tert-butoxycarbonyl)amino)acetate (4.71 g, 13.8 mmol, 91% yield) as a colorless oil. LC-MS (LC-ES) M+H=341.

### D. Racemic Tert-Butyl ((3R,3aR,6aS)-2-oxooctahydrocyclopenta[b]pyrrol-3-yl)carbamate and Tert-Butyl ((3S,3aS,6aR)-2-oxooctahydrocyclopenta[b]pyrrol-3-yl)carbamate and Racemic Tert-Butyl ((3R,3aS,6aR)-2-oxooctahydrocyclopenta[b]pyrrol-3-yl)carbamate and Tert-Butyl ((3S,3aR,6aS)-2-oxooctahydrocyclopenta[b]pyrrol-3-yl)carbamate

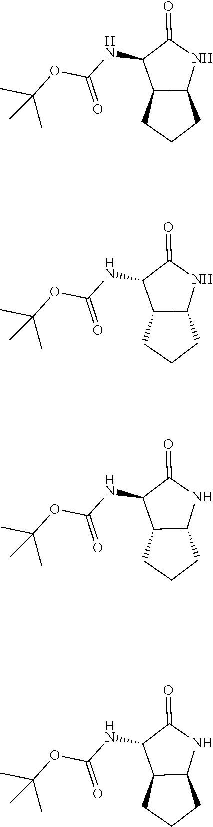 [SMILES: CC(C)(C)OC(=O)N[C@H]1C(=O)N[C@H]2CCC[C@H]21 ; CC(C)(C)OC(=O)N[C@@H]1C(=O)N[C@@H]2CCC[C@H]12 ; CC(C)(C)OC(=O)N[C@H]1C(=O)N[C@@H]2CCC[C@@H]21 ; CC(C)(C)OC(=O)N[C@@H]1C(=O)N[C@H]2CCC[C@H]21]

Zinc (dust, <10 micron) (4.521 g, 69.1 mmol) was added to a mixture of racemic (R)-tert-butyl 2-((1R,2R)-2-azidocyclopentyl)-2-((tert-butoxycarbonyl)amino)acetate and (S)-tert-butyl 2-((1 S,2S)-2-azidocyclopentyl)-2-((tert-butoxycarbonyl)amino)acetate as well as racemic (R)-tert-butyl 2-((1 S,2S)-2-azidocyclopentyl)-2-((tert-butoxycarbonyl)amino)acetate and (S)-tert-butyl 2-((1R,2R)-2-azidocyclopentyl)-2-((tert-butoxycarbonyl)amino)acetate (4.71 g, 13.84 mmol) in acetic acid (150 mL) under nitrogen and the reaction mixture was stirred for ˜18 hours 55 minutes, then the reaction mixture was filtered over a pad of Celite® and the filter cake was rinsed with acetic acid. The filtrate was concentrated. The residue was partitioned between in dichloromethane (100 mL) and 1 M potassium carbonate solution (50 mL). The layers were separated and the aqueous layer was extracted once more with dichloromethane (50 mL). The organic layers were dried over magnesium sulfate, filtered over a pad of Celite®, and the filtrate concentrated to an oil. Toluene (25 mL) was added to the residue and the mixture was put onto a pre-heated 80° C. heating block and heated for 4 days. The reaction mixture was concentrated. Dichloromethane was added to the residue and the mixture was absorbed onto silica gel. The material was purified by silica gel chromatography, eluting with (3:1 ethyl acetate:ethanol):hexanes (0:1 to 1:1) and the mixed fractions were repurified to give racemic tert-butyl ((3R,3aR,6aS)-2-oxooctahydrocyclopenta[b]pyrrol-3-yl)carbamate and tert-butyl ((3S,3aS,6aR)-2-oxooctahydrocyclopenta[b]pyrrol-3-yl)carbamate (0.612 g, 2.55 mmol, 18% yield) as a cream-colored powder and racemic tert-butyl ((3R,3aS,6aR)-2-oxooctahydrocyclopenta[b]pyrrol-3-yl)carbamate and tert-butyl ((3S,3aR,6aS)-2-oxooctahydrocyclopenta[b]pyrrol-3-yl)carbamate (1.004 g, 4.18 mmol, 30% yield) as a pale tan powder.

### Racemic Tert-Butyl ((3R,3aR,6aS)-2-oxooctahydrocyclopenta[b]pyrrol-3-yl)carbamate and Tert-Butyl ((3S,3aS,6aR)-2-oxooctahydrocyclopenta[b]pyrrol-3-yl)carbamate

¹H NMR (400 MHz, CD₃SOCD₃) δ 1.34-1.40 (m, 2H), 1.38 (s, 9H), 1.40-1.64 (m, 4H), 2.68-2.78 (m, 1H), 3.82-3.90 (m, 1H), 4.23 (t, J=9 Hz, 1H), 7.05 (br d, J=9 Hz, 1H), 7.68 (br s, 1H); LC-MS (LC-ES) M+H=241.

### Racemic Tert-Butyl ((3R,3aS,6aR)-2-oxooctahydrocyclopenta[b]pyrrol-3-yl)carbamate and Tert-Butyl ((3S,3aR,6aS)-2-oxooctahydrocyclopenta[b]pyrrol-3-yl)carbamate

¹H NMR (400 MHz, CD₃SOCD₃) δ 1.36 (s, 9H), 1.40-1.70 (m, 6H), 2.42-2.50 (m, 1H), 3.53 (dd, J=8, 5 Hz, 1H), 3.86 (t, J=6 Hz, 1H), 7.18 (br d, J=9 Hz, 1H), 7.77 (br s, 1H); LC-MS (LC-ES) M+H=241.

### E. Racemic (3R,3aR,6aR)-3-Aminohexahydrocyclopenta[b]pyrrol-2(1H)-one hydrochloride and (3S,3aS,6aS)-3-Aminohexahydrocyclopenta[b]pyrrol-2(1H)-one Hydrochloride

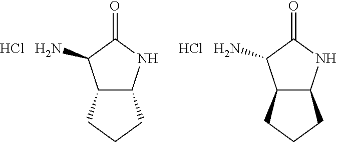

4 M Hydrochloric acid in 1,4-dioxane (8 mL, 32.0 mmol) was added to racemic tert-butyl ((3R,3aS,6aR)-2-oxooctahydrocyclopenta[b]pyrrol-3-yl)carbamate and tert-butyl ((3S,3aR,6aS)-2-oxooctahydrocyclopenta[b]pyrrol-3-yl)carbamate (1.004 g, 4.18 mmol) in dichloromethane (40 mL). After stirring for ˜15 hours 5 minutes, the reaction mixture was concentrated. dichloromethane was added and the mixture was concentrated again to give racemic (3R,3aR,6aR)-3-aminohexahydrocyclopenta[b]pyrrol-2(1-)-one hydrochloride and (3S,3aS,6aS)-3-aminohexahydrocyclopenta[b]pyrrol-2(1H)-one hydrochloride (0.751 g, 4.18 mmol, 100% yield assuming 98% pure by weight) as a cream-colored powder. LC-MS (LC-ES) M+H=141.

Intermediate 35

### Racemic ((3S,3aS,6aR)-3-Aminotetrahydro-1H-furo[3,4-b]pyrrol-2(3H)-one Hydrochloride and (3R,3aR,6aS)-3-Aminotetrahydro-1H-furo[3,4-b]pyrrol-2(3H)-one Hydrochloride

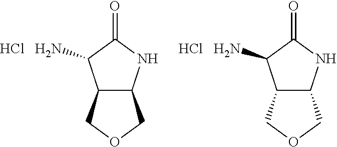

### A. Racemic (R)-tert-Butyl 2-amino-2-((3S,4R)-4-hydroxytetrahydrofuran-3-yl)acetate and (S)-tert-Butyl 2-amino-2-((3R,4S)-4-hydroxytetrahydrofuran-3-yl)acetate and Racemic (R)-tert-Butyl 2-amino-2-((3R,4S)-4-hydroxytetrahydrofuran-3-yl)acetate and (S)-tert-Butyl 2-amino-2-((3S,4R)-4-hydroxytetrahydrofuran-3-yl)acetate

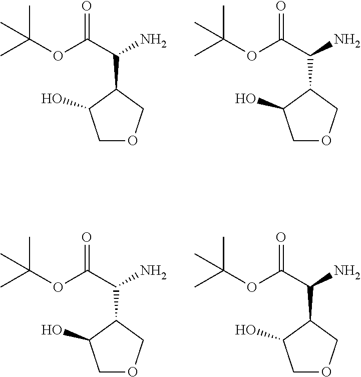

tert-Butyl 2-((diphenylmethylene)amino)acetate (6.01 g, 20.35 mmol) was added to 3,6-dioxabicyclo[3.1.0]hexane (1.74 g, 20.21 mmol) in tetrahydrofuran (200 mL) and the reaction mixture was kept under nitrogen and cooled on a dry ice/acetone bath. Then, 1 M lithium hexamethyldisilazide in tetrahydrofuran (21 mL, 21.00 mmol) was added, followed by boron trifluoride diethyl etherate (2.5 mL, 20.26 mmol). After ˜40 minutes, the dry ice/acetone bath was removed. After an additional ˜15 hours 50 minutes, the reaction was quenched with 10% citric acid solution (200 mL). After ˜30 minutes, the reaction was warmed to 55° C. on a heating block for ˜3 hours 50 minutes. It was a biphasic mixture. The reaction mixture was diluted with hexanes (200 mL). The layers were separated and the aqueous layer was washed with hexanes (50 mL, 2×). The organic layers were discarded. 6 M Sodium hydroxide (50 mL) was added to the aqueous layer to bring the pH ˜10. The aqueous layer was extracted with dichloromethane (50 mL, 4×). The organic layers were dried with magnesium sulfate, filtered, and concentrated to give a mixture of racemic (R)-tert-butyl 2-amino-2-((3S,4R)-4-hydroxytetrahydrofuran-3-yl)acetate and (S)-tert-butyl 2-amino-2-((3R,4S)-4-hydroxytetrahydrofuran-3-yl)acetate as well as racemic (R)-tert-butyl 2-amino-2-((3R,4S)-4-hydroxytetrahydrofuran-3-yl)acetate and (S)-tert-butyl 2-amino-2-((3S,4R)-4-hydroxytetrahydrofuran-3-yl)acetate (1.94 g, 8.93 mmol, 44% yield) as a tan oil which was carried on to the next reaction. LC-MS (LC-ES) M+H=218.

### B. Racemic (R)-tert-Butyl 2-((tert-butoxycarbonyl)amino)-2-((3S,4R)-4-hydroxytetrahydrofuran-3-yl)acetate and (S)-tert-Butyl 2-((tert-butoxycarbonyl)amino)-2-((3R,4S)-4-hydroxytetrahydrofuran-3-yl)acetate and Racemic (R)-tert-Butyl 2-((tert-butoxycarbonyl)amino)-2-((3R,4S)-4-hydroxytetrahydrofuran-3-yl)acetate and (S)-tert-Butyl 2-((tert-butoxycarbonyl)amino)-2-((3S,4R)-4-hydroxytetrahydrofuran-3-yl)acetate

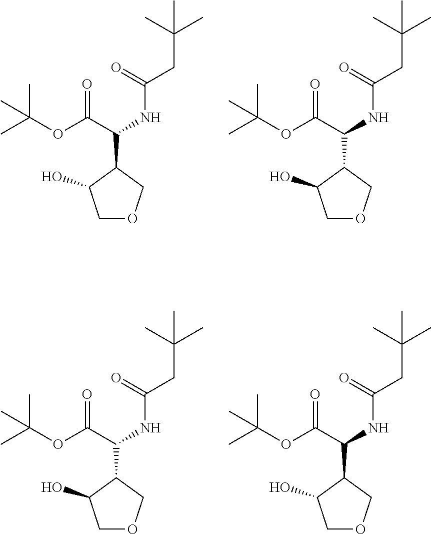 [SMILES: CC(C)(C)CC(=O)N[C@@H](C(=O)OC(C)(C)C)[C@H]1COC[C@@H]1O ; CC(C)(C)CC(=O)N[C@H](C(=O)OC(C)(C)C)[C@@H]1COC[C@H]1O ; CC(C)(C)CC(=O)N[C@@H](C(=O)OC(C)(C)C)[C@@H]1COC[C@H]1O ; CC(C)(C)CC(=O)N[C@H](C(=O)OC(C)(C)C)[C@H]1COC[C@@H]1O]

A solution of the di-tert-butyl dicarbonate (2.152 g, 9.86 mmol) in dichloromethane (5 mL) was added to a mixture of racemic (R)-tert-butyl 2-amino-2-((3S,4R)-4-hydroxytetrahydrofuran-3-yl)acetate and (S)-tert-butyl 2-amino-2-((3R,4S)-4-hydroxytetrahydrofuran-3-yl)acetate as well as racemic (R)-tert-butyl 2-amino-2-((3R,4S)-4-hydroxytetrahydrofuran-3-yl)acetate and (S)-tert-butyl 2-amino-2-((3S,4R)-4-hydroxytetrahydrofuran-3-yl)acetate (1.94 g, 8.93 mmol) in dichloromethane (85 mL). After stirring for ˜2 hours 45 minutes, more di-tert-butyl dicarbonate (2.152 g, 9.86 mmol) in dichloromethane (5 mL) was added. After an additional ˜1 hour 55 minutes, the reaction mixture was concentrated. Dichloromethane was added to the residue and the mixture was absorbed onto silica gel. The material was purified by silica gel chromatography, eluting with (ethyl acetate:hexanes (0:1 to 3:2) to give a mixture of racemic (R)-tert-butyl 2-((tert-butoxycarbonyl)amino)-2-((3S,4R)-4-hydroxytetrahydrofuran-3-yl)acetate and (S)-tert-butyl 2-((tert-butoxycarbonyl)amino)-2-((3R,4S)-4-hydroxytetrahydrofuran-3-yl)acetate as well as racemic (R)-tert-butyl 2-((tert-butoxycarbonyl)amino)-2-((3R,4S)-4-hydroxytetrahydrofuran-3-yl)acetate and (S)-tert-butyl 2-((tert-butoxycarbonyl)amino)-2-((3S,4R)-4-hydroxytetrahydrofuran-3-yl)acetate (2.072 g, 6.53 mmol, 73% yield) as a pale tan oil. LC-MS (LC-ES) M+H=318.

### C. Racemic (R)-tert-Butyl 2-((3R,4S)-4-azidotetrahydrofuran-3-yl)-2-((tert-butoxycarbonyl)amino)acetate and (S)-tert-Butyl 2-((3S,4R)-4-azidotetrahydrofuran-3-yl)-2-((tert-butoxycarbonyl)amino)acetate and Racemic (R)-tert-Butyl 2-((3S,4R)-4-azidotetrahydrofuran-3-yl)-2-((tert-butoxycarbonyl)amino)acetate and (S)-tert-Butyl 2-((3R,4S)-4-azidotetrahydrofuran-3-yl)-2-((tert-butoxycarbonyl)amino)acetate

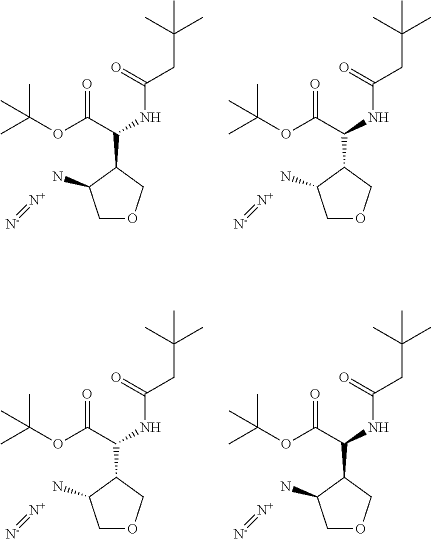

Triphenylphosphine (1.903 g, 7.26 mmol) was added to a mixture of racemic (R)-tert-butyl 2-((tert-butoxycarbonyl)amino)-2-((3S,4R)-4-hydroxytetrahydrofuran-3-yl)acetate and (S)-tert-butyl 2-((tert-butoxycarbonyl)amino)-2-((3R,4S)-4-hydroxytetrahydrofuran-3-yl)acetate as well as racemic (R)-tert-butyl 2-((tert-butoxycarbonyl)amino)-2-((3R,4S)-4-hydroxytetrahydrofuran-3-yl)acetate and (S)-tert-butyl 2-((tert-butoxycarbonyl)amino)-2-((3S,4R)-4-hydroxytetrahydrofuran-3-yl)acetate (2.072 g, 6.53 mmol) in tetrahydrofuran (65 mL). The reaction was kept under nitrogen and cooled on an ice bath. A solution of diisopropyl azodicarboxylate (1.40 mL, 7.20 mmol) in tetrahydrofuran (4 mL) was added over ˜3½ minutes. After ˜20 minutes, a solution of the diphenylphosphoryl azide (1.55 mL, 7.19 mmol) in tetrahydrofuran (3 mL) was added. After ˜2 hours 20 minutes, the ice bath was removed. After stirring ˜15 hours 40 minutes, the reaction mixture was diluted with hexanes (65 mL) and concentrated. Dichloromethane was added to the residue and the mixture was absorbed onto silica gel. The material was purified by silica gel chromatography, eluting with ethyl acetate:hexanes (0:1 to 1:1) to give racemic (R)-tert-butyl 2-((3R,4S)-4-azidotetrahydrofuran-3-yl)-2-((tert-butoxycarbonyl)amino)acetate and (S)-tert-butyl 2-((3S,4R)-4-azidotetrahydrofuran-3-yl)-2-((tert-butoxycarbonyl)amino)acetate as well as racemic (R)-tert-butyl 2-((3S,4R)-4-azidotetrahydrofuran-3-yl)-2-((tert-butoxycarbonyl)amino)acetate and (S)-tert-butyl 2-((3R,4S)-4-azidotetrahydrofuran-3-yl)-2-((tert-butoxycarbonyl)amino)acetate (2.120 g, 6.19 mmol, 95% yield) as a colorless oil. LC-MS (LC-ES) M+H=343.

### D. Racemic Tert-Butyl ((3R,3aR,6aR)-2-oxohexahydro-1H-furo[3,4-b]pyrrol-3-yl)carbamate and Tert-Butyl ((3S,3aS,6aS)-2-oxohexahydro-1H-furo[3,4-b]pyrrol-3-yl)carbamate and Racemic Tert-Butyl ((3R,3aS,6aS)-2-oxohexahydro-1H-furo[3,4-b]pyrrol-3-yl)carbamate and tert-Butyl ((3S,3aR,6aR)-2-oxohexahydro-1H-furo[3,4-b]pyrrol-3-yl)carbamate

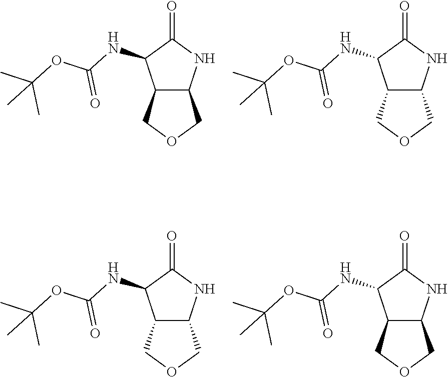

Zinc (dust, <10 micron) (2.033 g, 31.1 mmol) was added to racemic (R)-tert-butyl 2-((3R,4S)-4-azidotetrahydrofuran-3-yl)-2-((tert-butoxycarbonyl)amino)acetate and (S)-tert-butyl 2-((3S,4R)-4-azidotetrahydrofuran-3-yl)-2-((tert-butoxycarbonyl)amino)acetate as well as racemic (R)-tert-butyl 2-((3S,4R)-4-azidotetrahydrofuran-3-yl)-2-((tert-butoxycarbonyl)amino)acetate and (S)-tert-butyl 2-((3R,4S)-4-azidotetrahydrofuran-3-yl)-2-((tert-butoxycarbonyl)amino)acetate (2.120 g, 6.19 mmol) in acetic acid (80 mL) and the reaction mixture was stirred under nitrogen for ˜16 hours 5 minutes. Then, the reaction mixture was filtered over a pad of Celite® and the filter cake was rinsed with acetic acid. The filtrate was concentrated. The residue was partitioned between dichloromethane (100 mL) and 1 M potassium carbonate (50 mL). The layers were separated and the aqueous layer was extracted once more with dichloromethane (50 mL). The organic layers were dried over magnesium sulfate, filtered, and the filtrate was concentrated to an oil. Toluene (20 mL) was added to the residue and the mixture was heated at 80° C. for ˜17 hours 50 minutes. There were solids present. The solids were filtered off and rinsed with toluene. The filtrate was concentrated and the residue was combined with the filtered solids using dichloromethane and methanol. The mixture was absorbed onto silica gel. The material was purified by silica gel chromatography, eluting with ((3:1) ethyl acetate:ethanol):hexanes (0:1 to 0:1), then repurified by silica gel chromatography, eluting with methanol:dichloromethane (0:1 to 1:9) with the mixed fractions resubmitted to the same conditions to ultimately give racemic tert-butyl ((3R,3aR,6aR)-2-oxohexahydro-1H-furo[3,4-b]pyrrol-3-yl)carbamate and tert-butyl ((3S,3aS,6aS)-2-oxohexahydro-1H-furo[3,4-b]pyrrol-3-yl)carbamate (0.274 g, 1.13 mmol, 18% yield) as a cream-colored powder and racemic tert-butyl ((3R,3aS,6aS)-2-oxohexahydro-1H-furo[3,4-b]pyrrol-3-yl)carbamate and tert-butyl ((3S,3aR,6aR)-2-oxohexahydro-1H-furo[3,4-b]pyrrol-3-yl)carbamate (0.317 g, 1.31 mmol, 21% yield) as a cream-colored powder.

### Racemic Tert-Butyl ((3R,3aR,6aR)-2-oxohexahydro-1H-furo[3,4-b]pyrrol-3-yl)carbamate and Tert-Butyl ((3S,3aS,6aS)-2-oxohexahydro-1H-furo[3,4-b]pyrrol-3-yl)carbamate

¹H NMR (400 MHz, CD₃SOCD₃) δ 1.38 (s, 9H), 2.94-3.04 (m, 1H), 3.42-3.50 (m, 2H), 3.63 (br d, J=10 Hz, 1H), 3.81 (dd, J=10, 5 Hz, 1H), 4.02 (dd, J=7, 4 Hz, 1H), 4.33 (t, J=9 Hz, 1H), 7.14 (d, J=8 Hz, 1H), 7.89 (br s, 1H); LC-MS (LC-ES) M+H=243.

### Racemic Tert-Butyl ((3R,3aS,6aS)-2-oxohexahydro-1H-furo[3,4-b]pyrrol-3-yl)carbamate and Tert-Butyl ((3S,3aR,6aR)-2-oxohexahydro-1H-furo[3,4-b]pyrrol-3-yl)carbamate

¹H NMR (400 MHz, CD₃SOCD₃) δ 1.37 (s, 9H), 2.69 (br q, J=6 Hz, 1H), 3.38 (dd, J=10, 5 Hz, 1H), 3.54 (dd, J=10, 6 Hz, 1H), 3.59 (br d, J=10 Hz, 1H), 3.65 (dd, J=8, Hz, 1H), 3.75 (br d, J=9 Hz, 1H), 4.05 (dd, J=8, 5 Hz, 1H), 7.28 (d, J=8 Hz, 1H), 7.97 (br s, 1H); LC-MS (LC-ES) M+H=243.

### E. Racemic ((3S,3aS,6aR)-3-Aminotetrahydro-1H-furo[3,4-b]pyrrol-2(3H)-one Hydrochloride and (3R,3aR,6aS)-3-Aminotetrahydro-1H-furo[3,4-b]pyrrol-2(3H)-one Hydrochloride

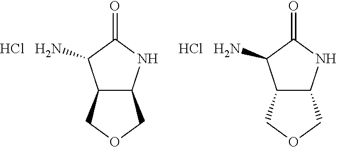

4 M Hydrochloric acid in 1,4-dioxane (2.5 mL, 10.00 mmol) was added to racemic tert-butyl ((3R,3aS,6aS)-2-oxohexahydro-1H-furo[3,4-b]pyrrol-3-yl)carbamate and tert-butyl ((3S,3aR,6aR)-2-oxohexahydro-1H-furo[3,4-b]pyrrol-3-yl)carbamate (0.317 g, 1.308 mmol) in dichloromethane (13 mL). After stirring for ˜16 hours 30 minutes, the reaction mixture was concentrated. Dichloromethane was added and the reaction mixture was concentrated again to give racemic ((3S,3aS,6aR)-3-aminotetrahydro-1H-furo[3,4-b]pyrrol-2(3H)-one hydrochloride and (3R,3aR,6aS)-3-aminotetrahydro-1H-furo[3,4-b]pyrrol-2(3H)-one hydrochloride (0.241 g, 1.31 mmol, 100% yield assuming 97% pure by weight) as a pale tan powder. LC-MS (LC-ES) M+H=143.

Intermediate 36

### Racemic 7-Amino-2-oxa-5-azaspiro[3.4]octan-6-one

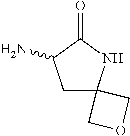

### A. Benzyl (3-(hydroxymethyl)oxetan-3-yl)carbamate

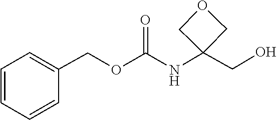 [SMILES: O=C(NC1(CO)COC1)OCc1ccccc1]

Triethylamine (2.197 mL, 7.88 mmol) was added to 3-(((benzyloxy)carbonyl)amino)oxetane-3-carboxylic acid (1.98 g, 7.88 mmol) in tetrahydrofuran (35.0 mL) at 0° C., followed by isopropyl chloroformate (7.88 mL, 7.88 mmol) and the reaction was stirred for 30 minutes. Then, the reaction mixture was filtered into sodium borohydride (0.388 g, 10.25 mmol) in water (4.38 mL) and the reaction mixture was stirred for eighteen hours. The reaction mixture was filtered, saturated sodium bicarbonate added, extracted with ethyl acetate, washed with saturated sodium bicarbonate and saturated sodium chloride, dried over magnesium sulfate, filtered, and concentrated. The residue was purified by silica gel chromatography, eluting with ethyl acetate:hexanes (1:4 to 4:1) to give benzyl (3-(hydroxymethyl)oxetan-3-yl)carbamate (0.5606 g, 2.245 mmol, 28.5% yield). ¹H NMR (400 MHz, CD₃SOCD₃) δ 3.58 (d, J=6 Hz, 2H), 4.43 (ABq, J_{AB}=6 Hz, Δv_{AB}=19 Hz, 4H), 4.99 (s, 2H), 5.08 (br t, J=6 Hz, 1H), 7.26-7.40 (m, 5H), 7.78 (br s, 1H); LC-MS (LC-ES) M+H=238.

### B. Benzyl (3-formyloxetan-3-yl)carbamate

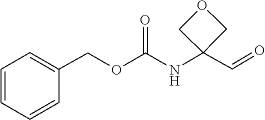 [SMILES: O=CC1(NC(=O)OCc2ccccc2)COC1]

(2,2,6,6-Tetramethylpiperidin-1-yl)oxyl (TEMPO) (0.018 g, 0.118 mmol) was added to benzyl (3-(hydroxymethyl)oxetan-3-yl)carbamate (0.5606 g, 2.363 mmol) in dichloromethane (11.81 mL) at 0° C., followed by a 2 M aqueous solution of potassium chloride (0.118 mL, 0.236 mmol). Then, sodium bicarbonate (0.119 g, 1.418 mmol) was dissolved in bleach (sodium hypochlorite (3.65 mL, 3.54 mmol)) and this solution was added to the reaction mixture, which was stirred for two hours at 0° C. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (1:4 to 3:2) to give benzyl (3-formyloxetan-3-yl)carbamate (0.3337 g, 1.348 mmol, 57.0% yield). ¹H NMR (400 MHz, CD₃SOCD₃) δ 4.60 (ABq, J_{AB}=7 Hz, Δv_{AB}=49 Hz, 4H), 5.05 (s, 2H), 7.30-7.40 (m, 5H), 8.62 (br s, 1H), 9.60 (s, 1H); LC-MS (LC-ES) M+H=236.

### C. Methyl (Z)-2-(((benzyloxy)carbonyl)amino)-3-(3-(((benzyloxy)carbonyl)amino)oxetan-3-yl)acrylate

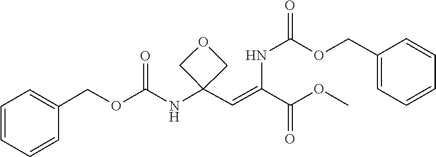 [SMILES: COC(=O)/C(=C/C1(NC(=O)OCc2ccccc2)COC1)NC(=O)OCc1ccccc1]

Methyl 2-(((benzyloxy)carbonyl)amino)-2-(dimethoxyphosphoryl)acetate (0.987 g, 2.98 mmol) was added to benzyl (3-formyloxetan-3-yl)carbamate (0.3337 g, 1.419 mmol) in dichloromethane (7.09 mL) at room temperature under a nitrogen atmosphere. Then, 1,8-diazabicyclo[5.4.0]undec-7-ene (0.403 mL, 2.70 mmol) was added and the reaction mixture was stirred for sixteen hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 3:2) to give methyl (Z)-2-(((benzyloxy)carbonyl)amino)-3-(3-(((benzyloxy)carbonyl)amino)oxetan-3-yl)acrylate (0.2711 g, 0.585 mmol, 41.2% yield). ¹H NMR (400 MHz, CD₃SOCD₃) δ 3.65 (s, 3H), 4.60 (ABq, J_{AB}=7 Hz, Δv_{AB}=23 Hz, 4H), 5.00 (s, 2H), 5.06 (s, 2H), 6.52 (br s, 1H), 7.26-7.40 (m, 10H), 8.24 (br s, 1H), 8.66 (br s, 1H); LC-MS (LC-ES) M+H=441.

### D. Racemic 7-Amino-2-oxa-5-azaspiro[3.4]octan-6-one

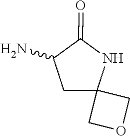

Palladium on carbon (0.033 g, 0.031 mmol) was added to methyl (Z)-2-(((benzyloxy)carbonyl)amino)-3-(3-(((benzyloxy)carbonyl)amino)oxetan-3-yl)acrylate (0.2711 g, 0.616 mmol) in methanol (6.16 mL) at 25° C. under nitrogen atmosphere. Then, the reaction vessel was fitted with a hydrogen balloon and the vessel was repeatedly evacuated and purged with hydrogen, then stirred for sixteen hours. Then, the vessel was repeatedly evacuated and purged with nitrogen, filtered through Celite®, and concentrated to give crude 7-amino-2-oxa-5-azaspiro[3.4]octan-6-one (0.0868 g, 0.488 mmol, 79% yield), which was carried forward into the next reaction. ¹H NMR (400 MHz, CD₃SOCD₃) δ 1.87 (dd, J=13, 9 Hz, 1H), 2.00 (br s, 2H), 2.68 (dd, J=13, 8 Hz, 1H), 3.30 (dd, J=10, 8 Hz, 1H), 4.51 (ABq, J_{AB}=6 Hz, Δv_{AB}=20 Hz, 2H), 4.54 (ABq, J_{AB}=6 Hz, Δv_{AB}=44 Hz, 2H), 8.51 (br s, 1H); LC-MS (LC-ES) M+H=143.

Intermediate 37

### Racemic (3S,3aR,6aR)-3-Aminohexahydrocyclopenta[b]pyrrol-2(1H)-one Hydrochloride and (3R,3aS,6aS)-3-Aminohexahydrocyclopenta[b]pyrrol-2(1H)-one Hydrochloride

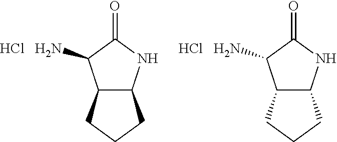

4 M Hydrochloric acid in 1,4-dioxane (5 mL, 20.00 mmol) was added to racemic tert-butyl ((3R,3aR,6aS)-2-oxooctahydrocyclopenta[b]pyrrol-3-yl)carbamate and tert-butyl ((3S,3aS,6aR)-2-oxooctahydrocyclopenta[b]pyrrol-3-yl)carbamate (0.612 g, 2.55 mmol, Intermediate 34D) in dichloromethane (25 mL). After ˜14 hours 35 minutes, the reaction mixture was concentrated. Dichloromethane was added and the mixture was concentrated again to give racemic (3S,3aR,6aR)-3-aminohexahydrocyclopenta[b]pyrrol-2(1H)-one hydrochloride and (3R,3aS,6aS)-3-aminohexahydrocyclopenta[b]pyrrol-2(1H)-one hydrochloride (0.464 g, 2.55 mmol, 100% yield assuming 97% pure by weight) as a cream-colored powder. LC-MS (LC-ES) M+H=141.

Intermediate 38

### (3R,5R)-3-Amino-5-methylpyrrolidin-2-one Hydrochloride and (3S,5R)-3-Amino-5-methylpyrrolidin-2-one Hydrochloride

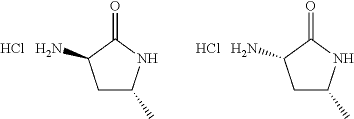

### A. Benzyl (R)-(1-oxopropan-2-yl)carbamate

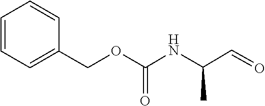

(2,2,6,6-Tetramethylpiperidin-1-yl)oxyl (TEMPO) (0.103 g, 0.660 mmol) was added to benzyl (R)-(1-hydroxypropan-2-yl)carbamate (2.76 g, 13.19 mmol) in dichloromethane (66.0 mL) at 0° C., followed by a 2 M aqueous solution of potassium chloride (0.660 mL, 1.319 mmol). Then, sodium bicarbonate (0.665 g, 7.91 mmol) was dissolved in bleach (sodium hypochlorite (20.35 mL, 19.79 mmol)) and this solution was added to the reaction mixture, which was stirred for one hour at 0° C. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (0:1 to 2:3) to give benzyl (R)-(1-oxopropan-2-yl)carbamate (0.5982 g, 2.74 mmol, 20.79% yield). The major product was the carboxylic acid. A small amount of epimerization of the R-chiral center occurs somewhere along the synthetic sequence. ¹H NMR (400 MHz, CD₃SOCD₃) δ 1.15 (d, J=7 Hz, 3H), 3.96 (p, J=7 Hz, 1H), 5.04 (s, 2H), 7.28-7.40 (m, 5H), 7.77 (br d, J=7 Hz, 1H), 9.45 (s, 1H); LC-MS (LC-ES) M+H=208.

### B. Methyl (R,Z)-4-(((benzyloxy)carbonyl)amino)-2-((tert-butoxycarbonyl)amino)pent-2-enoate

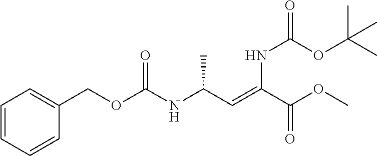 [SMILES: COC(=O)/C(=C/[C@@H](C)NC(=O)OCc1ccccc1)NC(=O)OC(C)(C)C]

Methyl 2-((tert-butoxycarbonyl)amino)-2-(dimethoxyphosphoryl)acetate (2.63 g, 8.85 mmol) was added to benzyl (R)-(1-oxopropan-2-yl)carbamate (0.8734 g, 4.21 mmol, from multiple batches) in dichloromethane (42.1 mL) at room temperature under a nitrogen atmosphere. Then, 1,8-diazabicyclo[5.4.0]undec-7-ene (1.198 mL, 8.01 mmol) was added and the reaction mixture was stirred for sixty-six hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 3:2) to give methyl (R,2)-4-(((benzyloxy)carbonyl)amino)-2-((tert-butoxycarbonyl)amino)pent-2-enoate (1.36 g, 3.41 mmol, 81% yield). A small amount of epimerization of the R-chiral center occurs somewhere along the synthetic sequence. ¹H NMR (400 MHz, CD₃SOCD₃) δ 1.12 (d, J=7 Hz, 3H), 1.37 (s, 9H), 3.65 (s, 3H), 4.39 (q, J=8 Hz, 1H), 4.99 (s, 2H), 6.06 (br s, 1H), 7.26-7.38 (m, 5H), 7.55 (br d, J=8 Hz, 1H), 8.54 (br s, 1H); LC-MS (LC-ES) M+H−(CH₃)₃COCO+H=279.

### C. tert-Butyl ((3R,5R)-5-methyl-2-oxopyrrolidin-3-yl)carbamate and Tert-Butyl ((3S,5R)-5-methyl-2-oxopyrrolidin-3-yl)carbamate

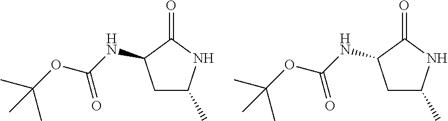

Palladium on carbon (0.191 g, 0.180 mmol) was added to methyl (R,Z)-4-(((benzyloxy)carbonyl)amino)-2-((tert-butoxycarbonyl)amino)pent-2-enoate (1.36 g, 3.59 mmol) in methanol (35.9 mL) at 25° C. under nitrogen atmosphere. Then, the reaction vessel was fitted with a hydrogen balloon and the vessel was repeatedly evacuated and purged with hydrogen, then stirred for forty-two hours. Then, the vessel was repeatedly evacuated and purged with nitrogen, filtered through Celite®, and concentrated. The resulting residue was purified by reverse phase HPLC, eluting with acetonitrile:water with 0.1% ammonium hydroxide (0:1 to 1:0) to give an unequal diastereomeric mixture of tert-butyl ((3R,5R)-5-methyl-2-oxopyrrolidin-3-yl)carbamate and tert-butyl ((3S,5R)-5-methyl-2-oxopyrrolidin-3-yl)carbamate (0.6693 g, 2.97 mmol, 83% yield) as a mixture of predominantly the 3S,5R-diastereomer (˜5:1 by NMR). A small amount of epimerization of the 5R-chiral center occurs somewhere along the synthetic sequence. ¹H NMR (400 MHz, CD₃SOCD₃) δ 1.07 (d, J=6 Hz, 3H), 1.37 (s, 9H), 1.30-1.40 (m, 1H), 2.30-2.38 (m, 1H), 3.40-3.50 (m, 1H), 4.04 (dt, J=12, 9 Hz, 1H), 6.98 (br d, J=9 Hz, 1H), 7.81 (br s, 1H); LC-MS (LC-ES) M+H=215.

### D. (3R,5R)-3-Amino-5-methylpyrrolidin-2-one Hydrochloride and (3S,5R)-3-Amino-5-methylpyrrolidin-2-one Hydrochloride

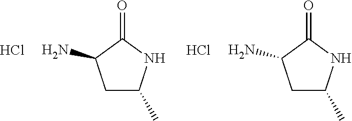

4.0 M Hydrochloric acid (3.90 mL, 15.62 mmol) in dioxane was added to an unequal diastereomeric mixture of tert-butyl ((3R,5R)-5-methyl-2-oxopyrrolidin-3-yl)carbamate and tert-butyl ((3S,5R)-5-methyl-2-oxopyrrolidin-3-yl)carbamate (0.6693 g, 3.12 mmol) in methanol (3.90 mL) at room temperature and the reaction mixture was stirred for sixteen hours. The reaction mixture was concentrated to give an unequal diastereomeric mixture of (3R,5R)-3-amino-5-methylpyrrolidin-2-one hydrochloride and (3S,5R)-3-amino-5-methylpyrrolidin-2-one hydrochloride (0.4900 g, 3.09 mmol, 99% yield) as a mixture of predominantly the 3S,5R-diastereomer (˜5:1 by NMR). A small amount of epimerization of the 5R-chiral center occurs somewhere along the synthetic sequence. ¹H NMR (400 MHz, CD₃SOCD₃) δ 1.13 (d, J=6 Hz, 3H), 1.42-1.52 (m, 1H), 2.46-2.56 (m, 1H), 3.56-3.64 (m, 1H), 3.84-3.96 (m, 1H), 8.39 (br s, 3H), 8.40 (br s, 1H); LC-MS (LC-ES) M+H=115.

Intermediate 39

### 2-(3-Methoxyphenyl)thiazole-5-carboxylic Acid

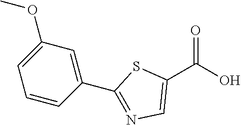 [SMILES: COc1cccc(-c2ncc(C(=O)O)s2)c1]

### A. Ethyl 2-(3-methoxyphenyl)thiazole-5-carboxylate

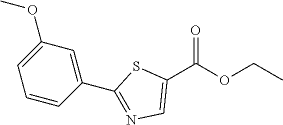 [SMILES: CCOC(=O)c1cnc(-c2cccc(OC)c2)s1]

(3-Methoxyphenyl)boronic acid (0.359 g, 2.364 mmol) was added to ethyl 2-bromothiazole-5-carboxylate (0.5074 g, 2.149 mmol) in 1,4-dioxane (10.75 mL) at room temperature, followed by potassium carbonate (0.594 g, 4.30 mmol) and the reaction mixture was purged with nitrogen. Then, bis(triphenylphosphine)palladium(II) chloride (0.151 g, 0.215 mmol) was added and the reaction mixture was heated at 85° C. for sixteen hours. After cooling, the reaction mixture was poured into saturated sodium chloride, extracted with dichloromethane, dried over magnesium sulfate, filtered, and concentrated. The residue was purified by silica gel chromatography, eluting with ethyl acetate:heptane (0:1 to 3:7) to give ethyl 2-(3-methoxyphenyl)thiazole-5-carboxylate (0.1563 g, 0.564 mmol, 26.2% yield). ¹H NMR (400 MHz, CD₃SOCD₃) δ 1.32 (t, J=7 Hz, 3H), 3.84 (s, 3H), 4.34 (q, J=7 Hz, 2H), 7.14 (ddd, J=8, 3, 1 Hz, 1H), 7.46 (t, J=8 Hz, 1H), 7.54 (dd, J=2, 2 Hz, 1H), 7.60 (ddd, J=8, 2, 1 Hz, 1H), 8.50 (br s, 1H); LC-MS (LC-ES) M+H=264.

### B. 2-(3-Methoxyphenyl)thiazole-5-carboxylic Acid

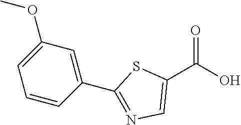 [SMILES: COc1cccc(-c2ncc(C(=O)O)s2)c1]

Lithium hydroxide monohydrate (0.075 g, 1.781 mmol) was added to ethyl 2-(3-methoxyphenyl)thiazole-5-carboxylate (0.1563 α. 0.594 mmol) in methanol (9.50 mL) and water (2.374 mL) at room temperature and the reaction mixture was stirred three hours. The reaction mixture was concentrated. The reaction mixture was dissolved in ethyl acetate and washed with 10% citric acid, dried over magnesium sulfate, filtered, and concentrated to give 2-(3-methoxyphenyl)thiazole-5-carboxylic acid (0.1329 g, 0.537 mmol, 90% yield). ¹H NMR (400 MHz, CD₃SOCD₃) δ 3.83 (s, 3H), 7.13 (ddd, J=8, 3, 1 Hz, 1H), 7.45 (t, J=8 Hz, 1H), 7.52 (dd, J=2, 2 Hz, 1H), 7.57 (ddd, J=8, 2, 1 Hz, 1H), 8.41 (s, 1H), 12.46 (br s, 1H); LC-MS (LC-ES) M+H=236.

Intermediate 40

### 2-(3-Hydroxyphenyl)thiazole-5-carboxylic Acid

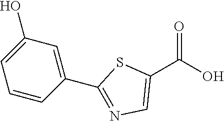 [SMILES: O=C(O)c1cnc(-c2cccc(O)c2)s1]

Boron tribromide (3.23 mL, 3.23 mmol) was added to 2-(3-methoxyphenyl)thiazole-5-carboxylic acid (0.0761 g, 0.323 mmol, Intermediate 39) in dichloromethane (6.47 mL) at −78° C. Then, the reaction mixture was allowed to warm to room temperature and stirred for sixty-six hours. Methanol was slowly added to the reaction mixture, followed by water, then the reaction mixture was concentrated. The resulting residue was purified by reverse phase HPLC chromatography, eluting with acetonitrile:water with 0.1% ammonium hydroxide (0:1 to 1:0), then concentrated. The residue was dissolved in methanol and acidified with 4.0 M hydrochloric acid in dioxane, then concentrated to give 2-(3-hydroxyphenyl)thiazole-5-carboxylic acid (0.0642 g, 0.276 mmol, 85% yield). ¹H NMR (400 MHz, CD₃SOCD₃) δ 6.94 (ddd, J=9, 2, 1 Hz, 1H), 7.04 (s, 1H), 7.17 (s, 1H), 7.30 (s, 1H), 7.32 (t, J=8 Hz, 1H), 7.38-7.42 (m, 1H), 9.89 (br s, 1H); LC-MS (LC-ES) M+H=222.

Intermediate 41

### 3-Amino-1-(4-methoxybenzyl)-3-methylpyrrolidin-2-one hydroiodide

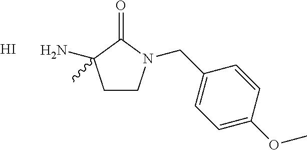

### A. 1-(4-Methoxybenzyl)pyrrolidin-2-one

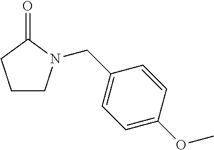

Sodium hydride (1.268 g, 31.7 mmol) was added to pyrrolidin-2-one (2.57 g, 30.2 mmol) in 1,4-dioxane (101 mL) at 0° C., followed by para-methoxybenzyl chloride (4.30 mL, 31.7 mmol) and tetrabutylammonium bromide (0.097 g, 0.302 mmol) and the reaction mixture was stirred at 80° C. for sixteen hours. The reaction mixture was quenched with water, extracted with ethyl acetate, dried over magnesium sulfate, filtered, and concentrated. The residue was purified by silica gel chromatography, eluting with ethyl acetate:hexanes (3:7) to give 1-(4-methoxybenzyl)pyrrolidin-2-one (4.73 g, 21.89 mmol, 72.5% yield). ¹H NMR (400 MHz, CD₃SOCD₃) δ 1.82-1.94 (m, 2H), 2.25 (t, J=8 Hz, 2H), 3.17 (t, J=7 Hz, 2H), 3.72 (s, 3H), 4.27 (s, 2H), 6.88 (d, J=9 Hz, 2H), 7.13 (d, J=9 Hz, 2H); LC-MS (LC-ES) M+H=206.

### B. Ethyl 1-(4-methoxybenzyl)-3-methyl-2-oxopyrrolidine-3-carboxylate

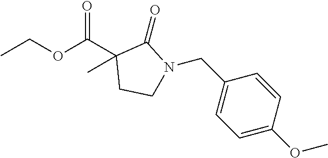 [SMILES: CCOC(=O)C1(C)CCN(Cc2ccc(OC)cc2)C1=O]

A solution of 2.0 M lithium diisopropylamide (13.83 mL, 27.7 mmol) in tetrahydrofuran was added dropwise to 1-(4-methoxybenzyl)pyrrolidin-2-one (4.73 g, 23.04 mmol) in tetrahydrofuran (115 mL) at −78° C. under nitrogen and the solution was stirred for 30 minutes. Then, ethyl chloroformate (2.434 mL, 25.3 mmol) was added and the reaction mixture was stirred for 30 minutes. Then, lithium diisopropylamide (13.83 mL, 27.7 mmol) was added and the reaction mixture was stirred for five minutes. Then, iodomethane (1.729 mL, 27.7 mmol) was added and the reaction mixture was allowed to warm to room temperature and stirred for two hours. It was quenched with 10% citric acid, extracted with ethyl acetate, dried over magnesium sulfate, filtered and concentrated. The residue was purified by silica gel chromatography, eluting with ethyl acetate:hexanes (3:7 to 7:3) to give ethyl 1-(4-methoxybenzyl)-3-methyl-2-oxopyrrolidine-3-carboxylate (5.47 g, 17.84 mmol, 77% yield). ¹H NMR (400 MHz, CD₃SOCD₃) δ 1.11 (t, J=7 Hz, 3H), 1.27 (s, 3H), 1.88 (ddd, J=16, 9, 7 Hz, 1H), 2.32 (ddd, J=13, 9, 5 Hz, 1H), 3.14-3.28 (m, 2H), 3.72 (s, 3H), 4.00-4.12 (m, 2H), 4.33 (ABq, J_{AB}=15 Hz, Δv_{AB}=81 Hz, 2H), 6.89 (d, J=9 Hz, 2H), 7.14 (d, J=9 Hz, 2H); LC-MS (LC-ES) M+H=292.

### C. 1-(4-Methoxybenzyl)-3-methyl-2-oxopyrrolidine-3-carboxylic Acid

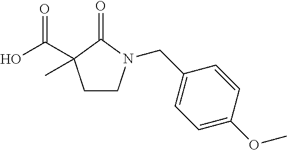 [SMILES: COc1ccc(CN2CCC(C)(C(=O)O)C2=O)cc1]

Sodium hydroxide (18.77 mL, 37.5 mmol) was added to ethyl 1-(4-methoxybenzyl)-3-methyl-2-oxopyrrolidine-3-carboxylate (5.47 g, 18.77 mmol) in tetrahydrofuran (31.3 mL) and methanol (15.65 mL) at room temperature, and the reaction was stirred for two hours. The reaction mixture was treated with 1 M hydrochloric acid to pH=˜1 and then the reaction mixture was extracted with ethyl acetate, dried over magnesium sulfate, filtered, and concentrated to give 1-(4-methoxybenzyl)-3-methyl-2-oxopyrrolidine-3-carboxylic acid (5.91 g, 17.96 mmol, 96% yield). ¹H NMR (400 MHz, CD₃SOCD₃) δ 1.29 (s, 3H), 1.83 (ddd, J=15, 8, 7 Hz, 1H), 2.34 (ddd, J=12, 8, 4 Hz, 1H), 3.20-3.28 (m, 2H), 3.72 (s, 3H), 4.32 (ABq, J_{AB}=15 Hz, Δv_{AB}=49 Hz, 2H),6.88 (d, J=9 Hz, 2H), 7.14 (d, J=9 Hz, 2H), 12.66 (br s, 1H); LC-MS (LC-ES) M+H=264.

### D. Tert-Butyl (1-(4-methoxybenzyl)-3-methyl-2-oxopyrrolidin-3-yl)carbamate and Methyl (1-(4-methoxybenzyl)-3-methyl-2-oxopyrrolidin-3-yl)carbamate

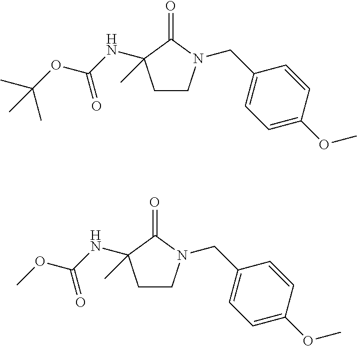

Triethylamine (6.88 mL, 49.4 mmol) was added to 1-(4-methoxybenzyl)-3-methyl-2-oxopyrrolidine-3-carboxylic acid (5.91 g, 22.45 mmol) in toluene (49.9 mL) and tert-butanol (24.94 mL) (likely contaminated with methanol) at room temperature, followed by diphenyl phosphoryl azide (4.84 mL, 22.45 mmol) and the reaction mixture was stirred for 1 hour at 50° C., then heated for sixteen hours at 100° C. The reaction mixture was quenched with water and concentrated. Then, saturated sodium bicarbonate was added and the reaction mixture was extracted with ethyl acetate, dried over magnesium sulfate, filtered, and concentrated, The residue was purified by silica gel chromatography, eluting with ethyl acetate:heptanes (3:7 to 1:0) to give tert-butyl (1-(4-methoxybenzyl)-3-methyl-2-oxopyrrolidin-3-yl)carbamate (0.1549 g, 0.440 mmol, 1.960% yield) and methyl (1-(4-methoxybenzyl)-3-methyl-2-oxopyrrolidin-3-yl)carbamate (1.49 g, 4.84 mmol, 21.57% yield) as a side product likely resulting from trapping of the intermediate isocyanate with methanol in the tert-butanol. Impure fractions were repurified by silica gel chromatography, eluting with ethyl acetate:heptanes (3:7 to 1:0) to give more methyl (1-(4-methoxybenzyl)-3-methyl-2-oxopyrrolidin-3-yl)carbamate (0.9830 g, 3.19 mmol, 14.23% yield).

### tert-Butyl (1-(4-methoxybenzyl)-3-methyl-2-oxopyrrolidin-3-yl)carbamate

¹H NMR (400 MHz, CD₃SOCD₃) δ 1.12 (s, 3H), 1.36 (s, 9H), 1.72-1.84 (m, 1H), 2.30-2.40 (m, 1H), 2.96-3.16 (m, 2H), 3.72 (s, 3H), 4.28 (ABq, J_{AB}=14 Hz, Δv_{AB}=49 Hz, 2H), 6.88 (d, J=9 Hz, 2H), 7.03 (br s, 1H), 7.15 (d, J=9 Hz, 2H); LC-MS (LC-ES) M+H=335.

### Methyl (1-(4-methoxybenzyl)-3-methyl-2-oxopyrrolidin-3-yl)carbamate

¹H NMR (400 MHz, CD₃SOCD₃) δ 1.16 (s, 3H), 1.76-1.84 (m, 1H), 2.30-2.40 (m, 1H), 3.00-3.16 (m, 2H), 3.50 (s, 3H), 3.73 (s, 3H), 4.28 (ABq, J_{AB}=15 Hz, Δv_{AB}=63 Hz, 2H), 6.89 (d, J=9 Hz, 2H), 7.16 (d, J=9 Hz, 2H), 7.43 (br s, 1H); LC-MS (LC-ES) M+H=293.

### E. 3-Amino-1-(4-methoxybenzyl)-3-methylpyrrolidin-2-one Hydroiodide

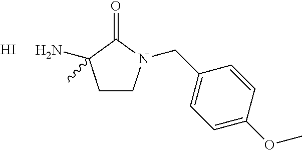

Iodotrimethylsilane (2.58 mL, 13.67 mmol) was added to methyl (1-(4-methoxybenzyl)-3-methyl-2-oxopyrrolidin-3-yl)carbamate (1.03 g, 3.52 mmol) in dichloromethane (17.62 mL) at room temperature and the reaction was stirred for sixteen hours at room temperature and concentrated to give crude 3-amino-1-(4-methoxybenzyl)-3-methylpyrrolidin-2-one hydroiodide (1.39 g, 3.42 mmol, 97% yield). ¹H NMR (400 MHz, CD₃SOCD₃) δ 1.34 (s, 3H), 2.04-2.12 (m, 2H), 3.20-3.28 (m, 2H), 3.74 (s, 3H), 4.36 (s, 2H), 6.92 (d, J=9 Hz, 2H), 7.18 (d, J=9 Hz, 2H), 8.42 (br s, 3H); LC-MS (LC-ES) M+H=235.

Intermediate 42

### 3-Amino-4-methylpyrrolidin-2-one Hydrochloride

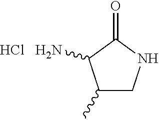

### A. Racemic Benzyl (2-hydroxypropyl)carbamate

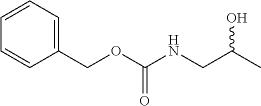

Sodium carbonate (3.10 g, 29.3 mmol) was added to 1-aminopropan-2-ol (2.0 g, 26.6 mmol) in water (22.19 mL) at 0° C., followed by benzyl chloroformate (4.18 mL, 29.3 mmol) and the reaction mixture was stirred for 3 hours at 0° C. Then, the reaction mixture was extracted with dichloromethane, dried over magnesium sulfate, filtered, and concentrated. The residue was purified via silica gel chromatography, eluting with ethyl acetate:heptane (0:1 to 1:0) to give racemic benzyl (2-hydroxypropyl)carbamate (5.61 g, 25.5 mmol, 96% yield). ¹H NMR (400 MHz, CD₃SOCD₃) δ 0.99 (d, J=6 Hz, 3H), 2.84-2.98 (m, 2H), 3.56-3.66 (m, 1H), 4.60 (d, J=5 Hz, 1H), 5.00 (s, 2H), 7.16 (br t, J=6 Hz, 1H), 7.26-7.38 (m, 5H); LC-MS (LC-ES) M+H=210.

### B. Benzyl (2-oxopropyl)carbamate

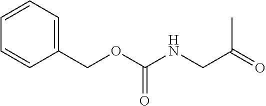

Dess-Martin periodinane (11.94 g, 28.2 mmol) was added to racemic benzyl (2-hydroxypropyl)carbamate (5.61 g, 26.8 mmol) in dichloromethane (89 mL) at 0° C. and the reaction mixture was stirred for sixteen hours at room temperature. The reaction mixture was quenched with saturated sodium thiosulfate and saturated sodium bicarbonate, extracted with dichloromethane, dried over magnesium sulfate, filtered, and concentrated. The residue was purified by silica gel chromatography, eluting with ethyl acetate:hexanes (1:1 to 3:7) to give benzyl (2-oxopropyl)carbamate (5.24 g, 24.02 mmol, 90% yield). ¹H NMR (400 MHz, CD₃SOCD₃) δ 2.05 (s, 3H), 3.83 (d, J=6 Hz, 2H), 5.02 (s, 2H), 7.26-7.40 (m, 5H), 7.48 (br t, J=6 Hz, 1H); LC-MS (LC-ES) M+H=208.

### C. Methyl 4-(((benzyloxy)carbonyl)amino)-2-((tert-butoxycarbonyl)amino)-3-methylbut-2-enoate

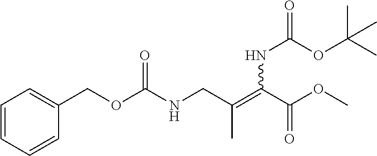 [SMILES: COC(=O)C(NC(=O)OC(C)(C)C)=C(C)CNC(=O)OCc1ccccc1]

Methyl 2-((tert-butoxycarbonyl)amino)-2-(dimethoxyphosphoryl)acetate (15.78 g, 53.1 mmol) was added to benzyl (2-oxopropyl)carbamate (5.24 g, 25.3 mmol) in dichloromethane (126 mL) at room temperature under a nitrogen atmosphere. Then, 1,8-diazabicyclo[5.4.0]undec-7-ene (7.18 mL, 48.0 mmol) was added and the reaction mixture was stirred for sixty-four hours. Saturated ammonium chloride was added and the reaction mixture was extracted with dichloromethane, dried over magnesium sulfate, filtered, and concentrated. The residue was purified by silica gel chromatography, eluting with ethyl acetate:hexanes (1:9 to 3:2) to give a 1.3:1 mixture of geometric isomers of methyl 4-(((benzyloxy)carbonyl)amino)-2-((tert-butoxycarbonyl)amino)-3-methylbut-2-enoate (4.60 g, 11.55 mmol, 45.7% yield). ¹H NMR (400 MHz, CD₃SOCD₃) δ 1.39 & 1.40 (s, 9H), 1.79 & 1.91 (s, 3H), 3.64-3.72 (m, 2H), 3.61 & 4.22, 5.03 & 5.24 (s, 2H), 7.28-7.46 (m, 5H), 7.57 (br t, J=7 Hz, 1H), 8.38 & 8.45 (br s, 1H); LC-MS (LC-ES) M+H−CO₂tBu=279.

### D. tert-Butyl (4-methyl-2-oxopyrrolidin-3-yl)carbamate

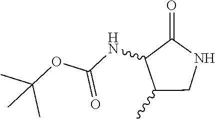

Palladium on carbon (0.647 g, 0.608 mmol) was added to a 1.3:1 mixture of isomers of methyl 4-(((benzyloxy)carbonyl)amino)-2-((tert-butoxycarbonyl)amino)-3-methylbut-2-enoate (4.60 g, 12.16 mmol) in methanol (60.8 mL) at 25° C. under nitrogen atmosphere. Then, the reaction vessel was fitted with a hydrogen balloon and the vessel was repeatedly evacuated and purged with hydrogen, then stirred for sixteen hours. Then, the vessel was repeatedly evacuated and purged with nitrogen, filtered through Celite®, and concentrated. The resulting residue was purified by silica gel chromatography, eluting with ethyl acetate:hexanes (2:3 to 1:0) to give a ˜6:1 racemic mixture of cis:trans isomers of tert-butyl (4-methyl-2-oxopyrrolidin-3-yl)carbamate (1.32 g, 5.85 mmol, 48.1% yield). ¹H NMR (400 MHz, CD₃SOCD₃, major cis isomer) δ 0.82 (d, J=7 Hz, 3H), 1.39 (s, 9H), 1.36-1.42 (m, 1H), 2.79 (ddd, J=10, 3, 1 Hz, 1H), 3.28-3.34 (m, 1H), 4.07 (t, J=8 Hz, 1H), 6.90 (br d, J=9 Hz, 1H), 7.70 (br s, 1H); LC-MS (LC-ES) M+H=215.

### E. 3-Amino-4-methylpyrrolidin-2-one Hydrochloride

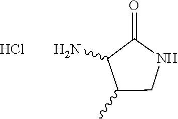

4.0 M Hydrochloric acid (7.70 mL, 30.8 mmol) in dioxane was added to a ˜6:1 mixture of cis:trans isomers of tert-butyl (4-methyl-2-oxopyrrolidin-3-yl)carbamate (1.32 g, 6.16 mmol) in methanol (7.70 mL) at room temperature and the reaction mixture was stirred for sixteen hours. The reaction mixture was concentrated to give a racemic ˜6:1 mixture of cis:trans isomers of 3-amino-4-methylpyrrolidin-2-one hydrochloride (1.10 g, 6.14 mmol, 100% yield). ¹H NMR (400 MHz, CD₃SOCD₂ major cis isomer) δ 0.98 (d, J=7 Hz, 3H), 2.60-2.72 (m, 1H), 2.88 (dt, J=10, 2 Hz, 1H), 3.41 (dd, J=10, 6 Hz, 1H), 3.90-4.00 (m, 1H), 8.39 (br s, 3H), 8.74 (br s, 1H); LC-MS (LC-ES) M+H=115.

Intermediate 43

### 2-(3-chloro-5-fluorophenyl)thiazole-5-carbothioic O-acid

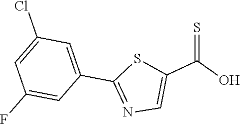 [SMILES: OC(=S)c1cnc(-c2cc(F)cc(Cl)c2)s1]

Lawesson's reagent (2,4-bis(4-methoxyphenyl)-1,3,2,4-dithiadiphosphetane 2,4-disulfide (0.204 g, 0.504 mmol)) was added to 2-(3-chloro-5-fluorophenyl)thiazole-5-carboxylic acid (0.2595 g, 1.007 mmol) in toluene (10.07 mL) at room temperature, then the reaction mixture was heated at reflux for two hours. The reaction mixture was cooled and concentrated. The residue was purified by silica gel chromatography, eluting with ethyl acetate:hexanes (0:1 to 2:3) to give 2-(3-chloro-5-fluorophenyl)thiazole-5-carbothioic O-acid (0.0525 g, 0.182 mmol, 18.09% yield). ¹H NMR (400 MHz, CD₃SOCD₃) δ 7.59 (dt, J=9, 2 Hz, 1H), 7.77 (ddd, J=9, 2, 2 Hz, 1H), 7.85 (t, J=2 Hz, 1H), 8.14 (s, 1H); LC-MS (LC-ES) M+H=274.

Intermediate 44

### Lithium 2-(3-chloro-5-fluorophenyl)-1,3-selenazole-5-carboxylate

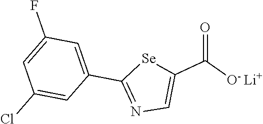

### A. 3-Chloro-5-fluorobenzoselenoamide

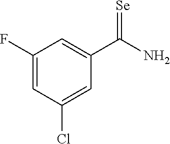 [SMILES: NC(=[Se])c1cc(F)cc(Cl)c1]

Woollin's reagent (2,4-diphenyl-1,3,2,4-diselenadiphosphetane 2,4-diselenide) (1.007 g, 1.893 mmol)) was added to 3-chloro-5-fluorobenzonitrile (0.5888 g, 3.79 mmol) in toluene (37.9 mL) at room temperature, then the reaction mixture was heated at reflux for sixteen hours. The reaction mixture was cooled, water (2 mL) added, heated to reflux for 1 hour, and concentrated. The residue was purified by silica gel chromatography, eluting with ethyl acetate:hexanes (0:1 to 1:1) to give 3-chloro-5-fluorobenzoselenoamide (0.5589 g, 2.245 mmol, 59.3% yield). ¹H NMR (400 MHz, CD₃SOCD₃) δ 7.62 (s, 1H), 7.63 (s, 1H), 7.75 (s, 1H), 10.37 (br s, 1H), 11.07 (br s, 1H); LC-MS (LC-ES) M+H=236.

### B. Ethyl 2-(3-chloro-5-fluorophenyl)-1,3-selenazole-5-carboxylate

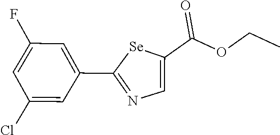 [SMILES: CCOC(=O)c1cnc(-c2cc(F)cc(Cl)c2)[se]1]

Ethyl 2-chloro-3-oxopropanoate (0.356 g, 2.363 mmol) was added to 3-chloro-5-fluorobenzoselenoamide (0.5589 g, 2.363 mmol) in acetonitrile (23.63 mL) at room temperature and the reaction mixture was heated at 80° C. for three hours. The reaction mixture was concentrated. The residue was purified by RP HPLC, eluting with acetonitrile:water with 0.1% ammonium hydroxide (5:95 to 100:0) to give ethyl 2-(3-chloro-5-fluorophenyl)-1,3-selenazole-5-carboxylate (0.4946 g, 1.413 mmol, 59.8% yield). ¹H NMR (400 MHz, CD₃SOCD₃) δ 1.31 (t, J=7 Hz, 3H), 4.32 (q, J=7 Hz, 2H), 7.69 (dt, J=10, 2 Hz, 1H), 7.88 (ddd, J=10, 2, 2 Hz, 1H), 7.95 (t, J=1 Hz, 1H), 8.53 (s, 1H); LC-MS (LC-ES) M+H=332.

### C. Lithium 2-(3-chloro-5-fluorophenyl)-1,3-selenazole-5-carboxylate

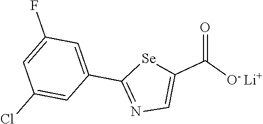

Lithium hydroxide (0.107 g, 4.46 mmol) was added to ethyl 2-(3-chloro-5-fluorophenyl)-1,3-selenazole-5-carboxylate (0.4946 g, 1.487 mmol) in methanol (11.90 mL) and water (2.97 mL) at room temperature and the reaction mixture was stirred sixteen hours at 50° C. Then, the reaction mixture was concentrated to give lithium 2-(3-chloro-5-fluorophenyl)-1,3-selenazole-5-carboxylate (0.4802 g, 1.469 mmol, 99% yield). ¹H NMR (400 MHz, CD₃SOCD₃) δ 7.53 (dt, J=9, 2 Hz, 1H), 7.68 (ddd, J=9, 2, 2 Hz, 1H), 7.75 (t, J=2 Hz, 1H), 7.84 (s, 1H); LC-MS (LC-ES) M+H=304.

Intermediate 45

### 3-Amino-5,5-dimethylpyrrolidine-2-thione Hydrochloride

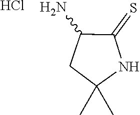

### A. tert-Butyl (5,5-dimethyl-2-thioxopyrrolidin-3-yl)carbamate

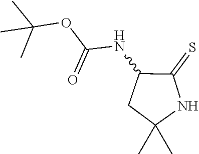 [SMILES: CC1(C)CC(NC(=O)OC(C)(C)C)C(=S)N1]

Lawesson's reagent (2,4-bis(4-methoxyphenyl)-1,3,2,4-dithiadiphosphetane 2,4-disulfide (0.275 g, 0.679 mmol)) was added to tert-butyl (5,5-dimethyl-2-oxopyrrolidin-3-yl)carbamate (0.3102 g, 1.359 mmol, Intermediate 12F) in toluene (13.59 mL) at room temperature, then the reaction mixture was heated at 85° C. for sixteen hours. The reaction mixture was cooled, water (2 mL) added, heated to reflux for 1 hour, saturated sodium bicarbonate added, extracted with dichloromethane, dried with magnesium sulfate, filtered, and concentrated. The residue was purified by silica gel chromatography, eluting with methanol:ethyl acetate (1:4 to 1:0) to give tert-butyl (5,5-dimethyl-2-thioxopyrrolidin-3-yl)carbamate (0.2501 g, 0.972 mmol, 71.6% yield). ¹H NMR (400 MHz, CD₃SOCD₃) δ 1.20 (s, 3H), 1.23 (s, 3H), 1.38 (s, 9H), 1.72 (t, J=11 Hz, 1H), 2.25 (dd, J=12, 9 Hz, 1H), 4.44 (q, J=10 Hz, 1H), 6.97 (br d, J=9 Hz, 1H), 10.38 (br s, 1H); LC-MS (LC-ES) M+H-tBuOCO=145.

### B. 3-Amino-5,5-dimethylpyrrolidine-2-thione Hydrochloride

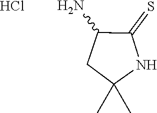

4.0 M Hydrochloric acid (1.279 mL, 5.12 mmol) in dioxane was added to tert-butyl (5,5-dimethyl-2-thioxopyrrolidin-3-yl)carbamate (0.2501 g, 1.024 mmol) in 1,4-dioxane (1.279 mL) at room temperature and the reaction mixture was stirred for sixty-six hours. The reaction mixture was concentrated to give 3-amino-5,5-dimethylpyrrolidine-2-thione hydrochloride (0.1836 g, 0.965 mmol, 94% yield). ¹H NMR (400 MHz, CD₃SOCD₃) δ 1.26 (s, 3H), 1.29 (s, 3 H), 1.85 (dd, J=13, 10 Hz, 1H), 2.40 (dd, J=13, 8 Hz, 1H), 4.33 (dd, J=10, 9 Hz, 1H), 8.47 (br s, 3H), 10.95 (br s, 1H); LC-MS (LC-ES) M+H=145.

### Example 1

### (S)-2-(Benzofuran-7-yl)-N-(2-oxopyrrolidin-3-yl)thiazole-5-carboxamide

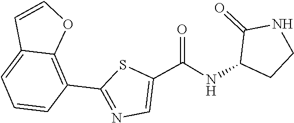 [SMILES: O=C(N[C@H]1CCNC1=O)c1cnc(-c2cccc3ccoc23)s1]

2-(Benzofuran-7-yl)thiazole-5-carboxylic acid (0.0619 g, 0.252 mmol, Intermediate 1) and (S)-3-aminopyrrolidin-2-one (0.0301 g, 0.301 mmol, AstaTech) were dissolved in N,N-dimethylformamide (1 mL). N,N-Diisopropylethylamine (0.130 mL, 0.744 mmol) was added, followed by 1-((dimethylamino)(dimethyliminio)methyl)-1H-[1,2,3]triazolo[4,5-b]pyridine 3-oxide hexafluorophosphate(V) (0.1138 g, 0.299 mmol). The mixture was stirred at room temperature for 2 hours and water (50 mL) and brine (20 mL) were added. The mixture was extracted with ethyl acetate (15 mL, 3×). The combined organics were dried over magnesium sulfate, filtered, and concentrated. The residue was purified by silica gel chromatography, eluting with isopropanol:ethyl acetate (1:9) to provide (S)-2-(benzofuran-7-yl)-N-(2-oxopyrrolidin-3-yl)thiazole-5-carboxamide (0.0593 g, 0.181 mmol, 72% yield) as a white solid. ¹H NMR (400 MHz, CD₃SOCD₃) δ 1.93-2.10 (m, 1H), 2.33-2.45 (m, 1H), 3.18-3.30 (m, 2H), 4.51-4.63 (m, 1H), 7.17 (d, J=2 Hz, 1H), 7.45 (t, J=8 Hz, 1H), 7.87 (d, J=7 Hz, 1H), 7.94 (s, 1H), 8.18 (d, J=7 Hz, 1H), 8.26 (d, J=2 Hz, 1H), 8.59 (s, 1H), 9.03 (d, J=8 Hz, 1H); LC-MS (LC-ES) for C₁₆H₁₃ClN₃O₃S M+H=328.

### Example 2

Racemic 2-(3-Chlorophenyl)-N-(2-oxopyrrolidin-3-yl)thiazole-5-carboxamide

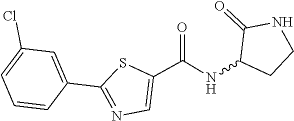 [SMILES: O=C(NC1CCNC1=O)c1cnc(-c2cccc(Cl)c2)s1]

1-((Dimethylamino)(dimethyliminio)methyl)-1H-[1,2,3]triazolo[4,5-b]pyridine 3-oxide hexafluorophosphate(V) (0.083 g, 0.219 mmol) was added to 2-(3-chlorophenyl)thiazole-5-carboxylic acid (0.050 g, 0.209 mmol, Intermediate 2) and N,N-dimethylformamide (2 mL) was added, followed by N,N-diisopropylethylamine (0.040 mL, 0.229 mmol) and the mixture was stirred 1 hour under nitrogen. Racemic 3-aminopyrrolidin-2-one (0.023 g, 0.229 mmol) was added in one portion and stirring was continued overnight. The mixture was poured into saturated sodium bicarbonate and extracted with ethyl acetate (3×). The combined organics were washed with water and saturated sodium chloride, dried over sodium sulfate, filtered, and concentrated in vacuo. The residue was purified by silica gel chromatography, eluting with (ethyl acetate:ethanol (3:1)):hexanes (0:1 to 1:0) to afford racemic 2-(3-chlorophenyl)-N-(2-oxopyrrolidin-3-yl)thiazole-5-carboxamide (0.0225 g, 0.070 mmol, 33.5% yield) as a colorless solid. ¹H NMR (400 MHz, CD₃SOCD₃) δ 1.92-2.06 (m, 1H), 2.32-2.42 (m, 1H), 3.18-3.30 (m, 2H), 4.53 (dt, J=10, 9 Hz, 1H), 7.56 (t, J=8 Hz, 1H), 7.62 (ddd, J=8, 2, 1 Hz, 1H), 7.96 (br s, 1H), 7.96 (dt, J=8, 2 Hz, 1H), 8.03 (t, J=2 Hz, 1H), 8.51 (s, 1H), 9.04 (d, J=8 Hz, 1H); LC-MS (LC-ES) for C₁₄H₁₂ClN₃O₂S M+H=322.

### Example 3

### (S)-2-(3-Chlorophenyl)-N-(2-oxopyrrolidin-3-yl)thiazole-5-carboxamide

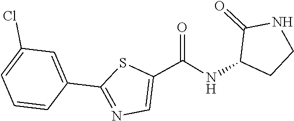 [SMILES: O=C(N[C@H]1CCNC1=O)c1cnc(-c2cccc(Cl)c2)s1]

(S)-3-Aminopyrrolidin-2-one (0.036 g, 0.357 mmol) was added to a solution of 2-(3-chlorophenyl)thiazole-5-carboxylic acid (0.0778 g, 0.325 mmol, Intermediate 2) in N,N-dimethylformamide (2 mL), followed by 1-((dimethylamino)(dimethyliminio)methyl)-1H-[1,2,3]triazolo[4,5-b]pyridine 3-oxide hexafluorophosphate(V) (0.148 g, 0.390 mmol). Then, N,N-diisopropylethylamine (0.068 mL, 0.390 mmol) was added and the reaction mixture was stirred at room temperature. After 2 hours, the solution was poured into saturated sodium bicarbonate (10 mL) and water (10 mL) and extracted with ethyl acetate (3×). The combined organics were washed with water and saturated sodium chloride, dried over sodium sulfate, filtered, and concentrated onto Celite®. Purification by RP HPLC, eluting with acetonitrile:water (3:7 to 1:3), afforded (S)-2-(3-chlorophenyl)-N-(2-oxopyrrolidin-3-yl)thiazole-5-carboxamide (0.0860 g, 0.267 mmol, 82% yield) as a colorless solid. ¹H NMR (400 MHz, CD₃SOCD₃) δ 1.92-2.06 (m, 1H), 2.32-2.42 (m, 1H), 3.18-3.30 (m, 2H), 4.53 (dt, J=10, 9 Hz, 1H), 7.56 (t, J=8 Hz, 1H), 7.62 (ddd, J=8, 2, 1 Hz, 1H), 7.96 (br s, 1H), 7.96 (dt, J=8, 2 Hz, 1H), 8.03 (t, J=2 Hz, 1H), 8.51 (s, 1H), 9.04 (d, J=8 Hz, 1H); LC-MS (LC-ES) for C₁₄H₁₂ClN₃O₂S M+H=322.

### Example 4

(S)-N-(2-Oxopyrrolidin-3-v)-2-(3-(trifluoromethyl)phenyl)thiazole-5-carboxamide

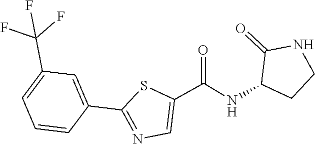 [SMILES: O=C(N[C@H]1CCNC1=O)c1cnc(-c2cccc(C(F)(F)F)c2)s1]

1-((Dimethylamino)(dimethyliminio)methyl)-1H-[1,2,3]triazolo[4,5-b]pyridine 3-oxide hexafluorophosphate(V) (0.143 g, 0.376 mmol) was added to a solution of 2-(3-(trifluoromethyl)phenyl)thiazole-5-carboxylic acid (0.0857 g, 0.314 mmol, Intermediate 3) and (S)-3-aminopyrrolidin-2-one (0.035 g, 0.345 mmol) in N,N-dimethylformamide (2 mL). Then, N,N-diisopropylethylamine (0.066 mL, 0.376 mmol) was added and the reaction mixture was stirred at room temperature. After two hours, the solution was poured into saturated sodium bicarbonate (10 mL) and water (10 mL) and extracted with ethyl acetate (3×). The combined organics were washed with water and saturated sodium chloride, dried over sodium sulfate, filtered, and concentrated onto Celite®. This residue was purified via reverse phase HPLC, eluting with acetonitrile:water (3:7 to 1:3) to afford (S)-N-(2-oxopyrrolidin-3-yl)-2-(3-(trifluoromethyl)phenyl)thiazole-5-carboxamide (0.0963 g, 0.271 mmol, 86% yield) as a colorless solid. ¹H NMR (400 MHz, CD₃SOCD₃) δ 1.92-2.06 (m, 1H), 2.32-2.42 (m, 1H), 3.18-3.30 (m, 2H), 4.54 (q, J=8 Hz, 1H), 7.76 (t, J=8 Hz, 1H), 7.91 (d, J=8 Hz, 1H), 7.96 (br s, 1H), 8.29 (d, J=1 Hz, 1H), 8.30 (s, 1H), 8.54 (s, 1H), 9.06 (br d, J=8 Hz, 1H); LC-MS (LC-ES) for C₁₅H₁₂F₃N₃O₂S M+H=356.

### Example 5

### (S)-N-(2-Oxopyrrolidin-3-yl)-2-(m-tolyl)thiazole-5-carboxamide

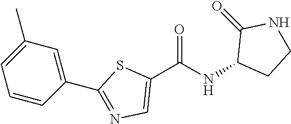 [SMILES: Cc1cccc(-c2ncc(C(=O)N[C@H]3CCNC3=O)s2)c1]

(S)-3-Aminopyrrolidin-2-one (0.041 g, 0.411 mmol) and 1-((dimethylamino)(dimethyliminio)methyl)-1H-[1,2,3]triazolo[4,5-b]pyridine 3-oxide hexafluorophosphate(V) (0.171 g, 0.449 mmol) were added to a solution of 2-(m-tolyl)thiazole-5-carboxylic acid (0.082 g, 0.374 mmol, Intermediate 4) in N,N-dimethylformamide (2 mL). Then, N,N-diisopropylethylamine (0.078 mL, 0.449 mmol) was added and the mixture was stirred at room temperature. After 2 hours, the solution was poured into saturated sodium bicarbonate (10 mL) and water (10 mL) and extracted with ethyl acetate (3×). The combined organics were washed with water and saturated sodium chloride, dried over sodium sulfate, filtered, and concentrated onto Celite®. This residue was purified via reverse phase HPLC, eluting with acetonitrile:water (3:7 to 3:1) to afford (S)-N-(2-oxopyrrolidin-3-yl)-2-(m-tolyl)thiazole-5-carboxamide (0.0870 g, 0.289 mmol, 77% yield) as a colorless solid. ¹H NMR (400 MHz, CD₃SOCD₃) δ 1.92-2.06 (m, 1H), 2.32-2.42 (m, 1H), 2.39 (s, 3H), 3.18-3.30 (m, 2H), 4.53 (dt, J=10, 9 Hz, 1H), 7.35 (d, J=8 Hz, 1H), 7.41 (t, J=8 Hz, 1H), 7.79 (d, J=8 Hz, 1H), 7.82 (s, 1H), 7.94 (s, 1H), 8.47 (s, 1H), 8.98 (d, J=8 Hz, 1H); LC-MS (LC-ES) for C₁₅H₁₅N₃O₂S M+H=302.

### Example 6

### (S)-2-(3-Chloro-5-fluorophenyl)-N-(2-oxopyrrolidin-3-yl)thiazole-5-carboxamide

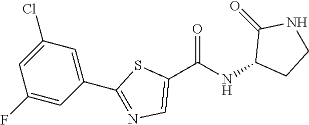 [SMILES: O=C(N[C@H]1CCNC1=O)c1cnc(-c2cc(F)cc(Cl)c2)s1]

2-(3-Chloro-5-fluorophenyl)thiazole-5-carboxylic acid (0.04 g, 0.155 mmol, Intermediate 5) and (S)-3-aminopyrrolidin-2-one (0.023 g, 0.233 mmol) were suspended in ethyl acetate (0.9 mL), and N,N-diisopropylethylamine (0.1 mL, 0.573 mmol) was added and the reaction mixture became homogeneous. Then, n-propylphosphonic acid anhydride (50% in ethyl acetate) (0.2 mL, 0.336 mmol) was slowly added and the reaction mixture was stirred overnight at room temperature. It was quenched with saturated aqueous sodium bicarbonate and stirred at room temperature for 10 minutes, then extracted with ethyl acetate (2×), washed with brine, dried over sodium sulfate, filtered, and concentrated under reduced pressure to provide (S)-2-(3-chloro-5-fluorophenyl)-N-(2-oxopyrrolidin-3-yl)thiazole-5-carboxamide (44 mg, 0.127 mmol, 82% yield) as a tan solid. ¹H NMR (400 MHz, CD₃OD) δ 2.12-2.28 (m, 1H), 2.52-2.62 (m, 1H), 3.38-3.50 (m, 2H), 4.73 (dd, J=10, 9 Hz, 1H), 7.41 (dt, J=9, 2 Hz, 1H), 7.76 (dt, J=9, 2 Hz, 1H), 7.90 (s, 1H), 8.43 (s, 1H); LC-MS (LC-ES) for C₁₄H₁₁₁ClFN₃O₂S M+H=340.

### Example 7

### (S)-2-(5-Chloro-2-fluorophenyl)-N-(2-oxopyrrolidin-3-yl)thiazole-5-carboxamide

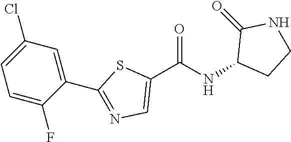 [SMILES: O=C(N[C@H]1CCNC1=O)c1cnc(-c2cc(Cl)ccc2F)s1]

2-(5-chloro-2-fluorophenyl)thiazole-5-carboxylic acid (0.05 g, 0.194 mmol, Intermediate 6) and (S)-3-aminopyrrolidin-2-one (0.029 g, 0.291 mmol) were suspended in ethyl acetate (1.2 mL), then N,N-diisopropylethylamine (0.12 mL, 0.687 mmol) was added and the reaction mixture became homogeneous. n-Propylphosphonic acid anhydride (50% in ethyl acetate) (0.25 mL, 0.420 mmol) was added and the reaction mixture was allowed to stir at room temperature for 30 minutes before diluting it with methanol. The mixture was purified by reverse phase HPLC, eluting with acetonitrile:water with 0.1% ammonium hydroxide (1:19 to 19:1). While the material was sitting in a vial, a precipitant formed and the solvents were decanted into a new vial to continue the purification by HPLC resulting in a white solid. The precipitant was triturated with methanol and filtered to yield a white solid. The solids were then combined to yield (S)-2-(5-chloro-2-fluorophenyl)-N-(2-oxopyrrolidin-3-yl)thiazole-5-carboxamide (40.5 mg, 0.117 mmol, 60.2% yield). ¹H NMR (400 MHz, CD₃SOCD₃) δ 1.92-2.08 (m, 1H), 2.34-2.46 (m, 1H), 3.20-3.32 (m, 2H), 4.56 (dt, J=11, 9 Hz, 1H), 7.57 (dd, J=11, 9 Hz, 1H), 7.69 (ddd, J=9, 4, 3 Hz, 1H), 7.96 (s, 1H), 8.25 (dd, J=6, 3 Hz, 1H), 8.60 (d, J=2 Hz, 1H), 9.09 (d, J=9 Hz, 1H); LC-MS (LC-ES) for C₁₄H₁₁ClFN₃O₂S M+H=340.

### Example 8

### (S)-2-(3-Chloro-2-fluorophenyl)-N-(2-oxopyrrolidin-3-yl)thiazole-5-carboxamide

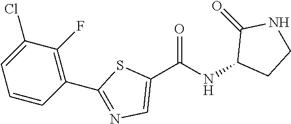 [SMILES: O=C(N[C@H]1CCNC1=O)c1cnc(-c2cccc(Cl)c2F)s1]

2-(3-chloro-2-fluorophenyl)thiazole-5-carboxylic acid (0.05 g, 0.194 mmol, Intermediate 7) and (S)-3-aminopyrrolidin-2-one (0.029 g, 0.291 mmol) were suspended in ethyl acetate (1.2 mL), then N,N-diisopropylethylamine (0.12 mL, 0.687 mmol) was added and the reaction mixture became homogeneous. Then, n-propylphosphonic acid anhydride (50% in ethyl acetate) (0.25 mL, 0.420 mmol) was slowly added and the reaction mixture was stirred at room temperature overnight. It was quenched with saturated aqueous sodium bicarbonate and stirred at room temperature for 10 minutes. It was extracted with ethyl acetate (2×). The combined organics were washed with brine, dried over sodium sulfate, filtered, and concentrated under reduced pressure. The resulting tan solid was purified by reverse phase HPLC, eluting with acetonitrile:water with 0.1% ammonium hydroxide (1:19 to 19:1) to afford (S)-2-(3-chloro-2-fluorophenyl)-N-(2-oxopyrrolidin-3-yl)thiazole-5-carboxamide (39.6 mg, 0.114 mmol, 58.9% yield) as a white solid. ¹H NMR (400 MHz, CD₃OD) δ 2.14-2.28 (m, 1H), 2.54-2.64 (m, 1H), 3.38-3.50 (m, 2H), 4.73 (dd, J=10, 9 Hz, 1H), 7.37 (td, J=8, 1 Hz, 1H), 7.67 (ddd, J=8, 7, 1 Hz, 1H), 8.26 (ddd, J=8, 7, 2 Hz, 1H), 8.49 (d, J=3 Hz, 1H); LC-MS (LC-ES) for C₁₄H₁₁ClFN₃O₂S M+H=340.

### Example 9

### (S)-2-(3,5-Dichlorophenyl)-N-(2-oxopyrrolidin-3-yl)thiazole-5-carboxamide

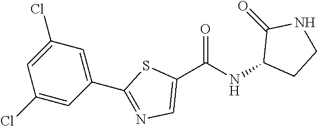 [SMILES: O=C(N[C@H]1CCNC1=O)c1cnc(-c2cc(Cl)cc(Cl)c2)s1]

Tetrakis(triphenylphosphine)palladium(0) (0.028 g, 0.024 mmol) was added to (S)-2-bromo-N-(2-oxopyrrolidin-3-yl)thiazole-5-carboxamide (0.07 g, 0.241 mmol, Intermediate 8), (3,5-dichlorophenyl)boronic acid (0.092 g, 0.483 mmol), and a 2.0 M aqueous solution of sodium carbonate (0.362 mL, 0.724 mmol) in acetonitrile (2.4 mL). The vessel was sealed and evacuated of atmosphere and flushed with nitrogen (3×) before placing in a preheated heating mantle at 90° C. for 1 hour (LC-MS of the reaction showed completion). The reaction mixture was cooled to room temperature and filtered through a pad of Celite®, washed with ethyl acetate and concentrated under reduced pressure. The residue was taken up in ethyl acetate and water and the layers were separated. The organic phase was washed with brine, dried over sodium sulfate, filtered, and concentrated under reduced pressure. The residue was purified by reverse phase HPLC, eluting with acetonitrile:water with 0.1% ammonium hydroxide (1:19 to 19:1) to afford a white solid that was contaminated with triphenylphosphine oxide. The impure material was further purified by silica gel chromatography, eluting with (3:1 ethyl acetate:ethanol):heptane (1:4 to 7:3) to give (S)-2-(3,5-dichlorophenyl)-N-(2-oxopyrrolidin-3-yl)thiazole-5-carboxamide (13.6 mg, 0.037 mmol, 15.51% yield) as a white solid. Another batch of product was recovered in later fractions as (S)-2-(3,5-dichlorophenyl)-N-(2-oxopyrrolidin-3-yl)thiazole-5-carboxamide (21 mg, 0.058 mmol, 23.95% yield). ¹H NMR (400 MHz, CD₃OD) δ 2.12-2.26 (m, 1H), 2.52-2.64 (m, 1H), 3.38-3.50 (m, 2H), 4.73 (dd, J=10, 9 Hz, 1H), 7.63 (t, J=2 Hz, 1H), 7.99 (d, J=2 Hz, 2H), 8.43 (s, 1H); LC-MS (LC-ES) for C₁₄H₁₁Cl2N₃O₂S M+H=356.

### Example 10

### (S)-2-(3-(Difluoromethyl)phenyl)-N-(2-oxopyrrolidin-3-yl)thiazole-5-carboxamide

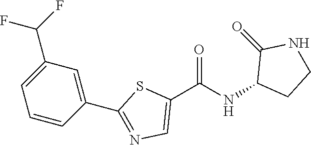 [SMILES: O=C(N[C@H]1CCNC1=O)c1cnc(-c2cccc(C(F)F)c2)s1]

N,N-Diisopropylethylamine (0.068 mL, 0.392 mmol) was added to a solution of 2-(3-(difluoromethyl)phenyl)thiazole-5-carboxylic acid (0.0834 g, 0.327 mmol, Intermediate 9), (S)-3-aminopyrrolidin-2-one (0.036 g, 0.359 mmol) and 1-((dimethylamino)(dimethyliminio)methyl)-1H-[1,2,3]triazolo[4,5-b]pyridine 3-oxide hexafluorophosphate(V) (0.149 g, 0.392 mmol) in N,N-dimethylformamide (2 mL) and the mixture was stirred at room temperature. After 2 hours, the mixture was poured into saturated aqueous sodium bicarbonate (20 mL) and water (5 mL) and extracted with ethyl acetate (3×). The combined organic layers were washed with water and saturated aqueous sodium chloride, dried over sodium sulfate, and concentrated in vacuo. The resulting residue was purified by reverse phase HPLC, eluting with acetonitrile:water (1:4 to 4:1) to give (S)-2-(3-(difluoromethyl)phenyl)-N-(2-oxopyrrolidin-3-yl)thiazole-5-carboxamide (0.0624 g, 0.185 mmol, 56.6% yield) as a colorless solid. ¹H NMR (400 MHz, CD₃SOCD₃) δ 1.92-2.08 (m, 1H), 2.30-2.44 (m, 1H), 3.18-3.30 (m, 2H), 4.46-4.58 (m, 1H), 7.15 (t, J=56 Hz, 1H), 7.69 (t, J=8 Hz, 1H), 7.74 (d, J=8 Hz, 1H), 7.95 (s, 1H), 8.15 (d, J=8 Hz, 1H), 8.20 (s, 1H), 8.52 (s, 1H), 9.04 (d, J=5 Hz, 1H); LC-MS (LC-ES) for C₁₅H₁₃F₂N₃O₂S M+H=338.

### Example 11

### 2-(3-Chloro-5-fluorophenyl)-N-((3S,4R)-4-methyl-2-oxopyrrolidin-3-yl)thiazole-5-carboxamide

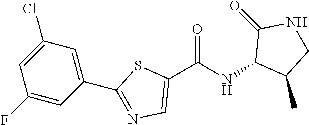 [SMILES: C[C@@H]1CNC(=O)[C@H]1NC(=O)c1cnc(-c2cc(F)cc(Cl)c2)s1]

N,N-Diisopropylethylamine (0.059 mL, 0.340 mmol) was added to an stirring solution of 2-(3-chloro-5-fluorophenyl)thiazole-5-carboxylic acid (70 mg, 0.272 mmol, Intermediate 5) in N,N-dimethylformamide (1.5 mL), then 1-((dimethylamino)(dimethyliminio)methyl)-1H-[1,2,3]triazolo[4,5-b]pyridine 3-oxide hexafluorophosphate(V) (124 mg, 0.326 mmol) was added. After stirring for ˜2 minutes, (3S,4R)-3-amino-4-methylpyrrolidin-2-one (38.8 mg, 0.340 mmol, Intermediate 10) and additional N,N-diisopropylethylamine (0.059 mL, 0.340 mmol) were added. LC-MS after 5 min shows a major peak for product and no starting material. After ˜1 hour, water (˜5 mL) was added dropwise to the reaction mixture. A yellow solid precipitated out. The mixture was stirred for ˜20 minutes. The solids were collected by filtration, washed sequentially with water and with hexanes, and dried at 50° C. under high vacuum overnight to give 2-(3-chloro-5-fluorophenyl)-N-((3S,4R)-4-methyl-2-oxopyrrolidin-3-yl)thiazole-5-carboxamide (79 mg, 0.212 mmol, 78% yield) as a light yellow solid. ¹H NMR (400 MHz, CD₃SOCD₃) δ 1.10 (d, J=7 Hz, 3H), 2.30-2.48 (m, 1H), 2.89 (t, J=10 Hz, 1H), 3.28-3.38 (m, 1H), 4.23 (dd, J=11, 9 Hz, 1H), 7.67 (dt, J=9, 2 Hz, 1H), 7.86 (dt, J=9, 2 Hz, 1H), 7.91 (s, 1H), 7.92 (dd, J=2, 2 Hz, 1H), 8.55 (s, 1H), 9.00 (d, J=9 Hz, 1H); LC-MS (LC-ES) for C₁₅H₁₃ClFN₃O₂S M+H=354.

### Example 12

### (S)-2-(3-(Difluoromethyl)-5-fluorophenyl)-N-(2-oxopyrrolidin-3-yl)thiazole-5-carboxamide

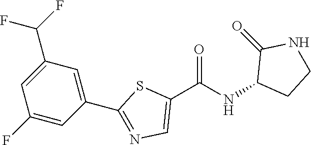 [SMILES: O=C(N[C@H]1CCNC1=O)c1cnc(-c2cc(F)cc(C(F)F)c2)s1]

N,N-Diisopropylethylamine (0.043 mL, 0.247 mmol) was added to a solution of 2-(3-(difluoromethyl)-5-fluorophenyl)thiazole-5-carboxylic acid (0.0563 g, 0.206 mmol, Intermediate 11), (S)-3-aminopyrrolidin-2-one (0.023 g, 0.227 mmol) and 1-((dimethylamino)(dimethyliminio)methyl)-1H-[1,2,3]triazolo[4,5-b]pyridine 3-oxide hexafluorophosphate(V) (0.086 g, 0.227 mmol) in N,N-dimethylformamide (2 mL) and the mixture was stirred at room temperature. After 4 hours, the mixture was poured into saturated sodium bicarbonate (20 mL) and water (5 mL) and extracted with ethyl acetate (3×). The combined organics were washed with water and saturated sodium chloride, dried over sodium sulfate, and concentrated onto Celite®. This powder was purified by reverse phase HPLC, eluting with acetonitrile:water (1:4 to 1:0) to give (S)-2-(3-(difluoromethyl)-5-fluorophenyl)-N-(2-oxopyrrolidin-3-yl)thiazole-5-carboxamide (0.0369 g, 0.104 mmol, 50.4% yield) as a colorless solid. ¹H NMR (400 MHz, CD₃SOCD₃) δ 1.92-2.06 (m, 1H), 2.32-2.44 (m, 1H), 3.16-3.28 (m, 2H), 4.54 (dt, J=10, 9 Hz, 1H), 7.16 (t, J=55 Hz, 1H), 7.64 (d, J=9 Hz, 1H), 7.96 (s, 1H), 8.01 (d, J=9 Hz, 1H), 8.06 (s, 1H), 8.54 (s, 1H), 9.07 (d, J=8 Hz, 1H); LC-MS (LC-ES) for C₁₅H₁₂F₃N₃O₂S M+H=356.

### Example 13

### 2-(3-(Difluoromethyl)-5-fluorophenyl)-N-((3S,4R)-4-methyl-2-oxopyrrolidin-3-yl)thiazole-5-carboxamide

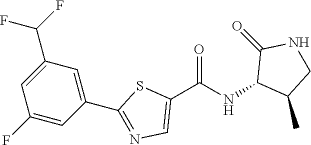 [SMILES: C[C@@H]1CNC(=O)[C@H]1NC(=O)c1cnc(-c2cc(F)cc(C(F)F)c2)s1]

N,N-Diisopropylethylamine (0.104 mL, 0.596 mmol) was added to a solution of 2-(3-(difluoromethyl)-5-fluorophenyl)thiazole-5-carboxylic acid (0.136 g, 0.497 mmol, Intermediate 11), (3S,4R)-3-amino-4-methylpyrrolidin-2-one (0.062 g, 0.547 mmol, Intermediate 10) and 1-((dimethylamino)(dimethyliminio)methyl)-1H-[1,2,3]triazolo[4,5-b]pyridine 3-oxide hexafluorophosphate(V) (0.208 g, 0.547 mmol) in N,N-dimethylformamide (3 mL) and the mixture was stirred at room temperature. After 2 hours, the mixture was poured into saturated sodium bicarbonate (20 mL) and water (5 mL) and extracted with ethyl acetate (3×). The combined organics were washed with water and saturated sodium chloride, dried over sodium sulfate, and concentrated onto Celite®. This powder was purified by reverse phase HPLC, eluting with acetonitrile:water (1:4 to 1:0) to give 2-(3-(difluoromethyl)-5-fluorophenyl)-N-((3S,4R)-4-methyl-2-oxopyrrolidin-3-yl)thiazole-5-carboxamide (0.124 g, 0.336 mmol, 67.5% yield) as a colorless solid. ¹H NMR (400 MHz, CD₃SOCD₃) δ 1.09 (d, J=6 Hz, 3H), 2.32-2.46 (m, 1H), 2.89 (t, J=10 Hz, 1H), 3.28-3.38 (m, 1H), 4.23 (dd, J=11, 9 Hz, 1H), 7.16 (t, J=56 Hz, 1H), 7.64 (d, J=9 Hz, 1H), 7.92 (s, 1H), 8.01 (dd, J=8, 1 Hz, 1H), 8.06 (s, 1H), 8.55 (s, 1H), 9.00 (d, J=9 Hz, 1H); LC-MS (LC-ES) for C₁₆H₁₄F₃N₃O₂S M+H=370.

### Example 14

### Racemic 2-(3-Chloro-5-fluorophenyl)-N-(5,5-dimethyl-2-oxopyrrolidin-3-yl)thiazole-5-carboxamide

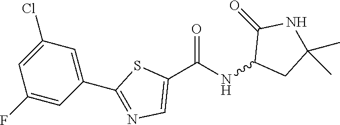 [SMILES: CC1(C)CC(NC(=O)c2cnc(-c3cc(F)cc(Cl)c3)s2)C(=O)N1]

N,N-Diisopropylethylamine (0.521 mL, 2.99 mmol) was added to 2-(3-chloro-5-fluorophenyl)thiazole-5-carboxylic acid (0.1282 g, 0.498 mmol, Intermediate 5) in N,N-dimethylformamide (2.488 mL) at room temperature. Then, racemic 3-amino-5,5-dimethylpyrrolidin-2-one hydrochloride (0.082 g, 0.498 mmol, Intermediate 12) was added and the reaction mixture was stirred for five minutes. Then, n-propylphosphonic acid anhydride (0.592 mL, 0.995 mmol) was added and the reaction mixture was stirred for sixteen hours. The reaction mixture was concentrated. The resulting residue was purified by reverse phase HPLC, eluting with acetonitrile:water with 0.1% ammonium hydroxide (5:95 to 100:0) to give racemic 2-(3-chloro-5-fluorophenyl)-N-(5,5-dimethyl-2-oxopyrrolidin-3-yl)thiazole-5-carboxamide (0.1370 g, 0.354 mmol, 71.1% yield). ¹H NMR (400 MHz, CD₃SOCD₃) δ 1.22 (s, 3H), 1.26 (s, 3H), 1.82 (t, J=12 Hz, 1H), 2.27 (dd, J=12, 10 Hz, 1H), 4.69 (q, J=9 Hz, 1H), 7.63 (d, J=9 Hz, 1H), 7.83 (d, J=9 Hz, 1H), 7.90 (s, 1H), 8.08 (s, 1H), 8.51 (s, 1H), 9.02 (d, J=9 Hz, 1H); LC-MS (LC-ES) for C₁₆H₁₅ClFN₃O₂S M+H=368.

### Examples 15 & 16

### (R)-2-(3-Chloro-5-fluorophenyl)-N-(5,5-dimethyl-2-oxopyrrolidin-3-yl)thiazole-5-carboxamide; and

### (S)-2-(3-Chloro-5-fluorophenyl)-N-(5,5-dimethyl-2-oxopyrrolidin-3-yl)thiazole-5-carboxamide

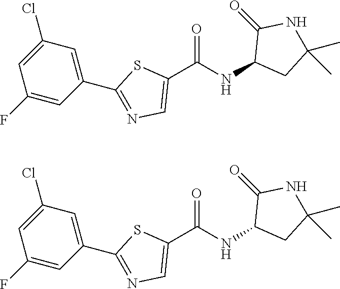 [SMILES: CC1(C)C[C@@H](NC(=O)c2cnc(-c3cc(F)cc(Cl)c3)s2)C(=O)N1 ; CC1(C)C[C@H](NC(=O)c2cnc(-c3cc(F)cc(Cl)c3)s2)C(=O)N1]

Racemic 2-(3-chloro-5-fluorophenyl)-N-(5,5-dimethyl-2-oxopyrrolidin-3-yl)thiazole-5-carboxamide (15.60 g, 42.4 mmol, Example 14, from multiple batches) was separated into its enantiomers via supercritical fluid chromatography on a chiral Chiralpak IB column, eluting with methanol:carbon dioxide (1:4) to give the first eluting enantiomer, which was then further purified via silica gel chromatography, eluting with ethyl acetate to give (R)-2-(3-chloro-5-fluorophenyl)-N-(5,5-dimethyl-2-oxopyrrolidin-3-yl)thiazole-5-carboxamide (5.86 g, 15.13 mmol, 35.7% yield) (OD column t_{R}=4.6 minutes, >99% ee). The second eluting enantiomer was further purified via chiral chromatography on a chiral Chiralpak OD column, eluting with ethanol:heptane (15:85), then via silica gel chromatography, eluting with ethyl acetate to give (S)-2-(3-chloro-5-fluorophenyl)-N-(5,5-dimethyl-2-oxopyrrolidin-3-yl)thiazole-5-carboxamide (5.58 g, 14.41 mmol, 34.0% yield) (OD column t_{R}=8.0 minutes, >99% ee). The structures were assigned by retention times on a chiral OD column, eluting with ethanol:heptane (15:85) with 0.1% isopropylamine (A previous batch was assigned by vibrational circular dichroism and correlated to these retention times).

### (R)-2-(3-Chloro-5-fluorophenyl)-N-(5,5-dimethyl-2-oxopyrrolidin-3-yl)thiazole-5-carboxamide—Example 15

¹H NMR (400 MHz, CD₃SOCD₃) δ 1.22 (s, 3H), 1.25 (s, 3H), 1.81 (dd, J=12, 11 Hz, 1H), 2.27 (dd, J=12, 9 Hz, 1H), 4.70 (dt, J=11, 9 Hz, 1H), 7.65 (dt, J=9, 2 Hz, 1H), 7.84 (ddd, J=9, 2, 1 Hz, 1H), 7.91 (t, J=2 Hz, 1H), 8.12 (br s, 1H), 8.51 (s, 1H), 9.06 (br d, J=8 Hz, 1H); LC-MS (LC-ES) for C₁₆H₁₅ClFN₃O₂S M+H=368.

### (S)-2-(3-Chloro-5-fluorophenyl)-N-(5,5-dimethyl-2-oxopyrrolidin-3-yl)thiazole-5-carboxamide—Example 16

¹H NMR (400 MHz, CD₃SOCD₃) δ 1.22 (s, 3H), 1.25 (s, 3H), 1.81 (dd, J=12, 11 Hz, 1H), 2.27 (dd, J=12, 9 Hz, 1H), 4.70 (dt, J=10, 9 Hz, 1H), 7.65 (dt, J=9, 2 Hz, 1H), 7.84 (ddd, J=9, 2, 1 Hz, 1H), 7.91 (t, J=2 Hz, 1H), 8.12 (br s, 1H), 8.51 (s, 1H), 9.06 (br d, J=9 Hz, 1H); LC-MS (LC-ES) for C₁₆H₁₅ClFN₃O₂S M+H=368.

Alternative Method

### Example 16

### (S)-2-(3-Chloro-5-fluorophenyl)-N-(5,5-dimethyl-2-oxopyrrolidin-3-yl)thiazole-5-carboxamide

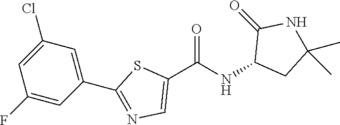 [SMILES: CC1(C)C[C@H](NC(=O)c2cnc(-c3cc(F)cc(Cl)c3)s2)C(=O)N1]

2-(3-Chloro-5-fluorophenyl)thiazole-5-carboxylic acid (38.49 g, 149 mmol, Intermediate 5) was added to (S)-3-amino-5,5-dimethylpyrrolidin-2-one hydrochloride (22.32 g, 136 mmol, Intermediate 13) and tetrahydrofuran (560 mL) and N,N-dimethylformamide (56 mL) were then added Diisopropylethylamine (59 mL, 339 mmol) was added to this suspension under nitrogen and put into a cool water bath (−8° C.). Then, 1-((dimethylamino)(dimethyliminio)methyl)-1H-[1,2,3]triazolo[4,5-b]pyridine 3-oxide hexafluorophosphate(V) (61.94 g, 163 mmol) was added. After ˜10 minutes, the bath was removed. After another ˜35 minutes, the reaction was quenched with 1 M potassium carbonate (280 mL). The layers were separated and the organics were washed once more with 1 M potassium carbonate (280 mL). 10% Citric acid (280 mL) was added to the organics, but the solution was homogeneous and no layers formed. The mixture was diluted with hexanes (280 mL). Three layers formed. The lower aqueous layer was removed. The combined organics and middle layer were washed once with 10% citric acid (280 mL). A solid formed. The solids were filtered off and dried under vacuum. The organic portion was partially concentrated to precipitate more solids. The solids were filtered off and dried under vacuum. All the aqueous washes were combined. There were some solids present so they were filtered off and air-dried. Partial evaporation of the filtrate yielded more solids. The solids were filtered off and rinsed with water then air-dried. These 4 crops of product were combined with the crops of product from another similar reaction (˜184 mmol total) with methanol (1000 mL) and the suspension was warmed on a 55° C. water bath until a solution formed. QuadraSil MP (Mercaptopropyl, 20.48 g, 1.0-1.5 mmol/g) was added and the mixture was stirred for ˜2 hours 15 minutes. The reaction mixture was again warmed on the 55° C. water bath and the solids were filtered off and rinsed with methanol. The filtrate was filtered again over a pad of Celite® and the resulting clear solution was concentrated to a slurry. While warm, the white solids were filtered off from the tan liquid (˜100 mL volume) and rinsed with a little methanol. The filtrate was partially concentrated to generate an additional crop of solids. While warm, the solids were filtered off from the liquid and rinsed with a little methanol. The solids were air-dried for several hours then further dried under vacuum for 4 days to give (S)-2-(3-chloro-5-fluorophenyl)-N-(5,5-dimethyl-2-oxopyrrolidin-3-yl)thiazole-5-carboxamide (61.60 g, 168 mmol, 91% yield).

Alternative Method

### (S)-2-(3-Chloro-5-fluorophenyl)-N-(5,5-dimethyl-2-oxopyrrolidin-3-yl)thiazole-5-carboxamide

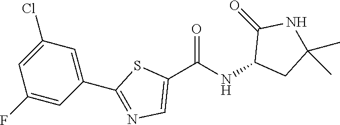 [SMILES: CC1(C)C[C@H](NC(=O)c2cnc(-c3cc(F)cc(Cl)c3)s2)C(=O)N1]

An oven-dried 250 mL flask with stir bar was charged with 2-(3-chloro-5-fluorophenyl)thiazole-5-carboxylic acid (2.57 g, 9.97 mmol, Intermediate 5) and dichloromethane (25 mL). The slurry was cooled in an ice bath under nitrogen and oxalyl chloride (0.917 mL, 10.47 mmol) was added dropwise. N,N-dimethylformamide (1 drop) was added and the mixture was stirred for about 5 minutes. A reflux condenser was attached, and the mixture was brought to reflux under nitrogen. A small portion of oxalyl chloride (0.09 mL) was added after 90 minutes and the reaction mixture was returned to reflux. After 2.5 hours, the light yellow solution was cooled to room temperature. A second flask with stir bar was charged with (S)-3-amino-5,5-dimethylpyrrolidin-2-one hydrochloride monohydrate (1.822 g, 9.97 mmol, Intermediate 13), dichloromethane (25 mL) and N,N-diisopropylethylamine (3.83 mL, 21.94 mmol). The reaction mixture was cooled in an ice bath and the above acid chloride solution was added via dropping addition funnel. The cooling bath was removed, and the mixture was stirred at room temperature. An additional portion of (S)-3-amino-5,5-dimethylpyrrolidin-2-one hydrochloride monohydrate (0.180 g) was added after 1 hour and stirring continued (LCMS after 1 hour and 15 minutes indicated complete conversion). The reaction mixture was concentrated in vacuo. The residue was partitioned between 2-methyltetrahydrofuran and water and the layers were separated. The aqueous layer was extracted with 2-methyltetrahydrofuran (2×). The combined organic layers were washed with saturated sodium chloride, dried over sodium sulfate, filtered, and concentrated in vacuo. The residue was taken up in a minimal amount of 2-methyltetrahydrofuran and methanol and adsorbed onto Celite®. Purification by silica gel chromatography, eluting with (3:1 ethyl acetate:ethanol):heptane (1:3 to 1:1) to give (S)-2-(3-chloro-5-fluorophenyl)-N-(5,5-dimethyl-2-oxopyrrolidin-3-yl)thiazole-5-carboxamide (3.22 g, 8.75 mmol, 88% yield) as a colorless solid. ¹H NMR (400 MHz, CD₃SOCD₃) δ 1.23 (s, 3H), 1.27 (s, 3H), 1.83 (dd, J=12, 11 Hz, 1H), 2.28 (dd, J=12, 9 Hz, 1H), 4.70 (dt, J=11, 9 Hz, 1H), 7.63 (dt, J=9, 2 Hz, 1H), 7.82 (ddd, J=9, 2, 1 Hz, 1H), 7.89 (t, J=2 Hz, 1H), 8.09 (br s, 1H), 8.52 (s, 1H), 9.02 (br d, J=8 Hz, 1H); LC-MS (LC-ES) for C₁₆H₁₅ClFN₃O₂S M+H=368.

### Example 17

### (S)-N-(1-(2-Amino-2-oxoethyl)-2-oxopyrrolidin-3-yl)-2-(3,5-difluorophenyl)thiazole-5-carboxamide

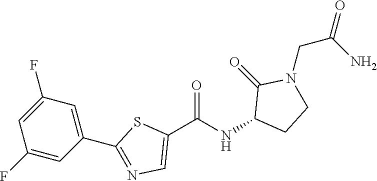 [SMILES: NC(=O)CN1CC[C@H](NC(=O)c2cnc(-c3cc(F)cc(F)c3)s2)C1=O]

(S)-2-(3-amino-2-oxopyrrolidin-1-yl)acetamide (31.0 mg, 0.197 mmol, Liang, C.; Gao, S.; Li, Z. Preparation of indolylidenemethylpyrrolecarboxamides as inhibitors of VEGFR, PDEGFR, KIT, Flt-1, Flt-3, Flt-4, and RET kinase with reduced inhibition of AMPK. PCT Int. Appl. WO 033562, 2008; Chem. Abstr. 2008, 148, 379474) was added to a solution of 2-(3,5-difluorophenyl)thiazole-5-carboxylic acid (40 mg, 0.164 mmol, Intermediate 14) in N,N-dimethylformamide (2 mL), followed by N,N-Diisopropylethylamine (0.086 mL, 0.493 mmol) and 1-((dimethylamino)(dimethyliminio)methyl)-1H-[1,2,3]triazolo[4,5-b]pyridine 3-oxide hexafluorophosphate(V) (94 mg, 0.246 mmol) at room temperature under argon. The resulting reaction mixture was stirred at 27° C. for 40 minutes. The reaction mixture was diluted with cold water (5 mL) and stirred for 20 minutes, then the solid was filtered off and dried. This residue was purified by preparative HPLC, eluting with acetonitrile:10 mM aqueous ammonium bicarbonate (0:1 to 9:5) to give (S)-N-(1-(2-amino-2-oxoethyl)-2-oxopyrrolidin-3-yl)-2-(3,5-difluorophenyl)thiazole-5-carboxamide (0.0052 g, 0.013 mmol, 8.2% yield) as an off white solid. ¹H NMR (400 MHz, CD₃SOCD₃) δ 1.94-2.08 (m, 1H), 2.34-2.44 (m, 1H), 3.34-3.46 (m, 2H), 3.81 (ABq, J_{AB}=17 Hz, Δv_{AB}=50 Hz, 2H), 4.63 (q, J=9 Hz, 1H), 7.16 (br s, 1H), 7.34 (br s, 1H), 7.45 (tt, J=9, 2 Hz, 1H), 7.68-7.86 (m, 2H), 8.51 (s, 1H), 9.16 (d, J=8 Hz, 1H); LC-MS (LC-ES) for C₁₆H₁₄F₂N₄O₃S M+H=381.

### Example 18

### Racemic 2-(3-(Difluoromethyl)-5-fluorophenyl)-N-(5,5-dimethyl-2-oxopyrrolidin-3-yl)thiazole-5-carboxamide

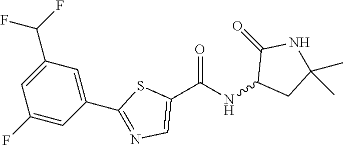 [SMILES: CC1(C)CC(NC(=O)c2cnc(-c3cc(F)cc(C(F)F)c3)s2)C(=O)N1]

N,N-Diisopropylethylamine (0.211 mL, 1.208 mmol) was added to 2-(3-(difluoromethyl)-5-fluorophenyl)thiazole-5-carboxylic acid (0.1500 g, 0.549 mmol, Intermediate 11), racemic 3-amino-5,5-dimethylpyrrolidin-2-one hydrochloride (0.099 g, 0.604 mmol, Intermediate 12), and 1-((dimethylamino)(dimethyliminio)methyl)-1H-[1,2,3]triazolo[4,5-b]pyridine 3-oxide hexafluorophosphate(V) (0.230 g, 0.604 mmol) in N,N-dimethylformamide (3 mL) and the reaction mixture was stirred overnight at room temperature. The reaction mixture was poured into saturated sodium bicarbonate (20 mL) and water (5 mL), then extracted with ethyl acetate (3×). The combined organics were washed with water and saturated sodium chloride, dried over sodium sulfate, and concentrated onto Celite®. This residue was purified by reverse phase HPLC, eluting with acetonitrile:water (1:4 to 100:0) to give racemic 2-(3-(difluoromethyl)-5-fluorophenyl)-N-(5,5-dimethyl-2-oxopyrrolidin-3-yl)thiazole-5-carboxamide (0.1715 g, 0.447 mmol, 81% yield) as a colorless foam. ¹H NMR (400 MHz, CD₃SOCD₃) δ 1.23 (s, 3H), 1.26 (s, 3H), 1.83 (dd, J=12, 11 Hz, 1H), 2.28 (dd, J=12, 9 Hz, 1H), 4.71 (dt, J=10, 9 Hz, 1H), 7.16 (t, J=56 Hz, 1H), 7.64 (dd, J=9, 1 Hz, 1H), 8.02 (dd, J=9, 1 Hz, 1H), 8.07 (m, J=1 Hz, 1H), 8.13 (s, 1H), 8.54 (s, 1H), 9.07 (d, J=8 Hz, 1H); LC-MS (LC-ES) for C₁₇H₁₆F₃N₃O₂S M+H=384.

### Example 19

### (S)-2-(3-Chloro-5-fluorophenyl)-N-(4,4-dimethyl-2-oxopyrrolidin-3-yl)thiazole-5-carboxamide

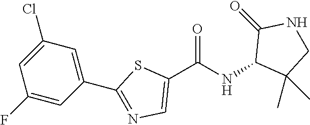 [SMILES: CC1(C)CNC(=O)[C@H]1NC(=O)c1cnc(-c2cc(F)cc(Cl)c2)s1]

N,N-Diisopropylethylamine (0.244 mL, 1.399 mmol) was added to 2-(3-chloro-5-fluorophenyl)thiazole-5-carboxylic acid (0.0721 g, 0.280 mmol, Intermediate 5) in dichloromethane (2.80 mL) at room temperature. Then, (S)-3-amino-4,4-dimethylpyrrolidin-2-one (0.054 g, 0.420 mmol, Camps, P.; Munoz-Torrero, D.; Rull, J.; Mayoral, J. A.; Calvet, T.; Font-Bardia, M. Straightforward preparation of enantiopure 3-amino-4,4-dimethylpyrrolidin-2-one and its derivatives Tetrahedron: Asymmetry 2010, 21, 2124-2135) was added and the reaction mixture was stirred for five minutes. Then, n-propylphosphonic acid anhydride (0.333 mL, 0.560 mmol) was added and the reaction mixture was stirred for sixteen hours. The reaction mixture was concentrated. The resulting residue was purified by RP HPLC, eluting with acetonitrile:water with 0.1% ammonium hydroxide (5:95 to 100:0), then further purified by silica gel chromatography, eluting with methanol:dichloromethane (0:1 to 1:4) to give (S)-2-(3-chloro-5-fluorophenyl)-N-(4,4-dimethyl-2-oxopyrrolidin-3-yl)thiazole-5-carboxamide (0.0903 g, 0.233 mmol, 83% yield). ¹H NMR (400 MHz, CD₃SOCD₃) δ 0.97 (s, 3H), 1.11 (s, 3H), 2.95 (dd, J=9, 2 Hz, 1H), 3.10 (d, J=9 Hz, 1H), 4.49 (d, J=9 Hz, 1H), 7.65 (dt, J=9, 2 Hz, 1H), 7.84 (ddd, J=9, 2, 2 Hz, 1H), 7.91 (t, J=2 Hz, 1H), 7.96 (br s, 1H), 8.69 (s, 1H), 8.91 (d, J=9 Hz, 1H); LC-MS (LC-ES) for C₁₆H₁₅ClFN₃O₂S M+H=368.

### Example 20

### Racemic 2-(3-Chloro-5-fluorophenyl)-N-(5-oxo-4-azaspiro[2.4]heptan-6-Yl)thiazole-5-carboxamide

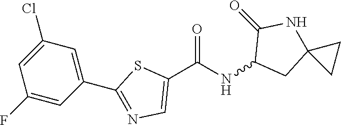 [SMILES: O=C(NC1CC2(CC2)NC1=O)c1cnc(-c2cc(F)cc(Cl)c2)s1]

N,N-Diisopropylethylamine (0.732 mL, 4.19 mmol) was added to 2-(3-chloro-5-fluorophenyl)thiazole-5-carboxylic acid (0.2159 g, 0.838 mmol, Intermediate 5) in dichloromethane (8.38 mL) at room temperature. Then, racemic 6-amino-4-azaspiro[2.4]heptan-5-one hydrochloride (0.170 g, 1.047 mmol, Intermediate 15) was added and the reaction mixture was stirred for five minutes. Then, n-propylphosphonic acid anhydride (0.998 mL, 1.676 mmol) was added and the reaction mixture was stirred for two hours. Saturated sodium bicarbonate was added to the reaction mixture and it was extracted with dichloromethane, dried over magnesium sulfate, filtered, and concentrated. The resulting residue was purified by silica gel chromatography, eluting with ethyl acetate:hexanes (3:2 to 1:0) to give racemic 2-(3-chloro-5-fluorophenyl)-N-(5-oxo-4-azaspiro[2.4]heptan-6-yl)thiazole-5-carboxamide (0.1163 g, 0.302 mmol, 36.0% yield). ¹H NMR (400 MHz, CD₃SOCD₃) δ 0.56-0.64 (m, 2H), 0.66-0.76 (m, 1H), 0.78-0.86 (m, 1H), 2.19 (dd, J=12, 9 Hz, 1H), 2.30 (dd, J=12, 10 Hz, 1H), 4.73 (q, J=9 Hz, 1H), 7.65 (dt, J=9, 2 Hz, 1H), 7.84 (ddd, J=9, 2, 2 Hz, 1H), 7.91 (t, J=1 Hz, 1H), 8.05 (br s, 1H), 8.53 (s, 1H), 9.17 (d, J=8 Hz, 1H); LC-MS (LC-ES) for C₁₆H₁₃ClFN₃O₂S M+H=366.

### Examples 21 & 22

### (S)-2-(3-Chloro-5-fluorophenyl)-N-(5-oxo-4-azaspiro[2.4]heptan-6-yl)thiazole-5-carboxamide; and

### (R)-2-(3-Chloro-5-fluorophenyl)-N-(5-oxo-4-azaspiro[2.4]heptan-6-yl)thiazole-5-carboxamide

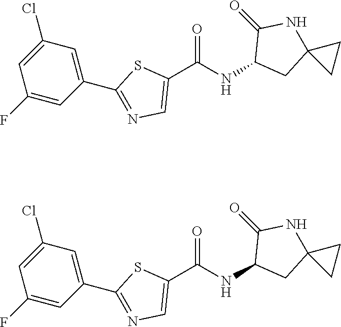 [SMILES: O=C(N[C@H]1CC2(CC2)NC1=O)c1cnc(-c2cc(F)cc(Cl)c2)s1 ; O=C(N[C@@H]1CC2(CC2)NC1=O)c1cnc(-c2cc(F)cc(Cl)c2)s1]

Racemic 2-(3-chloro-5-fluorophenyl)-N-(5-oxo-4-azaspiro[2.4]heptan-6-yl)thiazole-5-carboxamide (0.0996 g, 0.272 mmol, Example 20) was separated into its enantiomers on a chiral Chiralpak IC column, eluting with ethanol:heptane (1:3) with 0.1% isopropylamine to give (S)-2-(3-chloro-5-fluorophenyl)-N-(5-oxo-4-azaspiro[2.4]heptan-6-yl)thiazole-5-carboxamide (0.0397 g, 0.103 mmol, 37.9% yield) as the first enantiomer to elute (IC column t_{R}=8.2 minutes, >99% ee) and (R)-2-(3-chloro-5-fluorophenyl)-N-(5-oxo-4-azaspiro[2.4]heptan-6-yl)thiazole-5-carboxamide (0.0378 g, 0.098 mmol, 36.1% yield) as the last enantiomer to elute (IC column t_{R}=9.9 minutes, 97% ee). The structures were assigned by retention order on an OD column in analogy to Examples 15 & 16.

### (S)-2-(3-Chloro-5-fluorophenyl)-N-(5-oxo-4-azaspiro[2.4]heptan-6-yl)thiazole-5-carboxamide

¹H NMR (400 MHz, CD₃SOCD₃) δ 0.56-0.64 (m, 2H), 0.66-0.76 (m, 1H), 0.78-0.86 (m, 1H), 2.19 (dd, J=12, 9 Hz, 1H), 2.30 (dd, J=12, 10 Hz, 1H), 4.73 (q, J=9 Hz, 1H), 7.65 (dt, J=9, 2 Hz, 1H), 7.84 (ddd, J=9, 2, 2 Hz, 1H), 7.91 (t, J=1 Hz, 1H), 8.05 (br s, 1H), 8.53 (s, 1H), 9.17 (d, J=8 Hz, 1H); LC-MS (LC-ES) for C₁₆H₁₃ClFN₃O₂S M+H=366.

### (R)-2-(3-Chloro-5-fluorophenyl)-N-(5-oxo-4-azaspiro[2.4]heptan-6-yl)thiazole-5-carboxamide

¹H NMR (400 MHz, CD₃SOCD₃) δ 0.56-0.64 (m, 2H), 0.66-0.76 (m, 1H), 0.78-0.86 (m, 1H), 2.19 (dd, J=12, 9 Hz, 1H), 2.30 (dd, J=12, 10 Hz, 1H), 4.73 (q, J=9 Hz, 1H), 7.65 (dt, J=9, 2 Hz, 1H), 7.84 (ddd, J=9, 2, 2 Hz, 1H), 7.91 (t, J=1 Hz, 1H), 8.05 (br s, 1H), 8.53 (s, 1H), 9.17 (d, J=8 Hz, 1H); LC-MS (LC-ES) for C₁₆H₁₃ClFN₃O₂S M+H=366.

### Example 23

Racemic 2-(3-Chloro-5-fluorophenyl)-N-(4,4-diethyl-2-oxopyrrolidin-3-yl)thiazole-5-carboxamide

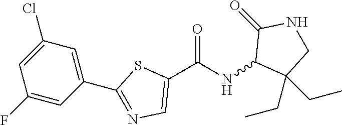 [SMILES: CCC1(CC)CNC(=O)C1NC(=O)c1cnc(-c2cc(F)cc(Cl)c2)s1]

N,N-Diisopropylethylamine (0.542 mL, 3.10 mmol) was added to 2-(3-chloro-5-fluorophenyl)thiazole-5-carboxylic acid (0.1598 g, 0.620 mmol, Intermediate 5) in dichloromethane (6.20 mL) at room temperature. Then, racemic 3-amino-4,4-diethylpyrrolidin-2-one (0.145 g, 0.930 mmol, Enamine) was added and the reaction mixture was stirred for five minutes. Then, n-propylphosphonic acid anhydride (0.738 mL, 1.240 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with ethyl acetate:hexanes (4:1 to 1:0) to give racemic 2-(3-chloro-5-fluorophenyl)-N-(4,4-diethyl-2-oxopyrrolidin-3-yl)thiazole-5-carboxamide (0.1303 g, 0.313 mmol, 50.4% yield). ¹H NMR (400 MHz, CD₃SOCD₃) δ 0.79 (t, J=7 Hz, 3H), 0.83 (t, J=7 Hz, 3H), 1.32-1.54 (m, 4H), 2.97 (d, J=10 Hz, 1H), 3.05 (dd, J=10, 1 Hz, 1H), 4.50 (d, J=9 Hz, 1H), 7.65 (dt, J=9, 2 Hz, 1H), 7.84 (ddd, J=9, 2, 2 Hz, 1H), 7.91 (t, J=2 Hz, 1H), 7.93 (br s, 1H), 8.64 (s, 1H), 8.91 (d, J=9 Hz, 1H); LC-MS (LC-ES) for C₁₈H₁₉ClFN₃O₂S M+H=396.

### Examples 24 & 25

### (R)-2-(3-Chloro-5-fluorophenyl)-N-(4,4-diethyl-2-oxopyrrolidin-3-yl)thiazole-5-carboxamide; and

### (S)-2-(3-Chloro-5-fluorophenyl)-N-(4,4-diethyl-2-oxopyrrolidin-3-yl)thiazole-5-carboxamide

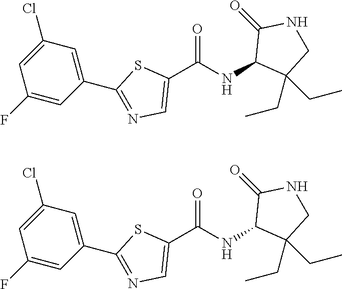 [SMILES: CCC1(CC)CNC(=O)[C@@H]1NC(=O)c1cnc(-c2cc(F)cc(Cl)c2)s1 ; CCC1(CC)CNC(=O)[C@H]1NC(=O)c1cnc(-c2cc(F)cc(Cl)c2)s1]

Racemic 2-(3-chloro-5-fluorophenyl)-N-(4,4-diethyl-2-oxopyrrolidin-3-yl)thiazole-5-carboxamide (0.1173 g, 0.296 mmol, Example 23) was separated into its enantiomers on a chiral Chiralpak OD-H column, eluting with methanol with 0.1% isopropylamine to give (R)-2-(3-chloro-5-fluorophenyl)-N-(4,4-diethyl-2-oxopyrrolidin-3-yl)thiazole-5-carboxamide (0.0379 g, 0.091 mmol, 30.7% yield) as the first enantiomer to elute (t_{R}=3.0 minutes, >99% ee) and (S)-2-(3-chloro-5-fluorophenyl)-N-(4,4-diethyl-2-oxopyrrolidin-3-yl)thiazole-5-carboxamide (0.0288 g, 0.069 mmol, 23.32% yield) as the last enantiomer to elute (t_{R}=4.1 minutes, 90% ee). The structures were assigned by vibrational circular dichroism.

### (R)-2-(3-Chloro-5-fluorophenyl)-N-(4,4-diethyl-2-oxopyrrolidin-3-yl)thiazole-5-carboxamide

¹H NMR (400 MHz, CD₃SOCD₃) δ 0.79 (t, J=7 Hz, 3H), 0.84 (t, J=7 Hz, 3H), 1.32-1.54 (m, 4H), 2.97 (d, J=10 Hz, 1H), 3.05 (dd, J=10, 1 Hz, 1H), 4.50 (d, J=9 Hz, 1H), 7.64 (dt, J=9, 2 Hz, 1H), 7.83 (ddd, J=9, 2, 2 Hz, 1H), 7.90 (t, J=2 Hz, 1H), 7.91 (br s, 1H), 8.64 (s, 1H), 8.88 (d, J=9 Hz, 1H); LC-MS (LC-ES) for C₁₈H₁₉ClFN₃O₂S M+H=396.

### (S)-2-(3-Chloro-5-fluorophenyl)-N-(4,4-diethyl-2-oxopyrrolidin-3-yl)thiazole-5-carboxamide

¹H NMR (400 MHz, CD₃SOCD₃) δ 0.79 (t, J=7 Hz, 3H), 0.84 (t, J=7 Hz, 3H), 1.32-1.54 (m, 4H), 2.97 (d, J=10 Hz, 1H), 3.05 (dd, J=10, 1 Hz, 1H), 4.50 (d, J=9 Hz, 1H), 7.64 (dt, J=9, 2 Hz, 1H), 7.83 (ddd, J=9, 2, 2 Hz, 1H), 7.90 (t, J=2 Hz, 1H), 7.91 (br s, 1H), 8.64 (s, 1H), 8.88 (d, J=9 Hz, 1H); LC-MS (LC-ES) for C₁₈H₁₉ClFN₃O₂S M+H=396.

### Example 26

### (R)-2-(3-Chloro-5-fluorophenyl)-N-(2-oxopyrrolidin-3-yl)thiazole-5-carboxamide

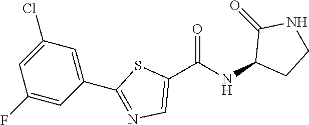 [SMILES: O=C(N[C@@H]1CCNC1=O)c1cnc(-c2cc(F)cc(Cl)c2)s1]

N,N-Diisopropylethylamine (0.148 mL, 0.850 mmol) was added to 2-(3-chloro-5-fluorophenyl)thiazole-5-carboxylic acid (0.0438 g, 0.170 mmol, Intermediate 5) in dichloromethane (1.700 mL) at room temperature. Then, (R)-3-aminopyrrolidin-2-one (0.026 g, 0.255 mmol, AstaTech) was added and the reaction mixture was stirred for five minutes. Then, n-propylphosphonic acid anhydride (0.202 mL, 0.340 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 1:4) to give (R)-2-(3-chloro-5-fluorophenyl)-N-(2-oxopyrrolidin-3-yl)thiazole-5-carboxamide (0.0396 g, 0.111 mmol, 65.1% yield). ¹H NMR (400 MHz, CD₃SOCD₃) δ 1.90-2.04 (m, 1H), 2.28-2.42 (m, 1H), 3.16-3.28 (m, 2H), 4.52 (dt, J=10, 9 Hz, 1H), 7.64 (dt, J=9, 2 Hz, 1H), 7.83 (ddd, J=9, 2, 1 Hz, 1H), 7.94 (t, J=1 Hz, 1H), 7.94 (br s, 1H), 8.51 (s, 1H), 9.06 (d, J=8 Hz, 1H); LC-MS (LC-ES) for C₁₄H₁₁ClFN₃O₂S M+H=341.

### Example 27

### Racemic 2-(3-Chloro-5-fluorophenyl)-N-(6-oxo-5-azaspiro[3.4]octan-7-v)thiazole-5-carboxamide

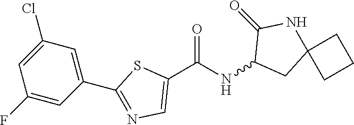 [SMILES: O=C(NC1CC2(CCC2)NC1=O)c1cnc(-c2cc(F)cc(Cl)c2)s1]

N,N-Diisopropylethylamine (0.379 mL, 2.169 mmol) was added to 2-(3-chloro-5-fluorophenyl)thiazole-5-carboxylic acid (0.1118 g, 0.434 mmol, Intermediate 5) in dichloromethane (4.34 mL) at room temperature. Then, racemic 7-amino-5-azaspiro[3.4]octan-6-one hydrochloride (0.115 g, 0.651 mmol, Intermediate 16) was added and the reaction mixture was stirred for five minutes. Then, n-propylphosphonic acid anhydride (0.517 mL, 0.868 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 1:4) to give racemic 2-(3-chloro-5-fluorophenyl)-N-(6-oxo-5-azaspiro[3.4]octan-7-yl)thiazole-5-carboxamide (0.1346 g, 0.337 mmol, 78% yield). ¹H NMR (400 MHz, CD₃SOCD₃) δ 1.58-1.72 (m, 2H), 1.92-2.02 (m, 2H), 2.04 (q, J=10 Hz, 1H), 2.12-2.20 (m, 1H), 2.30 (q, J=10 Hz, 1H), 2.65 (dd, J=12, 8 Hz, 1H), 4.57 (dt, J=11, 9 Hz, 1H), 7.65 (dt, J=9, 2 Hz, 1H), 7.84 (ddd, J=9, 2, 2 Hz, 1H), 7.91 (t, J=1 Hz, 1H), 8.45 (br s, 1H), 8.51 (s, 1H), 9.07 (d, J=8 Hz, 1H); LC-MS (LC-ES) for C₁₇H₁₅ClFN₃O₂S M+H=380.

### Examples 28 & 29

### (R)-2-(3-Chloro-5-fluorophenyl)-N-(6-oxo-5-azaspiro[3.4]octan-7-yl)thiazole-5-carboxamide; and

### (S)-2-(3-Chloro-5-fluorophenyl)-N-(6-oxo-5-azaspiro[3.4]octan-7-yl)thiazole-5-carboxamide

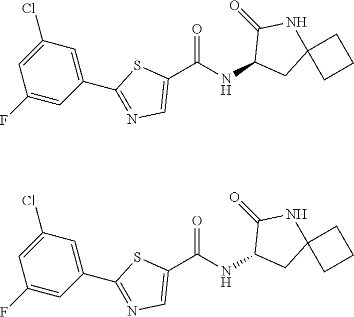 [SMILES: O=C(N[C@@H]1CC2(CCC2)NC1=O)c1cnc(-c2cc(F)cc(Cl)c2)s1 ; O=C(N[C@H]1CC2(CCC2)NC1=O)c1cnc(-c2cc(F)cc(Cl)c2)s1]

Racemic 2-(3-chloro-5-fluorophenyl)-N-(6-oxo-5-azaspiro[3.4]octan-7-yl)thiazole-5-carboxamide (0.1145 g, 0.301 mmol, Example 27) was separated into its enantiomers on a chiral Chiralpak OD column, eluting with ethanol:heptane (15:85) with 0.1% isopropylamine to give (R)-2-(3-chloro-5-fluorophenyl)-N-(6-oxo-5-azaspiro[3.4]octan-7-yl)thiazole-5-carboxamide (0.0486 g, 0.122 mmol, 40.3% yield) as the first enantiomer to elute (t_{R}=5.0 minutes, >99% ee) and (S)-2-(3-chloro-5-fluorophenyl)-N-(6-oxo-5-azaspiro[3.4]octan-7-yl)thiazole-5-carboxamide (0.0492 g, 0.123 mmol, 40.8% yield) as the last enantiomer to elute (t_{R}=7.6 minutes, >99% ee). The structures were assigned by analogy to Examples 15 & 16.

### (R)-2-(3-Chloro-5-fluorophenyl)-N-(6-oxo-5-azaspiro[3.4]octan-7-yl)thiazole-5-carboxamide

¹H NMR (400 MHz, CD₃SOCD₃) δ 1.58-1.72 (m, 2H), 1.92-2.02 (m, 2H), 2.04 (q, J=10 Hz, 1H), 2.10-2.22 (m, 1H), 2.31 (q, J=10 Hz, 1H), 2.66 (dd, J=12, 8 Hz, 1H), 4.57 (dt, J=11, 9 Hz, 1H), 7.64 (dt, J=9, 2 Hz, 1H), 7.84 (ddd, J=9, 2, 1 Hz, 1H), 7.90 (t, J=2 Hz, 1H), 8.43 (br s, 1H), 8.51 (s, 1H), 9.05 (d, J=8 Hz, 1H); LC-MS (LC-ES) for C₁₇H₁₅ClFN₃O₂S M+H=380.

### (S)-2-(3-Chloro-5-fluorophenyl)-N-(6-oxo-5-azaspiro[3.4]octan-7-yl)thiazole-5-carboxamide

¹H NMR (400 MHz, CD₃SOCD₃) δ 1.58-1.72 (m, 2H), 1.92-2.02 (m, 2H), 2.04 (q, J=10 Hz, 1H), 2.10-2.22 (m, 1H), 2.31 (q, J=10 Hz, 1H), 2.66 (dd, J=12, 8 Hz, 1H), 4.57 (dt, J=11, 8 Hz, 1H), 7.64 (dt, J=9, 2 Hz, 1H), 7.83 (ddd, J=9, 2, 2 Hz, 1H), 7.90 (t, J=2 Hz, 1H), 8.43 (br s, 1H), 8.51 (s, 1H), 9.05 (d, J=8 Hz, 1H); LC-MS (LC-ES) for C₁₇H₁₅ClFN₃O₂S M+H=380.

### Example 30

### (S)-2-(3-Chlorophenyl)-4-methyl-N-(2-oxopyrrolidin-3-yl)thiazole-5-carboxamide

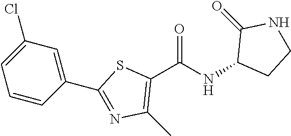 [SMILES: Cc1nc(-c2cccc(Cl)c2)sc1C(=O)N[C@H]1CCNC1=O]

N,N-Diisopropylethylamine (0.264 mL, 1.514 mmol) was added to 2-(3-chlorophenyl)-4-methylthiazole-5-carboxylic acid (0.0768 g, 0.303 mmol, Intermediate 17) in dichloromethane (3.03 mL) at room temperature. Then, (S)-3-aminopyrrolidin-2-one (0.045 g, 0.454 mmol) was added and the reaction mixture was stirred for five minutes. Then, n-propylphosphonic acid anhydride (0.360 mL, 0.605 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 1:4) to give (S)-2-(3-chlorophenyl)-4-methyl-N-(2-oxopyrrolidin-3-yl)thiazole-5-carboxamide (0.0692 g, 0.196 mmol, 64.7% yield). ¹H NMR (400 MHz, CD₃SOCD₃) δ 1.94-2.06 (m, 1H), 2.28-2.38 (m, 1H), 2.62 (s, 3H), 3.18-3.24 (m, 2H), 4.49 (dt, J=10, 9 Hz, 1H), 7.55 (t, J=8 Hz, 1H), 7.60 (dt, J=8, 2 Hz, 1H), 7.88 (dt, J=8, 2 Hz, 1H), 7.88 (br s, 1H), 7.96 (t, J=2 Hz, 1H), 8.54 (d, J=8 Hz, 1H); LC-MS (LC-ES) for C₁₅H₁₄ClN₃O₂S M+H=336.

### Example 31

### (S)-2-(4-Methyl-1H-pyrazol-1-yl)-N-(2-oxopyrrolidin-3-yl)thiazole-5-carboxamide

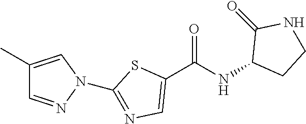 [SMILES: Cc1cnn(-c2ncc(C(=O)N[C@H]3CCNC3=O)s2)c1]

N,N-Diisopropylethylamine (0.299 mL, 1.713 mmol) was added to 2-(4-methyl-1H-pyrazol-1-yl)thiazole-5-carboxylic acid (0.0717 g, 0.343 mmol, Intermediate 18) in dichloromethane (1.713 mL) and N,N-dimethylformamide (1.713 mL) at room temperature. Then, (S)-3-aminopyrrolidin-2-one (0.051 g, 0.514 mmol) was added and the reaction mixture was stirred for five minutes. Then, n-propylphosphonic acid anhydride (0.408 mL, 0.685 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-2-(4-methyl-1H-pyrazol-1-yl)-N-(2-oxopyrrolidin-3-yl)thiazole-5-carboxamide (0.0132 g, 0.043 mmol, 12.56% yield). ¹H NMR (400 MHz, CD₃SOCD₃) δ 1.88-2.02 (m, 1H), 2.09 (s, 3H), 2.30-2.40 (m, 1H), 3.16-3.26 (m, 2H), 4.49 (q, J=9 Hz, 1H), 7.75 (s, 1H), 7.93 (br s, 1H), 8.20 (s, 1H), 8.31 (s, 1H), 8.93 (d, J=8 Hz, 1H); LC-MS (LC-ES) for C₁₂H₁₃ClFN₃O₂S M+H=292.

### Example 32

### (S)-2-(4-Methyl-1H-imidazol-1-yl)-N-(2-oxopyrrolidin-3-yl)thiazole-5-carboxamide

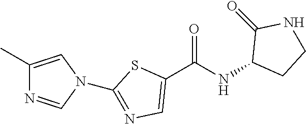 [SMILES: Cc1cn(-c2ncc(C(=O)N[C@H]3CCNC3=O)s2)cn1]

N,N-Diisopropylethylamine (0.309 mL, 1.771 mmol) was added to 2-(4-methyl-1H-imidazol-1-yl)thiazole-5-carboxylic acid (0.0741 g, 0.354 mmol, Intermediate 19) in N,N-dimethylformamide (1.771 mL) at room temperature. Then, (S)-3-aminopyrrolidin-2-one (0.053 g, 0.531 mmol) was added and the reaction mixture was stirred for five minutes. Then, n-propylphosphonic acid anhydride (0.422 mL, 0.708 mmol) was added and the reaction mixture was stirred for sixteen hours. Then, the reaction mixture was concentrated and the resulting residue was purified by reverse phase HPLC, eluting with acetonitrile:water with 0.1% ammonium hydroxide (0:1 to 1:0) to give (S)-2-(4-methyl-1H-imidazol-1-yl)-N-(2-oxopyrrolidin-3-yl)thiazole-5-carboxamide (0.0463 g, 0.151 mmol, 42.6% yield). ¹H NMR (400 MHz, CD₃SOCD₃) δ 1.88-2.02 (m, 1H), 2.15 (d, J=1 Hz, 3H), 2.30-2.40 (m, 1H), 3.18-3.28 (m, 2H), 4.50 (dt, J=10, 9 Hz, 1H), 7.58 (t, J=1 Hz, 1H), 7.95 (s, 1H), 8.22 (s, 1H), 8.35 (d, J=2 Hz, 1H), 9.01 (d, J=8 Hz, 1H); LC-MS (LC-ES) for C₁₂H₁₃ClFN₃O₂S M+H=292.

### Example 33

### (S)-N-(2-Oxopyrrolidin-3-yl)-2-phenylthiazole-5-carboxamide

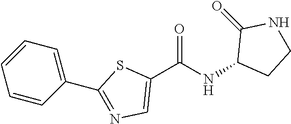 [SMILES: O=C(N[C@H]1CCNC1=O)c1cnc(-c2ccccc2)s1]

N,N-Diisopropylethylamine (0.308 mL, 1.766 mmol) was added to 2-phenylthiazole-5-carboxylic acid (0.0725 g, 0.353 mmol, Intermediate 20) in dichloromethane (3.53 mL) at room temperature. Then, (S)-3-aminopyrrolidin-2-one (0.053 g, 0.530 mmol) was added and the reaction mixture was stirred for five minutes. Then, n-propylphosphonic acid anhydride (0.421 mL, 0.707 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give (S)-N-(2-oxopyrrolidin-3-yl)-2-phenylthiazole-5-carboxamide (0.0283 g, 0.094 mmol, 26.5% yield) as well as some impure fractions that were discarded. ¹H NMR (400 MHz, CD₃SOCD₃) δ 1.92-2.04 (m, 1H), 2.30-2.42 (m, 1H), 3.20-3.28 (m, 2H), 4.52 (dt, J=10, 9 Hz, 1H), 7.50-7.58 (m, 3H), 7.94 (s, 1H), 7.96-8.02 (m, 2H), 8.47 (s, 1H), 8.98 (d, J=8 Hz, 1H); LC-MS (LC-ES) for C₁₄H₁₃N₃O₂S M+H=288.

### Example 34

### Racemic 2-(3-Chloro-5-fluorophenyl)-N-((3R,4S,5S)-4,5-dimethyl-2-oxopyrrolidin-3-yl)thiazole-5-carboxamide; and

### 2-(3-Chloro-5-fluorophenyl)-N-((3S,4R,5R)-4,5-dimethyl-2-oxopyrrolidin-3-yl)thiazole-5-carboxamide

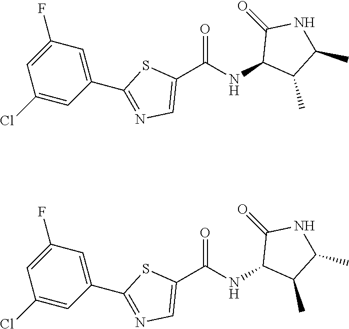 [SMILES: C[C@H]1[C@H](C)NC(=O)[C@@H]1NC(=O)c1cnc(-c2cc(F)cc(Cl)c2)s1 ; C[C@H]1[C@H](NC(=O)c2cnc(-c3cc(F)cc(Cl)c3)s2)C(=O)N[C@@H]1C]

N,N-Diisopropylethylamine (0.442 mL, 2.53 mmol) was added to 2-(3-chloro-5-fluorophenyl)thiazole-5-carboxylic acid (0.1303 g, 0.506 mmol, Intermediate 5) in dichloromethane (5.06 mL) at room temperature. Then, racemic (3R,4R,5S)-3-amino-4,5-dimethylpyrrolidin-2-one hydrochloride and (3S,4S,5R)-3-amino-4,5-dimethylpyrrolidin-2-one hydrochloride (0.125 g, 0.759 mmol, Intermediate 21) was added and the reaction mixture was stirred for five minutes. Then, n-propylphosphonic acid anhydride (0.602 mL, 1.011 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 1:4) to give racemic 2-(3-chloro-5-fluorophenyl)-N-((3R,4S,5S)-4,5-dimethyl-2-oxopyrrolidin-3-yl)thiazole-5-carboxamide and 2-(3-chloro-5-fluorophenyl)-N-((3S,4R,5R)-4,5-dimethyl-2-oxopyrrolidin-3-yl)thiazole-5-carboxamide (0.1418 g, 0.366 mmol, 72.4% yield). ¹H NMR (400 MHz, CD₃SOCD₃) δ 1.06 (d, J=6 Hz, 3H), 1.15 (d, J=6 Hz, 3H), 1.78-1.90 (m, 1H), 3.14-3.24 (m, 1H), 4.30 (dd, J=11, 8 Hz, 1H), 7.64 (dt, J=9, 2 Hz, 1 H), 7.83 (ddd, J=9, 2, 2 Hz, 1H), 7.90 (t, J=1 Hz, 1H), 7.98 (s, 1H), 8.52 (s, 1H), 8.96 (d, J=9 Hz, 1H); LC-MS (LC-ES) for C₁₆H₁₅N₃O₂S M+H=368.

### Examples 35 & 36

### 2-(3-Chloro-5-fluorophenyl)-N-((3R,4S,5S)-4,5-dimethyl-2-oxopyrrolidin-3-yl)thiazole-5-carboxamide; and

### 2-(3-Chloro-5-fluorophenyl)-N-((3S,4R,5R)-4,5-dimethyl-2-oxopyrrolidin-3-yl)thiazole-5-carboxamide

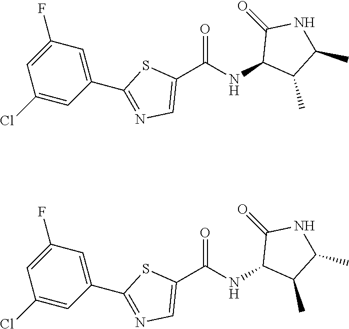 [SMILES: C[C@H]1[C@H](C)NC(=O)[C@@H]1NC(=O)c1cnc(-c2cc(F)cc(Cl)c2)s1 ; C[C@H]1[C@H](NC(=O)c2cnc(-c3cc(F)cc(Cl)c3)s2)C(=O)N[C@@H]1C]

Racemic 2-(3-chloro-5-fluorophenyl)-N-((3R,4S,5S)-4,5-dimethyl-2-oxopyrrolidin-3-yl)thiazole-5-carboxamide and 2-(3-chloro-5-fluorophenyl)-N-((3S,4R,5R)-4,5-dimethyl-2-oxopyrrolidin-3-yl)thiazole-5-carboxamide (0.1254 g, 0.341 mmol, Example 34) was separated into its enantiomers on a chiral Chiralpak CC4 column, eluting with ethanol:heptane (3:7) with 0.1% isopropylamine to give 2-(3-chloro-5-fluorophenyl)-N-((3R,4S,5S)-4,5-dimethyl-2-oxopyrrolidin-3-yl)thiazole-5-carboxamide (0.0513 g, 0.132 mmol, 38.9% yield) as the first enantiomer to elute (t_{R}=6.5 minutes, >99% ee) and 2-(3-chloro-5-fluorophenyl)-N-((3S,4R,5R)-4,5-dimethyl-2-oxopyrrolidin-3-yl)thiazole-5-carboxamide (0.0507 g, 0.131 mmol, 38.4% yield) as the last enantiomer to elute (t_{R}=7.7 minutes, >99% ee). The structures were assigned by vibrational circular dichroism.

### 2-(3-Chloro-5-fluorophenyl)-N-((3R,4S,5S)-4,5-dimethyl-2-oxopyrrolidin-3-yl)thiazole-5-carboxamide

¹H NMR (400 MHz, CD₃SOCD₃) δ 1.07 (d, J=6 Hz, 3H), 1.15 (d, J=6 Hz, 3H), 1.80-1.90 (m, 1H), 3.16-3.26 (m, 1H), 4.30 (dd, J=11, 9 Hz, 1H), 7.65 (dt, J=9, 2 Hz, 1H), 7.85 (ddd, J=9, 2, 2 Hz, 1H), 7.91 (t, J=2 Hz, 1H), 8.01 (s, 1H), 8.53 (s, 1H), 8.98 (d, J=9 Hz, 1H); LC-MS (LC-ES) for C₁₆H₁₅N₃O₂S M+H=368.

### 2-(3-Chloro-5-fluorophenyl)-N-((3S,4R,5R)-4,5-dimethyl-2-oxopyrrolidin-3-yl)thiazole-5-carboxamide

¹H NMR (400 MHz, CD₃SOCD₃) δ 1.06 (d, J=6 Hz, 3H), 1.15 (d, J=6 Hz, 3H), 1.78-1.90 (m, 1H), 3.16-3.24 (m, 1H), 4.30 (dd, J=11, 9 Hz, 1H), 7.64 (dt, J=9, 2 Hz, 1H), 7.84 (ddd, J=9, 2, 2 Hz, 1H), 7.90 (t, J=2 Hz, 1H), 8.00 (s, 1H), 8.52 (s, 1H), 8.97 (d, J=9 Hz, 1H); LC-MS (LC-ES) for C₁₆H₁₅N₃O₂S M+H=368.

### Example 37

### (S)-2-(3-Bromophenyl)-N-(2-oxopyrrolidin-3-yl)thiazole-5-carboxamide

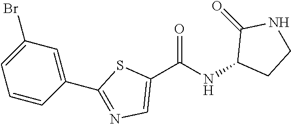 [SMILES: O=C(N[C@H]1CCNC1=O)c1cnc(-c2cccc(Br)c2)s1]

N,N-Diisopropylethylamine (0.230 mL, 1.318 mmol) was added to 2-(3-bromophenyl)thiazole-5-carboxylic acid (0.0749 g, 0.264 mmol, Intermediate 22) in dichloromethane (2.64 mL) at room temperature. Then, (S)-3-aminopyrrolidin-2-one (0.040 g, 0.395 mmol) was added and the reaction mixture was stirred for five minutes. Then, n-propylphosphonic acid anhydride (0.314 mL, 0.527 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-2-(3-bromophenyl)-N-(2-oxopyrrolidin-3-yl)thiazole-5-carboxamide (0.0394 g, 0.102 mmol, 38.8% yield). ¹H NMR (400 MHz, CD₃SOCD₃) δ 1.92-2.04 (m, 1H), 2.30-2.42 (m, 1H), 3.18-3.26 (m, 2H), 4.51 (dt, J=10, 9 Hz, 1H), 7.48 (t, J=8 Hz, 1H), 7.73 (ddd, J=8, 2, 1 Hz, 1H), 7.97 (s, 1H), 7.98 (ddd, J=9, 2, 1 Hz, 1H), 8.15 (t, J=1 Hz, 1H), 8.49 (s, 1H), 9.01 (d, J=8 Hz, 1H); LC-MS (LC-ES) for C₁₄H₁₂BrN₃O₂S M+H=366.

### Example 38

### (S)-N-(2-Oxopyrrolidin-3-yl)-2-(pyridin-4-yl)thiazole-5-carboxamide

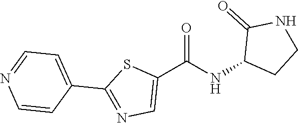 [SMILES: O=C(N[C@H]1CCNC1=O)c1cnc(-c2ccncc2)s1]

N,N-Diisopropylethylamine (0.379 mL, 2.172 mmol) was added to 2-(pyridin-4-yl)thiazole-5-carboxylic acid (0.0896 g, 0.434 mmol, Intermediate 23) in dichloromethane (4.34 mL) at room temperature. Then, (S)-3-aminopyrrolidin-2-one (0.065 g, 0.652 mmol) was added and the reaction mixture was stirred for five minutes. Then, n-propylphosphonic acid anhydride (0.517 mL, 0.869 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-N-(2-oxopyrrolidin-3-yl)-2-(pyridin-4-yl)thiazole-5-carboxamide (0.0431 g, 0.142 mmol, 32.7% yield). ¹H NMR (400 MHz, CD₃SOCD₃) δ 1.92-2.06 (m, 1H), 2.32-2.44 (m, 1H), 3.18-3.28 (m, 2H), 4.53 (dt, J=10, 9 Hz, 1H), 7.90-7.96 (m, 3H), 8.57 (s, 1H), 8.72-8.76 (m, 2H), 9.08 (d, J=8 Hz, 1H); LC-MS (LC-ES) for C₁₃H₁₂N₄O₂S M+H=289.

### Example 39

### (S)-N-(2-Oxopyrrolidin-3-yl)-2-(pyridin-2-yl)thiazole-5-carboxamide

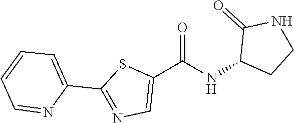 [SMILES: O=C(N[C@H]1CCNC1=O)c1cnc(-c2ccccn2)s1]

N,N-Diisopropylethylamine (0.360 mL, 2.063 mmol) was added to 2-(pyridin-2-yl)thiazole-5-carboxylic acid (0.0851 g, 0.413 mmol, Intermediate 24) in N,N-dimethylformamide (4.13 mL) at room temperature. Then, (S)-3-aminopyrrolidin-2-one (0.062 g, 0.619 mmol) was added and the reaction mixture was stirred for five minutes. Then, n-propylphosphonic acid anhydride (0.491 mL, 0.825 mmol) was added and the reaction mixture was stirred for sixty-six hours. The reaction mixture was concentrated. The resulting residue was purified by reverse phase HPLC, eluting with acetonitrile:water with 0.1% ammonium hydroxide (0:1 to 1:0) to give (S)-N-(2-oxopyrrolidin-3-yl)-2-(pyridin-2-yl)thiazole-5-carboxamide (0.0481 g, 0.158 mmol, 38.4% yield). ¹H NMR (400 MHz, CD₃SOCD₃) δ 1.92-2.06 (m, 1H), 2.32-2.42 (m, 1H), 3.20-3.28 (m, 2H), 4.51 (dt, J=10, 9 Hz, 1H), 7.55 (ddd, J=8, 5, 1 Hz, 1H), 7.92 (br s, 1H), 7.99 (dt, J=8, 2 Hz, 1H), 8.16 (d, J=8 Hz, 1H), 8.52 (s, 1H), 8.67 (ddd, J=5, 2, 1 Hz, 1H), 8.99 (d, J=8 Hz, 1H); LC-MS (LC-ES) for C₁₃H₁₂N₄O₂S M+H=289.

### Example 40

### (S)-N-(2-Oxopyrrolidin-3-yl)-2-(pyridin-3-yl)thiazole-5-carboxamide

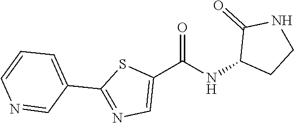 [SMILES: O=C(N[C@H]1CCNC1=O)c1cnc(-c2cccnc2)s1]

N,N-Diisopropylethylamine (0.360 mL, 2.063 mmol) was added to 2-(pyridin-3-yl)thiazole-5-carboxylic acid (0.0851 g, 0.413 mmol, Intermediate 25) in N,N-dimethylformamide (4.13 mL) at room temperature. Then, (S)-3-aminopyrrolidin-2-one (0.062 g, 0.619 mmol) was added and the reaction mixture was stirred for five minutes. Then, n-propylphosphonic acid anhydride (0.491 mL, 0.825 mmol) was added and the reaction mixture was stirred for three hours. The reaction mixture was concentrated. The resulting residue was purified by reverse phase HPLC, eluting with acetonitrile:water with 0.1% ammonium hydroxide (0:1 to 1:0) to give (S)-N-(2-oxopyrrolidin-3-yl)-2-(pyridin-3-yl)thiazole-5-carboxamide (0.0290 g, 0.096 mmol, 23.15% yield). ¹H NMR (400 MHz, CD₃SOCD₃) δ 1.92-2.04 (m, 1H), 2.30-2.42 (m, 1H), 3.18-3.26 (m, 2H), 4.52 (dt, J=10, 9 Hz, 1H), 7.56 (ddd, J=8, 5, 1 Hz, 1H), 7.92 (br s, 1H), 8.35 (ddd, J=8, 2, 2 Hz, 1H), 8.53 (s, 1H), 8.70 (dd, J=5, 2 Hz, 1H), 9.03 (d, J=8 Hz, 1H), 9.17 (dd, J=2, 1 Hz, 1H); LC-MS (LC-ES) for C₁₃H₁₂N₄O₂S M+H=289.

### Example 41

### 2-(3-Chloro-5-fluorophenyl)-N-((3R,5S)-5-methyl-2-oxopyrrolidin-3-yl)thiazole-5-carboxamide

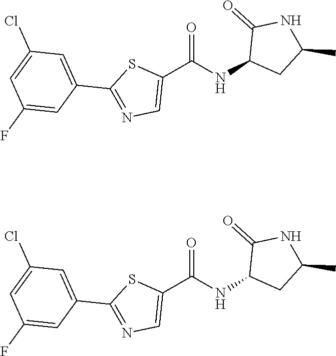 [SMILES: C[C@H]1C[C@@H](NC(=O)c2cnc(-c3cc(F)cc(Cl)c3)s2)C(=O)N1 ; C[C@H]1C[C@H](NC(=O)c2cnc(-c3cc(F)cc(Cl)c3)s2)C(=O)N1]

N,N-Diisopropylethylamine (0.396 mL, 2.268 mmol) was added to 2-(3-chloro-5-fluorophenyl)thiazole-5-carboxylic acid (0.1169 g, 0.454 mmol, Intermediate 5) in dichloromethane (4.54 mL) at room temperature. Then, an unequal diastereomeric mixture of (3R,5S)-3-amino-5-methylpyrrolidin-2-one hydrochloride and (3S,5S)-3-amino-5-methylpyrrolidin-2-one hydrochloride (0.102 g, 0.681 mmol, Intermediate 26) was added and the reaction mixture was stirred for five minutes. Then, n-propylphosphonic acid anhydride (0.540 mL, 0.907 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 1:4) to give a mixture of diastereomers (96.2 mg, 11.6:1 ratio ˜77% ee) which were separated on a chiral Chiralpak OD-H column, eluting with ethanol:heptane (15:85) with 0.1% isopropylamine to give 2-(3-chloro-5-fluorophenyl)-N-((3R,5S)-5-methyl-2-oxopyrrolidin-3-yl)thiazole-5-carboxamide (0.0579 g, 0.155 mmol, 34.3% yield) (t_{R}=6.4 minutes, >99% ee) as the first major diastereomer to elute and 2-(3-chloro-5-fluorophenyl)-N-((3S,5S)-5-methyl-2-oxopyrrolidin-3-yl)thiazole-5-carboxamide (0.0006 mg, 0.0016 μmol, 0.00036% yield) as the last major diastereomer to elute (t_{R}=9.3 minutes, 99% ee, contains 25% of diastereomer). The minor enantiomers were discarded.

### 2-(3-Chloro-5-fluorophenyl)-N-((3R,5S)-5-methyl-2-oxopyrrolidin-3-yl)thiazole-5-carboxamide

¹H NMR (400 MHz, CD₃SOCD₃) δ 1.15 (d, J=6 Hz, 3H), 1.52 (dt, J=12, 9 Hz, 1H), 2.46-2.54 (m, 1H), 3.54-3.62 (m, 1H), 4.59 (dt, J=11, 9 Hz, 1H), 7.64 (dt, J=9, 2 Hz, 1H), 7.83 (ddd, J=9, 2, 1 Hz, 1H), 7.90 (t, J=2 Hz, 1H), 8.03 (s, 1H), 8.51 (s, 1H), 9.02 (d, J=9 Hz, 1H); LC-MS (LC-ES) for C₁₅H₁₃ClFN₃O₂S M+H=354.

### 2-(3-Chloro-5-fluorophenyl)-N-((3S,5S)-5-methyl-2-oxopyrrolidin-3-yl)thiazole-5-carboxamide

¹H NMR (400 MHz, CD₃SOCD₃) δ 1.15 (d, J=6 Hz, 3H), 1.98-2.06 (m, 1H), 2.10-2.20 (m, 1H), 3.64-3.74 (m, 1H), 4.59 (q, J=8 Hz, 1H), 7.64 (dt, J=9, 2 Hz, 1H), 7.83 (ddd, J=9, 2, 1 Hz, 1H), 7.90 (t, J=2 Hz, 1H), 8.08 (s, 1H), 8.51 (s, 1H), 9.06 (d, J=9 Hz, 1H); LC-MS (LC-ES) for C₁₅H₁₃ClFN₃O₂S M+H=354.

### Example 42

### (S)-2-(4-Methylpyrimidin-2-yl)-N-(2-oxopyrrolidin-3-yl)thiazole-5-carboxamide

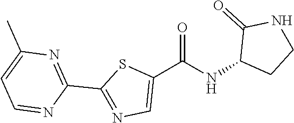 [SMILES: Cc1ccnc(-c2ncc(C(=O)N[C@H]3CCNC3=O)s2)n1]

N,N-Diisopropylethylamine (0.308 mL, 1.765 mmol) was added to 2-(4-methylpyrimidin-2-yl)thiazole-5-carboxylic acid (0.0781 g, 0.353 mmol, Intermediate 27) in N,N-dimethylformamide (3.53 mL) at room temperature. Then, (S)-3-aminopyrrolidin-2-one (0.053 g, 0.530 mmol) was added and the reaction mixture was stirred for five minutes. Then, n-propylphosphonic acid anhydride (0.420 mL, 0.706 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-2-(4-methylpyrimidin-2-yl)-N-(2-oxopyrrolidin-3-yl)thiazole-5-carboxamide (0.0109 g, 0.034 mmol, 9.67% yield). ¹H NMR (400 MHz, CD₃SOCD₃) δ 1.94-2.06 (m, 1H), 2.30-2.42 (m, 1H), 2.56 (s, 3H), 3.18-3.28 (m, 2H), 4.52 (dt, J=10, 9 Hz, 1H), 7.49 (d, J=5 Hz, 1H), 7.92 (br s, 1H), 8.57 (s, 1H), 8.79 (d, J=5 Hz, 1H), 9.05 (d, J=8 Hz, 1H); LC-MS (LC-ES) for C₁₃H₁₃N₅O₂S M+H=304.

### Example 43

### (S)-2-(3-Cyanophenyl)-N-(2-oxopyrrolidin-3-yl)thiazole-5-carboxamide

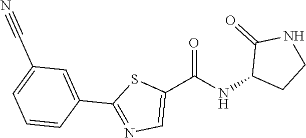 [SMILES: N#Cc1cccc(-c2ncc(C(=O)N[C@H]3CCNC3=O)s2)c1]

N,N-Diisopropylethylamine (0.286 mL, 1.635 mmol) was added to 2-(3-cyanophenyl)thiazole-5-carboxylic acid (0.0753 g, 0.327 mmol, Intermediate 28) in N,N-dimethylformamide (3.27 mL) at room temperature. Then, (S)-3-aminopyrrolidin-2-one (0.049 g, 0.491 mmol) was added and the reaction mixture was stirred for five minutes. Then, n-propylphosphonic acid anhydride (0.389 mL, 0.654 mmol) was added and the reaction mixture was stirred for two hours. The reaction mixture was concentrated. The resulting residue was purified by reverse phase HPLC, eluting with acetonitrile:water with 0.1% ammonium hydroxide (0:1 to 1:0) to give (S)-2-(3-cyanophenyl)-N-(2-oxopyrrolidin-3-yl)thiazole-5-carboxamide (0.0292 g, 0.089 mmol, 27.2% yield). ¹H NMR (400 MHz, CD₃SOCD₃) δ 1.92-2.04 (m, 1H), 2.32-2.42 (m, 1H), 3.18-3.28 (m, 2H), 4.52 (dt, J=10, 9 Hz, 1H), 7.73 (t, J=8 Hz, 1H), 7.92 (br s, 1H), 7.99 (dt, J=8, 1 Hz, 1H), 8.31 (ddd, J=8, 2, 1 Hz, 1H), 8.42 (t, J=1 Hz, 1H), 8.52 (s, 1H), 9.04 (d, J=8 Hz, 1H); LC-MS (LC-ES) for C₁₅H₁₂N₄O₂S M+H=313.

### Example 44

### (S)-N-(2-Oxopyrrolidin-3-yl)-2-(p-tolyl)thiazole-5-carboxamide

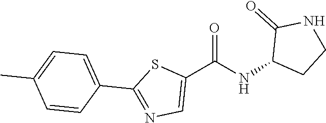 [SMILES: Cc1ccc(-c2ncc(C(=O)N[C@H]3CCNC3=O)s2)cc1]

N,N-Diisopropylethylamine (0.300 mL, 1.719 mmol) was added to 2-(p-tolyl)thiazole-5-carboxylic acid (0.0754 g, 0.344 mmol, Intermediate 29) in N,N-dimethylformamide (3.44 mL) at room temperature. Then, (S)-3-aminopyrrolidin-2-one (0.052 g, 0.516 mmol) was added and the reaction mixture was stirred for five minutes. Then, n-propylphosphonic acid anhydride (0.409 mL, 0.688 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-N-(2-oxopyrrolidin-3-yl)-2-(p-tolyl)thiazole-5-carboxamide (0.0195 g, 0.061 mmol, 17.88% yield). ¹H NMR (400 MHz, CD₃SOCD₃) δ 1.92-2.04 (m, 1H), 2.36 (s, 3H), 2.30-2.42 (m, 1H), 3.18-3.28 (m, 2H), 4.51 (dt, J=10, 9 Hz, 1H), 7.33 (d, J=8 Hz, 2H), 7.88 (d, J=8 Hz, 2H), 7.91 (br s, 1H), 8.43 (s, 1H), 8.93 (d, J=8 Hz, 1H); LC-MS (LC-ES) for C₁₅H₁₅N₃O₂S M+H=302.

### Example 45

### (S)-2-(3-Fluorophenyl)-N-(2-oxopyrrolidin-3-yl)thiazole-5-carboxamide

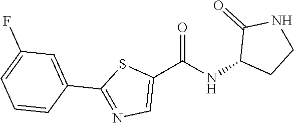 [SMILES: O=C(N[C@H]1CCNC1=O)c1cnc(-c2cccc(F)c2)s1]

N,N-Diisopropylethylamine (0.309 mL, 1.767 mmol) was added to 2-(3-fluorophenyl)thiazole-5-carboxylic acid (0.0789 g, 0.353 mmol, Intermediate 30) in N,N-dimethylformamide (3.53 mL) at room temperature. Then, (S)-3-aminopyrrolidin-2-one (0.053 g, 0.530 mmol) was added and the reaction mixture was stirred for five minutes. Then, n-propylphosphonic acid anhydride (0.421 mL, 0.707 mmol) was added and the reaction mixture was stirred for sixty-six hours. The reaction mixture was concentrated. The resulting residue was purified by reverse phase HPLC, eluting with acetonitrile:water with 0.1% ammonium hydroxide (0:1 to 1:0) to give (S)-2-(3-fluorophenyl)-N-(2-oxopyrrolidin-3-yl)thiazole-5-carboxamide (0.0743 g, 0.231 mmol, 65.4% yield). ¹H NMR (400 MHz, CD₃SOCD₃) δ 1.92-2.04 (m, 1H), 2.32-2.42 (m, 1H), 3.18-3.28 (m, 2H), 4.52 (dt, J=10, 9 Hz, 1H), 7.38 (ddt, J=9, 2, 1 Hz, 1H), 7.57 (dt, J=9, 6 Hz, 1H), 7.80 (ddd, J=10, 2, 1 Hz, 1H), 7.84 (ddd, J=8, 2, 1 Hz, 1H), 7.91 (br s, 1H), 8.49 (s, 1H), 9.00 (d, J=8 Hz, 1H); LC-MS (LC-ES) for C₁₄H₁₂FN₃O₂S M+H=306.

### Example 46

### (S)-2-(6-Methylpyridin-2-yl)-N-(2-oxopyrrolidin-3-yl)thiazole-5-carboxamide

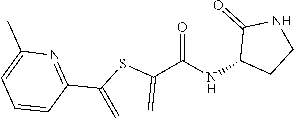 [SMILES: C=C(SC(=C)c1cccc(C)n1)C(=O)N[C@H]1CCNC1=O]

N,N-Diisopropylethylamine (0.290 mL, 1.662 mmol) was added to 2-(6-methylpyridin-2-yl)thiazole-5-carboxylic acid (0.0732 g, 0.332 mmol, Intermediate 31) in N,N-dimethylformamide (3.32 mL) at room temperature. Then, (S)-3-aminopyrrolidin-2-one (0.050 g, 0.499 mmol) was added and the reaction mixture was stirred for five minutes. Then, n-propylphosphonic acid anhydride (0.396 mL, 0.665 mmol) was added and the reaction mixture was stirred for four hours. The reaction mixture was concentrated. The resulting residue was purified by reverse phase HPLC, eluting with acetonitrile:water with 0.1% ammonium hydroxide (0:1 to 1:0) to give (S)-2-(6-methylpyridin-2-yl)-N-(2-oxopyrrolidin-3-yl)thiazole-5-carboxamide (0.0893 g, 0.281 mmol, 84% yield). ¹H NMR (400 MHz, CD₃SOCD₃) δ 1.92-2.06 (m, 1H), 2.30-2.42 (m, 1H), 2.54 (s, 3H), 3.18-3.28 (m, 2H), 4.51 (dt, J=10, 9 Hz, 1H), 7.40 (br d, J=7 Hz, 1H), 7.87 (t, J=8 Hz, 1H), 7.90 (br s, 1H), 7.96 (br d, J=8 Hz, 1H), 8.49 (s, 1H), 8.94 (d, J=8 Hz, 1H); LC-MS (LC-ES) for C₁₄H₁₄N₄O₂S M+H=303.

### Example 47

### (S)-2-(4-Methylpyridin-2-yl)-N-(2-oxopyrrolidin-3-yl)thiazole-5-carboxamide

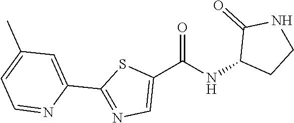 [SMILES: Cc1ccnc(-c2ncc(C(=O)N[C@H]3CCNC3=O)s2)c1]

N,N-Diisopropylethylamine (0.307 mL, 1.759 mmol) was added to 2-(4-methylpyridin-2-yl)thiazole-5-carboxylic acid (0.0775 g, 0.352 mmol, Intermediate 32) in N,N-dimethylformamide (3.52 mL) at room temperature. Then, (S)-3-aminopyrrolidin-2-one (0.053 g, 0.528 mmol) was added and the reaction mixture was stirred for five minutes. Then, n-propylphosphonic acid anhydride (0.419 mL, 0.704 mmol) was added and the reaction mixture was stirred for sixteen hours. The reaction mixture was concentrated. The resulting residue was purified by reverse phase HPLC, eluting with acetonitrile:water with 0.1% ammonium hydroxide (0:1 to 1:0) to give (S)-2-(4-methylpyridin-2-yl)-N-(2-oxopyrrolidin-3-yl)thiazole-5-carboxamide (0.0912 g, 0.287 mmol, 81% yield). ¹H NMR (400 MHz, CD₃SOCD₃) δ 1.92-2.06 (m, 1H), 2.30-2.42 (m, 1H), 2.42 (s, 3H), 3.18-3.28 (m, 2H), 4.51 (dt, J=10, 9 Hz, 1H), 7.37 (ddd, J=5, 2, 1 Hz, 1H), 7.90 (br s, 1H), 8.01 (dt, J=2, 1 Hz, 1H), 8.50 (s, 1H), 8.52 (d, J=1 Hz, 1H), 8.96 (d, J=8 Hz, 1H); LC-MS (LC-ES) for C₁₄H₁₄N₄O₂S M+H=303.

### Example 48

### (S)-2-(3-(Difluoromethyl)-5-methylphenyl)-N-(2-oxopyrrolidin-3-yl)thiazole-5-carboxamide

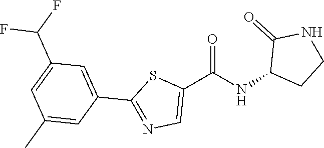 [SMILES: Cc1cc(-c2ncc(C(=O)N[C@H]3CCNC3=O)s2)cc(C(F)F)c1]

N,N-Diisopropylethylamine (0.255 mL, 1.460 mmol) was added to 2-(3-(difluoromethyl)-5-methylphenyl)thiazole-5-carboxylic acid (0.0786 g, 0.292 mmol, Intermediate 33) in N,N-dimethylformamide (2.92 mL) at room temperature. Then, (S)-3-aminopyrrolidin-2-one (0.044 g, 0.438 mmol) was added and the reaction mixture was stirred for five minutes. Then, n-propylphosphonic acid anhydride (0.348 mL, 0.584 mmol) was added and the reaction mixture was stirred for six hours. The reaction mixture was concentrated. The resulting residue was purified by reverse phase HPLC, eluting with acetonitrile:water with 0.1% ammonium hydroxide (0:1 to 1:0) to give (S)-2-(3-(difluoromethyl)-5-methylphenyl)-N-(2-oxopyrrolidin-3-yl)thiazole-5-carboxamide (0.0870 g, 0.235 mmol, 81% yield). ¹H NMR (400 MHz, CD₃SOCD₃) δ 1.92-2.04 (m, 1H), 2.32-2.42 (m, 1H), 2.45 (s, 3H), 3.18-3.28 (m, 2H), 4.52 (dt, J=10, 9 Hz, 1H), 7.09 (t, J=66 Hz, 1H), 7.55 (br s, 1H), 7.92 (br s, 1H), 7.98 (br s, 2H), 8.50 (s, 1H), 9.00 (d, J=8 Hz, 1H); LC-MS (LC-ES) for C₁₆H₁₅F₂N₃O₂S M+H=352.

### Example 49

### Racemic 2-(3-Chloro-5-fluorophenyl)-N-((3R,3aS,6aR)-2-oxooctahydrocyclopenta[b]pyrrol-3-yl)thiazole-5-carboxamide; and

### 2-(3-Chloro-5-fluorophenyl)-N-((3S,3aR,6aS)-2-oxooctahydrocyclopenta[b]pyrrol-3-yl)thiazole-5-carboxamide

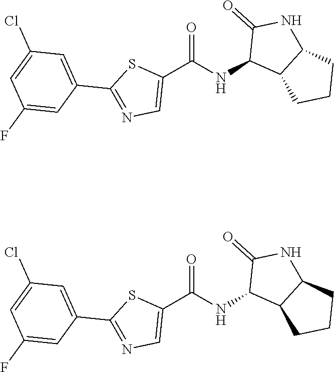 [SMILES: O=C(N[C@H]1C(=O)N[C@@H]2CCC[C@@H]21)c1cnc(-c2cc(F)cc(Cl)c2)s1 ; O=C(N[C@@H]1C(=O)N[C@H]2CCC[C@H]21)c1cnc(-c2cc(F)cc(Cl)c2)s1]

N,N-Diisopropylethylamine (0.351 mL, 2.008 mmol) was added to 2-(3-chloro-5-fluorophenyl)thiazole-5-carboxylic acid (0.1035 g, 0.402 mmol, Intermediate 5) in dichloromethane (4.02 mL) at room temperature. Then, racemic (3R,3aR,6aR)-3-aminohexahydrocyclopenta[b]pyrrol-2(1H)-one hydrochloride and (3S,3aS,6aS)-3-aminohexahydrocyclopenta[b]pyrrol-2(1H)-one hydrochloride (0.106 g, 0.603 mmol, Intermediate 34) was added and the reaction mixture was stirred for five minutes. Then, n-propylphosphonic acid anhydride (0.478 mL, 0.803 mmol) was added and the reaction mixture was stirred for four hours. Saturated sodium bicarbonate was added to the reaction mixture and it was extracted with dichloromethane, dried over magnesium sulfate, filtered, and concentrated. The resulting residue was purified by silica gel chromatography, eluting with methanol:dichloromethane (0:1 to 3:7) to give a racemic mixture of 2-(3-chloro-5-fluorophenyl)-N-((3R,3aS,6aR)-2-oxooctahydrocyclopenta[b]pyrrol-3-yl)thiazole-5-carboxamide and 2-(3-chloro-5-fluorophenyl)-N-((3S,3aR,6aS)-2-oxooctahydrocyclopenta[b]pyrrol-3-yl)thiazole-5-carboxamide (0.1321 g, 0.330 mmol, 82% yield). ¹H NMR (400 MHz, CD₃SOCD₃) δ 1.52-1.64 (m, 4H), 1.64-1.74 (m, 2H), 2.60-2.68 (m, 1H), 3.96-4.02 (m, 1H), 4.02-4.10 (m, 1H), 7.64 (dt, J=9, 2 Hz, 1H), 7.83 (ddd, J=9, 2, 1 Hz, 1H), 7.90 (t, J=2 Hz, 1H), 8.02 (br s, 1H), 8.50 (s, 1H), 9.22 (d, J=8 Hz, 1H); LC-MS (LC-ES) for C₁₇H₁₅ClFN₃O₂S M+H=380.

### Examples 50 & 51

### 2-(3-Chloro-5-fluorophenyl)-N-((3R,3aS,6aR)-2-oxooctahydrocyclopenta[b]pyrrol-3-yl)thiazole-5-carboxamide and 2-(3-Chloro-5-fluorophenyl)-N-((3S,3aR,6aS)-2-oxooctahydrocyclopenta[b]pyrrol-3-yl)thiazole-5-carboxamide

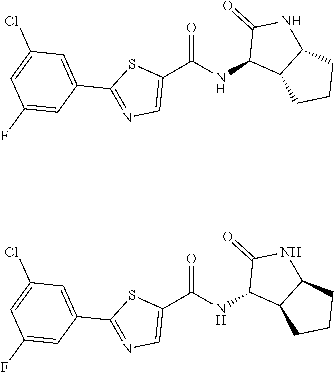 [SMILES: O=C(N[C@H]1C(=O)N[C@@H]2CCC[C@@H]21)c1cnc(-c2cc(F)cc(Cl)c2)s1 ; O=C(N[C@@H]1C(=O)N[C@H]2CCC[C@H]21)c1cnc(-c2cc(F)cc(Cl)c2)s1]

Racemic 2-(3-chloro-5-fluorophenyl)-N-((3R,3aS,6aR)-2-oxooctahydrocyclopenta[b]pyrrol-3-yl)thiazole-5-carboxamide and 2-(3-chloro-5-fluorophenyl)-N-((3S,3aR,6aS)-2-oxooctahydrocyclopenta[b]pyrrol-3-yl)thiazole-5-carboxamide (0.1204 g, 0.317 mmol) (Example 49) was separated into its enantiomers on a chiral Chiralpak OD-H column, eluting with ethanol:heptane (1:3) with 0.1% isopropylamine to give 2-(3-chloro-5-fluorophenyl)-N-((3R,3aS,6aR)-2-oxooctahydrocyclopenta[b]pyrrol-3-yl)thiazole-5-carboxamide (0.0449 g, 0.112 mmol, 35.4% yield) as the first enantiomer (t_{R}=4.3 minutes, 99% ee) to elute and 2-(3-chloro-5-fluorophenyl)-N-((3S,3aR,6aS)-2-oxooctahydrocyclopenta[b]pyrrol-3-yl)thiazole-5-carboxamide (0.0447 g, 0.112 mmol, 35.3% yield) as the last enantiomer (t_{R}=5.4 minutes, 92% ee) to elute. The structures were assigned by vibrational circular dichroism.

### 2-(3-chloro-5-fluorophenyl)-N-((3R,3aS,6aR)-2-oxooctahydrocyclopenta[b]pyrrol-3-yl)thiazole-5-carboxamide

¹H NMR (400 MHz, CD₃SOCD₃) δ 1.52-1.64 (m, 4H), 1.64-1.74 (m, 2H), 2.60-2.68 (m, 1H), 3.96-4.02 (m, 1H), 4.05 (dd, J=8, 6 Hz, 1H), 7.64 (dt, J=9, 2 Hz, 1H), 7.83 (ddd, J=9, 2, 1 Hz, 1H), 7.90 (t, J=2 Hz, 1H), 8.02 (br s, 1H), 8.50 (s, 1H), 9.22 (d, J=8 Hz, 1H); LC-MS (LC-ES) for C₁₇H₁₅ClFN₃O₂S M+H=380.

### 2-(3-chloro-5-fluorophenyl)-N-((3S,3aR,6aS)-2-oxooctahydrocyclopenta[b]pyrrol-3-yl)thiazole-5-carboxamide

¹H NMR (400 MHz, CD₃SOCD₃) δ 1.52-1.64 (m, 4H), 1.64-1.74 (m, 2H), 2.60-2.68 (m, 1H), 3.96-4.02 (m, 1H), 4.05 (dd, J=8, 6 Hz, 1H), 7.64 (dt, J=9, 2 Hz, 1H), 7.83 (ddd, J=9, 2, 1 Hz, 1H), 7.90 (t, J=2 Hz, 1H), 8.02 (br s, 1H), 8.50 (s, 1H), 9.22 (d, J=8 Hz, 1H); LC-MS (LC-ES) for C₁₇H₁₅ClFN₃O₂S M+H=380.

### Example 52

### Racemic 2-(3-Chloro-5-fluorophenyl)-N-((3S,3aR,6aR)-2-oxohexahydro-1H-furo[3,4-b]pyrrol-3-yl)thiazole-5-carboxamide; and

### 2-(3-Chloro-5-fluorophenyl)-N-((3R,3aS,6aS)-2-oxohexahydro-1H-furo[3,4-b]pyrrol-3-yl)thiazole-5-carboxamide

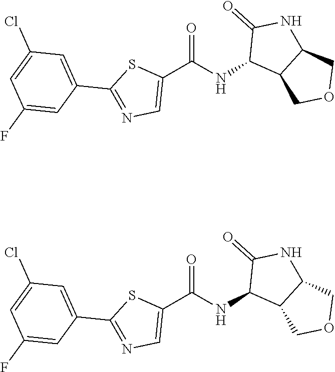 [SMILES: O=C(N[C@@H]1C(=O)N[C@H]2COC[C@H]21)c1cnc(-c2cc(F)cc(Cl)c2)s1 ; O=C(N[C@H]1C(=O)N[C@@H]2COC[C@@H]21)c1cnc(-c2cc(F)cc(Cl)c2)s1]

N,N-Diisopropylethylamine (0.345 mL, 1.977 mmol) was added to 2-(3-chloro-5-fluorophenyl)thiazole-5-carboxylic acid (0.1019 g, 0.395 mmol, Intermediate 5) in dichloromethane (3.95 mL) at room temperature. Then, racemic ((3S,3aS,6aR)-3-aminotetrahydro-1H-furo[3,4-b]pyrrol-2(3H)-one hydrochloride and (3R,3aR,6aS)-3-aminotetrahydro-1H-furo[3,4-b]pyrrol-2(3H)-one hydrochloride (0.092 g, 0.514 mmol, Intermediate 35) was added and the reaction mixture was stirred for five minutes. Then, n-propylphosphonic acid anhydride (0.471 mL, 0.791 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:dichloromethane (0:1 to 3:7) to give racemic 2-(3-chloro-5-fluorophenyl)-N-((3S,3aR,6aR)-2-oxohexahydro-1H-furo[3,4-b]pyrrol-3-yl)thiazole-5-carboxamide and 2-(3-chloro-5-fluorophenyl)-N-((3R,3aS,6aS)-2-oxohexahydro-1H-furo[3,4-b]pyrrol-3-yl)thiazole-5-carboxamide (0.1333 g, 0.332 mmol, 84% yield). ¹H NMR (400 MHz, CD₃SOCD₃) δ 2.82-2.90 (m, 1H), 3.44 (dd, J=9, 5 Hz, 1H), 3.59 (dd, J=9, 6 Hz, 1H), 3.66 (br d, J=9 Hz, 1H), 3.89 (br d, J=9 Hz, 1H), 4.10-4.22 (m, 2H), 7.64 (dt, J=9, 2 Hz, 1H), 7.83 (ddd, J=9, 2, 1 Hz, 1H), 7.90 (t, J=2 Hz, 1H), 8.19 (br s, 1H), 8.51 (s, 1H), 9.30 (d, J=8 Hz, 1H); LC-MS (LC-ES) for C₁₆H₁₃ClFN₃O₃S M+H=382.

### Examples 53 & 54

### 2-(3-Chloro-5-fluorophenyl)-N-((3S,3aR,6aR)-2-oxohexahydro-1H-furo[3,4-b]pyrrol-3-yl)thiazole-5-carboxamide; and

### 2-(3-Chloro-5-fluorophenyl)-N-((3R,3aS,6aS)-2-oxohexahydro-1H-furo[3,4-b]pyrrol-3-yl)thiazole-5-carboxamide

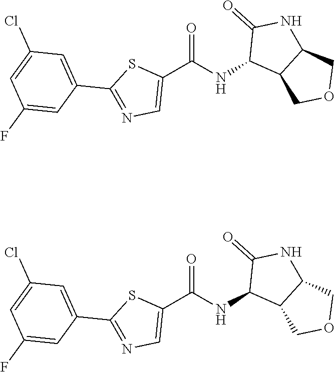 [SMILES: O=C(N[C@@H]1C(=O)N[C@H]2COC[C@H]21)c1cnc(-c2cc(F)cc(Cl)c2)s1 ; O=C(N[C@H]1C(=O)N[C@@H]2COC[C@@H]21)c1cnc(-c2cc(F)cc(Cl)c2)s1]

Racemic 2-(3-chloro-5-fluorophenyl)-N-((3S,3aR,6aR)-2-oxohexahydro-1H-furo[3,4-b]pyrrol-3-yl)thiazole-5-carboxamide and 2-(3-chloro-5-fluorophenyl)-N-((3R,3aS,6aS)-2-oxohexahydro-1H-furo[3,4-b]pyrrol-3-yl)thiazole-5-carboxamide (0.1241 g, 0.325 mmol, Example 52) was separated into its enantiomers on a chiral Chiralpak IC column, eluting with methanol:acetonitrile (1:4) with 0.1% isopropylamine to give 2-(3-chloro-5-fluorophenyl)-N-((3S,3aR,6aR)-2-oxohexahydro-1H-furo[3,4-b]pyrrol-3-yl)thiazole-5-carboxamide (0.0548 g, 0.136 mmol, 42.0% yield) as the first enantiomer to elute (t_{R}=3.7 minutes, >99% ee) and 2-(3-chloro-5-fluorophenyl)-N-((3R,3aS,6aS)-2-oxohexahydro-1H-furo[3,4-b]pyrrol-3-yl)thiazole-5-carboxamide (0.0556 g, 0.138 mmol, 42.6% yield) as the last enantiomer to elute (t_{R}=4.4 minutes, 98% ee).

### 2-(3-Chloro-5-fluorophenyl)-N-((3S,3aR,6aR)-2-oxohexahydro-1H-furo[3,4-b]pyrrol-3-yl)thiazole-5-carboxamide

¹H NMR (400 MHz, CD₃SOCD₃) δ 2.82-2.90 (m, 1H), 3.44 (dd, J=10, 5 Hz, 1H), 3.59 (dd, J=9, 6 Hz, 1H), 3.67 (br d, J=10 Hz, 1H), 3.90 (br d, J=9 Hz, 1H), 4.13 (dd, J=8, 5 Hz, 1H), 4.16-4.20 (m, 1H), 7.66 (dt, J=9, 2 Hz, 1H), 7.85 (ddd, J=9, 2, 1 Hz, 1H), 7.91 (t, J=2 Hz, 1H), 8.22 (br s, 1H), 8.52 (s, 1H), 9.33 (d, J=8 Hz, 1H); LC-MS (LC-ES) for C₁₆H₁₃ClFN₃O₃S M+H=382.

### 2-(3-Chloro-5-fluorophenyl)-N-((3R,3aS,6aS)-2-oxohexahydro-1H-furo[3,4-b]pyrrol-3-yl)thiazole-5-carboxamide

¹H NMR (400 MHz, CD₃SOCD₃) δ 2.82-2.90 (m, 1H), 3.44 (dd, J=10, 5 Hz, 1H), 3.59 (dd, J=9, 6 Hz, 1H), 3.66 (br d, J=9 Hz, 1H), 3.89 (br d, J=9 Hz, 1H), 4.13 (dd, J=8, 5 Hz, 1H), 4.16-4.20 (m, 1H), 7.65 (dt, J=9, 2 Hz, 1H), 7.84 (ddd, J=9, 2, 1 Hz, 1H), 7.91 (t, J=2 Hz, 1H), 8.22 (br s, 1H), 8.51 (s, 1H), 9.33 (d, J=8 Hz, 1H); LC-MS (LC-ES) for C₁₆H₁₃ClFN₃O₃S M+H=382.

### Example 55

### Racemic 2-(3-Chloro-5-fluorophenyl)-N-(6-oxo-2-oxa-5-azaspiro[3.4]octan-7-yl)thiazole-5-carboxamide

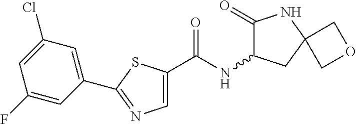 [SMILES: O=C(NC1CC2(COC2)NC1=O)c1cnc(-c2cc(F)cc(Cl)c2)s1]

N,N-Diisopropylethylamine (0.334 mL, 1.919 mmol) was added to 2-(3-chloro-5-fluorophenyl)thiazole-5-carboxylic acid (0.1236 g, 0.480 mmol, Intermediate 5) in dichloromethane (4.80 mL) at room temperature. Then, racemic 7-amino-2-oxa-5-azaspiro[3.4]octan-6-one hydrochloride (0.086 g, 0.480 mmol, Intermediate 36) was added and the reaction mixture was stirred for five minutes. Then, 1-((dimethylamino)(dimethyliminio)methyl)-1H-[1,2,3]triazolo[4,5-b]pyridine 3-oxide hexafluorophosphate(V) (0.219 g, 0.576 mmol) was added and the reaction mixture was stirred for three hours. 10% Aqueous citric acid was added and the reaction mixture was extracted with dichloromethane, washed with saturated sodium bicarbonate, dried over magnesium sulfate, filtered, and concentrated. The resulting residue was purified by reverse phase HPLC, eluting with acetonitrile:water with 0.1% ammonium hydroxide (0:1 to 1:0) to give racemic 2-(3-chloro-5-fluorophenyl)-N-(6-oxo-2-oxa-5-azaspiro[3.4]octan-7-yl)thiazole-5-carboxamide (0.0975 g, 0.243 mmol, 50.6% yield). ¹H NMR (400 MHz, CD₃SOCD₃) δ 2.24 (dd, J=13, 9 Hz, 1H), 2.85 (dd, J=13, 9 Hz, 1H), 4.52 (dt, J=11, 9 Hz, 1H), 4.57 (ABq, J_{AB}=6 Hz, Δv_{AB}=32 Hz, 2H), 4.63 (ABq, J_{AB}=7 Hz, Δv_{AB}=39 Hz, 2H), 7.64 (dt, J=9, 2 Hz, 1H), 7.83 (ddd, J=9, 2, 2 Hz, 1H), 7.90 (t, J=2 Hz, 1H), 8.48 (s, 1H), 8.85 (br s, 1H), 9.11 (d, J=8 Hz, 1H); LC-MS (LC-ES) for C₁₆H₁₃ClFN₃O₃S M+H=382.

### Examples 56 & 57

### (R)-2-(3-Chloro-5-fluorophenyl)-N-(6-oxo-2-oxa-5-azaspiro[3.4]octan-7-yl)thiazole-5-carboxamide; and

### (S)-2-(3-Chloro-5-fluorophenyl)-N-(6-oxo-2-oxa-5-azaspiro[3.4]octan-7-yl)thiazole-5-carboxamide

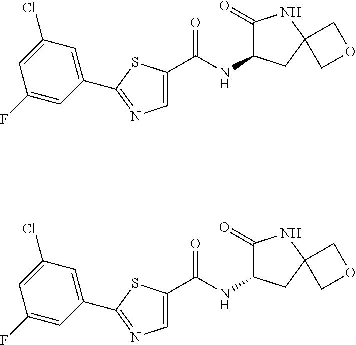 [SMILES: O=C(N[C@@H]1CC2(COC2)NC1=O)c1cnc(-c2cc(F)cc(Cl)c2)s1 ; O=C(N[C@H]1CC2(COC2)NC1=O)c1cnc(-c2cc(F)cc(Cl)c2)s1]

Racemic 2-(3-chloro-5-fluorophenyl)-N-(6-oxo-2-oxa-5-azaspiro[3.4]octan-7-yl)thiazole-5-carboxamide (0.0878 g, 0.230 mmol, Example 55) was separated into its enantiomers on a chiral Chiralpak OD-H column, eluting with ethanol:heptane (1:3) with 0.1% isopropylamine to give (R)-2-(3-chloro-5-fluorophenyl)-N-(6-oxo-2-oxa-5-azaspiro[3.4]octan-7-yl)thiazole-5-carboxamide (0.0274 g, 0.068 mmol, 29.6% yield) as the first enantiomer to elute (t_{R}=7.6 minutes, 99% ee) and (S)-2-(3-chloro-5-fluorophenyl)-N-(6-oxo-2-oxa-5-azaspiro[3.4]octan-7-yl)thiazole-5-carboxamide (0.0298 g, 0.074 mmol, 32.2% yield) as the last enantiomer to elute (t_{R}=10.3 minutes, 98% ee). The structures were assigned via vibrational circular dichroism.

### (R)-2-(3-Chloro-5-fluorophenyl)-N-(6-oxo-2-oxa-5-azaspiro[3.4]octan-7-yl)thiazole-5-carboxamide

¹H NMR (400 MHz, CD₃SOCD₃) δ 2.24 (dd, J=13, 9 Hz, 1H), 2.85 (dd, J=13, 9 Hz, 1H), 4.52 (dt, J=11, 9 Hz, 1H), 4.57 (ABq, J_{AB}=6 Hz, Δv_{AB}=32 Hz, 2H), 4.63 (ABq, J_{AB}=⁷ Hz, Δv_{AB}=39 Hz, 2H), 7.64 (dt, J=9, 2 Hz, 1H), 7.83 (ddd, J=9, 2, 2 Hz, 1H), 7.90 (t, J=2 Hz, 1H), 8.48 (s, 1H), 8.85 (br s, 1H), 9.11 (d, J=8 Hz, 1H); LC-MS (LC-ES) for C₁₆H₁₃ClFN₃O₃S M+H=382.

### (S)-2-(3-Chloro-5-fluorophenyl)-N-(6-oxo-2-oxa-5-azaspiro[3.4]octan-7-yl)thiazole-5-carboxamide

¹H NMR (400 MHz, CD₃SOCD₃) δ 2.25 (dd, J=13, 9 Hz, 1H), 2.86 (dd, J=13, 9 Hz, 1H), 4.53 (dt, J=11, 9 Hz, 1H), 4.58 (ABq, J_{AB}=6 Hz, Δv_{AB}=32 Hz, 2H), 4.63 (ABq, J_{AB}=7 Hz, Δv_{AB}=39 Hz, 2H), 7.65 (dt, J=9, 2 Hz, 1H), 7.84 (ddd, J=9, 2, 2 Hz, 1H), 7.91 (t, J=2 Hz, 1H), 8.49 (s, 1H), 8.86 (br s, 1H), 9.12 (d, J=8 Hz, 1H); LC-MS (LC-ES) for C₁₆H₁₃ClFN₃O₃S M+H=382.

### Example 58

### Racemic 2-(3-Chloro-5-fluorophenyl)-N-((3R,3aR,6aS)-2-oxooctahydrocyclopenta[b]pyrrol-3-yl)thiazole-5-carboxamide; and

### 2-(3-Chloro-5-fluorophenyl)-N-((3S,3aS,6aR)-2-oxooctahydrocyclopenta[b]pyrrol-3-yl)thiazole-5-carboxamide

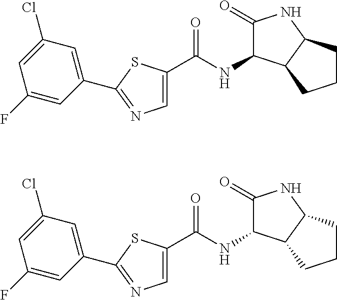 [SMILES: O=C(N[C@H]1C(=O)N[C@H]2CCC[C@H]21)c1cnc(-c2cc(F)cc(Cl)c2)s1 ; O=C(N[C@@H]1C(=O)N[C@@H]2CCC[C@H]12)c1cnc(-c2cc(F)cc(Cl)c2)s1]

N,N-Diisopropylethylamine (0.349 mL, 2.001 mmol) was added to 2-(3-chloro-5-fluorophenyl)thiazole-5-carboxylic acid (0.1031 g, 0.400 mmol, Intermediate 5) in dichloromethane (4.00 mL) at room temperature. Then, racemic (3s,3aR,6aR)-3-aminohexahydrocyclopenta[b]pyrrol-2(1H)-one hydrochloride and (3R,3aS,6aS)-3-aminohexahydrocyclopenta[b]pyrrol-2(1H)-one hydrochloride (0.092 g, 0.520 mmol, Intermediate 37) was added and the reaction mixture was stirred for five minutes. Then, n-propylphosphonic acid anhydride (0.476 mL, 0.800 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:dichloromethane (0:1 to 3:7) to give racemic 2-(3-chloro-5-fluorophenyl)-N-((3R,3aR,6aS)-2-oxooctahydrocyclopenta[b]pyrrol-3-yl)thiazole-5-carboxamide and 2-(3-chloro-5-fluorophenyl)-N-((3S,3aS,6aR)-2-oxooctahydrocyclopenta[b]pyrrol-3-yl)thiazole-5-carboxamide (0.1084 g, 0.271 mmol, 67.8% yield). ¹H NMR (400 MHz, CD₃SOCD₃) δ 1.36-1.56 (m, 2H), 1.58-1.68 (m, 4H), 2.86-2.96 (m, 1H), 3.98 (dt, J=6, 4 Hz, 1H), 4.71 (dd, J=9, 8 Hz, 1H), 7.64 (dt, J=9, 2 Hz, 1H), 7.83 (ddd, J=9, 2, 1 Hz, 1H), 7.91 (t, J=2 Hz, 1H), 7.93 (br s, 1H), 8.68 (s, 1H), 9.09 (d, J=8 Hz, 1H); LC-MS (LC-ES) for C₁₇H₁₅ClFN₃O₂S M+H=380.

### Examples 59 & 60

### 2-(3-Chloro-5-fluorophenyl)-N-((3R,3aR,6aS)-2-oxooctahydrocyclopenta[b]pyrrol-3-yl)thiazole-5-carboxamide; and

### 2-(3-Chloro-5-fluorophenyl)-N-((3S,3aS,6aR)-2-oxooctahydrocyclopenta[b]pyrrol-3-yl)thiazole-5-carboxamide

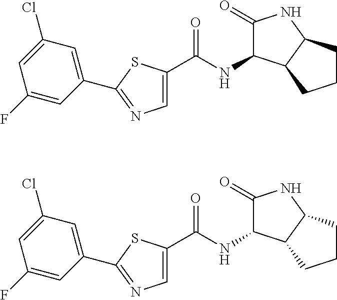 [SMILES: O=C(N[C@H]1C(=O)N[C@H]2CCC[C@H]21)c1cnc(-c2cc(F)cc(Cl)c2)s1 ; O=C(N[C@@H]1C(=O)N[C@@H]2CCC[C@H]12)c1cnc(-c2cc(F)cc(Cl)c2)s1]

Racemic 2-(3-chloro-5-fluorophenyl)-N-((3R,3aR,6aS)-2-oxooctahydrocyclopenta[b]pyrrol-3-yl)thiazole-5-carboxamide and 2-(3-chloro-5-fluorophenyl)-N-((3S,3aS,6aR)-2-oxooctahydrocyclopenta[b]pyrrol-3-yl)thiazole-5-carboxamide (0.1002 g, 0.264 mmol, Example 58) was separated into its enantiomers on a chiral Chiralpak OD-H column, eluting with ethanol:heptane (1:3) with 0.1% isopropylamine to give 2-(3-chloro-5-fluorophenyl)-N-((3R,3aR,6aS)-2-oxooctahydrocyclopenta[b]pyrrol-3-yl)thiazole-5-carboxamide (0.0431 g, 0.108 mmol, 40.9% yield) as the first enantiomer to elute (t_{R}=5.2 minutes, >99% ee) and 2-(3-chloro-5-fluorophenyl)-N-((3S,3aS,6aR)-2-oxooctahydrocyclopenta[b]pyrrol-3-yl)thiazole-5-carboxamide (0.0414 g, 0.104 mmol, 39.3% yield) as the last enantiomer to elute (t_{R}=8.0 minutes, >99% ee). The structures were assigned by vibrational circular dichroism.

### 2-(3-Chloro-5-fluorophenyl)-N-((3R,3aR,6aS)-2-oxooctahydrocyclopenta[b]pyrrol-3-yl)thiazole-5-carboxamide

¹H NMR (400 MHz, CD₃SOCD₃) δ 1.36-1.54 (m, 2H), 1.56-1.68 (m, 4H), 2.86-2.96 (m, 1H), 3.98 (dt, J=6, 4 Hz, 1H), 4.71 (dd, J=9, 8 Hz, 1H), 7.64 (dt, J=9, 2 Hz, 1H), 7.84 (ddd, J=9, 2, 1 Hz, 1H), 7.91 (t, J=2 Hz, 1H), 7.94 (br s, 1H), 8.68 (s, 1H), 9.09 (d, J=8 Hz, 1H); LC-MS (LC-ES) for C₁₇H₁₅ClFN₃O₂S M+H=380.

### 2-(3-Chloro-5-fluorophenyl)-N-((3S,3aS,6aR)-2-oxooctahydrocyclopenta[b]pyrrol-3-yl)thiazole-5-carboxamide

¹H NMR (400 MHz, CD₃SOCD₃) δ 1.36-1.54 (m, 2H), 1.56-1.68 (m, 4H), 2.86-2.96 (m, 1H), 3.98 (dt, J=6, 4 Hz, 1H), 4.71 (dd, J=9, 8 Hz, 1H), 7.64 (dt, J=9, 2 Hz, 1H), 7.84 (ddd, J=9, 2, 1 Hz, 1H), 7.91 (t, J=2 Hz, 1H), 7.94 (br s, 1H), 8.68 (s, 1H), 9.09 (d, J=8 Hz, 1H); LC-MS (LC-ES) for C₁₇H₁₅ClFN₃O₂S M+H=380.

### Examples 61 & 62

### 2-(3-Chloro-5-fluorophenyl)-N-((3R,5R)-5-methyl-2-oxopyrrolidin-3-yl)thiazole-5-carboxamide; and

### 2-(3-Chloro-5-fluorophenyl)-N-((3S,5R)-5-methyl-2-oxopyrrolidin-3-yl)thiazole-5-carboxamide

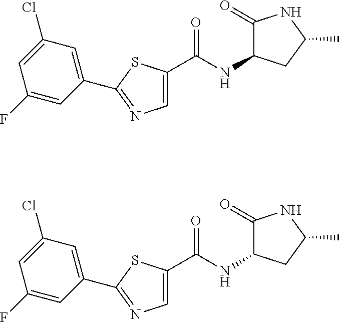 [SMILES: C[C@@H]1C[C@@H](NC(=O)c2cnc(-c3cc(F)cc(Cl)c3)s2)C(=O)N1 ; C[C@@H]1C[C@H](NC(=O)c2cnc(-c3cc(F)cc(Cl)c3)s2)C(=O)N1]

N,N-Diisopropylethylamine (0.512 mL, 2.93 mmol) was added to 2-(3-chloro-5-fluorophenyl)thiazole-5-carboxylic acid (0.1511 g, 0.586 mmol, Intermediate 5) in dichloromethane (5.86 mL) at room temperature. Then, an unequal diastereomeric mixture of (3R,5R)-3-amino-5-methylpyrrolidin-2-one hydrochloride and (3S,5R)-3-amino-5-methylpyrrolidin-2-one hydrochloride (0.132 g, 0.880 mmol, Intermediate 38) was added and the reaction mixture was stirred for five minutes. Then, n-propylphosphonic acid anhydride (0.698 mL, 1.173 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromotography, eluting with methanol:ethyl acetate (0:1 to 1:4) to give a mixture of diastereomers (126.2 mg, ˜9:1 ratio ˜84% ee), which were separated on a chiral Chiralpak OD column, eluting with ethanol:heptane (15:85) with 0.1% isopropylamine to give 2-(3-chloro-5-fluorophenyl)-N-((3R,5R)-5-methyl-2-oxopyrrolidin-3-yl)thiazole-5-carboxamide (0.0107 g, 0.029 mmol, 4.90% yield) as the first diasteromer (t_{R}=5.7 minutes, 85% ee) to elute and 2-(3-chloro-5-fluorophenyl)-N-((3S,5R)-5-methyl-2-oxopyrrolidin-3-yl)thiazole-5-carboxamide (0.0582 g, 0.165 mmol, 28.1% yield) as the last diastereomer (t_{R}=7.8 minutes, 99% ee) to elute. The minor enantiomers were discarded. The stereochemical assignments were based on comparison to their enantiomers in Example 41.

### 2-(3-Chloro-5-fluorophenyl)-N-((3R,5R)-5-methyl-2-oxopyrrolidin-3-yl)thiazole-5-carboxamide

¹H NMR (400 MHz, CD₃SOCD₃) δ 1.15 (d, J=6 Hz, 3H), 1.98-2.06 (m, 1H), 2.10-2.20 (m, 1H), 3.64-3.74 (m, 1H), 4.59 (q, J=8 Hz, 1H), 7.64 (dt, J=9, 2 Hz, 1H), 7.83 (ddd, J=9, 2, 1 Hz, 1H), 7.90 (t, J=2 Hz, 1H), 8.07 (br s, 1H), 8.51 (s, 1H), 9.06 (d, J=8 Hz, 1H); LC-MS (LC-ES) for C₁₅H₁₃ClFN₃O₂S M+H=354.

### 2-(3-Chloro-5-fluorophenyl)-N-((3S,5R)-5-methyl-2-oxopyrrolidin-3-yl)thiazole-5-carboxamide

¹H NMR (400 MHz, CD₃SOCD₃) δ 1.16 (d, J=6 Hz, 3H), 1.53 (dt, J=12, 9 Hz, 1H), 2.46-2.56 (m, 1H), 3.56-3.64 (m, 1H), 4.61 (dt, J=11, 9 Hz, 1H), 7.65 (dt, J=9, 2 Hz, 1H), 7.84 (ddd, J=9, 2, 1 Hz, 1H), 7.91 (t, J=1 Hz, 1H), 8.05 (s, 1H), 8.52 (s, 1H), 9.03 (d, J=9 Hz, 1H); LC-MS (LC-ES) for C₁₅H₁₃ClFN₃O₂S M+H=354.

### Example 63

### Racemic 2-(3-Chloro-5-fluorophenyl)-N-(1-methyl-2-oxopyrrolidin-3-yl)thiazole-5-carboxamide

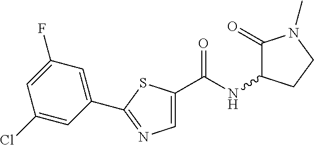 [SMILES: CN1CCC(NC(=O)c2cnc(-c3cc(F)cc(Cl)c3)s2)C1=O]

N,N-Diisopropylethylamine (0.308 mL, 1.764 mmol) was added to 2-(3-chloro-5-fluorophenyl)thiazole-5-carboxylic acid (0.0909 g, 0.353 mmol, Intermediate 5) in dichloromethane (3.53 mL) at room temperature. Then, 3-amino-1-methylpyrrolidin-2-one (0.052 g, 0.459 mmol) was added and the reaction mixture was stirred for five minutes. Then, n-propylphosphonic acid anhydride (0.420 mL, 0.706 mmol) was added and the reaction mixture was stirred for six hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give racemic 2-(3-chloro-5-fluorophenyl)-N-(1-methyl-2-oxopyrrolidin-3-yl)thiazole-5-carboxamide (0.1148 g, 0.308 mmol, 87% yield). ¹H NMR (400 MHz, CD₃SOCD₃) δ 1.86-1.98 (m, 1H), 2.30-2.40 (m, 1H), 2.77 (s, 3H), 3.30-3.36 (m, 2H), 4.56 (q, J=9 Hz, 1H), 7.65 (dt, J=9, 2 Hz, 1H), 7.84 (ddd, J=9, 2, 1 Hz, 1H), 7.90 (t, J=2 Hz, 1H), 8.50 (s, 1H), 9.11 (d, J=8 Hz, 1H); LC-MS (LC-ES) for C₁₅H₁₃ClFN₃O₂S M+H=354.

### Example 64

### (S)-2-(3-Chloro-5-fluorophenyl)-N-(1-methyl-2-oxopyrrolidin-3-yl)thiazole-5-carboxamide

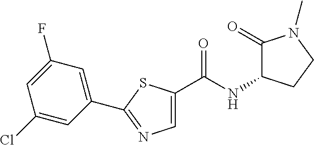 [SMILES: CN1CC[C@H](NC(=O)c2cnc(-c3cc(F)cc(Cl)c3)s2)C1=O]

N,N-Diisopropylethylamine (0.271 mL, 1.550 mmol) was added to 2-(3-chloro-5-fluorophenyl)thiazole-5-carboxylic acid (0.0799 g, 0.310 mmol, Intermediate 5) in dichloromethane (3.10 mL) at room temperature. Then, (S)-3-amino-1-methylpyrrolidin-2-one (0.046 g, 0.403 mmol) was added and the reaction mixture was stirred for five minutes. Then, n-propylphosphonic acid anhydride (0.369 mL, 0.620 mmol) was added and the reaction mixture was stirred for sixty-six hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give (S)-2-(3-chloro-5-fluorophenyl)-N-(1-methyl-2-oxopyrrolidin-3-yl)thiazole-5-carboxamide (0.0993 g, 0.267 mmol, 86% yield). ¹H NMR (400 MHz, CD₃SOCD₃) δ 1.86-1.98 (m, 1H), 2.30-2.40 (m, 1H), 2.78 (s, 3H), 3.30-3.36 (m, 2H), 4.57 (q, J=9 Hz, 1H), 7.65 (dt, J=9, 2 Hz, 1H), 7.84 (ddd, J=9, 2, 1 Hz, 1H), 7.91 (t, J=2 Hz, 1H), 8.51 (s, 1H), 9.12 (d, J=8 Hz, 1H); LC-MS (LC-ES) for C₁₅H₁₃ClFN₃O₂S M+H=354.

### Example 65

### Racemic 2-(3-Chloro-5-fluorophenyl)-N-methyl-N-(2-oxopyrrolidin-3-yl)thiazole-5-carboxamide

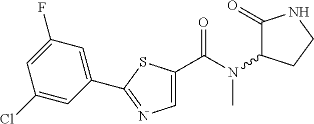 [SMILES: CN(C(=O)c1cnc(-c2cc(F)cc(Cl)c2)s1)C1CCNC1=O]

N,N-Diisopropylethylamine (0.344 mL, 1.968 mmol) was added to 2-(3-chloro-5-fluorophenyl)thiazole-5-carboxylic acid (0.1014 g, 0.394 mmol, Intermediate 5) in dichloromethane (3.94 mL) at room temperature. Then, racemic 3-(methylamino)pyrrolidin-2-one hydrochloride (0.083 g, 0.551 mmol) was added and the reaction mixture was stirred for five minutes. Then, n-propylphosphonic acid anhydride (0.469 mL, 0.787 mmol) was added and the reaction mixture was stirred for sixteen hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give racemic 2-(3-chloro-5-fluorophenyl)-N-methyl-N-(2-oxopyrrolidin-3-yl)thiazole-5-carboxamide (0.1283 g, 0.345 mmol, 88% yield). ¹H NMR (400 MHz, CD₃SOCD₃) δ 2.06-2.50 (m, 2H), 2.83 & 3.14 (br s, 2H), 3.25 (br s, 3H), 4.86 & 5.05 (br s, 1H), 7.65 (dt, J=9, 2 Hz, 1H), 7.84 (br d, J=9 Hz, 1H), 7.91 (br s, 1H), 8.03 & 8.08 (br s, 1H), 8.22 & 8.38 (br s, 1H); LC-MS (LC-ES) for C₁₅H₁₃ClFN₃O₂S M+H=354.

### Examples 66 & 67

### (S)-2-(3-Chloro-5-fluorophenyl)-N-methyl-N-(2-oxopyrrolidin-3-yl)thiazole-5-carboxamide; and

### (R)-2-(3-Chloro-5-fluorophenyl)-N-methyl-N-(2-oxopyrrolidin-3-yl)thiazole-5-carboxamide

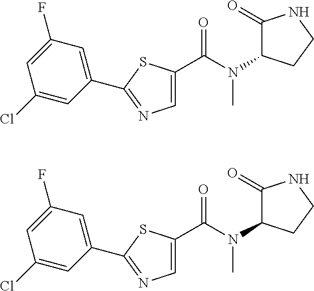 [SMILES: CN(C(=O)c1cnc(-c2cc(F)cc(Cl)c2)s1)[C@H]1CCNC1=O ; CN(C(=O)c1cnc(-c2cc(F)cc(Cl)c2)s1)[C@@H]1CCNC1=O]

Racemic 2-(3-chloro-5-fluorophenyl)-N-methyl-N-(2-oxopyrrolidin-3-yl)thiazole-5-carboxamide (0.1119 g, 0.316 mmol, Example 65) was separated into its enantiomers on a chiral Chiralpak CC4 column, eluting with ethanol:heptane (3:7) to give (S)-2-(3-chloro-5-fluorophenyl)-N-methyl-N-(2-oxopyrrolidin-3-yl)thiazole-5-carboxamide (0.0464 g, 0.125 mmol, 39.4% yield) as the first enantiomer to elute (t_{R}=7.3 minutes, >99% ee, [α]_{D}=−52.4° (C=0.50, MeCN, 24° C.)) and (R)-2-(3-chloro-5-fluorophenyl)-N-methyl-N-(2-oxopyrrolidin-3-yl)thiazole-5-carboxamide (0.0474 g, 0.127 mmol, 40.2% yield) as the last enantiomer to elute (t_{R}=9.1 minutes, 99% ee, [α]_{D}=+52.4° (C=0.50, MeCN, 24° C.)). The structures were assigned by vibrational circular dichroism.

### (S)-2-(3-Chloro-5-fluorophenyl)-N-methyl-N-(2-oxopyrrolidin-3-yl)thiazole-5-carboxamide

¹H NMR (400 MHz, CD₃SOCD₃) δ 2.06-2.50 (m, 2H), 2.83 & 3.14 (br s, 2H), 3.25 (br s, 3H), 4.87 & 5.05 (br s, 1H), 7.65 (dt, J=9, 2 Hz, 1H), 7.84 (br d, J=9 Hz, 1H), 7.90 (br s, 1H), 8.02 & 8.08 (br s, 1H), 8.22 & 8.38 (br s, 1H); LC-MS (LC-ES) for C₁₅H₁₃ClFN₃O₂S M+H=354.

### (R)-2-(3-Chloro-5-fluorophenyl)-N-methyl-N-(2-oxopyrrolidin-3-yl)thiazole-5-carboxamide

¹H NMR (400 MHz, CD₃SOCD₃) δ 2.06-2.50 (m, 2H), 2.83 & 3.14 (br s, 2H), 3.24 (br s, 3H), 4.86 & 5.05 (br s, 1H), 7.65 (dt, J=9, 2 Hz, 1H), 7.84 (br d, J=9 Hz, 1H), 7.90 (br s, 1H), 8.01 & 8.08 (br s, 1H), 8.21 & 8.38 (br s, 1H); LC-MS (LC-ES) for C₁₅H₁₃ClFN₃O₂S M+H=354.

### Example 68

### (S)-2-(3-Methoxyphenyl)-N-(2-oxopyrrolidin-3-yl)thiazole-5-carboxamide

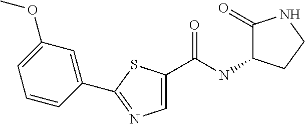 [SMILES: COc1cccc(-c2ncc(C(=O)N[C@H]3CCNC3=O)s2)c1]

N,N-Diisopropylethylamine (0.224 mL, 1.284 mmol) was added to 2-(3-methoxyphenyl)thiazole-5-carboxylic acid (0.0604 g, 0.257 mmol, Intermediate 39) in dichloromethane (2.57 mL) at room temperature. Then, (S)-3-aminopyrrolidin-2-one hydrochloride (0.049 g, 0.359 mmol) was added and the reaction mixture was stirred for five minutes. Then, n-propylphosphonic acid anhydride (0.306 mL, 0.513 mmol) was added and the reaction mixture was stirred for six hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give (S)-2-(3-methoxyphenyl)-N-(2-oxopyrrolidin-3-yl)thiazole-5-carboxamide (0.0125 g, 0.037 mmol, 14.57% yield). ¹H NMR (400 MHz, CD₃SOCD₃) δ 1.92-2.04 (m, 1H), 2.30-2.42 (m, 1H), 3.20-3.26 (m, 2H), 3.83 (br s, 3H), 4.52 (dt, J=11, 9 Hz, 1H), 7.10 (ddd, J=8, 3, 1 Hz, 1H), 7.43 (t, J=8 Hz, 1H), 7.51 (dd, J=2, 2 Hz, 1H), 7.55 (ddd, J=8, 2, 1 Hz, 1H), 7.93 (br s, 1H), 8.46 (br s, 1H), 8.98 (d, J=8 Hz, 1H); LC-MS (LC-ES) for C₁₅H₁₅N₃O₃S M+H=318.

### Example 69

### (S)-2-(3-Hydroxyphenyl)-N-(2-oxopyrrolidin-3-yl)thiazole-5-carboxamide

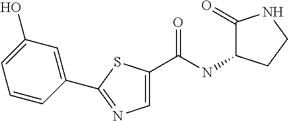 [SMILES: O=C(N[C@H]1CCNC1=O)c1cnc(-c2cccc(O)c2)s1]

N,N-Diisopropylethylamine (0.202 mL, 1.161 mmol) was added to 2-(3-hydroxyphenyl)thiazole-5-carboxylic acid (0.0642 g, 0.290 mmol, Intermediate 40) in N,N-dimethylformamide (2.90 mL) at room temperature. Then, 1-((dimethylamino)(dimethyliminio)methyl)-1H-[1,2,3]triazolo[4,5-b]pyridine 3-oxide hexafluorophosphate(V) (0.132 g, 0.348 mmol) was added and the reaction mixture was stirred for five minutes. Then, (S)-3-aminopyrrolidin-2-one hydrochloride (0.055 g, 0.406 mmol) was added and the reaction mixture was stirred for sixteen hours. The reaction mixture was concentrated. The resulting residue was purified by reverse phase HPLC chromatography, eluting with acetonitrile:water (0:1 to 1:0) with 0.1% ammonium hydroxide, then further purified by silica gel chromatography, eluting with methanol:ethyl acetate (0:1 to 2:3) to give (S)-2-(3-hydroxyphenyl)-N-(2-oxopyrrolidin-3-yl)thiazole-5-carboxamide (0.0521 g, 0.163 mmol, 56.2% yield). ¹H NMR (400 MHz, CD₃SOCD₃) δ 1.92-2.04 (m, 1H), 2.32-2.42 (m, 1H), 3.20-3.28 (m, 2H), 4.46-4.56 (m, 1H), 6.91 (ddd, J=8, 2, 1 Hz, 1H), 7.31 (t, J=8 Hz, 1H), 7.36-7.42 (m, 2H), 7.92 (br s, 1H), 8.43 (s, 1H), 8.95 (d, J=8 Hz, 1H), 9.87 (br s, 1H); LC-MS (LC-ES) for C₁₄H₁₃N₃O₃S M+H=304.

### Example 70

### Racemic 2-(3-Chloro-5-fluorophenyl)-N-(1-(4-methoxybenzyl)-3-methyl-2-oxopyrrolidin-3-yl)thiazole-5-carboxamide

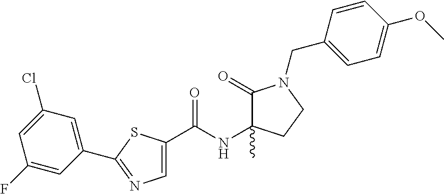 [SMILES: COc1ccc(CN2CCC(C)(NC(=O)c3cnc(-c4cc(F)cc(Cl)c4)s3)C2=O)cc1]

N,N-Diisopropylethylamine (0.388 mL, 2.229 mmol) was added to 2-(3-chloro-5-fluorophenyl)thiazole-5-carboxylic acid (0.1436 g, 0.557 mmol, Intermediate 5) in N,N-dimethylformamide (2.79 mL) at room temperature. Then, 1-((dimethylamino)(dimethyliminio)methyl)-1H-[1,2,3]triazolo[4,5-b]pyridine 3-oxide hexafluorophosphate(V) (0.254 g, 0.669 mmol) was added and the reaction mixture was stirred for five minutes. Then, 3-amino-1-(4-methoxybenzyl)-3-methylpyrrolidin-2-one hydroiodide (0.262 g, 0.725 mmol, Intermediate 41) was added and the reaction mixture was stirred for two hours. Saturated sodium bicarbonate was added and the reaction mixture was extracted with ethyl acetate, dried over magnesium sulfate, filtered, and concentrated. The resulting residue was purified by silica gel chromatography, eluting with ethyl acetate:heptanes (1:1 to 1:0) to give 2-(3-chloro-5-fluorophenyl)-N-(1-(4-methoxybenzyl)-3-methyl-2-oxopyrrolidin-3-yl)thiazole-5-carboxamide (0.2297 g, 0.460 mmol, 83% yield). ¹H NMR (400 MHz, CD₃SOCD₃) δ 1.36 (s, 3H), 1.88-1.96 (m, 1H), 2.44-2.52 (m, 1H), 3.08-3.24 (m, 2H), 3.74 (s, 3H), 4.32 (ABq, J_{AB}=15 Hz, Δv_{AB}=56 Hz, 2H), 6.91 (d, J=9 Hz, 2H), 7.22 (d, J=9 Hz, 2H), 7.65 (dt, J=9, 2 Hz, 1H), 7.84 (ddd, J=9, 2, 2 Hz, 1H), 7.91 (t, J=2 Hz, 1H), 8.62 (s, 1H), 8.76 (br s, 1H); LC-MS (LC-ES) for C₂₃H₂₁ClFN₃O₃S M+H=474.

### Example 71

### Racemic 2-(3-Chloro-5-fluorophenyl)-N-(3-methyl-2-oxopyrrolidin-3-yl)thiazole-5-carboxamide

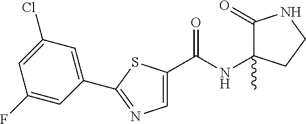 [SMILES: CC1(NC(=O)c2cnc(-c3cc(F)cc(Cl)c3)s2)CCNC1=O]

Trifluoromethanesulfonic acid (0.154 mL, 1.742 mmol) was added to 2-(3-chloro-5-fluorophenyl)-N-(1-(4-methoxybenzyl)-3-methyl-2-oxopyrrolidin-3-yl)thiazole-5-carboxamide (0.2064 g, 0.435 mmol, Example 70) in toluene (0.435 mL) at room temperature and the reaction mixture was stirred for four hours at 80° C. The reaction mixture was quenched with methanol, saturated sodium bicarbonate added, extracted with ethyl acetate, dried over magnesium sulfate, filtered, and concentrated. The residue was purified by silica gel chromatography, eluting with methanol:ethyl acetate (0:1 to 1:4) to give 2-(3-chloro-5-fluorophenyl)-N-(3-methyl-2-oxopyrrolidin-3-yl)thiazole-5-carboxamide (0.1705 g, 0.434 mmol, 100% yield). ¹H NMR (400 MHz, CD₃SOCD₃) δ 1.35 (s, 3H), 1.92-2.00 (m, 1H), 2.52-2.60 (m, 1H), 3.14-3.30 (m, 2H), 7.64 (dt, J=9, 2 Hz, 1H), 7.68 (br s, 1H), 7.83 (ddd, J=9, 2, 2 Hz, 1H), 7.90 (t, J=2 Hz, 1H), 8.59 (br s, 1H), 8.60 (s, 1H); LC-MS (LC-ES) for C₁₅H₁₃ClFN₃O₂S M+H=354.

### Examples 72 & 73

### (S)-2-(3-Chloro-5-fluorophenyl)-N-(3-methyl-2-oxopyrrolidin-3-yl)thiazole-5-carboxamide; and

### (R)-2-(3-Chloro-5-fluorophenyl)-N-(3-methyl-2-oxopyrrolidin-3-yl)thiazole-5-carboxamide

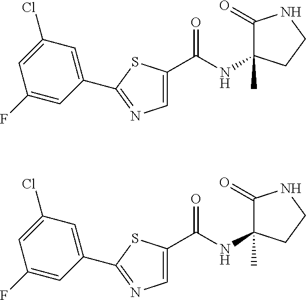 [SMILES: C[C@]1(NC(=O)c2cnc(-c3cc(F)cc(Cl)c3)s2)CCNC1=O ; C[C@@]1(NC(=O)c2cnc(-c3cc(F)cc(Cl)c3)s2)CCNC1=O]

Racemic 2-(3-chloro-5-fluorophenyl)-N-(3-methyl-2-oxopyrrolidin-3-yl)thiazole-5-carboxamide (0.1565 g, 0.442 mmol, Example 71) was separated into its enantiomers on a chiral Chromega CC4 column, eluting with methanol:acetonitrile (5:95) with 0.1% isopropylamine to give an impure first enantiomer (CC4 t_{R}=4.5 minutes), containing an achiral impurity, and a pure second enantiomer (CC4 t_{R}=5.1 minutes). The first enantiomer was repurified on a Chiralpak ID column, eluting with methanol:acetonitrile (2:3) with 0.1% isopropylamine to give pure (S)-2-(3-chloro-5-fluorophenyl)-N-(3-methyl-2-oxopyrrolidin-3-yl)thiazole-5-carboxamide (0.0378 g, 0.101 mmol, 22.95% yield) (CC4 t_{R}=4.5 minutes, ID t_{R}=5.6 minutes, >99% ee). Whereas the second enantiomer, (R)-2-(3-chloro-5-fluorophenyl)-N-(3-methyl-2-oxopyrrolidin-3-yl)thiazole-5-carboxamide (0.0448 g, 0.120 mmol, 27.2% yield) (CC4 t_{R}=5.1 minutes, ID t_{R}=8.1 minutes, 99% ee) did not need further purification. The structures were assigned by vibrational circular dichroism.

### (S)-2-(3-Chloro-5-fluorophenyl)-N-(3-methyl-2-oxopyrrolidin-3-yl)thiazole-5-carboxamide

¹H NMR (400 MHz, CD₃SOCD₃) δ 1.35 (s, 3H), 1.96 (ddd, J=12, 8, 2 Hz, 1H), 2.50-2.60 (m, 1H), 3.14-3.30 (m, 2H), 7.64 (dt, J=9, 2 Hz, 1H), 7.68 (br s, 1H), 7.83 (ddd, J=9, 2, 2 Hz, 1H), 7.90 (t, J=2 Hz, 1H), 8.59 (br s, 1H), 8.60 (s, 1H); LC-MS (LC-ES) for C₁₅H₁₃ClFN₃O₂S M+H=354.

### (R)-2-(3-Chloro-5-fluorophenyl)-N-(3-methyl-2-oxopyrrolidin-3-yl)thiazole-5-carboxamide

¹H NMR (400 MHz, CD₃SOCD₃) δ 1.35 (s, 3H), 1.96 (ddd, J=12, 7, 2 Hz, 1H), 2.50-2.60 (m, 1H), 3.14-3.30 (m, 2H), 7.64 (dt, J=9, 2 Hz, 1H), 7.68 (br s, 1H), 7.83 (ddd, J=9, 2, 2 Hz, 1H), 7.90 (t, J=2 Hz, 1H), 8.59 (br s, 1H), 8.60 (s, 1H); LC-MS (LC-ES) for C₁₅H₁₃ClFN₃O₂S M+H=354.

### Example 74

### Racemic cis-2-(3-Chloro-5-fluorophenyl)-N-(4-methyl-2-oxopyrrolidin-3-yl)thiazole-5-carboxamide; and

### Racemic trans-2-(3-Chloro-5-fluorophenyl)-N-(4-methyl-2-oxopyrrolidin-3-yl)thiazole-5-carboxamide

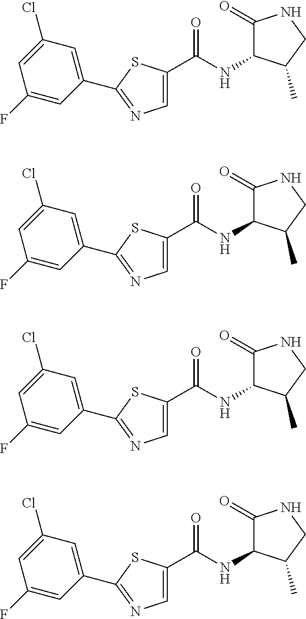 [SMILES: C[C@H]1CNC(=O)[C@H]1NC(=O)c1cnc(-c2cc(F)cc(Cl)c2)s1 ; C[C@@H]1CNC(=O)[C@@H]1NC(=O)c1cnc(-c2cc(F)cc(Cl)c2)s1 ; C[C@@H]1CNC(=O)[C@H]1NC(=O)c1cnc(-c2cc(F)cc(Cl)c2)s1 ; C[C@H]1CNC(=O)[C@@H]1NC(=O)c1cnc(-c2cc(F)cc(Cl)c2)s1]

N,N-Diisopropylethylamine (0.547 mL, 3.14 mmol) was added to 2-(3-chloro-5-fluorophenyl)thiazole-5-carboxylic acid (0.2022 g, 0.785 mmol, Intermediate 5) in N,N-dimethylformamide (2.62 mL) at room temperature. Then, 1-((dimethylamino)(dimethyliminio)methyl)-1H-[1,2,3]triazolo[4,5-b]pyridine 3-oxide hexafluorophosphate(V) (0.358 g, 0.942 mmol) was added and the reaction mixture was stirred for five minutes. Then, a racemic 6:1 mixture of cis:trans isomers of 3-amino-4-methylpyrrolidin-2-one hydrochloride (0.236 g, 0.785 mmol, Intermediate 42) were added and the reaction mixture was stirred for three hours. Saturated sodium bicarbonate was added and the reaction mixture was extracted with ethyl acetate, dried over magnesium sulfate, filtered, and concentrated. The resulting residue was purified by silica gel chromatography, eluting with methanol:ethyl acetate (0:1 to 1:9) to give racemic cis-2-(3-chloro-5-fluorophenyl)-N-(4-methyl-2-oxopyrrolidin-3-yl)thiazole-5-carboxamide (0.1027 g, 0.276 mmol, 35.1% yield) and racemic trans-2-(3-chloro-5-fluorophenyl)-N-(4-methyl-2-oxopyrrolidin-3-yl)thiazole-5-carboxamide (0.0104 g, 0.029 mmol, 3.75% yield) as well as some mixed fractions, which were discarded.

### Racemic cis-2-(3-Chloro-5-fluorophenyl)-N-(4-methyl-2-oxopyrrolidin-3-yl)thiazole-5-carboxamide

¹H NMR (400 MHz, CD₃SOCD₃) δ 0.89 (d, J=7 Hz, 3H), 2.60-2.68 (m, 1H), 2.86-2.92 (m, 1H), 3.43 (dd, J=10, 7 Hz, 1H), 4.59 (t, J=8 Hz, 1H), 7.64 (dt, J=9, 2 Hz, 1H), 7.84 (ddd, J=9, 2, 2 Hz, 1H), 7.90 (t, J=2 Hz, 1H), 7.94 (br s, 1H), 8.66 (s, 1H), 8.98 (d, J=8 Hz, 1H); LC-MS (LC-ES) for C₁₅H₁₃ClFN₃O₂S M+H=354.

### Racemic trans-2-(3-Chloro-5-fluorophenyl)-N-(4-methyl-2-oxopyrrolidin-3-yl)thiazole-5-carboxamide

¹H NMR (400 MHz, CD₃SOCD₃) δ 1.09 (d, J=7 Hz, 3H), 2.30-2.48 (m, 1H), 2.88 (t, J=9 Hz, 1H), 3.22-3.38 (m, 1H), 4.22 (dd, J=11, 9 Hz, 1H), 7.65 (dt, J=9, 2 Hz, 1H), 7.84 (ddd, J=9, 2, 2 Hz, 1H), 7.89 (br s, 1H), 7.91 (t, J=2 Hz, 1H), 8.53 (s, 1H), 8.98 (d, J=9 Hz, 1H); LC-MS (LC-ES) for C₁₅H₁₃ClFN₃O₂S M+H=354.

### Examples 75 & 76

### 2-(3-Chloro-5-fluorophenyl)-N-((3S,4S)-4-methyl-2-oxopyrrolidin-3-yl)thiazole-5-carboxamide; and

### 2-(3-Chloro-5-fluorophenyl)-N-((3R,4R)-4-methyl-2-oxopyrrolidin-3-yl)thiazole-5-carboxamide

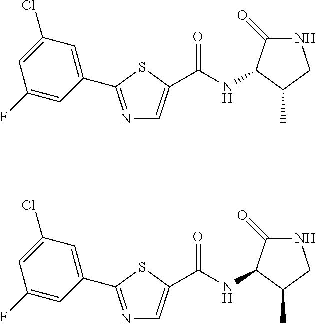 [SMILES: C[C@H]1CNC(=O)[C@H]1NC(=O)c1cnc(-c2cc(F)cc(Cl)c2)s1 ; C[C@@H]1CNC(=O)[C@@H]1NC(=O)c1cnc(-c2cc(F)cc(Cl)c2)s1]

Racemic cis 2-(3-chloro-5-fluorophenyl)-N-((3S,4S)-4-methyl-2-oxopyrrolidin-3-yl)thiazole-5-carboxamide and 2-(3-chloro-5-fluorophenyl)-N-((3R,4R)-4-methyl-2-oxopyrrolidin-3-yl)thiazole-5-carboxamide (0.0632 g, 0.179 mmol, Example 74) was separated into its enantiomers on a chiral Chromega CC4 column, eluting with ethanol:heptane (1:4) with 0.1% isopropylamine to give 2-(3-chloro-5-fluorophenyl)-N-((3S,4S)-4-methyl-2-oxopyrrolidin-3-yl)thiazole-5-carboxamide (0.0291 g, 0.078 mmol, 43.7% yield) as the first enantiomer to elute (CC4 t_{R}=5.8 minutes, >99% ee) and 2-(3-chloro-5-fluorophenyl)-N-((3R,4R)-4-methyl-2-oxopyrrolidin-3-yl)thiazole-5-carboxamide (0.0290 g, 0.078 mmol, 43.6% yield), as the last enantiomer to elute (CC4 t_{R}=8.1 minutes, 98% ee). The structures were assigned by vibrational circular dichroism.

### 2-(3-Chloro-5-fluorophenyl)-N-((3S,4S)-4-methyl-2-oxopyrrolidin-3-yl)thiazole-5-carboxamide

¹H NMR (400 MHz, CD₃SOCD₃) δ 0.89 (d, J=7 Hz, 3H), 2.60-2.68 (m, 1H), 2.86-2.92 (m, 1H), 3.43 (dd, J=10, 7 Hz, 1H), 4.60 (t, J=8 Hz, 1H), 7.65 (dt, J=9, 2 Hz, 1H), 7.84 (ddd, J=9, 2, 2 Hz, 1H), 7.91 (t, J=2 Hz, 1H), 7.97 (br s, 1H), 8.66 (s, 1H), 9.00 (d, J=8 Hz, 1H); LC-MS (LC-ES) for C₁₅H₁₃ClFN₃O₂S M+H=354.

### 2-(3-Chloro-5-fluorophenyl)-N-((3R,4R)-4-methyl-2-oxopyrrolidin-3-yl)thiazole-5-carboxamide

¹H NMR (400 MHz, CD₃SOCD₃) δ 0.89 (d, J=7 Hz, 3H), 2.60-2.68 (m, 1H), 2.86-2.92 (m, 1H), 3.43 (dd, J=10, 7 Hz, 1H), 4.59 (t, J=8 Hz, 1H), 7.65 (dt, J=9, 2 Hz, 1H), 7.84 (ddd, J=9, 2, 2 Hz, 1H), 7.91 (t, J=2 Hz, 1H), 7.95 (br s, 1H), 8.66 (s, 1H), 8.99 (d, J=8 Hz, 1H); LC-MS (LC-ES) for C₁₅H₁₃ClFN₃O₂S M+H=354.

### Example 77

### (S)-2-(3-Chloro-5-fluorophenyl)-N-(2-thioxopyrrolidin-3-yl)thiazole-5-carboxamide

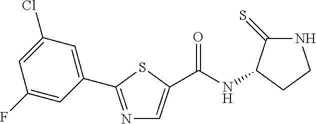 [SMILES: O=C(N[C@H]1CCNC1=S)c1cnc(-c2cc(F)cc(Cl)c2)s1]

Lawesson's reagent (2,4-bis(4-methoxyphenyl)-1,3,2,4-dithiadiphosphetane 2,4-disulfide (0.060 g, 0.148 mmol)) was added to (S)-2-(3-chloro-5-fluorophenyl)-N-(2-oxopyrrolidin-3-yl)thiazole-5-carboxamide (0.1005 g, 0.296 mmol, Example 6) in toluene (5.92 mL) at room temperature, then the reaction mixture was heated at 80° C. for four hours. The reaction mixture was cooled, water (2 mL) added, heated to reflux for 1 hour, saturated sodium bicarbonate added, extracted with dichloromethane, dried with magnesium sulfate, filtered, and concentrated. The residue was purified by silica gel chromatography, eluting with methanol:ethyl acetate (0:1 to 1:9) to give (S)-2-(3-chloro-5-fluorophenyl)-N-(2-thioxopyrrolidin-3-yl)thiazole-5-carboxamide (0.0669 g, 0.179 mmol, 60.4% yield). ¹H NMR (400 MHz, CD₃SOCD₃) δ 1.94-2.08 (m, 1H), 2.42-2.52 (m, 1H), 3.52 (dd, J=9, 4 Hz, 2H), 4.89 (dt, J=10, 9 Hz, 1H), 7.65 (dt, J=9, 2 Hz, 1H), 7.84 (ddd, J=9, 2, 2 Hz, 1H), 7.91 (t, J=2 Hz, 1H), 8.53 (s, 1H), 9.10 (d, J=9 Hz, 1H), 10.48 (br s, 1H); LC-MS (LC-ES) for C₁₄H₁₁ClFN₃OS₂ M+H=356.

### Example 78

### (S)-2-(3-chloro-5-fluorophenyl)-N-(2-selenoxopyrrolidin-3-yl)thiazole-5-carboxamide

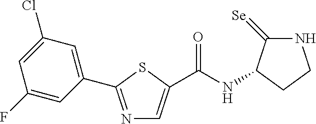 [SMILES: O=C(N[C@H]1CCNC1=[Se])c1cnc(-c2cc(F)cc(Cl)c2)s1]

Woollin's reagent (2,4-diphenyl-1,3,2,4-diselenadiphosphetane 2,4-diselenide (0.077 g, 0.145 mmol)) was added to (S)-2-(3-chloro-5-fluorophenyl)-N-(2-oxopyrrolidin-3-yl)thiazole-5-carboxamide (0.0982 g, 0.289 mmol, Example 6) in toluene (2.89 mL) at room temperature, then the reaction mixture was heated at reflux for six hours. The reaction mixture was cooled, water (2 mL) added, heated to reflux for 1 hour, saturated sodium bicarbonate added, extracted with dichloromethane, dried with magnesium sulfate, filtered, and concentrated. The residue was purified by silica gel chromatography, eluting with methanol:ethyl acetate (0:1 to 1:9) to give (S)-2-(3-chloro-5-fluorophenyl)-N-(2-selenoxopyrrolidin-3-yl)thiazole-5-carboxamide (0.0601 g, 0.142 mmol, 49.1% yield). ¹H NMR (400 MHz, CD₃SOCD₃) δ 1.96-2.06 (m, 1H), 2.44-2.56 (m, 1H), 3.36-3.46 (m, 1H), 3.48-3.56 (m, 1H), 4.85 (q, J=9 Hz, 1H), 7.66 (dt, J=9, 2 Hz, 1H), 7.85 (ddd, J=9, 2, 2 Hz, 1H), 7.92 (t, J=2 Hz, 1H), 8.54 (s, 1H), 9.17 (d, J=9 Hz, 1H), 11.40 (br s, 1H); LC-MS (LC-ES) for C₁₄H₁₁ClFN₃OSSe M+H=402.

### Example 79

### 2-(3-Chloro-5-fluorophenyl)-N-(2-oxoimidazolidin-1-yl)thiazole-5-carboxamide

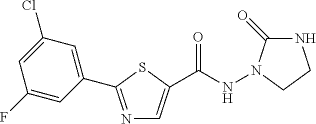 [SMILES: O=C(NN1CCNC1=O)c1cnc(-c2cc(F)cc(Cl)c2)s1]

N,N-Diisopropylethylamine (0.537 mL, 3.08 mmol) was added to 2-(3-chloro-5-fluorophenyl)thiazole-5-carboxylic acid (0.1585 g, 0.615 mmol, Intermediate 5) in dichloromethane (3.08 mL) at room temperature. Then, 1-aminoimidazolidin-2-one (0.100 g, 0.984 mmol, Enamine) was added and the reaction mixture was stirred for five minutes. Then, n-propylphosphonic acid anhydride (0.732 mL, 1.230 mmol) was added and the reaction mixture was stirred for two hours. Saturated sodium bicarbonate was added to the reaction mixture and it was extracted with dichloromethane, dried over magnesium sulfate, filtered, and concentrated. The resulting residue was purified by silica gel chromatography, eluting with methanol:ethyl acetate (0:1 to 3:7) to give 2-(3-chloro-5-fluorophenyl)-N-(2-oxoimidazolidin-1-yl)thiazole-5-carboxamide (0.1758 g, 0.490 mmol, 80% yield). ¹H NMR (400 MHz, CD₃SOCD₃) δ 3.33 (t, J=7 Hz, 2H), 3.58 (t, J=7 Hz, 2H), 6.94 (br s, 1H), 7.66 (dt, J=9, 2 Hz, 1H), 7.85 (ddd, J=9, 2, 2 Hz, 1H), 7.92 (t, J=2 Hz, 1H), 8.52 (br s, 1H), 10.70 (br s, 1H); LC-MS (LC-ES) for C₁₃H₁₀ClFN₄O₂S M+H=341.

### Example 80

### (S)-2-(3-chloro-5-fluorophenyl)-N-(2-oxopyrrolidin-3-yl)thiazole-5-carbothioamide

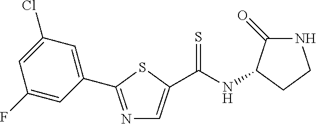 [SMILES: O=C1NCC[C@@H]1NC(=S)c1cnc(-c2cc(F)cc(Cl)c2)s1]

N,N-Diisopropylethylamine (0.167 mL, 0.959 mmol) was added to 2-(3-chloro-5-fluorophenyl)thiazole-5-carbothioic O-acid (0.0525 g, 0.192 mmol, Intermediate 43) in dichloromethane (0.959 mL) at room temperature. Then, (S)-3-aminopyrrolidin-2-one (0.029 g, 0.288 mmol, AstaTech) was added and the reaction mixture was stirred for five minutes. Then, n-propylphosphonic acid anhydride (0.228 mL, 0.384 mmol) was added and the reaction mixture was stirred for six hours. Saturated sodium bicarbonate was added to the reaction mixture and it was extracted with dichloromethane, dried over magnesium sulfate, filtered, and concentrated. The resulting residue was purified by reverse phase HPLC, eluting with acetonitrile:water with 0.1% ammonium hydroxide (0:1 to 1:0), then repurified by silica gel chromatography, eluting with methanol:ethyl acetate (0:1 to 1:4) to give (S)-2-(3-chloro-5-fluorophenyl)-N-(2-oxopyrrolidin-3-yl)thiazole-5-carbothioamide (0.0031 g, 8.28 μmol, 4.32% yield) as the minor product. ¹H NMR (400 MHz, CD₃SOCD₃) δ 1.94-2.06 (m, 1H), 2.46-2.58 (m, 1H), 3.30 (t, J=5 Hz, 2H), 5.27 (t, J=9 Hz, 1H), 7.65 (dt, J=9, 2 Hz, 1H), 7.84 (ddd, J=9, 2, 2 Hz, 1H), 7.91 (t, J=2 Hz, 1H), 8.12 (br s, 1H), 8.45 (s, 1H), 10.70 (br s, 1H); LC-MS (LC-ES) for C₁₄H₁₁ClFN₃OS₂ M+H=356.

### Example 81

### 2-(3-Chloro-5-fluorophenyl)-N-((3S,5S)-5-methyl-2-oxopyrrolidin-3-yl)thiazole-5-carboxamide

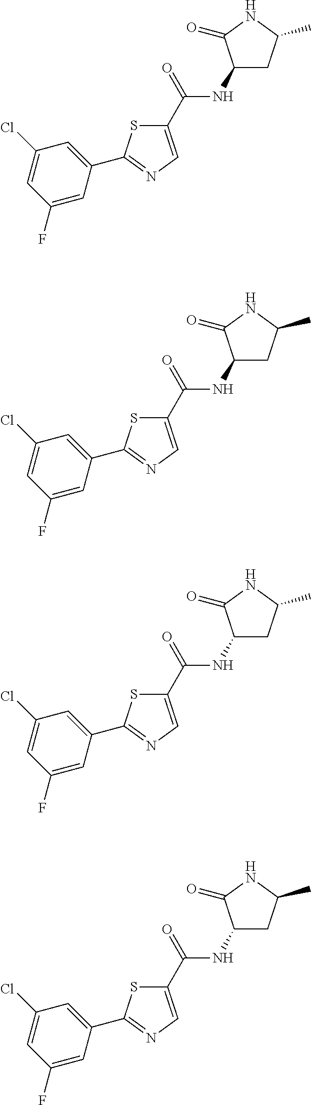 [SMILES: C[C@@H]1C[C@@H](NC(=O)c2cnc(-c3cc(F)cc(Cl)c3)s2)C(=O)N1 ; C[C@H]1C[C@@H](NC(=O)c2cnc(-c3cc(F)cc(Cl)c3)s2)C(=O)N1 ; C[C@@H]1C[C@H](NC(=O)c2cnc(-c3cc(F)cc(Cl)c3)s2)C(=O)N1 ; C[C@H]1C[C@H](NC(=O)c2cnc(-c3cc(F)cc(Cl)c3)s2)C(=O)N1]

N,N-Diisopropylethylamine (0.578 mL, 3.32 mmol) was added to 2-(3-chloro-5-fluorophenyl)thiazole-5-carboxylic acid (0.2139 g, 0.830 mmol, Intermediate 5) in dichloromethane (2.77 mL) at room temperature. Then, 1-((dimethylamino)(dimethyliminio)methyl)-1H-[1,2,3]triazolo[4,5-b]pyridine 3-oxide hexafluorophosphate(V) (0.379 g, 0.996 mmol) was added and the reaction mixture was stirred for five minutes. Then, (5S)-3-amino-5-methylpyrrolidin-2-one hydrochloride (0.163 g, 1.079 mmol, Intermediate 26) was added and the reaction mixture was stirred for sixteen hours. Saturated sodium bicarbonate was added and the reaction mixture was extracted with dichloromethane, dried over magnesium sulfate, filtered, and concentrated. The resulting residue was purified by silica gel chromatography, eluting with methanol:ethyl acetate (0:1 to 1:9) to give 2-(3-chloro-5-fluorophenyl)-N-((5S)-5-methyl-2-oxopyrrolidin-3-yl)thiazole-5-carboxamide (0.1998 g, 0.536 mmol, 64.6% yield) as an impure mixture of diastereomers with partial racemization of the (5S)-stereocenter. A 10:1 diastereomeric mixture of partially racemized 2-(3-chloro-5-fluorophenyl)-N-(5-methyl-2-oxopyrrolidin-3-yl)thiazole-5-carboxamide (0.1998 g, 0.565 mmol) (˜82% ee) was separated into its individual diastereomers on a chiral ChiralPak® AS-H column, eluting with methanol:acetonitrile (1:4) with 0.1% isopropylamine to give impure 2-(3-chloro-5-fluorophenyl)-N-((3R,5R)-5-methyl-2-oxopyrrolidin-3-yl)thiazole-5-carboxamide (0.0490 g, 0.014 mmol, 2.452% yield, see Example 61) (OD-H t_{R}=5.9 minutes, AS-H t_{R}=2.0 minutes, unknown ee, 10% pure:90% impurity with mass=385 g/mol), 2-(3-chloro-5-fluorophenyl)-N-((3R,5S)-5-methyl-2-oxopyrrolidin-3-yl)thiazole-5-carboxamide (0.0694 g, 0.186 mmol, 33.0% yield, see Example 41) (OD-H t_{R}=6.4 minutes, AS-H t_{R}=9.1 minutes, >99% ee), 2-(3-chloro-5-fluorophenyl)-N-((3S,5R)-5-methyl-2-oxopyrrolidin-3-yl)thiazole-5-carboxamide (0.0038 g, 10.20 μmol, 1.807% yield, see Example 62) (OD-H t_{R}=7.8 minutes, AS-H t_{R}=5.2 minutes, 97% ee), and 2-(3-chloro-5-fluorophenyl)-N-((3S,5S)-5-methyl-2-oxopyrrolidin-3-yl)thiazole-5-carboxamide (0.0058 g, 0.016 mmol, 2.76% yield) (OD-H t_{R}=9.3 minutes, AS-H t_{R}=3.9 minutes, 97% ee). The structures were previously prepared and separated by purification on an ChiralPak® OD-H column, eluting with ethanol:heptane (15:85) with 0.1% isopropylamine and assigned by NMR.

### 2-(3-Chloro-5-fluorophenyl)-N-((3R,5R)-5-methyl-2-oxopyrrolidin-3-yl)thiazole-5-carboxamide

See Example 61.

### 2-(3-Chloro-5-fluorophenyl)-N-((3R,5S)-5-methyl-2-oxopyrrolidin-3-yl)thiazole-5-carboxamide

See Example 41.

### 2-(3-Chloro-5-fluorophenyl)-N-((3S,5R)-5-methyl-2-oxopyrrolidin-3-yl)thiazole-5-carboxamide

See Example 62.

### 2-(3-Chloro-5-fluorophenyl)-N-((3S,5S)-5-methyl-2-oxopyrrolidin-3-yl)thiazole-5-carboxamide

¹H NMR (400 MHz, CD₃SOCD₃) δ 1.15 (d, J=6 Hz, 3H), 1.98-2.06 (m, 1H), 2.10-2.20 (m, 1H), 3.64-3.74 (m, 1H), 4.59 (q, J=9 Hz, 1H), 7.64 (dt, J=9, 2 Hz, 1H), 7.84 (ddd, J=9, 2, 2 Hz, 1H), 7.90 (t, J=2 Hz, 1H), 8.08 (s, 1H), 8.51 (s, 1H), 9.07 (d, J=8 Hz, 1H); LC-MS (LC-ES) for C₁₅H₁₃ClFN₃O₂S M+H=354.

### Example 82

### Racemic 2-(3-Chloro-5-fluorophenyl)-N-(5,5-dimethyl-2-oxopyrrolidin-3-yl)-1,3-selenazole-5-carboxamide

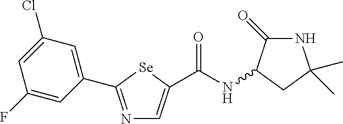 [SMILES: CC1(C)CC(NC(=O)c2cnc(-c3cc(F)cc(Cl)c3)[se]2)C(=O)N1]

N,N-Diisopropylethylamine (0.301 mL, 1.725 mmol) was added to lithium 2-(3-chloro-5-fluorophenyl)-1,3-selenazole-5-carboxylate (0.1071 g, 0.345 mmol, Intermediate 44) in dichloromethane (3.45 mL) at room temperature. Then, racemic 3-amino-5,5-dimethylpyrrolidin-2-one hydrochloride (0.085 g, 0.517 mmol, Intermediate 12) was added and the reaction mixture was stirred for five minutes. Then, n-propylphosphonic acid anhydride (0.411 mL, 0.690 mmol) was added and the reaction mixture was stirred for sixteen hours. The reaction mixture was concentrated. The resulting residue was purified by RP HPLC, eluting with acetonitrile:water with 0.1% ammonium hydroxide (5:95 to 100:0), then further purified by silica gel chromatography, eluting with ethyl acetate:methanol (0:1 to 3:7) to give racemic 2-(3-chloro-5-fluorophenyl)-N-(5,5-dimethyl-2-oxopyrrolidin-3-yl)-1,3-selenazole-5-carboxamide (0.0438 g, 0.100 mmol, 29.1% yield). ¹H NMR (400 MHz, CD₃SOCD₃) δ 1.21 (s, 3H), 1.25 (s, 3H), 1.82 (t, J=12 Hz, 1H), 2.25 (dd, J=12, 9 Hz, 1H), 4.67 (dt, J=10, 9 Hz, 1H), 7.65 (dt, J=9, 2 Hz, 1H), 7.84 (ddd, J=9, 2, 2 Hz, 1H), 7.91 (br t, J=2 Hz, 1H), 8.10 (s, 1H), 8.49 (s, 1H), 8.98 (d, J=8 Hz, 1H); LC-MS (LC-ES) for C₁₆H₁₅ClFN₃O₂Se M+H=415.

### Example 83

### Racemic 2-(3-Chloro-5-fluorophenyl)-N-(5,5-dimethyl-2-thioxopyrrolidin-3-yl)thiazole-5-carboxamide

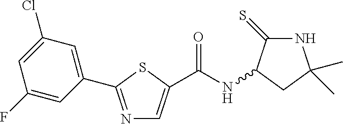 [SMILES: CC1(C)CC(NC(=O)c2cnc(-c3cc(F)cc(Cl)c3)s2)C(=S)N1]

N,N-Diisopropylethylamine (0.700 mL, 4.01 mmol) was added to 2-(3-chloro-5-fluorophenyl)thiazole-5-carboxylic acid (0.2064 g, 0.801 mmol, Intermediate 5) in dichloromethane (4.01 mL) at room temperature. Then, 3-amino-5,5-dimethylpyrrolidine-2-thione hydrochloride (0.181 g, 1.001 mmol, Intermediate 45) was added and the reaction mixture was stirred for five minutes. Then, n-propylphosphonic acid anhydride (0.954 mL, 1.602 mmol) was added and the reaction mixture was stirred for three hours. Saturated sodium bicarbonate was added to the reaction mixture and it was extracted with dichloromethane, dried over magnesium sulfate, filtered, and concentrated. The resulting residue was purified by silica gel chromatography, eluting with ethyl acetate:heptane (2:3 to 1:0) to give 2-(3-chloro-5-fluorophenyl)-N-(5,5-dimethyl-2-thioxopyrrolidin-3-yl)thiazole-5-carboxamide (0.2386 g, 0.590 mmol, 73.7% yield). ¹H NMR (400 MHz, CD₃SOCD₃) δ 1.28 (s, 3H), 1.32 (s, 3H), 1.88 (dd, J=12, 11 Hz, 1H), 2.38 (dd, J=12, 9 Hz, 1H), 5.03 (dt, J=10, 9 Hz, 1H), 7.64 (dt, J=9, 2 Hz, 1H), 7.84 (ddd, J=9, 2, 2 Hz, 1H), 7.91 (t, J=2 Hz, 1H), 8.52 (br s, 1H), 9.09 (d, J=9 Hz, 1H), 10.61 (br s, 1H); LC-MS (LC-ES) for C₁₆H₁₅ClFN₃OS₂ M+H=384.

### Examples 84 & 85

### (R)-2-(3-Chloro-5-fluorophenyl)-N-(5,5-dimethyl-2-thioxopyrrolidin-3-yl)thiazole-5-carboxamide and (S)-2-(3-Chloro-5-fluorophenyl)-N-(5,5-dimethyl-2-thioxopyrrolidin-3-yl)thiazole-5-carboxamide

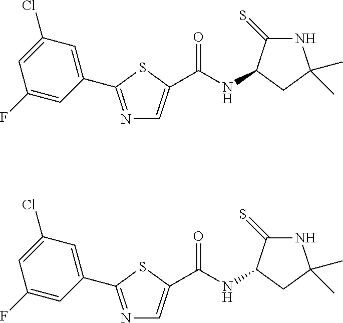 [SMILES: CC1(C)C[C@@H](NC(=O)c2cnc(-c3cc(F)cc(Cl)c3)s2)C(=S)N1 ; CC1(C)C[C@H](NC(=O)c2cnc(-c3cc(F)cc(Cl)c3)s2)C(=S)N1]

Racemic 2-(3-chloro-5-fluorophenyl)-N-(5,5-dimethyl-2-thioxopyrrolidin-3-yl)thiazole-5-carboxamide (0.2198 g, 0.573 mmol, Example 83) was separated into its enantiomers via supercritical fluid chromatography on a chiral Chiralpak IC column, eluting with ethanol:carbon dioxide (3:7) to give (R)-2-(3-chloro-5-fluorophenyl)-N-(5,5-dimethyl-2-thioxopyrrolidin-3-yl)thiazole-5-carboxamide (0.0970 g, 0.240 mmol, 41.9% yield) as the first enantiomer to elute (IC t_{R}=2.2 minutes, >99% ee) and (S)-2-(3-chloro-5-fluorophenyl)-N-(5,5-dimethyl-2-thioxopyrrolidin-3-yl)thiazole-5-carboxamide (0.0983 g, 0.243 mmol, 42.5% yield) as the last enantiomer to elute (IC t_{R}=2.9 minutes, 99% ee). The structures were assigned by vibrational circular dichroism.

### (R)-2-(3-Chloro-5-fluorophenyl)-N-(5,5-dimethyl-2-thioxopyrrolidin-3-yl)thiazole-5-carboxamide

¹H NMR (400 MHz, CD₃SOCD₃) δ 1.28 (s, 3H), 1.32 (s, 3H), 1.88 (dd, J=12, 11 Hz, 1H), 2.38 (dd, J=12, 9 Hz, 1H), 5.03 (dt, J=10, 9 Hz, 1H), 7.65 (dt, J=9, 2 Hz, 1H), 7.84 (ddd, J=9, 2, 2 Hz, 1H), 7.91 (t, J=2 Hz, 1H), 8.53 (br s, 1H), 9.09 (d, J=9 Hz, 1H), 10.61 (br s, 1H); LC-MS (LC-ES) for C₁₆H₁₅ClFN₃OS₂ M+H=384.

### (S)-2-(3-Chloro-5-fluorophenyl)-N-(5,5-dimethyl-2-thioxopyrrolidin-3-yl)thiazole-5-carboxamide

¹H NMR (400 MHz, CD₃SOCD₃) δ 1.28 (s, 3H), 1.32 (s, 3H), 1.88 (dd, J=12, 11 Hz, 1H), 2.38 (dd, J=12, 9 Hz, 1H), 5.03 (dt, J=10, 9 Hz, 1H), 7.65 (dt, J=9, 2 Hz, 1H), 7.84 (ddd, J=9, 2, 2 Hz, 1H), 7.91 (t, J=2 Hz, 1H), 8.53 (br s, 1H), 9.09 (d, J=9 Hz, 1H), 10.61 (br s, 1H); LC-MS (LC-ES) for C₁₈H₁₆ClFN₃OS₂ M+H=384.

### Example 86

### (S)-N-(2-Oxopyrrolidin-3-yl)-2-phenylthiazole-5-sulfonamide

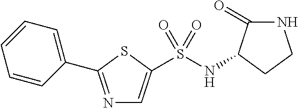 [SMILES: O=C1NCC[C@@H]1NS(=O)(=O)c1cnc(-c2ccccc2)s1]

Cyanuric chloride (0.027 g, 0.144 mmol) was added to 2-phenylthiazole-5-sulfonic acid (0.1054 g, 0.437 mmol) in acetonitrile (4.37 mL) at room temperature. Then, pyridine (0.106 mL, 1.311 mmol) was added, after five minutes (S)-3-aminopyrrolidin-2-one hydrochloride (0.060 g, 0.437 mmol) was added and the reaction mixture was stirred for sixteen hours at 75° C. Saturated sodium bicarbonate was added to the reaction mixture and it was extracted with dichloromethane, dried over magnesium sulfate, filtered, and concentrated. The resulting residue was purified by reverse phase HPLC, eluting with acetonitrile:water with 0.1% ammonium hydroxide (0:1 to 1:0) to give (S)-N-(2-oxopyrrolidin-3-yl)-2-phenylthiazole-5-sulfonamide (0.0333 g, 0.098 mmol, 22.39% yield). ¹H NMR (400 MHz, CD₃SOCD₃) δ 1.66-1.80 (m, 1H), 2.18-2.28 (m, 1H), 3.04-3.14 (m, 2H), 4.00 (dd, J=10, 9 Hz, 1H), 7.50-7.60 (m, 3H), 7.86 (br s, 1H), 7.96-8.02 (m, 2H), 8.35 (s, 1H), 8.65 (br s, 1H); LC-MS (LC-ES) for C₁₃H₁₃N₃O₃S₂ M+H=324.

### Example 87—Capsule Composition

An oral dosage form for administering the present invention is produced by filing a standard two piece hard gelatin capsule with the ingredients in the proportions shown in Table 1, below.

TABLE 1

| INGREDIENTS | AMOUNTS |
|---|---|
| (S)-2-(Benzofuran-7-yl)-N-(2-oxopyrrolidin-3-yl)thiazole-5- | |
| carboxamide (Compound of Example 1) | |
| Lactose | |
| Talc | |
| Magnesium Stearate | |

### Example 88—Injectable Parenteral Composition

An injectable form for administering the present invention is produced by stirring 1.7% by weight of 2-(3-Chlorophenyl)-N-(2-oxopyrrolidin-3-yl)thiazole-5-carboxamide (Compound of Example 2) in 10% by volume propylene glycol in water.

### Example 89 Tablet Composition The sucrose, calcium sulfate dihydrate and a H-PGDS inhibitor as shown in

Table 2 below, are mixed and granulated in the proportions shown with a 10% gelatin solution. The wet granules are screened, dried, mixed with the starch, talc and stearic acid, screened and compressed into a tablet.

TABLE 2

| INGREDIENTS | AMOUNTS |
|---|---|
| (S)-2-(3-Chlorophenyl)-N-(2-oxopyrrolidin-3-yl)thiazole-5- | |
| carboxamide (Compound of Example 3) | |
| calcium sulfate dihydrate | |
| sucrose | |
| starch | |
| talc | |
| stearic acid | |

### BIOLOGICAL ASSAYS

Meleza, C. et al (Analytical Biochemistry 2016, 511, 17-23) discloses the development of a scintillation proximity binding assay for high-throughput screening of hematopoietic prostaglandin D2 synthase. WO 2009140364 A2 discloses a fluorimetric assay for agents able to displace potent ligands of hematopoietic prostaglandin D synthase.

H-PGDS RapidFire™ High Throughput Mass Spectrometry Assay

The H-PGDS RapidFire™ mass spectrometric assay monitors conversion of prostaglandin H₂ (PGH₂) to prostaglandin D₂ (PGD₂) by haematopoietic prostaglandin D synthase (H-PGDS). In the assay format described here, the substrate (PGH₂) is formed in situ by the action of cyclooxygenase-2 on arachidonic acid. This first step is set up to be fast, and generates a burst of PGH₂ at ˜10 μM. The PGH₂ is then further converted to PGD₂ by the H-PGDS enzyme. The reaction mixed with tin (II) chloride in citric acid, which converts any remaining PGH₂ to the more stable PGF_{2α}. Plates are then read on the RapidFire™ high throughput solid phase extraction system (Agilent) which incorporates a solid phase extraction step coupled to a triple quadrupole mass spectrometer (AB SCIEX). Relative levels of PGD₂ and PGF_{2α}, which acts as a surrogate for substrate, are measured and a percent conversion calculated. Inhibitors are characterised as compounds which lower the conversion of PGH₂ to PGD₂.

Expression and Purification of H-PGDS Protein

Full length human H-PGDS cDNA (Invitrogen Ultimate ORF IOH13026) was amplified by PCR with the addition of a 5′ 6-His tag and TEV protease cleavage site. The PCR product was digested with NdeI and XhoI and ligated into pET22b+ (Merck Novagen®). Expression was carried out in E. coli strain BL21 (DE3*) using auto-induction Overnight Express™ Instant TB medium (Merck Novagen®) supplemented with 1% glycerol. The culture was first grown at 37° C. and the temperature was reduced to 25° C. when OD600 reached 2.0. Cells were harvested by centrifugation after a further 18 hours. 10 g of E. coli cell pellet was suspended to a total volume of 80 mL in lysis buffer (20 mM Tris-Cl pH 7.5, 300 mM NaCl, 20 mM imidazole, 5 mM p-mercaptoethanol, 10% glycerol). 1 mg/mL protease inhibitors (Protease Inhibitor Cocktail Set III, Merck Calbiochem®) and 1 mg/mL lysozyme were added to the cell suspension. The suspension was then sonicated for 5 min (UltraSonic Processor VCX 750, Cole-Parmer Instrument Co.) with a micro probe (50% amplitude, 10 sec on/off) and then centrifuged at 100,000 g for 90 minutes (at 4° C.). The supernatant was loaded onto a Ni-NTA HiTrap column (5 mL, GE Healthcare, pre-equilibrated in lysis buffer). The column was washed with 10 column volumes of lysis buffer and eluted with lysis buffer containing 500 mM imidazole. The pooled protein peak fractions were concentrated using a 10 kDa centrifugal filter at 3500 g and 4° C. (Amicon Ultra-15 centrifugal filter unit with Ultracel-10 membrane from Millipore). Further purification of the concentrated protein was carried out using gel filtration chromatography on a HiLoad 26/600 Superdex 75 preparative grade column (GE Healthcare Life Sciences) using 50 mM Tris pH 7.5, 50 mM NaCl, 1 mM dithiothreitol, 1 mM MgCl₂. Fractions containing the protein were pooled, concentrated as described above, and stored at −80° C.

Expression and Purification of Cyclooxygenase-2 (COX-2) Protein

The full length human COX-2 gene (accession number L15326) was amplified by PCR to generate an EcoRI—HindIII fragment containing an in-frame FLAG tag. This was subcloned into pFastBac 1 (Invitrogen). The COX-2 FLAG plasmid was recombined into the baculovirus genome according to the BAC-to-BAC protocol described by Invitrogen. Transfection into Spodoptera frugiperda (Sf9) insect cells was performed using Cellfectin (Invitrogen), according to the manufacturer's protocol. Super Sf9 cells were cultured in EX420 media (SAFC Biosciences) to a density of approximately 1.5×106 cells/mL within a wave bioreactor. Recombinant virus was added at a Multiplicity of Infection (MOI) of 5 and the culture was allowed to continue for 3 days. Cells were harvested using a continuous feed centrifuge run at 2500 g at a rate of approximately 2 L/min with cooling. The resultant cell slurry was re-centrifuged in pots (2500 g, 20 min, 4° C.) and the cell paste was stored at −80° C. 342 g of cell paste was re-suspended to a final volume of 1600 mL in a buffer of 20 mM Tris-Cl pH 7.4, 150 mM NaCl, 0.1 mM EDTA, 1.3% w/v n-octyl-β-D-glucopyranoside containing 20 Complete EDTA-free Protease Inhibitor Cocktail tablets (Roche Applied Science). The suspension was sonicated in 500 mL batches for 8×5 seconds at 10 u amplitude with the medium tip of an MSE probe sonicator and subsequently incubated at 4° C. for 90 minutes with gentle stirring. The lysate was centrifuged at 12000 rpm for 45 minutes at 4° C. in a Sorvall SLA1500 rotor. The supernatant (1400 mL) was added to 420 mL of 20 mM Tris-Cl pH 7.4, 150 mM NaCl, 0.1 mM EDTA to reduce the concentration of n-octyl-β-D-glucopyranoside to 1% w/v. The diluted supernatant was incubated overnight at 4° C. on a roller with 150 mL of anti-FLAG M2 agarose affinity gel (Aldrich-Sigma) which had been pre-equilibrated with 20 mM Tris-Cl pH 7.4, 150 mM NaCl, 0.1 mM EDTA, 1% w/v n-octyl-β-D-glucopyranoside (purification buffer). The anti-Flag M2 agarose beads were pelleted by centrifugation in 500 mL conical Corning centrifuge pots at 2000 rpm for 10 min at 4° C. in a Sorvall RC3 swing-out rotor. The supernatant (unbound fraction) was discarded and the beads were re-suspended to half the original volume in purification buffer and re-centrifuged as above. The beads were then packed into a BioRad Econo Column (5 cm diameter) and washed with 1500 mL of purification buffer at 4° C. Bound proteins were eluted with 100 μg/mL triple FLAG peptide (Aldrich-Sigma) in purification buffer. Six fractions each of 0.5 column volume were collected. After each 0.5 column volume of purification buffer was added into the column the flow was held for 10 minutes before elution. Fractions containing COX-2 were pooled resulting in a protein concentration of ˜1 mg/mL. The protein was further concentrated on Vivaspin 20 centrifugal concentrators (10 kDa cut-off) to 2.4 mg/mL and then stored at −80° C.

Test Compound Plate Preparation

Test compounds were diluted to 1 mM in DMSO and a 1:3, 11 point serial dilution was performed across a 384 well HiBase plate (Greiner Bio-one). 100 nL of this dilution series was then transferred into a 384 well v-base plate (Greiner Bio-one) using an Echo™ acoustic dispenser (Labcyte Inc) to create the assay plate. 100 nL of DMSO was added to each well in columns 6 and 18 for use as control columns.

Assay Method

5 μL of an enzyme solution containing 10 nM H-PGDS enzyme, 1.1 μM COX-2 enzyme and 2 mM reduced glutathione (Sigma-Aldrich), diluted in a buffer of 50 mM Tris-Cl pH 7.4, 10 mM MgCl₂ and 0.1% Pluronic F-127 (all Sigma-Aldrich) was added to each well of the plate except column 18 using a Multidrop Combi® dispenser (Thermo Fisher Scientific). 5 μL of enzyme solution without H-PGDS was added to each well in column 18 of the assay plate to generate 100% inhibition control wells.

Immediately after the addition of enzyme solution, 2.5 μL of a co-factor solution containing 4 μM Hemin (Sigma-Aldrich) diluted in buffer of 50 mM Tris-Cl pH 7.4 and 10 mM MgCl₂ (all Sigma-Aldrich), was added to each well using a Multidrop Combi® dispenser. 2.5 μL of substrate solution containing 80 μM arachidonic acid (Sigma-Aldrich) and 1 mM sodium hydroxide (Sigma-Aldrich) diluted in HPLC grade water (Sigma-Aldrich) was then added to each well using a Multidrop Combi® dispenser, to initiate the reaction.

The assay plates were incubated at room temperature for the duration of the linear phase of the reaction (usually 1 min 30 s-2 min, this timing should be checked on a regular basis). Precisely after this time, the reaction was quenched by the addition of 30 μL of quench solution containing 32.5 mM SnCl₂ (Sigma-Aldrich) in 200 mM citric acid (adjusted to pH 3.0 with 0.1 mM NaOH solution) to all wells using a Multidrop Combi® dispenser (Thermo Fisher Scientific). The SnCl₂ was initially prepared as a suspension at an equivalent of 600 mM in HPLC water (Sigma-Aldrich) and sufficient concentrated hydrochloric acid (Sigma-Aldrich) was added in small volumes until dissolved. The assay plates were centrifuged at 1000 rpm for 5 min prior to analysis.

The assay plates were analysed using a RapidFire™ high throughput solid phase extraction system (Agilent) coupled to a triple quadrupole mass spectrometer (AB SCIEX) to measure relative peak areas of PGF_{2α} and PGD₂ product. Peaks were integrated using the RapidFire™ integrator software before percentage conversion of substrate to PGD₂ product was calculated as shown below:

% Conversion=((PGD₂ peak area)/(PGD₂ peak area+PGF_{2α} peak area))×100.

Data were further analysed within Activitybase software (IDBS) using a four parameter curve fit of the following form:

$$y = \frac{a - d}{1 + {( x / c )}^{b}} + d$$

where a is the minimum, b is the Hill slope, c is the IC₅₀ and d is the maximum.

Data are presented as the mean pIC₅₀.

TABLE 3

| Example # | Potency Range |
|---|---|
| 1 | *** |
| 2 | *** |
| 3 | *** |
| 4 | *** |
| 5 | **** |
| 6 | **** |
| 7 | *** |
| 8 | *** |
| 9 | **** |
| 10 | *** |
| 11 | **** |
| 12 | **** |
| 13 | **** |
| 14 | *** |
| 15 | ** |
| 16 | *** |
| 17 | ** |
| 18 | *** |
| 19 | *** |
| 20 | *** |
| 21 | **** |
| 22 | ** |
| 23 | ** |
| 24 | * |
| 25 | *** |
| 26 | ** |
| 27 | *** |
| 28 | ** |
| 29 | **** |
| 30 | *** |
| 31 | * |
| 32 | * |
| 33 | *** |
| 34 | *** |
| 35 | ** |
| 36 | *** |
| 37 | *** |
| 38 | * |
| 39 | ** |
| 40 | ** |
| 41 | *** |
| 42 | ** |
| 43 | ** |
| 44 | * |
| 45 | *** |
| 46 | ** |
| 47 | *** |
| 48 | *** |
| 49 | **** |
| 50 | ** |
| 51 | **** |
| 52 | *** |
| 53 | *** |
| 54 | *** |
| 55 | **** |
| 56 | ** |
| 57 | **** |
| 58 | *** |
| 59 | ** |
| 60 | *** |
| 61 | ** |
| 62 | *** |
| 63 | *** |
| 64 | *** |
| 65 | * |
| 66 | * |
| 67 | — |
| 68 | *** |
| 69 | *** |
| 70 | ** |
| 71 | ** |
| 72 | *** |
| 73 | ** |
| 74 | *** |
| 75 | *** |
| 76 | * |
| 77 | **** |
| 78 | **** |
| 79 | *** |
| 80 | **** |
| 81 | **** |
| 82 | *** |
| 83 | **** |
| 84 | ** |
| 85 | **** |
| 86 | — |

Legend — = pIC₅₀ < 5.00,
* = pIC₅₀ 5.00-5.99
** = pIC₅₀ 6.00-6.99,
*** = pIC₅₀ 7.00-7.99,
**** = pIC₅₀ = 8.00-8.99,
***** = pIC₅₀ ≥ 9.00

In Vivo Assays for Functional Response to Muscle Injury

General procedure used.

Under anesthesia, the right hind limb of a mouse is restrained at the knee and the foot attached to a motorized footplate/force transducer. Needle electrodes are inserted into the upper limb, either side of the sciatic nerve and a current sufficient to elicit a maximal muscle contraction is applied. Muscle tension is produced by moving the footplate to lengthen the plantarflexor muscles while the limb is under maximal stimulation. This is repeated 60 times to fatigue the muscles of the lower limb. Anesthesia, limb immobilization and limb stimulation are then repeated at regular intervals to measure maximal isometric force in the recovering limb. 7 to 9 animals are tested for each test condition.

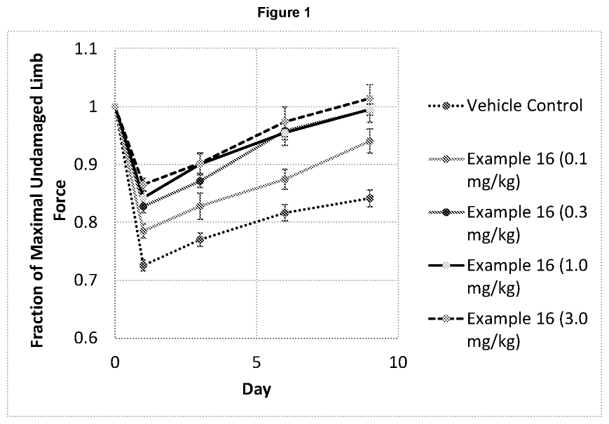

Eccentric contraction-induced muscle fatigue in vehicle-treated male C57Bl/6N mice, 10-12 weeks of age, significantly reduced (˜28%) maximal isometric torque 24 hours after injury and still exhibited a significant functional deficit after 9 days. In contrast, animals treated with the compound of Example 16 (PO) immediately prior to eccentric contraction challenge, exhibited dose-dependent protection from strength loss after 24-hours and full restoration of muscle function after 9 days of repeat dosing (QD) at all doses greater than 0.1 mg/kg. See FIG. 1.

### In Vivo Mast Cell Activation

Mice were randomized by body weight into 8 groups (n=8): Vehicle (0.5% HPML with 0.1% Tween80)+phosphate buffered saline (PBS), vehicle+compound 48/80 (0.75 mg/mL) and compound 48/80+Example 16 at various doses ranging from 0.01 mg/kg to 1.0 mg/kg.

C57BL mice were dosed orally with vehicle, or Example 16 at 0.01, 0.03, 0.1, 0.3, & 1.0 mg/kg. One hour later, blood samples were withdrawn via tail snip for measurement of drug levels, and mice were then terminally anesthetized with 2% isoflurane and given an intraperitoneal injection of 0.2 mL PBS or compound 48/80 solution (0.75 mg/mL, Sigma), followed by gentle massage of the abdomen. Mice were kept under anesthesia for 7 minutes prior to euthanasia. The abdominal cavity was then opened with a small incision and filled with 2 mL PBS and the abdomen was gently massaged for several seconds. One mL of peritoneal lavage fluid was removed, spun down (12,000 rpm for 2 min) and the supernatant was kept on dry ice and later used for measurement of PGD₂ and PGE₂ levels.

PGD₂ and PGE₂ LC/MS/MS Assay

Samples were thawed at room temperature and vortex-mixed. Standard stock solutions of PGD₂ and PGE₂ (Cayman Chemical, Ann Arbor, Mich.) were prepared at a concentration of 1 mg/mL in methanol. The stock solutions were used to prepare an intermediate standard stock solution containing both PGD₂ and PGE₂ at a concentration of 0.1 mg/mL in methanol. The intermediate standard stock solution was further diluted with methanol to obtain intermediate standard solutions (1-10,000 ng/mL). Standards of PGD₂ and PGE₂ (0.05-50 ng/mL) were prepared from the intermediate standard solutions in phosphate buffered saline pH=7.4 (1×) (Thermo Fisher Scientific, Waltham, Mass.). Acetonitrile (75 μL) containing internal standards (PGD₂-d9 and PGE₂-d9) (Cayman Chemical, Ann Arbor, Mich.) at a concentration of 1 ng/mL was added to a 96-well plate. An aliquot (75 μL) of each sample and standard was pipetted into the plate then vortex-mixed at 1500 rpm for 1 minute and centrifuged at 1840×g for 20 minutes. The supernatant (100 μL) was transferred to a clean 96-well plate containing 50 μL water. The plate was vortex-mixed for 30 seconds at 1000 rpm and analyzed by LC/MS/MS.

The analytical system consisted of a CTC HTS PAL autoinjector (Leap, Carrboro, N.C.), an Agilent 1290 Infinity binary pump and thermostated column compartment (Agilent Technologies, Santa Clara, Calif.) and an AB Sciex QTRAP 5500 mass spectrometer (AB Sciex, Framingham, Mass.). Samples (20 μL) were injected onto a 100×2.1 mm, 1.8 micron, Waters Acquity UPLC HSS T3 column (Agilent, Santa Clara, Calif.) maintained at 50° C. The mobile phase consisted of water containing 0.1% formic acid (Solvent A) and 100% acetonitrile containing 0.1% formic acid (Solvent B). An isocratic gradient elution at 0.750 mL/minute was used with a composition of 65% A:35% B over 4.0 minutes. Total run time was 4.0 minutes. PGD₂ eluted at 2.57 min and PGE₂ at 2.22 min. The internal standards PGD₂-d9 eluted at 2.51 min and PGE₂-d9 at 2.16 min. The analytes were detected by multiple reaction monitoring (MRM) in negative mode using Turbolon spray with the transitions of m/z 351/271 amu for PGD₂/PGE₂ and m/z 360/280 amu for PGD₂-d9/PGE₂-d9. Data were acquired, analyzed and quantified using Analyst software version 1.6.2 (AB Sciex, Framingham, Mass.).

The calibration curves for the PGD₂ and PGE₂ samples ranged from 0.05 to 50 ng/mL (10 concentrations with an n=2/concentration) and 20/20 were within the acceptable accuracy limits of ±20% of the nominal concentration. For the PGD₂ calibration curve, the correlation coefficient was 0.9991 using a 1/x² weighted linear regression analysis. For the PGE₂ calibration curve, the correlation coefficient was 0.9995 using a 1/x² weighted linear regression analysis.

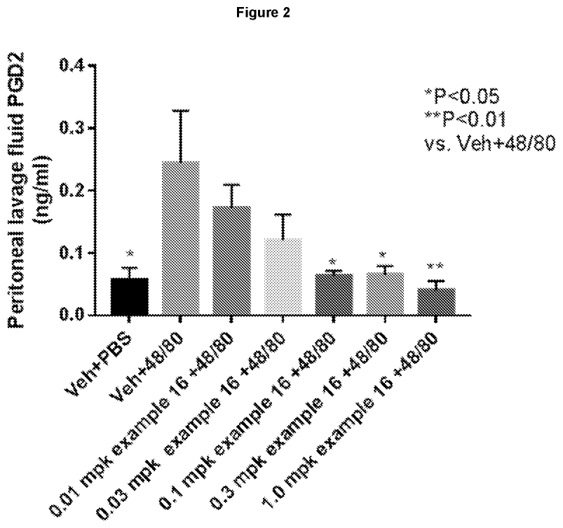

The effect of different doses of the H-PGDS inhibitor of Example 16 on prostaglandin D₂ generation following 48/80-induced mast cell degranulation in normal C57Bl6/N mice is depicted in FIG. 2. Doses were administered ˜1 hour prior to 48/80 (i.p.) injection, with peritoneal lavage collected 7-minutes afterwards. The data in FIG. 2 indicates that H-PGDS inhibition by the compound of Example 16 prevents 48/80-induced PGD₂ generation in lavage fluid of normal mice.

While the preferred embodiments of the invention are illustrated by the above, it is to be understood that the invention is not limited to the precise instructions herein disclosed and that the right to all modifications coming within the scope of the following claims is reserved.

1. A compound according to Formula (I) 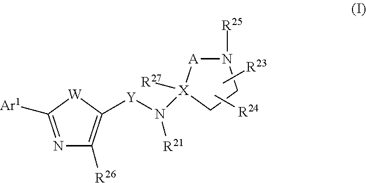
wherein:
Ar¹ is selected from: phenyl, benzofuranyl, pyrazolyl, imidazolyl, pyridinyl, and pyrimidinyl, each of which is optionally substituted with from 1 to 4 substituents independently selected from:
fluoro,
chloro,
bromo,
iodo,
C₁₋₃alkyl,
C₁₋₃alkyl substituted with from one to four substituents independently selected from: —OH, oxo, and fluoro,
—CN,
—OH,
cyclopropyl,
C₁₋₃alkoxy, and
C₁₋₃alkoxy substituted with from one to four substituents independently selected from: —OH, oxo, and fluoro;
W is selected from: S and Se,
X is selected from: C and N;
Y is selected from: —C(O)—, —C(S)—, —C(Se)—, —S(O)—, and —S(O₂)—;
A is selected from: —C(O)—, —C(S)—, —C(Se)—, and —S(O₂)—;
R²¹ is selected from: hydrogen and —CH₃;
R²³ and R²⁴ are attached to the same or different carbon atoms and are independently selected from:
hydrogen,
C₁₋₅alkyl,
C₁₋₅alkyl substituted with from one to four substituents independently selected from: —OH, oxo, —NH₂ and fluoro, or
R²³ and R²⁴ are attached to the same carbon and are taken together to form: cyclopropyl, cyclobutyl, cyclopentyl, oxetanyl, tetrahydrofuran, or tetrahydropyran, or
R²³ and R²⁴ are attached to different carbon atoms and are taken together to form: cyclopropyl, cyclobutyl, cyclopentyl, oxetanyl, tetrahydrofuran, or tetrahydropyran;
R²⁵ is selected from:
hydrogen,
C₁₋₅alkyl,
C₁₋₅alkyl substituted with from one to four substituents independently selected from: —OH, oxo, —NH₂ and fluoro, and
C₁₋₅alkylaryl, and
C₁₋₅alkylaryl substituted with from 1 to 3 substituents independently selected from
fluoro,
chloro,
bromo,
iodo,
C₁₋₃alkyl,
C₁₋₃alkyl substituted with from one to four substituents independently selected from: —OH, oxo, and fluoro,
—CN,
—OH,
cyclopropyl,
C₁₋₃alkoxy, and
C₁₋₃alkoxy substituted with from one to four substituents independently selected from: —OH, oxo, and fluoro;
R²⁶ is selected from: hydrogen and —CH₃; and
R²⁷ is absent when X is N or selected from: hydrogen and —CH₃;
or a pharmaceutically acceptable salt thereof.

2. The compound of claim 1 represented by the following Formula (II):F 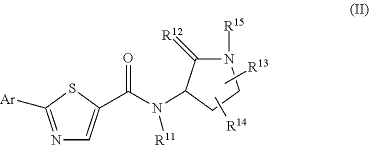
wherein:
Ar is selected from: phenyl, benzofuranyl, pyrazolyl, imidazolyl, pyridinyl, and pyrimidinyl, each of which is optionally substituted with from 1 to 4 substituents independently selected from:
fluoro,
chloro,
bromo,
iodo,
C₁₋₃alkyl,
C₁₋₃alkyl substituted with from one to four substituents independently selected from: —OH, oxo, and fluoro,
—CN,
—OH,
cyclopropyl,
C₁₋₃alkoxy, and
C₁₋₃alkoxy substituted with from one to four substituents independently selected from: —OH, oxo, and fluoro;
R¹¹ is selected from: hydrogen and —CH₃;
R¹² is selected from: O, S and Se;
R¹³ and R¹⁴ are attached to the same or different carbon atoms and are independently selected from:
hydrogen,
C₁₋₅alkyl,
C₁₋₅alkyl substituted with from one to four substituents independently selected from: —OH, oxo, —NH₂ and fluoro, or
R¹³ and R¹⁴ are attached to the same carbon and are taken together to form: cyclopropyl, cyclobutyl, cyclopentyl, oxetanyl, tetrahydrofuran, or tetrahydropyran, or
R¹³ and R¹⁴ are attached to different carbon atoms and are taken together to form: cyclopropyl, cyclobutyl, cyclopentyl, oxetanyl, tetrahydrofuran, or tetrahydropyran; and
R¹⁵ is selected from:
hydrogen,
C₁₋₅alkyl,
C₁₋₅alkyl substituted with from one to four substituents independently selected from: —OH, oxo, —NH₂ and fluoro, and
C₁₋₅alkylaryl, and
C₁₋₅alkylphenyl substituted with from 1 to 3 substituents independently selected from
fluoro,
chloro,
bromo,
iodo,
C₁₋₃alkyl,
C₁₋₃alkyl substituted with from one to four substituents independently selected from: —OH, oxo, and fluoro,
—CN,
—OH,
cyclopropyl,
C₁₋₃alkoxy, and
C₁₋₃alkoxy substituted with from one to four substituents independently selected from: —OH, oxo, and fluoro;
or a pharmaceutically acceptable salt thereof.

3. The compound of claim 1 represented by the following Formula (III): 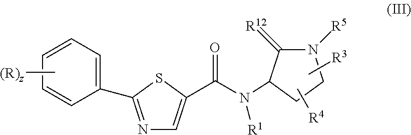
wherein:
R is selected from: fluoro, chloro, bromo, iodo, —CH₃, —CH₂F, —CHF₂, —CF₃, —CH₂CH₃, —CH₂CF₃, —CH₂CFH₂, —CH₂CF₂H, —CN, —OH, cyclopropyl and —OCH₃;
R¹ is selected from: hydrogen and —CH₃;
R² is selected from: O, S and Se;
R³ and R⁴ are attached to the same or different carbon atoms and are independently selected from: hydrogen, —CH₃, and —CH₂CH₃, or
R³ and R⁴ are attached to the same carbon and are taken together to form: cyclopropyl, cyclobutyl, or oxetanyl, or
R³ and R⁴ are attached to different carbon atoms and are taken together to form: cyclopentyl, or tetrahydrofuranyl;
R⁵ is selected from: hydrogen, —CH₃, —CH₂C(O)NH₂, and —CH₂-phenyl-O—CH₃; and
Z is an integer from 0 to 3;
or a pharmaceutically acceptable salt thereof.

4. The compound of claim 1 represented by the following Formula (IV) 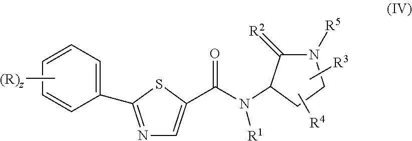
wherein:
R is independently selected from: fluoro, chloro, bromo, and iodo;
R¹ is selected from: hydrogen and —CH₃;
R² is O;
R³ and R⁴ are attached to the same or different carbon atoms and are independently selected from: hydrogen, —CH₃, and —CH₂CH₃;
R⁵ is selected from: hydrogen, and —CH₃; and
Z is an integer from 0 to 3;
or a pharmaceutically acceptable salt thereof.

5. The compound of claim 1 selected from:
(S)-2-(Benzofuran-7-yl)-N-(2-oxopyrrolidin-3-yl)thiazole-5-carboxamide;
Racemic 2-(3-Chlorophenyl)-N-(2-oxopyrrolidin-3-yl)thiazole-5-carboxamide;
(S)-2-(3-Chlorophenyl)-N-(2-oxopyrrolidin-3-yl)thiazole-5-carboxamide;
(S)-N-(2-Oxopyrrolidin-3-yl)-2-(3-(trifluoromethyl)phenyl)thiazole-5-carboxamide;
(S)-N-(2-Oxopyrrolidin-3-yl)-2-(m-tolyl)thiazole-5-carboxamide;
(S)-2-(3-Chloro-5-fluorophenyl)-N-(2-oxopyrrolidin-3-yl)thiazole-5-carboxamide;
(S)-2-(5-Chloro-2-fluorophenyl)-N-(2-oxopyrrolidin-3-yl)thiazole-5-carboxamide;
(S)-2-(3-Chloro-2-fluorophenyl)-N-(2-oxopyrrolidin-3-yl)thiazole-5-carboxamide;
(S)-2-(3,5-Dichlorophenyl)-N-(2-oxopyrrolidin-3-yl)thiazole-5-carboxamide;
(S)-2-(3-(Difluoromethyl)phenyl)-N-(2-oxopyrrolidin-3-yl)thiazole-5-carboxamide;
2-(3-Chloro-5-fluorophenyl)-N-((3S,4R)-4-methyl-2-oxopyrrolidin-3-yl)thiazole-5-carboxamide;
(S)-2-(3-(Difluoromethyl)-5-fluorophenyl)-N-(2-oxopyrrolidin-3-yl)thiazole-5-carboxamide;
2-(3-(Difluoromethyl)-5-fluorophenyl)-N-((3S,4R)-4-methyl-2-oxopyrrolidin-3-yl)thiazole-5-carboxamide;
Racemic 2-(3-Chloro-5-fluorophenyl)-N-(5,5-dimethyl-2-oxopyrrolidin-3-yl)thiazole-5-carboxamide;
(R)-2-(3-Chloro-5-fluorophenyl)-N-(5,5-dimethyl-2-oxopyrrolidin-3-yl)thiazole-5-carboxamide;
(S)-2-(3-Chloro-5-fluorophenyl)-N-(5,5-dimethyl-2-oxopyrrolidin-3-yl)thiazole-5-carboxamide;
(S)-N-(1-(2-Amino-2-oxoethyl)-2-oxopyrrolidin-3-yl)-2-(3,5-difluorophenyl)thiazole-5-carboxamide;
Racemic 2-(3-(Difluoromethyl)-5-fluorophenyl)-N-(5,5-dimethyl-2-oxopyrrolidin-3-yl)thiazole-5-carboxamide;
(S)-2-(3-Chloro-5-fluorophenyl)-N-(4,4-dimethyl-2-oxopyrrolidin-3-yl)thiazole-5-carboxamide;
Racemic 2-(3-Chloro-5-fluorophenyl)-N-(5-oxo-4-azaspiro[2.4]heptan-6-yl)thiazole-5-carboxamide;
(S)-2-(3-Chloro-5-fluorophenyl)-N-(5-oxo-4-azaspiro[2.4]heptan-6-yl)thiazole-5-carboxamide;
(R)-2-(3-Chloro-5-fluorophenyl)-N-(5-oxo-4-azaspiro[2.4]heptan-6-yl)thiazole-5-carboxamide;
Racemic 2-(3-Chloro-5-fluorophenyl)-N-(4,4-diethyl-2-oxopyrrolidin-3-yl)thiazole-5-carboxamide;
(R)-2-(3-Chloro-5-fluorophenyl)-N-(4,4-diethyl-2-oxopyrrolidin-3-yl)thiazole-5-carboxamide;
(S)-2-(3-Chloro-5-fluorophenyl)-N-(4,4-diethyl-2-oxopyrrolidin-3-yl)thiazole-5-carboxamide;
(R)-2-(3-Chloro-5-fluorophenyl)-N-(2-oxopyrrolidin-3-yl)thiazole-5-carboxamide;
Racemic 2-(3-Chloro-5-fluorophenyl)-N-(6-oxo-5-azaspiro[3.4]octan-7-yl)thiazole-5-carboxamide;
(R)-2-(3-Chloro-5-fluorophenyl)-N-(6-oxo-5-azaspiro[3.4]octan-7-yl)thiazole-5-carboxamide;
(S)-2-(3-Chloro-5-fluorophenyl)-N-(6-oxo-5-azaspiro[3.4]octan-7-yl)thiazole-5-carboxamide;
(S)-2-(3-Chlorophenyl)-4-methyl-N-(2-oxopyrrolidin-3-yl)thiazole-5-carboxamide;
(S)-2-(4-Methyl-1H-pyrazol-1-yl)-N-(2-oxopyrrolidin-3-yl)thiazole-5-carboxamide;
(S)-2-(4-Methyl-1H-imidazol-1-yl)-N-(2-oxopyrrolidin-3-yl)thiazole-5-carboxamide;
(S)-N-(2-Oxopyrrolidin-3-yl)-2-phenylthiazole-5-carboxamide;
Racemic 2-(3-Chloro-5-fluorophenyl)-N-((3R,4S,5S)-4,5-dimethyl-2-oxopyrrolidin-3-yl)thiazole-5-carboxamide and
2-(3-Chloro-5-fluorophenyl)-N-((3S,4R,5R)-4,5-dimethyl-2-oxopyrrolidin-3-yl)thiazole-5-carboxamide;
2-(3-Chloro-5-fluorophenyl)-N-((3R,4S,5S)-4,5-dimethyl-2-oxopyrrolidin-3-yl)thiazole-5-carboxamide;
2-(3-Chloro-5-fluorophenyl)-N-((3S,4R,5R)-4,5-dimethyl-2-oxopyrrolidin-3-yl)thiazole-5-carboxamide;
(S)-2-(3-Bromophenyl)-N-(2-oxopyrrolidin-3-yl)thiazole-5-carboxamide;
(S)-N-(2-Oxopyrrolidin-3-yl)-2-(pyridin-4-yl)thiazole-5-carboxamide;
(S)-N-(2-Oxopyrrolidin-3-yl)-2-(pyridin-2-yl)thiazole-5-carboxamide;
(S)-N-(2-Oxopyrrolidin-3-yl)-2-(pyridin-3-yl)thiazole-5-carboxamide;
2-(3-Chloro-5-fluorophenyl)-N-((3R,5S)-5-methyl-2-oxopyrrolidin-3-yl)thiazole-5-carboxamide;
(S)-2-(4-Methylpyrimidin-2-yl)-N-(2-oxopyrrolidin-3-yl)thiazole-5-carboxamide;
(S)-2-(3-Cyanophenyl)-N-(2-oxopyrrolidin-3-yl)thiazole-5-carboxamide;
(S)-N-(2-Oxopyrrolidin-3-yl)-2-(p-tolyl)thiazole-5-carboxamide;
(S)-2-(3-Fluorophenyl)-N-(2-oxopyrrolidin-3-yl)thiazole-5-carboxamide;
(S)-2-(6-Methylpyridin-2-yl)-N-(2-oxopyrrolidin-3-yl)thiazole-5-carboxamide;
(S)-2-(4-Methylpyridin-2-yl)-N-(2-oxopyrrolidin-3-yl)thiazole-5-carboxamide;
(S)-2-(3-(Difluoromethyl)-5-methylphenyl)-N-(2-oxopyrrolidin-3-yl)thiazole-5-carboxamide;
Racemic 2-(3-Chloro-5-fluorophenyl)-N-((3R,3aS,6aR)-2-oxooctahydrocyclopenta[b]pyrrol-3-yl)thiazole-5-carboxamide and
2-(3-Chloro-5-fluorophenyl)-N-((3S,3aR,6aS)-2-oxooctahydrocyclopenta[b]pyrrol-3-yl)thiazole-5-carboxamide;
2-(3-Chloro-5-fluorophenyl)-N-((3R,3aS,6aR)-2-oxooctahydrocyclopenta[b]pyrrol-3-yl)thiazole-5-carboxamide;
2-(3-Chloro-5-fluorophenyl)-N-((3S,3aR,6aS)-2-oxooctahydrocyclopenta[b]pyrrol-3-yl)thiazole-5-carboxamide;
Racemic 2-(3-Chloro-5-fluorophenyl)-N-((3S,3aR,6aR)-2-oxohexahydro-1H-furo[3,4-b]pyrrol-3-yl)thiazole-5-carboxamide and
2-(3-Chloro-5-fluorophenyl)-N-((3R,3aS,6aS)-2-oxohexahydro-1H-furo[3,4-b]pyrrol-3-yl)thiazole-5-carboxamide;
2-(3-Chloro-5-fluorophenyl)-N-((3S,3aR,6aR)-2-oxohexahydro-1H-furo[3,4-b]pyrrol-3-yl)thiazole-5-carboxamide;
2-(3-Chloro-5-fluorophenyl)-N-((3R,3aS,6aS)-2-oxohexahydro-1H-furo[3,4-b]pyrrol-3-yl)thiazole-5-carboxamide;
Racemic 2-(3-Chloro-5-fluorophenyl)-N-(6-oxo-2-oxa-5-azaspiro[3.4]octan-7-yl)thiazole-5-carboxamide;
(R)-2-(3-Chloro-5-fluorophenyl)-N-(6-oxo-2-oxa-5-azaspiro[3.4]octan-7-yl)thiazole-5-carboxamide;
(S)-2-(3-Chloro-5-fluorophenyl)-N-(6-oxo-2-oxa-5-azaspiro[3.4]octan-7-yl)thiazole-5-carboxamide;
Racemic 2-(3-Chloro-5-fluorophenyl)-N-((3R,3aR,6aS)-2-oxooctahydrocyclopenta[b]pyrrol-3-yl)thiazole-5-carboxamide and
2-(3-Chloro-5-fluorophenyl)-N-((3S,3aS,6aR)-2-oxooctahydrocyclopenta[b]pyrrol-3-yl)thiazole-5-carboxamide;
2-(3-Chloro-5-fluorophenyl)-N-((3R,3aR,6aS)-2-oxooctahydrocyclopenta[b]pyrrol-3-yl)thiazole-5-carboxamide;
2-(3-Chloro-5-fluorophenyl)-N-((3S,3aS,6aR)-2-oxooctahydrocyclopenta[b]pyrrol-3-yl)thiazole-5-carboxamide;
2-(3-Chloro-5-fluorophenyl)-N-((3R,5R)-5-methyl-2-oxopyrrolidin-3-yl)thiazole-5-carboxamide;
2-(3-Chloro-5-fluorophenyl)-N-((3S,5R)-5-methyl-2-oxopyrrolidin-3-yl)thiazole-5-carboxamide;
Racemic 2-(3-Chloro-5-fluorophenyl)-N-(1-methyl-2-oxopyrrolidin-3-yl)thiazole-5-carboxamide;
(S)-2-(3-Chloro-5-fluorophenyl)-N-(1-methyl-2-oxopyrrolidin-3-yl)thiazole-5-carboxamide;
Racemic 2-(3-Chloro-5-fluorophenyl)-N-methyl-N-(2-oxopyrrolidin-3-yl)thiazole-5-carboxamide;
(S)-2-(3-Chloro-5-fluorophenyl)-N-methyl-N-(2-oxopyrrolidin-3-yl)thiazole-5-carboxamide;
(R)-2-(3-Chloro-5-fluorophenyl)-N-methyl-N-(2-oxopyrrolidin-3-yl)thiazole-5-carboxamide;
(S)-2-(3-Methoxyphenyl)-N-(2-oxopyrrolidin-3-yl)thiazole-5-carboxamide;
(S)-2-(3-Hydroxyphenyl)-N-(2-oxopyrrolidin-3-yl)thiazole-5-carboxamide;
Racemic 2-(3-Chloro-5-fluorophenyl)-N-(1-(4-methoxybenzyl)-3-methyl-2-oxopyrrolidin-3-yl)thiazole-5-carboxamide;
Racemic 2-(3-Chloro-5-fluorophenyl)-N-(3-methyl-2-oxopyrrolidin-3-yl)thiazole-5-carboxamide;
(S)-2-(3-Chloro-5-fluorophenyl)-N-(3-methyl-2-oxopyrrolidin-3-yl)thiazole-5-carboxamide;
(R)-2-(3-Chloro-5-fluorophenyl)-N-(3-methyl-2-oxopyrrolidin-3-yl)thiazole-5-carboxamide;
Racemic 2-(3-Chloro-5-fluorophenyl)-N-((3S,4S)-4-methyl-2-oxopyrrolidin-3-yl)thiazole-5-carboxamide and Racemic
2-(3-Chloro-5-fluorophenyl)-N-((3S,4R)-4-methyl-2-oxopyrrolidin-3-yl)thiazole-5-carboxamide;
2-(3-Chloro-5-fluorophenyl)-N-((3S,4S)-4-methyl-2-oxopyrrolidin-3-yl)thiazole-5-carboxamide;
2-(3-Chloro-5-fluorophenyl)-N-((3R,4R)-4-methyl-2-oxopyrrolidin-3-yl)thiazole-5-carboxamide;
(S)-2-(3-Chloro-5-fluorophenyl)-N-(2-thioxopyrrolidin-3-yl)thiazole-5-carboxamide; (S)-2-(3-chloro-5-fluorophenyl)-N-(2-selenoxopyrrolidin-3-yl)thiazole-5-carboxamide;
2-(3-Chloro-5-fluorophenyl)-N-(2-oxoimidazolidin-1-yl)thiazole-5-carboxamide;
(S)-2-(3-chloro-5-fluorophenyl)-N-(2-oxopyrrolidin-3-yl)thiazole-5-carbothioamide;
2-(3-Chloro-5-fluorophenyl)-N-((3S,5S)-5-methyl-2-oxopyrrolidin-3-yl)thiazole-5-carboxamide;
Racemic 2-(3-Chloro-5-fluorophenyl)-N-(5,5-dimethyl-2-oxopyrrolidin-3-yl)-1,3-selenazole-5-carboxamide;
Racemic 2-(3-Chloro-5-fluorophenyl)-N-(5,5-dimethyl-2-thioxopyrrolidin-3-yl)thiazole-5-carboxamide;
(R)-2-(3-chloro-5-fluorophenyl)-N-(5,5-dimethyl-2-thioxopyrrolidin-3-yl)thiazole-5-carboxamide;
(S)-2-(3-chloro-5-fluorophenyl)-N-(5,5-dimethyl-2-thioxopyrrolidin-3-yl)thiazole-5-carboxamide; and
(S)-N-(2-oxopyrrolidin-3-yl)-2-phenylthiazole-5-sulfonamide;
or a pharmaceutically acceptable salt thereof.

6. A compound which is (S)-2-(3-Chloro-5-fluorophenyl)-N-(5,5-dimethyl-2-oxopyrrolidin-3-yl)thiazole-5-carboxamide.

7. A compound which is

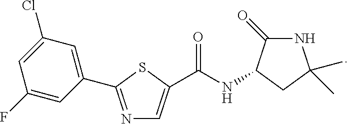 [SMILES: CC1(C)C[C@H](NC(=O)c2cnc(-c3cc(F)cc(Cl)c3)s2)C(=O)N1]

8.-11. (canceled)

12. A method for the treatment of disorders in which inhibition of H-PGDS is beneficial in a human comprising administering to the human in need thereof a therapeutically effective amount of a compound of Formula (I) or a pharmaceutically acceptable salt thereof according to claim 1.

13. A method for the treatment of allergic diseases and other inflammatory conditions such as asthma, aspirin-exacerbated respiratory disease (AERD), cough, chronic obstructive pulmonary disease (including chronic bronchitis and emphysema), bronchoconstriction, allergic rhinitis (seasonal or perennial), vasomotor rhinitis, rhinoconjunctivitis, allergic conjunctivitis, food allergy, hypersensitivity lung diseases, eosinophilic syndromes including eosinophilic asthma, eosinophilic pneumonitis, eosinophilic oesophagitis, eosinophilic granuloma, delayed-type hypersensitivity disorders, atherosclerosis, rheumatoid arthritis, pancreatitis, gastritis, inflammatory bowel disease, osteoarthritis, psoriasis, sarcoidosis, systemic lupus erythematosus (SLE), pulmonary fibrosis, respiratory distress syndrome, bronchiolitis, sinusitis, cystic fibrosis, actinic keratosis, skin dysplasia, chronic urticaria, eczema and all types of dermatitis including atopic dermatitis or contact dermatitis in a human comprising administering to the human in need thereof a therapeutically effective amount of a compound of Formula (I) or a pharmaceutically acceptable salt thereof according to claim 1.

14. A method for the treatment or prophylaxis of asthma in a human comprising administering to the human in need thereof a therapeutically effective amount of a compound of Formula (I) or a pharmaceutically acceptable salt thereof according to claim 1.

15. A method for the treatment of Duchenne muscular dystrophy in a human comprising administering to the human in need thereof a therapeutically effective amount of a compound of Formula (I) or a pharmaceutically acceptable salt thereof according to claim 1.

16. A pharmaceutical composition comprising a compound of Formula (I) or a pharmaceutically acceptable salt thereof according to claim 1 and one or more pharmaceutically acceptable carriers or excipients.

17.-19. (canceled)

20. A method for the treatment of neuromuscular-related conditions selected from: Duchenne muscular dystrophy (MD), Becker MD, congenital MD (Fukuyama), Dreifuss MD, limb girdle MD, fascioscapulohumeral MD, myotonic dystrophy type I (DM1 or Steinert's), myotonic dystrophy type II (DM2 or proximal myotonic myopathy), congenital myotonia, polymyositis, dermatomyositis, amyotrophic lateral sclerosis (ALS), muscle injury, surgery-related muscle injury, traumatic muscle injury, work-related skeletal muscle injury, overtraining-related muscle injury, muscle damage due to knee replacement, muscle damage due to anterior cruciate ligament (ACL) repair, muscle damage due to plastic surgery, muscle damage due to hip replacement surgery, muscle damage due to joint replacement surgery, muscle damage due to tendon repair surgery, muscle damage due to surgical repair of rotator cuff disease, muscle damage due to surgical repair of rotator cuff injury, muscle damage due to amputation, battlefield muscle injuries, auto accident-related muscle injuries, sports-related muscle injuries, muscle lacerations, traumatic injury due to blunt force contusions, traumatic injury due to shrapnel wounds, muscle pulls or tears, traumatic injury due to burns, acute muscle strains, chronic muscle strains, weight or force stress muscle injuries, repetitive stress muscle injuries, avulsion muscle injury, compartment syndrome, muscle injuries caused by highly repetitive motions, muscle injuries caused by forceful motions, muscle injuries caused by awkward postures, muscle injuries caused by prolonged and forceful mechanical coupling between the body and an object, muscle injuries caused by vibration, muscle injuries due to unrepaired or under-repaired muscle damage coincident with a lack of recovery or lack of an increase of physical work capacity, exercise-induced delayed onset muscle soreness (DOMS), wound healing and disuse atrophy in a human comprising administering to the human in need thereof a therapeutically effective amount of a compound of Formula (I) or a pharmaceutically acceptable salt thereof according to claim 1 or a pharmaceutical composition of claim 16.

21. (canceled)

22. A pharmaceutical composition comprising from 0.5 to 1,000 mg of a compound or pharmaceutically acceptable salt thereof as defined in claim 1, and from 0.5 to 1,000 mg of a pharmaceutically acceptable excipient.

23.-25. (canceled)
